(19) United States
(12) Patent Application Publication
(10) Pub. No.: US 2026/0344170 A1
(43) Pub. Date: Oct. 29, 2026

(54) LIGHT-EMITTING DEVICE INCLUDING AMINE-CONTAINING COMPOUND, ELECTRONIC APPARATUS AND ELECTRONIC EQUIPMENT EACH INCLUDING LIGHT-EMITTING DEVICE, AND AMINE-CONTAINING COMPOUND

(71) Applicant: Samsung Display Co., Ltd., Yongin-si (KR)

(72) Inventors: Eunjae JEONG, Yongin-si (KR); Beomjoon KIM, Yongin-si (KR); Jinyoung YUN, Yongin-si (KR)

(73) Assignee: Samsung Display Co., Ltd., Yongin-si (KR)

(21) Appl. No.: 19/643,396

(22) Filed: Apr. 9, 2026

(30) Foreign Application Priority Data
Apr. 14, 2025 (KR) 10-2025-0048366

(51) Int. Cl. H10K 50/11; H10K 50/15; H10K 59/12; H10K 59/40; H10K 85/30; H10K 85/40; H10K 85/60

(52) U.S. Cl. CPC H10K 50/11; H10K 50/15; H10K 59/12; H10K 59/40; H10K 85/324; H10K 85/342; H10K 85/346; H10K 85/348; H10K 85/40; H10K 85/622; H10K 85/623; H10K 85/626; H10K 85/633; H10K 85/636; H10K 85/654; H10K 85/6572; H10K 85/6574; H10K 85/6576; H10K 85/658

(57) ABSTRACT

Provided are a light-emitting device including an amine-containing compound represented by Formula 1, an electronic device and electronic equipment each including the light-emitting device, and the amine-containing compound represented by Formula 1:

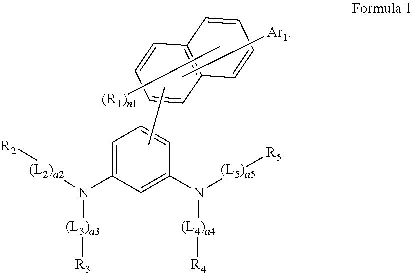

A detailed description of Formula 1 is provided in the specification.

### CROSS-REFERENCE TO RELATED APPLICATION

This application is based on and claims priority under 35 U.S.C. § 119 to Korean Patent Application No. 10-2025-0048366, filed on Apr. 14, 2025, in the Korean Intellectual Property Office, the disclosure of which is incorporated by reference herein in its entirety.

### BACKGROUND

### 1. Field

One or more example embodiments relate to a light-emitting device including an amine-containing compound, an electronic apparatus and electronic equipment each including the light-emitting device, and the amine-containing compound.

### 2. Background

From among light-emitting devices, self-emissive devices have wide viewing angles, high contrast ratios, short response times, and excellent characteristics in terms of luminance, driving voltage, and response speed.

In a light-emitting device, a first electrode is located on a substrate, and a hole transport region, an emission layer, an electron transport region, and a second electrode are sequentially arranged on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state to thereby generate light.

### SUMMARY

One or more example embodiments include a light-emitting device including an amine-containing compound, an electronic apparatus and electronic equipment each including the light-emitting device, and the amine-containing compound.

Additional aspects will be set forth in part in the description which follows and, in part, will be apparent from the description, or may be learned by practice of the presented embodiments of the disclosure.

According to one or more example embodiments, a light-emitting device includes
a first electrode,
a second electrode facing the first electrode,
an interlayer between the first electrode and the second electrode, the interlayer including an emission layer, and
an amine-containing compound represented by Formula 1:

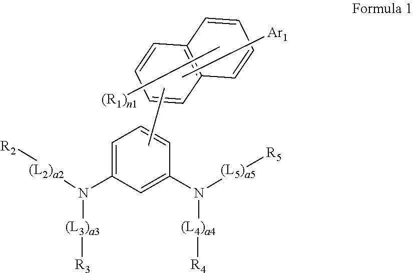

wherein, in Formula 1,
Ar may be a C₆-C₆₀ aryl group unsubstituted or substituted with at least one R₁₀ₐ,
L₂ to L₅ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a2 to a5 may each independently be 0, 1, 2, or 3,
when a2 is 0, *-(L₂)ₐ₂-*′ may be a single bond, when a3 is 0, *-(L₃)ₐ₃-*′ may be a single bond, when a4 is 0, *-(L₄)ₐ₄-*′ may be a single bond, and when a5 is 0, *-(L₅)ₐ₅-*′ may be a single bond,
n1 may be 0, 1, 2, 3, 4, 5, or 6,
R₁ to R₅ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
neighboring two or more of R₂ to R₅ may optionally be bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof.

According to one or more example embodiments, a light-emitting device includes
a first electrode;
a second electrode facing the first electrode;
m emitting units stacked between the first electrode and the second electrode; and

m−1 charge generation layers arranged between two neighboring emitting units from among the m emitting units,
wherein m is an integer of 2 or more,
the m emitting units each independently comprise an interlayer comprising an emission layer, and
in at least one of the m emitting units, the interlayer comprises an amine-containing compound represented by Formula 1

According to one or more embodiments, an electronic apparatus includes the light-emitting device.

According to one or more embodiments, electronic equipment includes the light-emitting device.

According to one or more embodiments, an amine-containing compound represented by Formula 1 is provided.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of a light-emitting device according to some example embodiments;

FIG. 2 is a schematic view of an electronic apparatus according to some example embodiments;

FIG. 3 is a schematic view of an electronic apparatus according to some example embodiments;

FIG. 4 is a block diagram of electronic equipment according to some example embodiments;

FIG. 5 is schematic views of electronic equipment according to one or more example embodiments;

FIG. 6 is a schematic perspective view of electronic equipment including a light-emitting device according to some example embodiments;

FIG. 7 is a diagram schematically illustrating the exterior of a vehicle as electronic equipment including a light-emitting device according to some example embodiments; and

FIGS. 8A to 8C are each a diagram schematically illustrating the interior of a vehicle according to one or more example embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

According to an aspect, provided is an amine-containing compound represented by Formula 1:

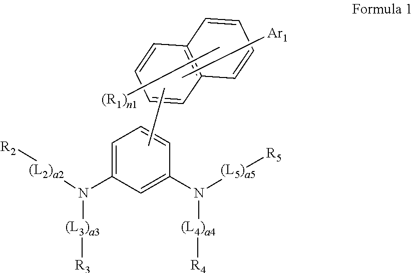

wherein, in Formula 1, Ar₁ may be a C₆-C₆₀ aryl group unsubstituted or substituted with at least one R₁₀ₐ.

In some example embodiments, Ar₁ may be a phenyl group unsubstituted or substituted with at least one R₁₀ₐ.

In some example embodiments, Ar₁ may be a phenyl group unsubstituted or substituted with at least one deuterium.

In some example embodiments, a group represented by

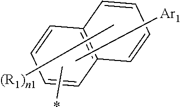

in Formula 1 may be a group represented by one of Formulae 1A to 1N:

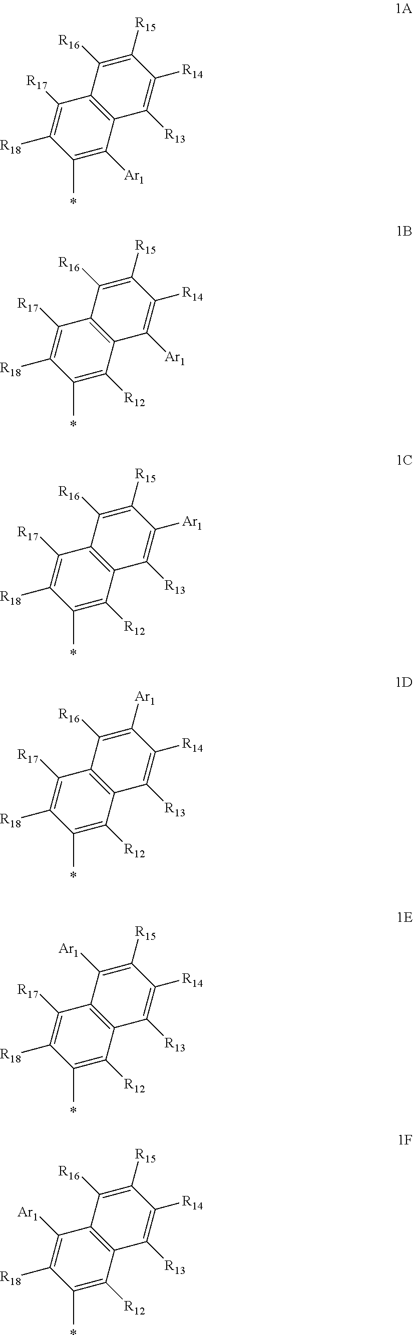

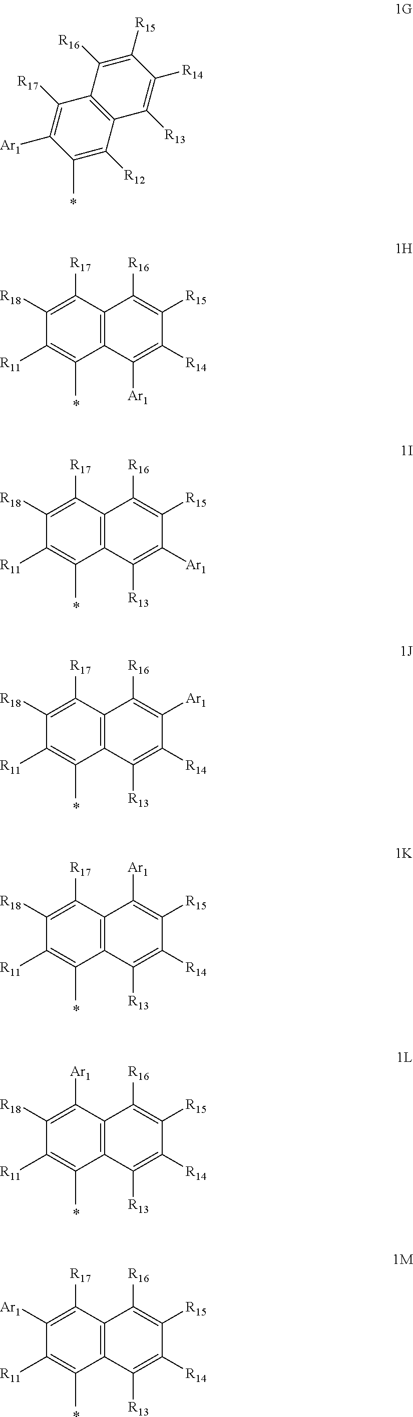

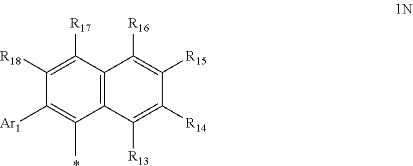

wherein, in Formulae 1A to 1N,
Ar₁ is the same as described herein,
R₁₁ to R₁₈ are each the same as described in connection with R₁, and
* indicates a binding site to a neighboring atom.

In some example embodiments, a group represented by

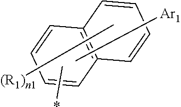

in Formula 1 may be a group represented by one of Formulae 1A to 1G.

In some example embodiments, a group represented by

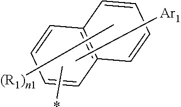

in Formula 1 may be a group represented by Formula 1A.

In Formula 1, L₂ to L₅ may each independently be a C₃-C₆₀ carbocyclic group unsubstituted or substituted with at least one R₁₀ₐ or a C₁-C₆₀ heterocyclic group unsubstituted or substituted with at least one R₁₀ₐ.

In some example embodiments, L₂ to L₅ may each independently be a benzene group unsubstituted or substituted with at least one R₁₀ₐ, a naphthalene group unsubstituted or substituted with at least one R₁₀ₐ, a carbazole group unsubstituted or substituted with at least one R₁₀ₐ, a fluorene group unsubstituted or substituted with at least one R₁₀ₐ, a dibenzofuran group unsubstituted or substituted with at least one R₁₀ₐ, or a dibenzothiophene group unsubstituted or substituted with at least one R₁₀ₐ.

As depicted in Formula 1, the

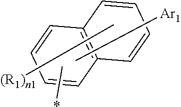

group may be bonded to any of the carbon atoms of the lower cyclic structure that is bonded to a Nitrogen atom.

In some example embodiments, L₂ to L₅ may each independently be a group represented by one of Formulae 3-1 to 3-3:

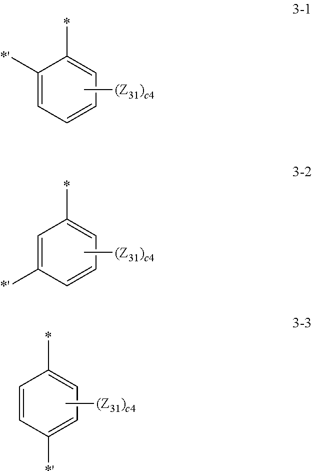

wherein, in Formulae 3-1 to 3-3,
Z₃₁ is the same as described herein in connection with R₁₀ₐ,
c4 may be 0, 1, 2, 3, or 4, and
* and *′ each indicate a binding site to a neighboring atom.

In Formula 1, a2 indicates the number of L₂, a3 indicates the number of L₃, a4 indicates the number of L₄, and a5 indicates the number of L₅. a2 to a5 may each independently be 0, 1, 2, or 3.

When a2 is 0, *-(L₂)ₐ₂-*′ may be a single bond, when a3 is 0, *-(L₃)ₐ₃-*′ may be a single bond, when a4 is 0, *-(L₄)ₐ₄-*′ may be a single bond, and when a5 is 0, *-(L₅)ₐ₅-*′ may be a single bond.

When a2 is 2 or more, L₂ in the number of a2 may be identical to or different from each other, when a3 is 2 or more, L₃ in the number of a3 may be identical to or different from each other, when a4 is 2 or more, L₄ in the number of a4 may be identical to or different from each other, and when a5 is 2 or more, L₅ in the number of a5 may be identical to or different from each other.

In some example embodiments, a2 to a5 may each independently be 0, 1, or 2. For example, a2 to a5 may each independently be 0 or 1.

In Formula 1, R₁ to R₅ may each independently be
hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂).

Neighboring two or more of R₂ to R₅ may optionally be bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ.

R₁₀ₐ and Q₁ to Q₃ are each the same as described in the specification.

In Formula 1, n1 indicates the number of R₁, and n1 may be 0, 1, 2, 3, 4, 5, or 6. When n1 is 2 or greater, R₁ in the number of n1 may be identical to or different from each other.

In some example embodiments, R₁ may be:
hydrogen, deuterium, —F, a C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a C₁-C₁₀ alkyl group, a cyclopentyl group, a cyclohexyl group, a cycloheptyl group, a cyclopentenyl group, a cyclohexenyl group, a cycloheptenyl group, or a combination thereof;
a cyclopentyl group, a cyclohexyl group, a cycloheptyl group, a cyclopentenyl group, a cyclohexenyl group, or a cycloheptenyl group, each unsubstituted or substituted with deuterium, —F, —CD₃, —CD₂H, —CDH₂, —CF₃, —CF₂H, —CFH₂, a C₁-C₂₀ alkyl group, a C₁-C₂₀ alkoxy group, a cyclopentyl group, a cyclohexyl group, a cycloheptyl group, a cyclopentenyl group, a cyclohexenyl group, a cycloheptenyl group, —Si(Q₃₁)(Q₃₂)(Q₃₃), —N(Q₃₁)(Q₃₂), or any combination thereof; or
—Si(Q₁)(Q₂)(Q₃) or —N(Q₁)(Q₂).

In some example embodiments, R₂ to R₅ may each independently be a C₆-C₆₀ aryl group unsubstituted or substituted with at least one R₁₀ₐ, a C₁-C₆₀ heteroaryl group unsubstituted or substituted with at least one R₁₀ₐ, a monovalent non-aromatic condensed polycyclic group unsubstituted or substituted with at least one R₁₀ₐ, or a monovalent non-aromatic condensed heteropolycyclic group unsubstituted or substituted with at least one R₁₀ₐ.

In some example embodiments, R₂ to R₅ may each independently be a group represented by one of Formulae 2-1 to 2-13:

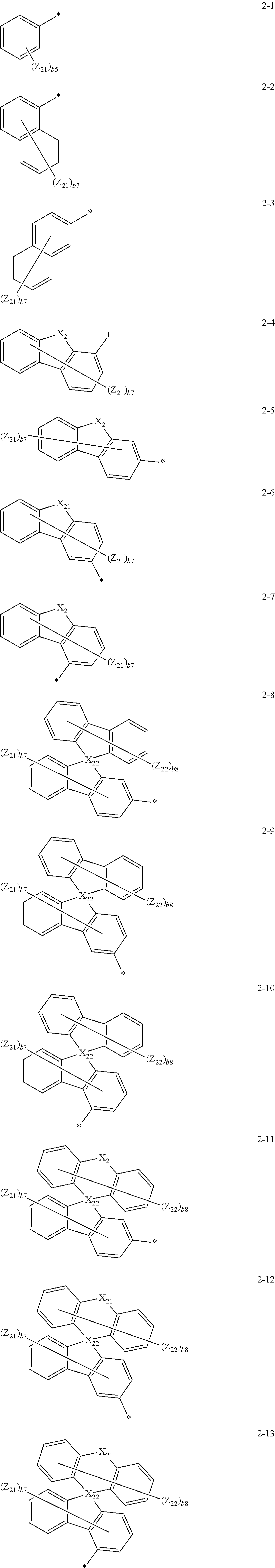

wherein, in Formulae 2-1 to 2-13,
X₂₁ may be C(Z₂₃)(Z₂₄), Si(Z₂₃)(Z₂₄), N(Z₂₃), P(Z₂₃), O, or S,
X₂₂ may be C or Si,
Z₂₁, Z₂₂, Z₂₃, and Z₂₄ are each the same as described in connection with R₁₀ₐ,
b5 may be 0, 1, 2, 3, 4, or 5,
b7 may be 0, 1, 2, 3, 4, 5, 6, or 7,
b8 may be 0, 1, 2, 3, 4, 5, 6, 7, or 8, and
* indicates a binding site to a neighboring atom.

In some example embodiments, the amine-containing compound may be represented by Formula 1-1:

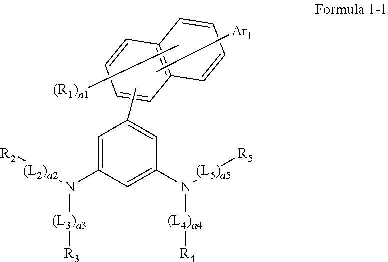

wherein Ar₁, L₂ to L₅, a2 to a5, n1, and R₁ to R₅ are each the same as described above.

Unless otherwise defined, in Formula 1, R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Unless otherwise defined, in Formula 1-1, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof.

In some example embodiments, the

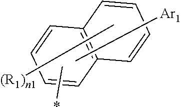

of Formula 1-1 may be bonded a particular carbon atom relative to the two nitrogen atoms (e.g., the

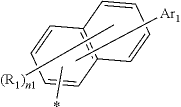

group is bonded to the 1 carbon atom, one nitrogen atom is bonded to the 3 carbon atom, and another nitrogen atom is bonded to the 5 carbon atom).

In some example embodiments, the amine-containing compound may be selected from Compounds 1 to 120:

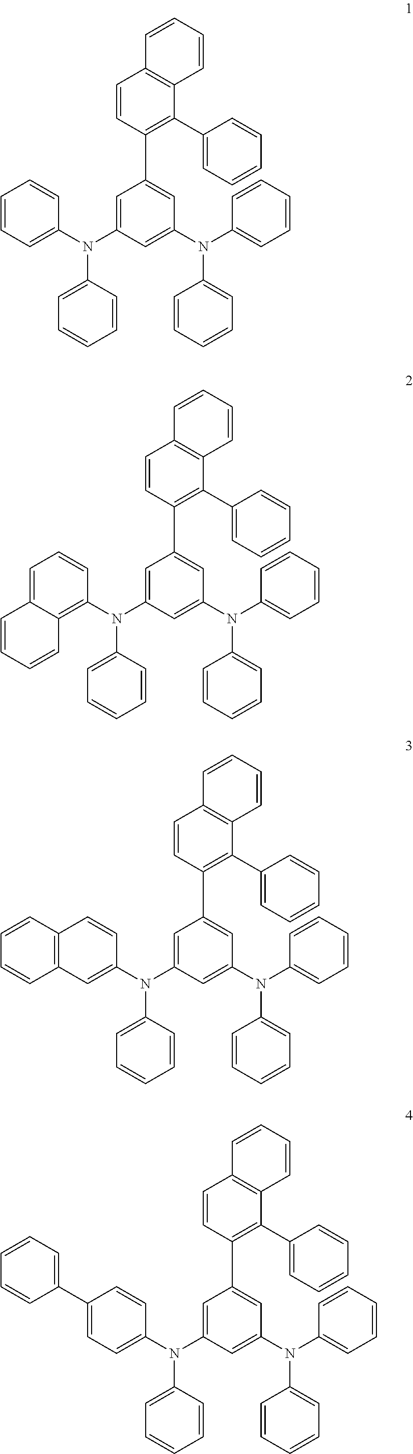

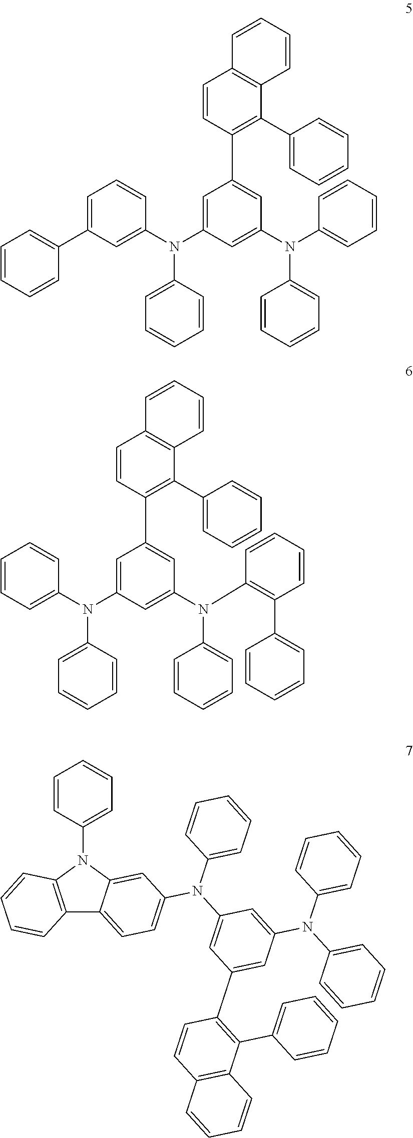

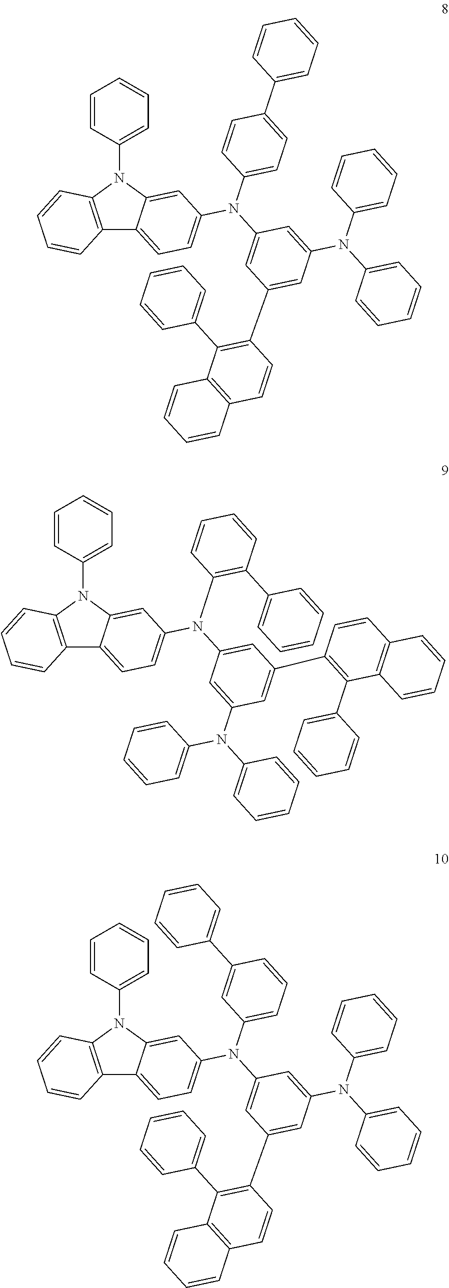 [SMILES: c1ccc(-c2ccc(N(c3cc(-c4ccc5ccccc5c4-c4ccccc4)cc(N(c4ccccc4)c4ccccc4)c3)c3ccc4c5ccccc5n(-c5ccccc5)c4c3)cc2)cc1 ; c1ccc(-c2ccccc2N(c2cc(-c3ccc4ccccc4c3-c3ccccc3)cc(N(c3ccccc3)c3ccccc3)c2)c2ccc3c4ccccc4n(-c4ccccc4)c3c2)cc1 ; c1ccc(-c2cccc(N(c3cc(-c4ccc5ccccc5c4-c4ccccc4)cc(N(c4ccccc4)c4ccccc4)c3)c3ccc4c5ccccc5n(-c5ccccc5)c4c3)c2)cc1]

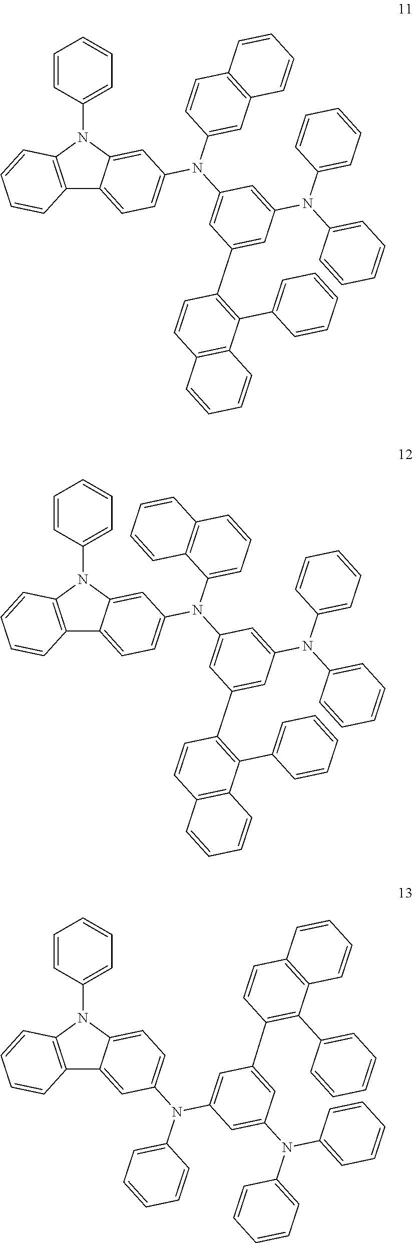 [SMILES: c1ccc(-c2c(-c3cc(N(c4ccccc4)c4ccccc4)cc(N(c4ccc5ccccc5c4)c4ccc5c6ccccc6n(-c6ccccc6)c5c4)c3)ccc3ccccc23)cc1 ; c1ccc(-c2c(-c3cc(N(c4ccccc4)c4ccccc4)cc(N(c4ccc5c6ccccc6n(-c6ccccc6)c5c4)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)c4ccccc4n5-c4ccccc4)c3)ccc3ccccc23)cc1]

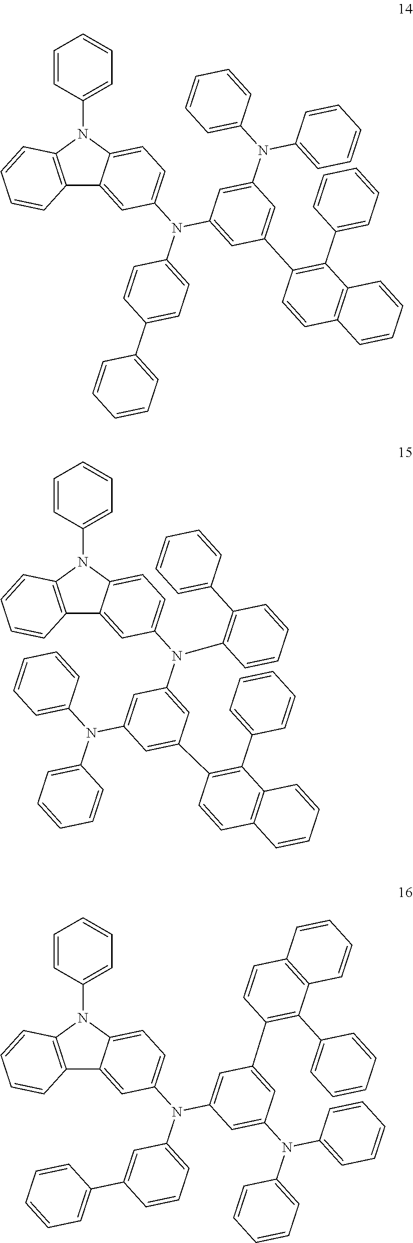 [SMILES: c1ccc(-c2ccc(N(c3cc(-c4ccc5ccccc5c4-c4ccccc4)cc(N(c4ccccc4)c4ccccc4)c3)c3ccc4c(c3)c3ccccc3n4-c3ccccc3)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)c2ccccc2n3-c2ccccc2)cc1 ; c1ccc(-c2cccc(N(c3cc(-c4ccc5ccccc5c4-c4ccccc4)cc(N(c4ccccc4)c4ccccc4)c3)c3ccc4c(c3)c3ccccc3n4-c3ccccc3)c2)cc1]

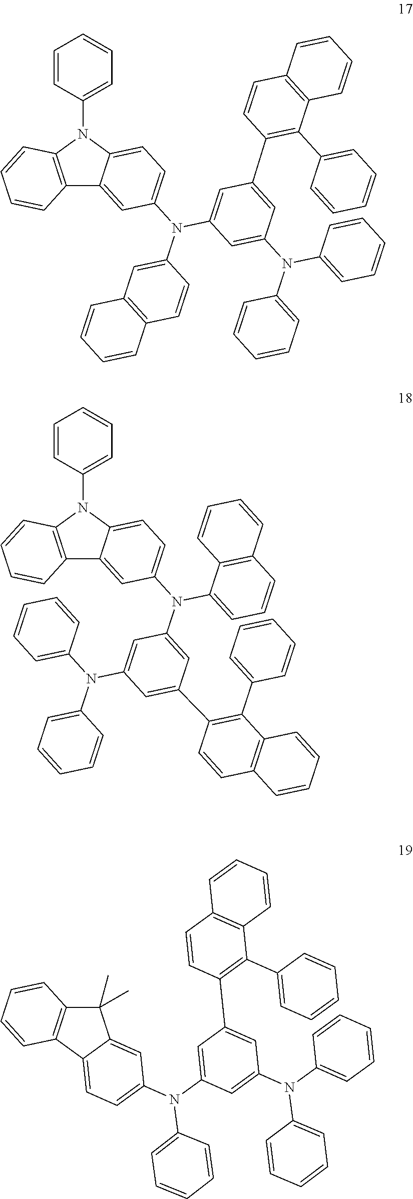 [SMILES: c1ccc(-c2c(-c3cc(N(c4ccccc4)c4ccccc4)cc(N(c4ccc5ccccc5c4)c4ccc5c(c4)c4ccccc4n5-c4ccccc4)c3)ccc3ccccc23)cc1 ; c1ccc(-c2c(-c3cc(N(c4ccccc4)c4ccccc4)cc(N(c4ccc5c(c4)c4ccccc4n5-c4ccccc4)c4cccc5ccccc45)c3)ccc3ccccc23)cc1 ; CC1(C)c2ccccc2-c2ccc(N(c3ccccc3)c3cc(-c4ccc5ccccc5c4-c4ccccc4)cc(N(c4ccccc4)c4ccccc4)c3)cc21]

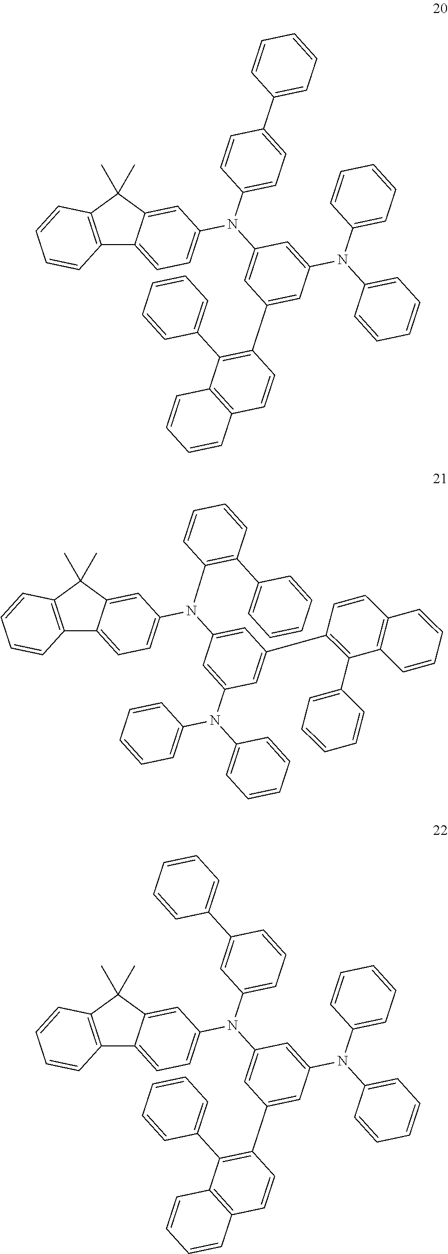 [SMILES: CC1(C)c2ccccc2-c2ccc(N(c3ccc(-c4ccccc4)cc3)c3cc(-c4ccc5ccccc5c4-c4ccccc4)cc(N(c4ccccc4)c4ccccc4)c3)cc21 ; CC1(C)c2ccccc2-c2ccc(N(c3cc(-c4ccc5ccccc5c4-c4ccccc4)cc(N(c4ccccc4)c4ccccc4)c3)c3ccccc3-c3ccccc3)cc21 ; CC1(C)c2ccccc2-c2ccc(N(c3cccc(-c4ccccc4)c3)c3cc(-c4ccc5ccccc5c4-c4ccccc4)cc(N(c4ccccc4)c4ccccc4)c3)cc21]

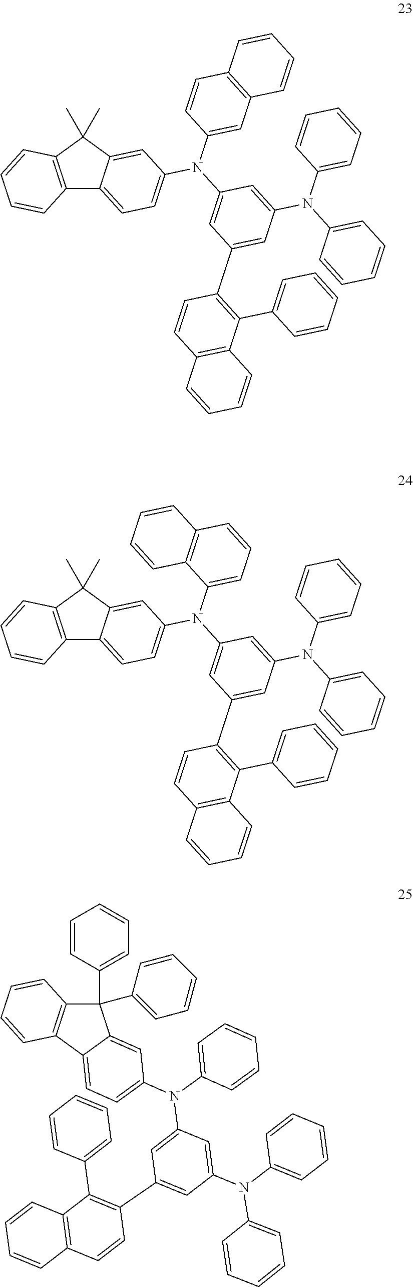 [SMILES: CC1(C)c2ccccc2-c2ccc(N(c3cc(-c4ccc5ccccc5c4-c4ccccc4)cc(N(c4ccccc4)c4ccccc4)c3)c3ccc4ccccc4c3)cc21 ; CC1(C)c2ccccc2-c2ccc(N(c3cc(-c4ccc5ccccc5c4-c4ccccc4)cc(N(c4ccccc4)c4ccccc4)c3)c3cccc4ccccc34)cc21 ; c1ccc(-c2c(-c3cc(N(c4ccccc4)c4ccccc4)cc(N(c4ccccc4)c4ccc5c(c4)C(c4ccccc4)(c4ccccc4)c4ccccc4-5)c3)ccc3ccccc23)cc1]

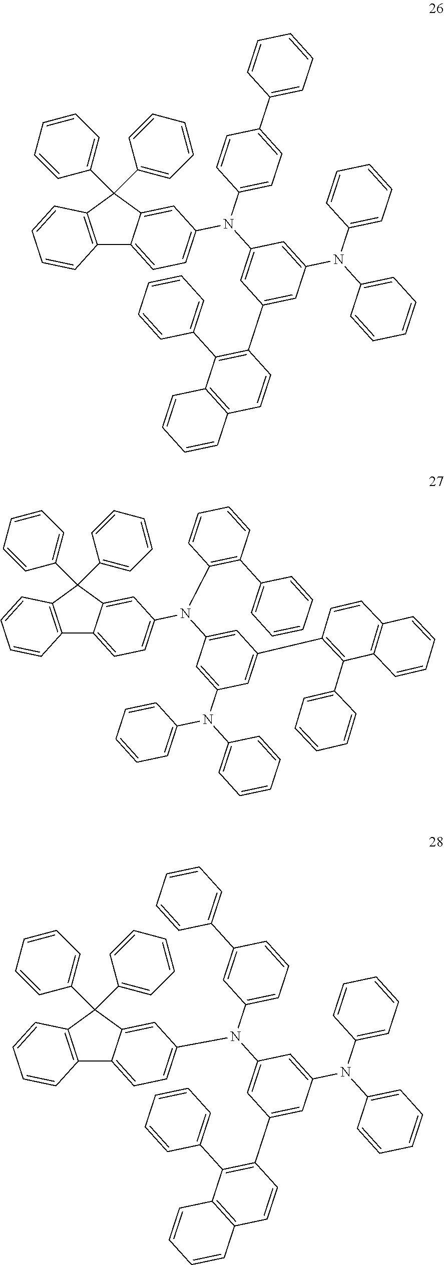 [SMILES: c1ccc(-c2ccc(N(c3cc(-c4ccc5ccccc5c4-c4ccccc4)cc(N(c4ccccc4)c4ccccc4)c3)c3ccc4c(c3)C(c3ccccc3)(c3ccccc3)c3ccccc3-4)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)C(c2ccccc2)(c2ccccc2)c2ccccc2-3)cc1 ; c1ccc(-c2cccc(N(c3cc(-c4ccc5ccccc5c4-c4ccccc4)cc(N(c4ccccc4)c4ccccc4)c3)c3ccc4c(c3)C(c3ccccc3)(c3ccccc3)c3ccccc3-4)c2)cc1]

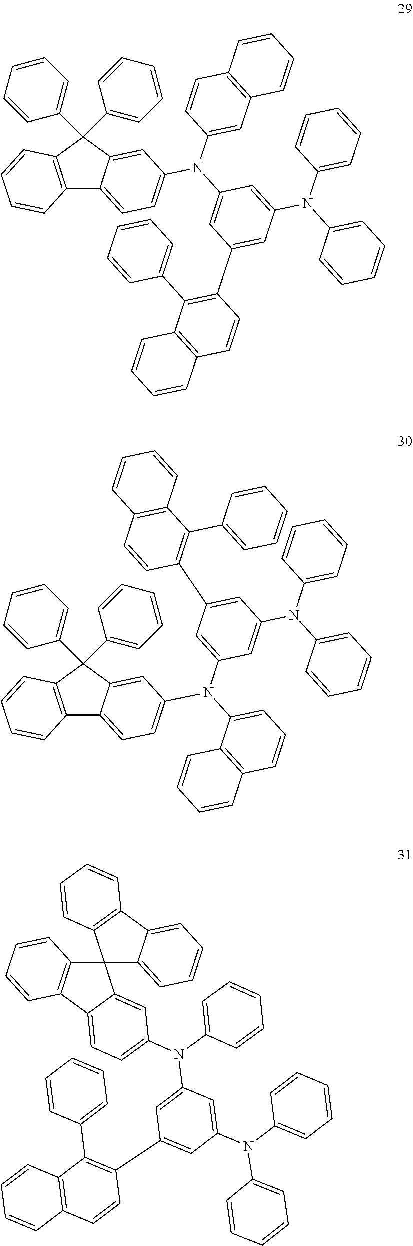 [SMILES: c1ccc(-c2c(-c3cc(N(c4ccccc4)c4ccccc4)cc(N(c4ccc5c(c4)C(c4ccccc4)(c4ccccc4)c4ccccc4-5)c4ccc5ccccc5c4)c3)ccc3ccccc23)cc1 ; c1ccc(-c2c(-c3cc(N(c4ccccc4)c4ccccc4)cc(N(c4ccc5c(c4)C(c4ccccc4)(c4ccccc4)c4ccccc4-5)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)C4(c6ccccc6-c6ccccc64)c4ccccc4-5)c3)ccc3ccccc23)cc1]

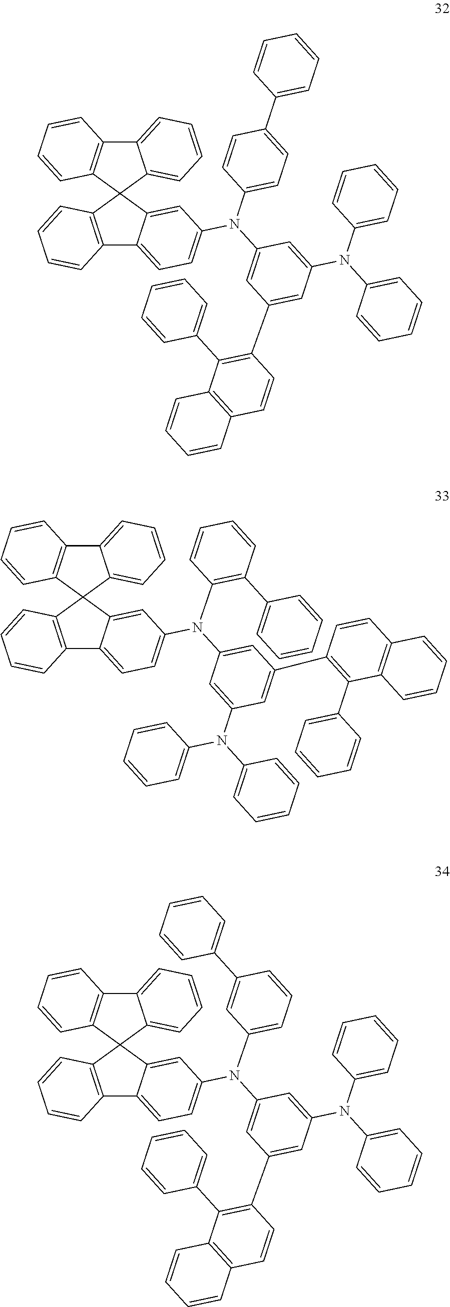 [SMILES: c1ccc(-c2ccc(N(c3cc(-c4ccc5ccccc5c4-c4ccccc4)cc(N(c4ccccc4)c4ccccc4)c3)c3ccc4c(c3)C3(c5ccccc5-c5ccccc53)c3ccccc3-4)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)C2(c4ccccc4-c4ccccc42)c2ccccc2-3)cc1 ; c1ccc(-c2cccc(N(c3cc(-c4ccc5ccccc5c4-c4ccccc4)cc(N(c4ccccc4)c4ccccc4)c3)c3ccc4c(c3)C3(c5ccccc5-c5ccccc53)c3ccccc3-4)c2)cc1]

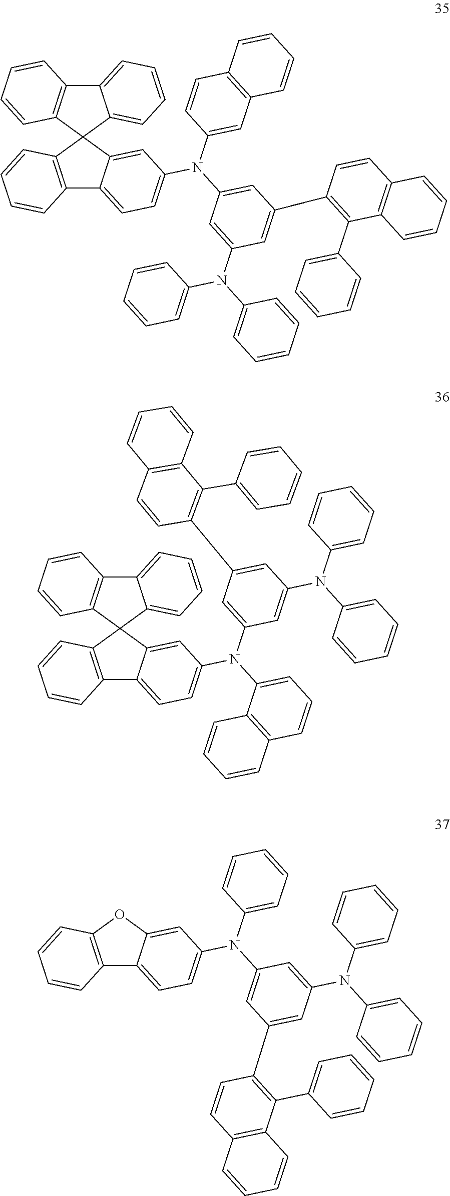 [SMILES: c1ccc(-c2c(-c3cc(N(c4ccccc4)c4ccccc4)cc(N(c4ccc5c(c4)C4(c6ccccc6-c6ccccc64)c4ccccc4-5)c4ccc5ccccc5c4)c3)ccc3ccccc23)cc1 ; c1ccc(-c2c(-c3cc(N(c4ccccc4)c4ccccc4)cc(N(c4ccc5c(c4)C4(c6ccccc6-c6ccccc64)c4ccccc4-5)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)oc4ccccc45)c3)ccc3ccccc23)cc1]

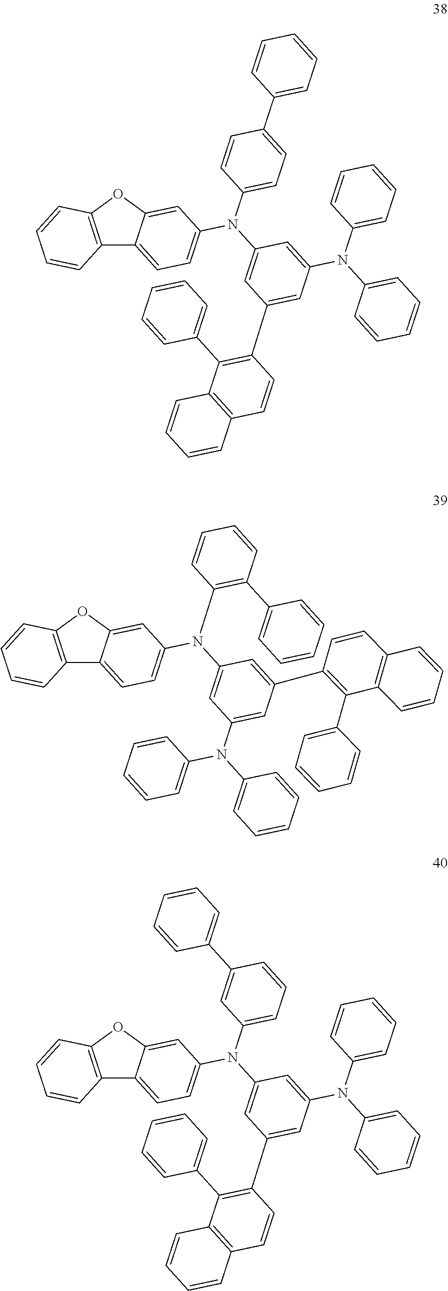 [SMILES: c1ccc(-c2ccc(N(c3cc(-c4ccc5ccccc5c4-c4ccccc4)cc(N(c4ccccc4)c4ccccc4)c3)c3ccc4c(c3)oc3ccccc34)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)oc2ccccc23)cc1 ; c1ccc(-c2cccc(N(c3cc(-c4ccc5ccccc5c4-c4ccccc4)cc(N(c4ccccc4)c4ccccc4)c3)c3ccc4c(c3)oc3ccccc34)c2)cc1]

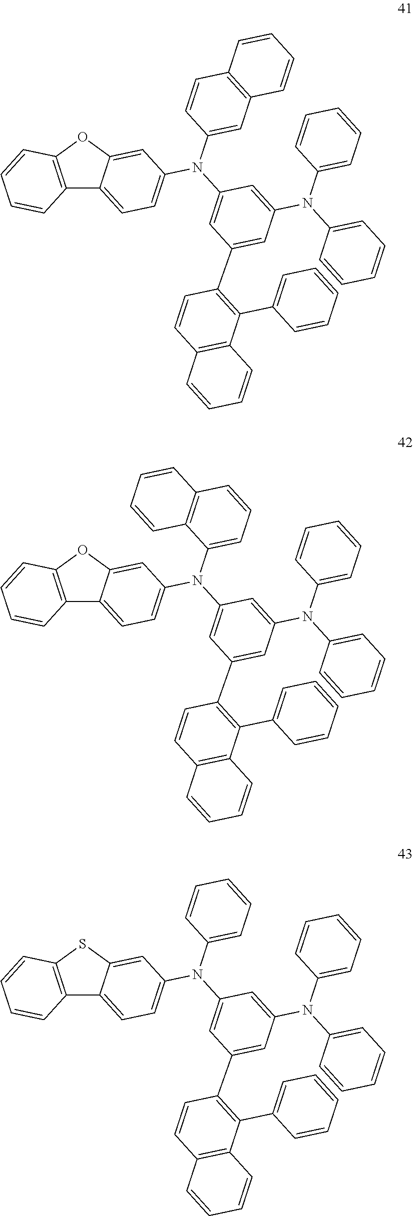 [SMILES: c1ccc(-c2c(-c3cc(N(c4ccccc4)c4ccccc4)cc(N(c4ccc5ccccc5c4)c4ccc5c(c4)oc4ccccc45)c3)ccc3ccccc23)cc1 ; c1ccc(-c2c(-c3cc(N(c4ccccc4)c4ccccc4)cc(N(c4ccc5c(c4)oc4ccccc45)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)sc4ccccc45)c3)ccc3ccccc23)cc1]

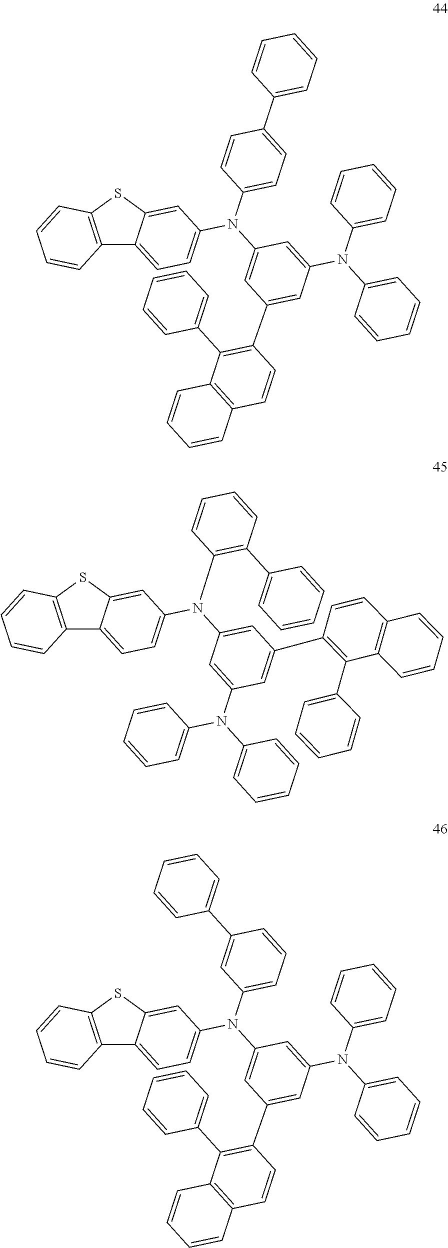 [SMILES: c1ccc(-c2ccc(N(c3cc(-c4ccc5ccccc5c4-c4ccccc4)cc(N(c4ccccc4)c4ccccc4)c3)c3ccc4c(c3)sc3ccccc34)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)sc2ccccc23)cc1 ; c1ccc(-c2cccc(N(c3cc(-c4ccc5ccccc5c4-c4ccccc4)cc(N(c4ccccc4)c4ccccc4)c3)c3ccc4c(c3)sc3ccccc34)c2)cc1]

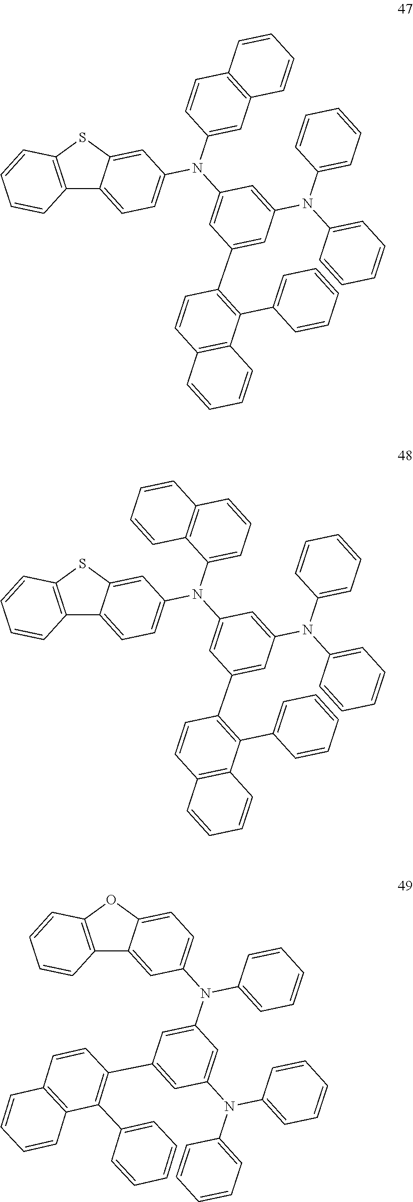 [SMILES: c1ccc(-c2c(-c3cc(N(c4ccccc4)c4ccccc4)cc(N(c4ccc5ccccc5c4)c4ccc5c(c4)sc4ccccc45)c3)ccc3ccccc23)cc1 ; c1ccc(-c2c(-c3cc(N(c4ccccc4)c4ccccc4)cc(N(c4ccc5c(c4)sc4ccccc45)c4cccc5ccccc45)c3)ccc3ccccc23)cc1 ; c1ccc(-c2c(-c3cc(N(c4ccccc4)c4ccccc4)cc(N(c4ccccc4)c4ccc5oc6ccccc6c5c4)c3)ccc3ccccc23)cc1]

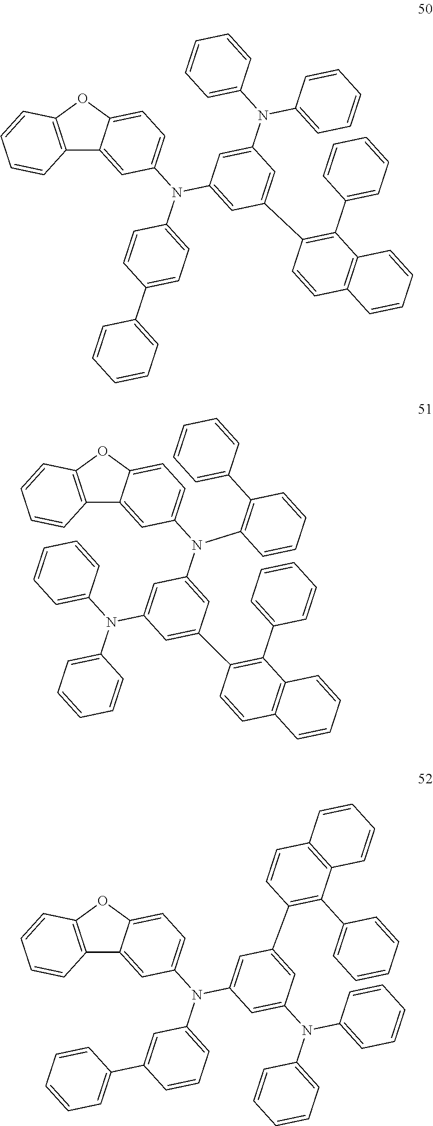 [SMILES: c1ccc(-c2ccc(N(c3cc(-c4ccc5ccccc5c4-c4ccccc4)cc(N(c4ccccc4)c4ccccc4)c3)c3ccc4oc5ccccc5c4c3)cc2)cc1 ; c1ccc(-c2ccccc2N(c2cc(-c3ccc4ccccc4c3-c3ccccc3)cc(N(c3ccccc3)c3ccccc3)c2)c2ccc3oc4ccccc4c3c2)cc1 ; c1ccc(-c2cccc(N(c3cc(-c4ccc5ccccc5c4-c4ccccc4)cc(N(c4ccccc4)c4ccccc4)c3)c3ccc4oc5ccccc5c4c3)c2)cc1]

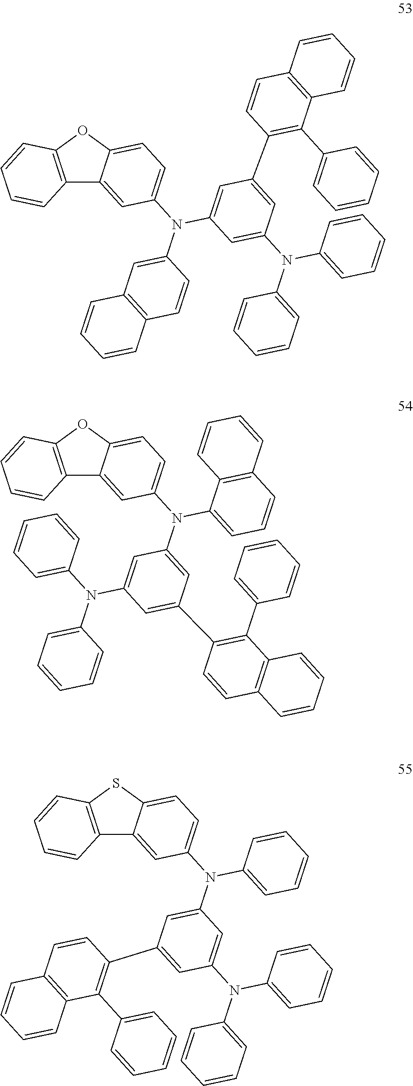 [SMILES: c1ccc(-c2c(-c3cc(N(c4ccccc4)c4ccccc4)cc(N(c4ccc5ccccc5c4)c4ccc5oc6ccccc6c5c4)c3)ccc3ccccc23)cc1 ; c1ccc(-c2c(-c3cc(N(c4ccccc4)c4ccccc4)cc(N(c4ccc5oc6ccccc6c5c4)c4cccc5ccccc45)c3)ccc3ccccc23)cc1 ; c1ccc(-c2c(-c3cc(N(c4ccccc4)c4ccccc4)cc(N(c4ccccc4)c4ccc5sc6ccccc6c5c4)c3)ccc3ccccc23)cc1]

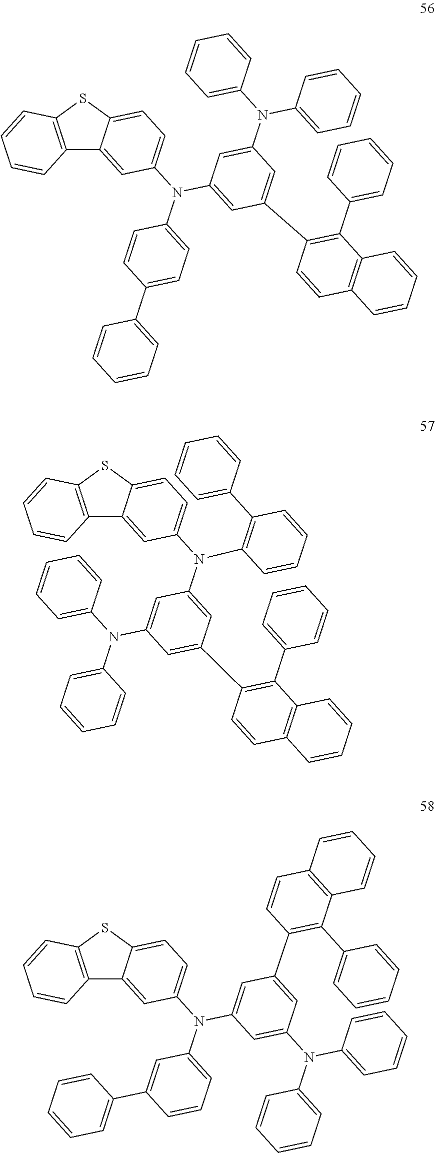 [SMILES: c1ccc(-c2ccc(N(c3cc(-c4ccc5ccccc5c4-c4ccccc4)cc(N(c4ccccc4)c4ccccc4)c3)c3ccc4sc5ccccc5c4c3)cc2)cc1 ; c1ccc(-c2ccccc2N(c2cc(-c3ccc4ccccc4c3-c3ccccc3)cc(N(c3ccccc3)c3ccccc3)c2)c2ccc3sc4ccccc4c3c2)cc1 ; c1ccc(-c2cccc(N(c3cc(-c4ccc5ccccc5c4-c4ccccc4)cc(N(c4ccccc4)c4ccccc4)c3)c3ccc4sc5ccccc5c4c3)c2)cc1]

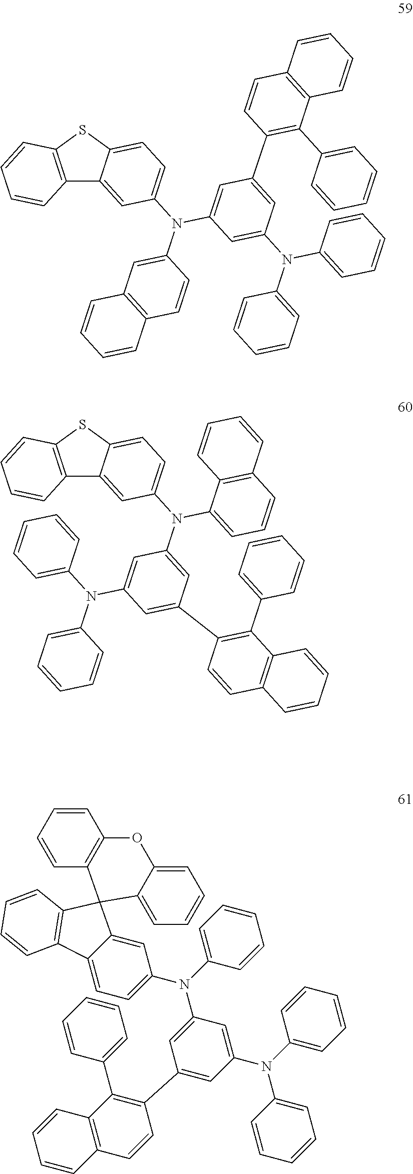 [SMILES: c1ccc(-c2c(-c3cc(N(c4ccccc4)c4ccccc4)cc(N(c4ccc5ccccc5c4)c4ccc5sc6ccccc6c5c4)c3)ccc3ccccc23)cc1 ; c1ccc(-c2c(-c3cc(N(c4ccccc4)c4ccccc4)cc(N(c4ccc5sc6ccccc6c5c4)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)C4(c6ccccc6Oc6ccccc64)c4ccccc4-5)c3)ccc3ccccc23)cc1]

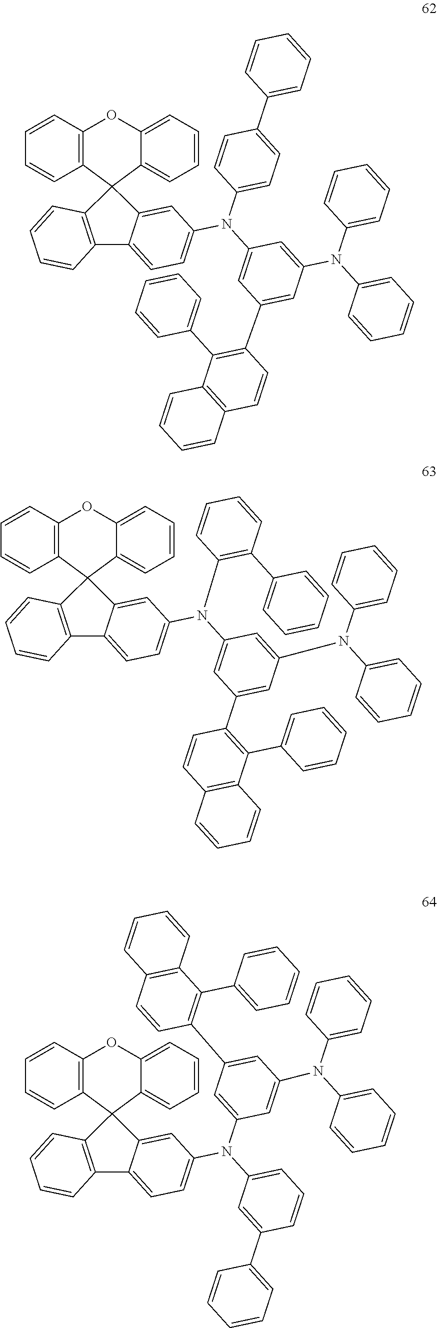 [SMILES: c1ccc(-c2ccc(N(c3cc(-c4ccc5ccccc5c4-c4ccccc4)cc(N(c4ccccc4)c4ccccc4)c3)c3ccc4c(c3)C3(c5ccccc5Oc5ccccc53)c3ccccc3-4)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)C2(c4ccccc4Oc4ccccc42)c2ccccc2-3)cc1 ; c1ccc(-c2cccc(N(c3cc(-c4ccc5ccccc5c4-c4ccccc4)cc(N(c4ccccc4)c4ccccc4)c3)c3ccc4c(c3)C3(c5ccccc5Oc5ccccc53)c3ccccc3-4)c2)cc1]

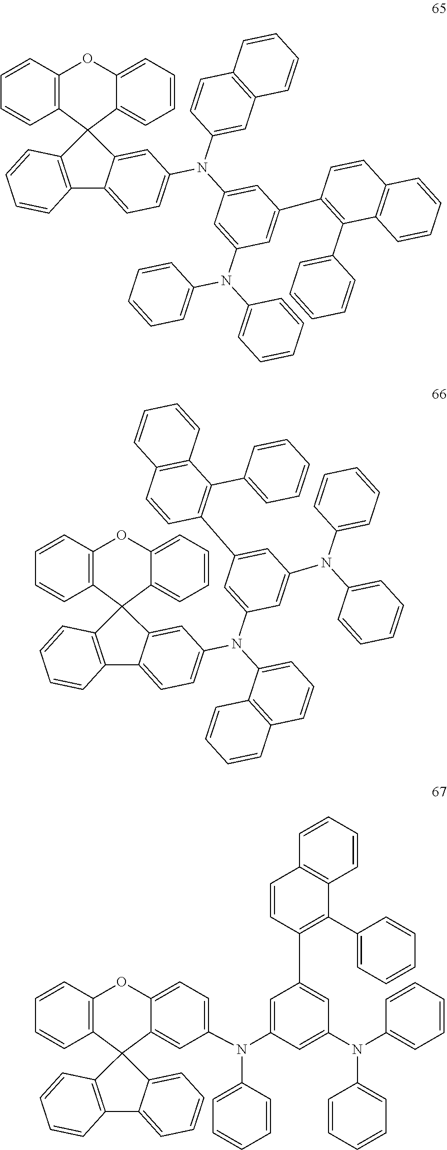 [SMILES: c1ccc(-c2c(-c3cc(N(c4ccccc4)c4ccccc4)cc(N(c4ccc5c(c4)C4(c6ccccc6Oc6ccccc64)c4ccccc4-5)c4ccc5ccccc5c4)c3)ccc3ccccc23)cc1 ; c1ccc(-c2c(-c3cc(N(c4ccccc4)c4ccccc4)cc(N(c4ccc5c(c4)C4(c6ccccc6Oc6ccccc64)c4ccccc4-5)c4cccc5ccccc45)c3)ccc3ccccc23)cc1 ; c1ccc(-c2c(-c3cc(N(c4ccccc4)c4ccccc4)cc(N(c4ccccc4)c4ccc5c(c4)C4(c6ccccc6O5)c5ccccc5-c5ccccc54)c3)ccc3ccccc23)cc1]

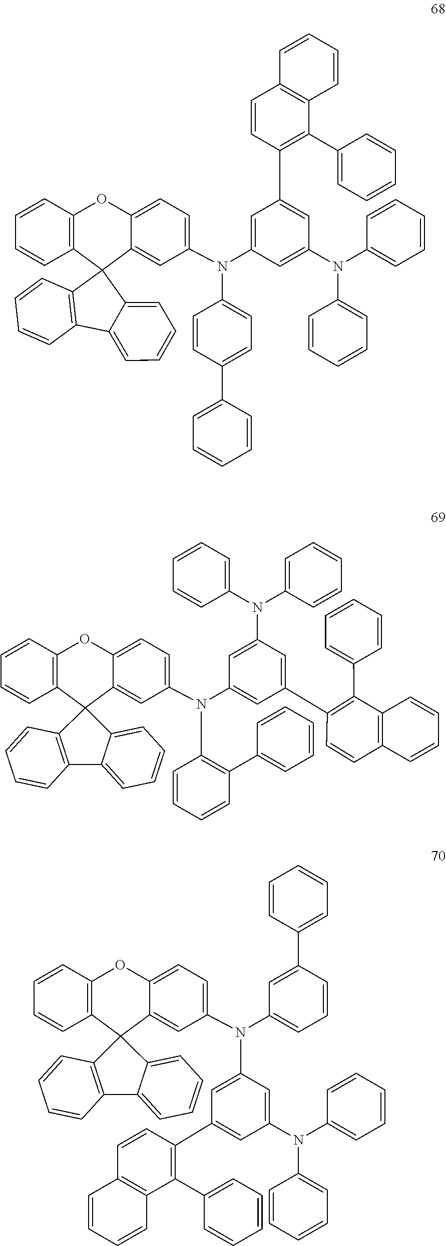 [SMILES: c1ccc(-c2ccc(N(c3cc(-c4ccc5ccccc5c4-c4ccccc4)cc(N(c4ccccc4)c4ccccc4)c3)c3ccc4c(c3)C3(c5ccccc5O4)c4ccccc4-c4ccccc43)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)C2(c4ccccc4O3)c3ccccc3-c3ccccc32)cc1 ; c1ccc(-c2cccc(N(c3cc(-c4ccc5ccccc5c4-c4ccccc4)cc(N(c4ccccc4)c4ccccc4)c3)c3ccc4c(c3)C3(c5ccccc5O4)c4ccccc4-c4ccccc43)c2)cc1]

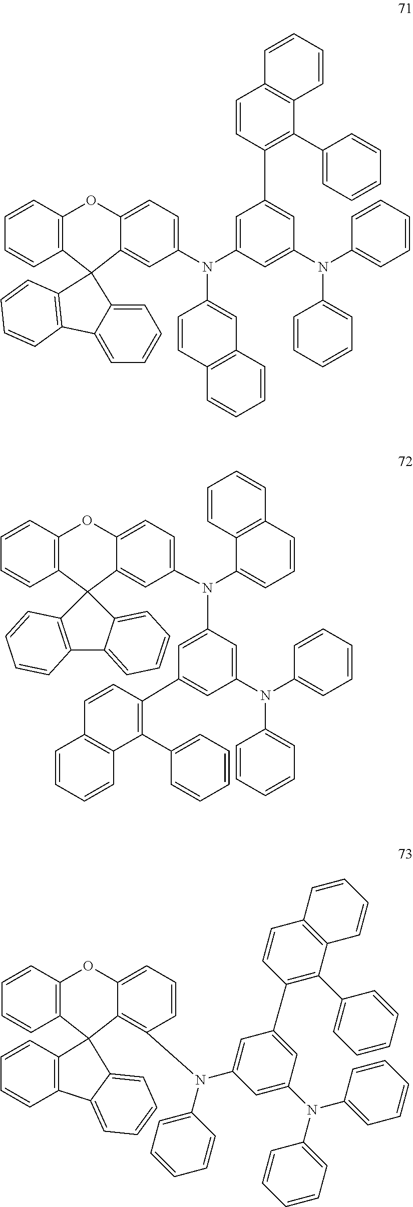 [SMILES: c1ccc(-c2c(-c3cc(N(c4ccccc4)c4ccccc4)cc(N(c4ccc5c(c4)C4(c6ccccc6O5)c5ccccc5-c5ccccc54)c4ccc5ccccc5c4)c3)ccc3ccccc23)cc1 ; c1ccc(-c2c(-c3cc(N(c4ccccc4)c4ccccc4)cc(N(c4ccc5c(c4)C4(c6ccccc6O5)c5ccccc5-c5ccccc54)c4cccc5ccccc45)c3)ccc3ccccc23)cc1 ; c1ccc(-c2c(-c3cc(N(c4ccccc4)c4ccccc4)cc(N(c4ccccc4)c4cccc5c4C4(c6ccccc6O5)c5ccccc5-c5ccccc54)c3)ccc3ccccc23)cc1]

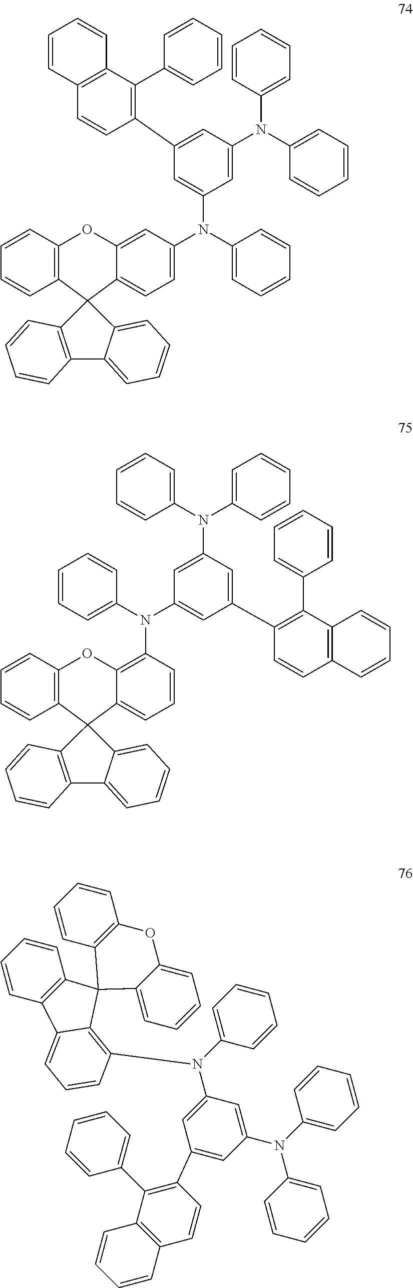 [SMILES: c1ccc(-c2c(-c3cc(N(c4ccccc4)c4ccccc4)cc(N(c4ccccc4)c4ccc5c(c4)Oc4ccccc4C54c5ccccc5-c5ccccc54)c3)ccc3ccccc23)cc1 ; c1ccc(-c2c(-c3cc(N(c4ccccc4)c4ccccc4)cc(N(c4ccccc4)c4cccc5c4Oc4ccccc4C54c5ccccc5-c5ccccc54)c3)ccc3ccccc23)cc1 ; c1ccc(-c2c(-c3cc(N(c4ccccc4)c4ccccc4)cc(N(c4ccccc4)c4cccc5c4C4(c6ccccc6Oc6ccccc64)c4ccccc4-5)c3)ccc3ccccc23)cc1]

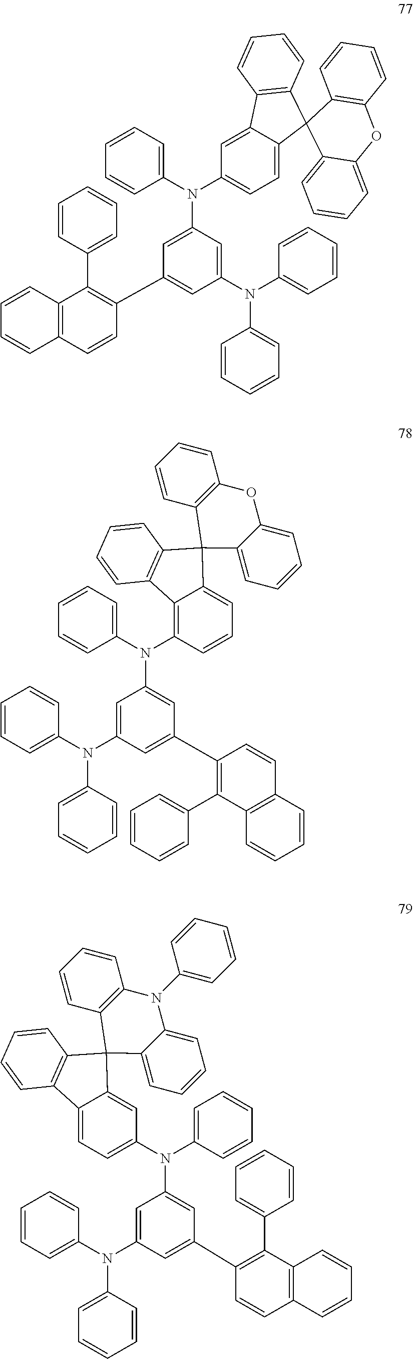 [SMILES: c1ccc(-c2c(-c3cc(N(c4ccccc4)c4ccccc4)cc(N(c4ccccc4)c4ccc5c(c4)-c4ccccc4C54c5ccccc5Oc5ccccc54)c3)ccc3ccccc23)cc1 ; c1ccc(-c2c(-c3cc(N(c4ccccc4)c4ccccc4)cc(N(c4ccccc4)c4cccc5c4-c4ccccc4C54c5ccccc5Oc5ccccc54)c3)ccc3ccccc23)cc1 ; c1ccc(-c2c(-c3cc(N(c4ccccc4)c4ccccc4)cc(N(c4ccccc4)c4ccc5c(c4)C4(c6ccccc6-5)c5ccccc5N(c5ccccc5)c5ccccc54)c3)ccc3ccccc23)cc1]

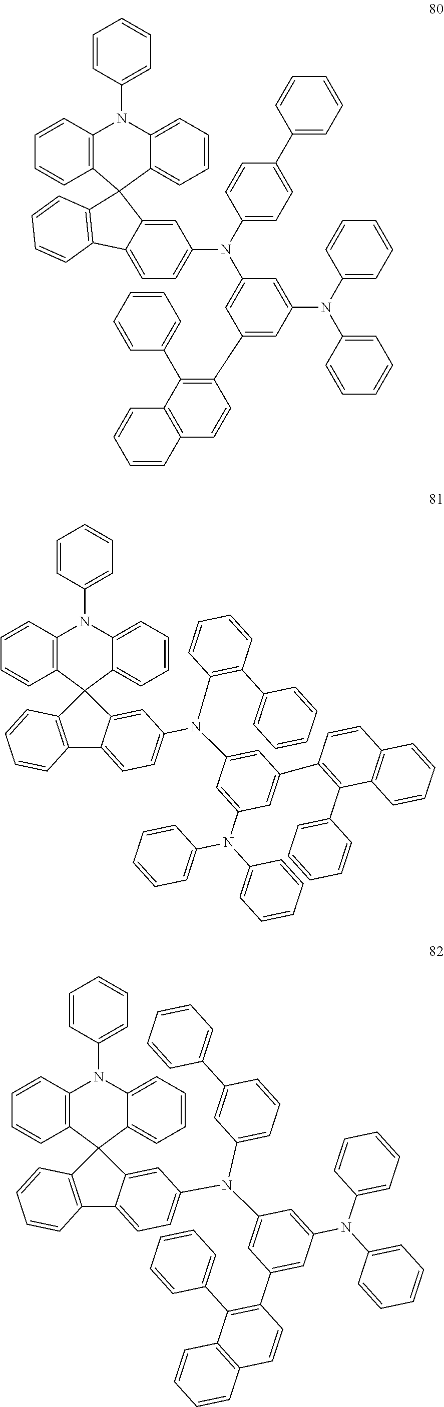 [SMILES: c1ccc(-c2ccc(N(c3cc(-c4ccc5ccccc5c4-c4ccccc4)cc(N(c4ccccc4)c4ccccc4)c3)c3ccc4c(c3)C3(c5ccccc5-4)c4ccccc4N(c4ccccc4)c4ccccc43)cc2)cc1 ; c1ccc(-c2ccccc2N(c2cc(-c3ccc4ccccc4c3-c3ccccc3)cc(N(c3ccccc3)c3ccccc3)c2)c2ccc3c(c2)C2(c4ccccc4-3)c3ccccc3N(c3ccccc3)c3ccccc32)cc1 ; c1ccc(-c2cccc(N(c3cc(-c4ccc5ccccc5c4-c4ccccc4)cc(N(c4ccccc4)c4ccccc4)c3)c3ccc4c(c3)C3(c5ccccc5-4)c4ccccc4N(c4ccccc4)c4ccccc43)c2)cc1]

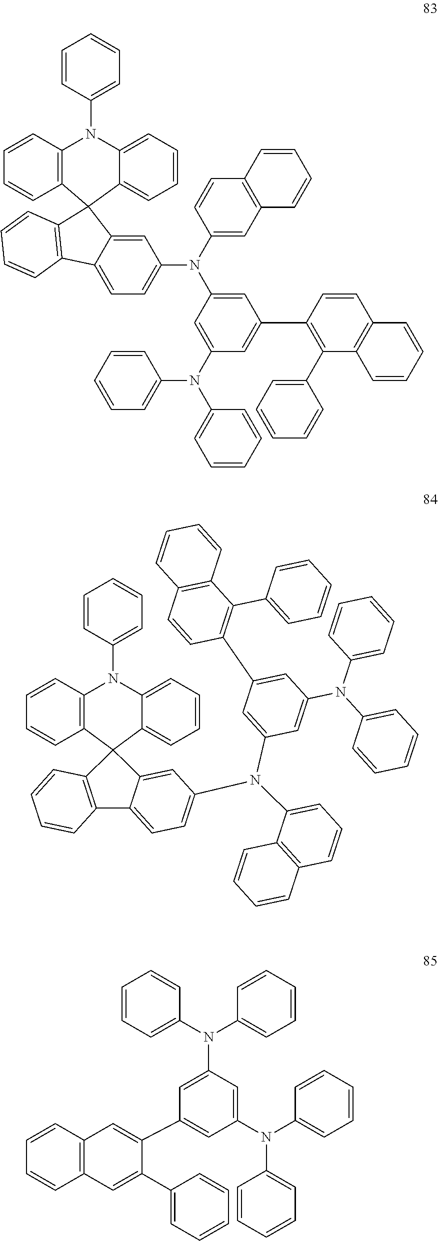 [SMILES: c1ccc(-c2c(-c3cc(N(c4ccccc4)c4ccccc4)cc(N(c4ccc5c(c4)C4(c6ccccc6-5)c5ccccc5N(c5ccccc5)c5ccccc54)c4ccc5ccccc5c4)c3)ccc3ccccc23)cc1 ; c1ccc(-c2c(-c3cc(N(c4ccccc4)c4ccccc4)cc(N(c4ccc5c(c4)C4(c6ccccc6-5)c5ccccc5N(c5ccccc5)c5ccccc54)c4cccc5ccccc45)c3)ccc3ccccc23)cc1 ; c1ccc(-c2cc3ccccc3cc2-c2cc(N(c3ccccc3)c3ccccc3)cc(N(c3ccccc3)c3ccccc3)c2)cc1]

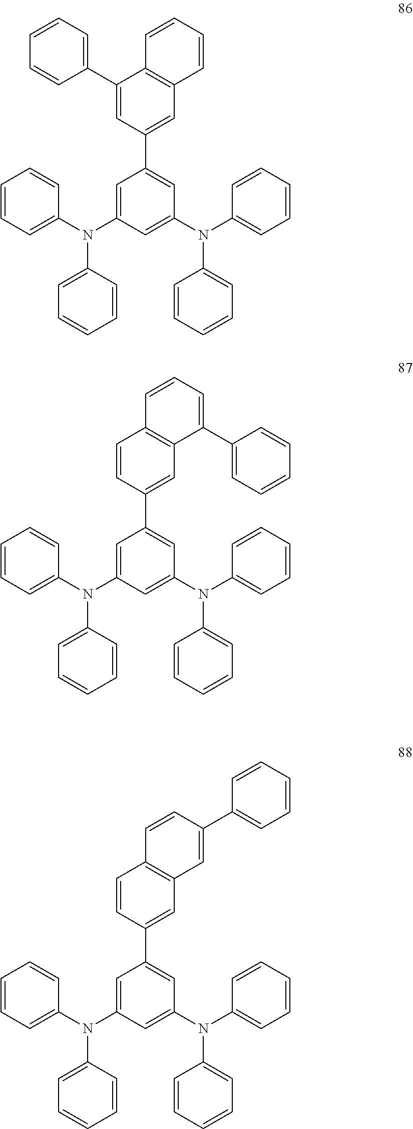 [SMILES: c1ccc(-c2cc(-c3cc(N(c4ccccc4)c4ccccc4)cc(N(c4ccccc4)c4ccccc4)c3)cc3ccccc23)cc1 ; c1ccc(-c2cccc3ccc(-c4cc(N(c5ccccc5)c5ccccc5)cc(N(c5ccccc5)c5ccccc5)c4)cc23)cc1 ; c1ccc(-c2ccc3ccc(-c4cc(N(c5ccccc5)c5ccccc5)cc(N(c5ccccc5)c5ccccc5)c4)cc3c2)cc1]

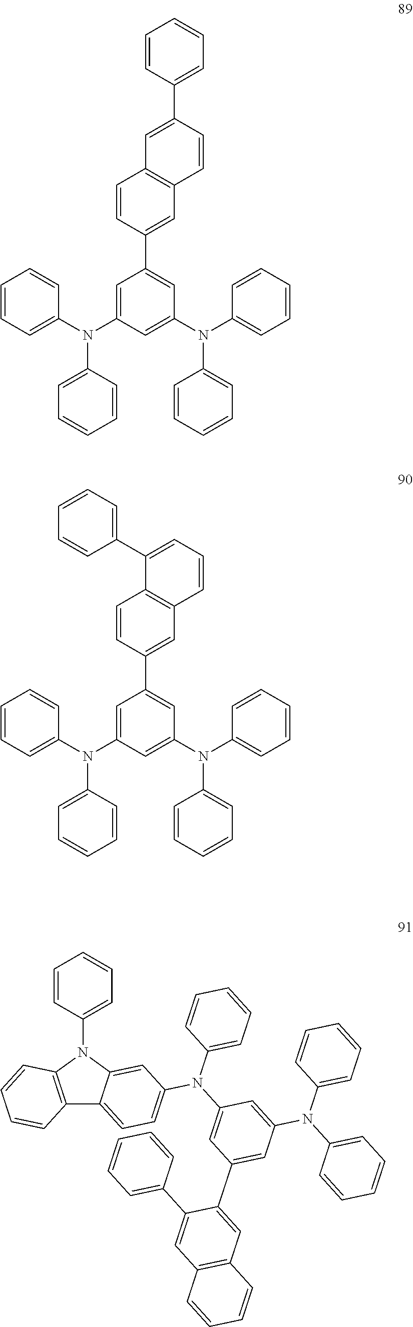 [SMILES: c1ccc(-c2ccc3cc(-c4cc(N(c5ccccc5)c5ccccc5)cc(N(c5ccccc5)c5ccccc5)c4)ccc3c2)cc1 ; c1ccc(-c2cccc3cc(-c4cc(N(c5ccccc5)c5ccccc5)cc(N(c5ccccc5)c5ccccc5)c4)ccc23)cc1 ; c1ccc(-c2cc3ccccc3cc2-c2cc(N(c3ccccc3)c3ccccc3)cc(N(c3ccccc3)c3ccc4c5ccccc5n(-c5ccccc5)c4c3)c2)cc1]

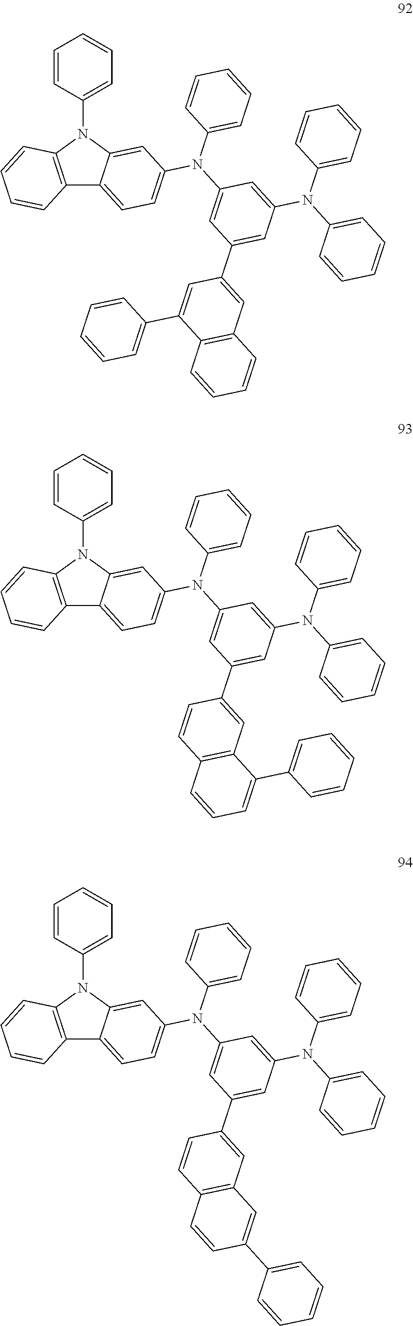 [SMILES: c1ccc(-c2cc(-c3cc(N(c4ccccc4)c4ccccc4)cc(N(c4ccccc4)c4ccc5c6ccccc6n(-c6ccccc6)c5c4)c3)cc3ccccc23)cc1 ; c1ccc(-c2cccc3ccc(-c4cc(N(c5ccccc5)c5ccccc5)cc(N(c5ccccc5)c5ccc6c7ccccc7n(-c7ccccc7)c6c5)c4)cc23)cc1 ; c1ccc(-c2ccc3ccc(-c4cc(N(c5ccccc5)c5ccccc5)cc(N(c5ccccc5)c5ccc6c7ccccc7n(-c7ccccc7)c6c5)c4)cc3c2)cc1]

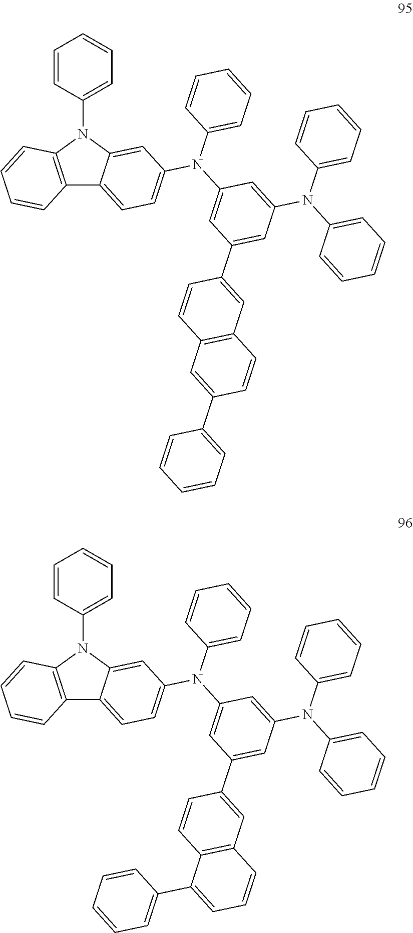 [SMILES: c1ccc(-c2ccc3cc(-c4cc(N(c5ccccc5)c5ccccc5)cc(N(c5ccccc5)c5ccc6c7ccccc7n(-c7ccccc7)c6c5)c4)ccc3c2)cc1 ; c1ccc(-c2cccc3cc(-c4cc(N(c5ccccc5)c5ccccc5)cc(N(c5ccccc5)c5ccc6c7ccccc7n(-c7ccccc7)c6c5)c4)ccc23)cc1]

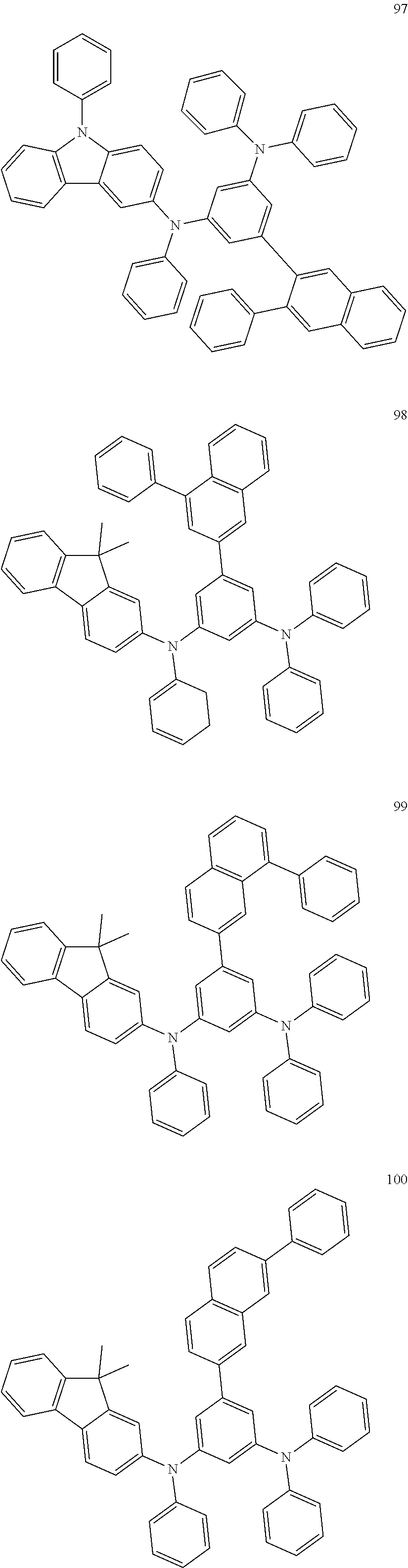 [SMILES: c1ccc(-c2cc3ccccc3cc2-c2cc(N(c3ccccc3)c3ccccc3)cc(N(c3ccccc3)c3ccc4c(c3)c3ccccc3n4-c3ccccc3)c2)cc1 ; CC1(C)c2ccccc2-c2ccc(N(C3=CC=CCC3)c3cc(-c4cc(-c5ccccc5)c5ccccc5c4)cc(N(c4ccccc4)c4ccccc4)c3)cc21 ; CC1(C)c2ccccc2-c2ccc(N(c3ccccc3)c3cc(-c4ccc5cccc(-c6ccccc6)c5c4)cc(N(c4ccccc4)c4ccccc4)c3)cc21 ; CC1(C)c2ccccc2-c2ccc(N(c3ccccc3)c3cc(-c4ccc5ccc(-c6ccccc6)cc5c4)cc(N(c4ccccc4)c4ccccc4)c3)cc21]

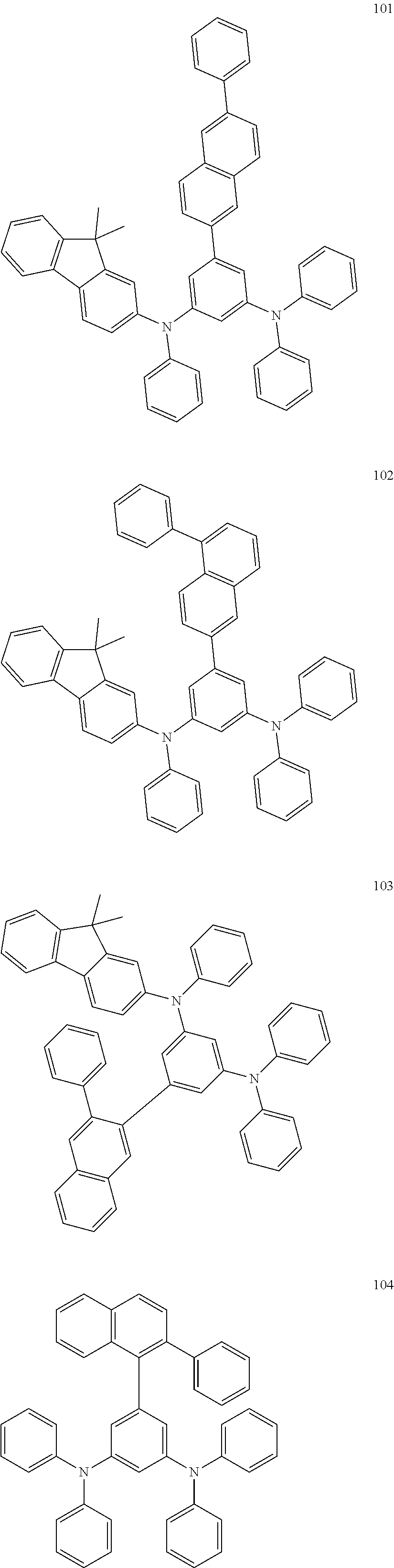 [SMILES: CC1(C)c2ccccc2-c2ccc(N(c3ccccc3)c3cc(-c4ccc5cc(-c6ccccc6)ccc5c4)cc(N(c4ccccc4)c4ccccc4)c3)cc21 ; CC1(C)c2ccccc2-c2ccc(N(c3ccccc3)c3cc(-c4ccc5c(-c6ccccc6)cccc5c4)cc(N(c4ccccc4)c4ccccc4)c3)cc21 ; CC1(C)c2ccccc2-c2ccc(N(c3ccccc3)c3cc(-c4cc5ccccc5cc4-c4ccccc4)cc(N(c4ccccc4)c4ccccc4)c3)cc21 ; c1ccc(-c2ccc3ccccc3c2-c2cc(N(c3ccccc3)c3ccccc3)cc(N(c3ccccc3)c3ccccc3)c2)cc1]

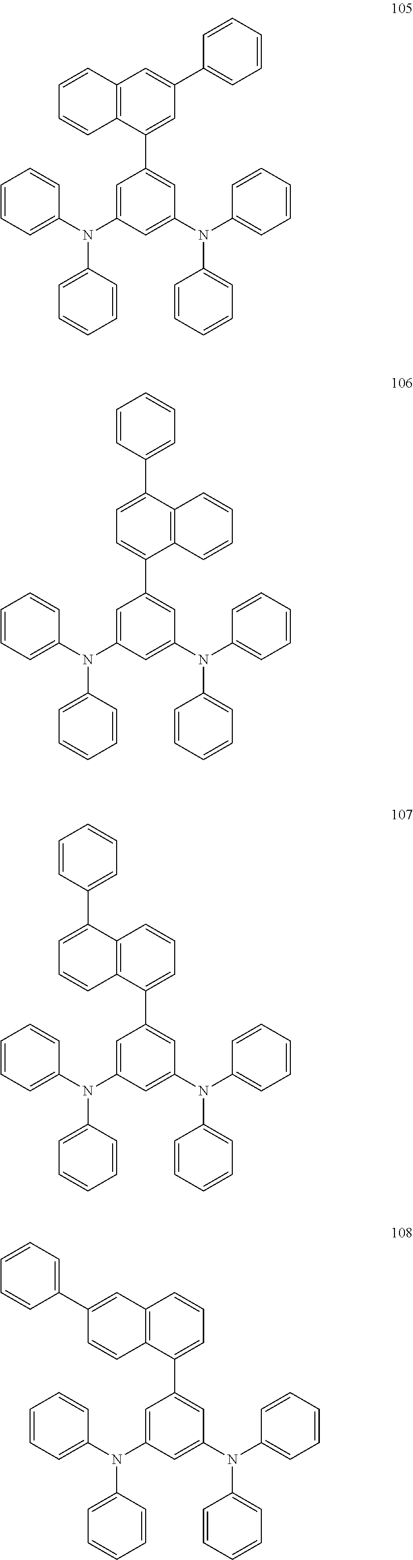 [SMILES: c1ccc(-c2cc(-c3cc(N(c4ccccc4)c4ccccc4)cc(N(c4ccccc4)c4ccccc4)c3)c3ccccc3c2)cc1 ; c1ccc(-c2ccc(-c3cc(N(c4ccccc4)c4ccccc4)cc(N(c4ccccc4)c4ccccc4)c3)c3ccccc23)cc1 ; c1ccc(-c2cccc3c(-c4cc(N(c5ccccc5)c5ccccc5)cc(N(c5ccccc5)c5ccccc5)c4)cccc23)cc1 ; c1ccc(-c2ccc3c(-c4cc(N(c5ccccc5)c5ccccc5)cc(N(c5ccccc5)c5ccccc5)c4)cccc3c2)cc1]

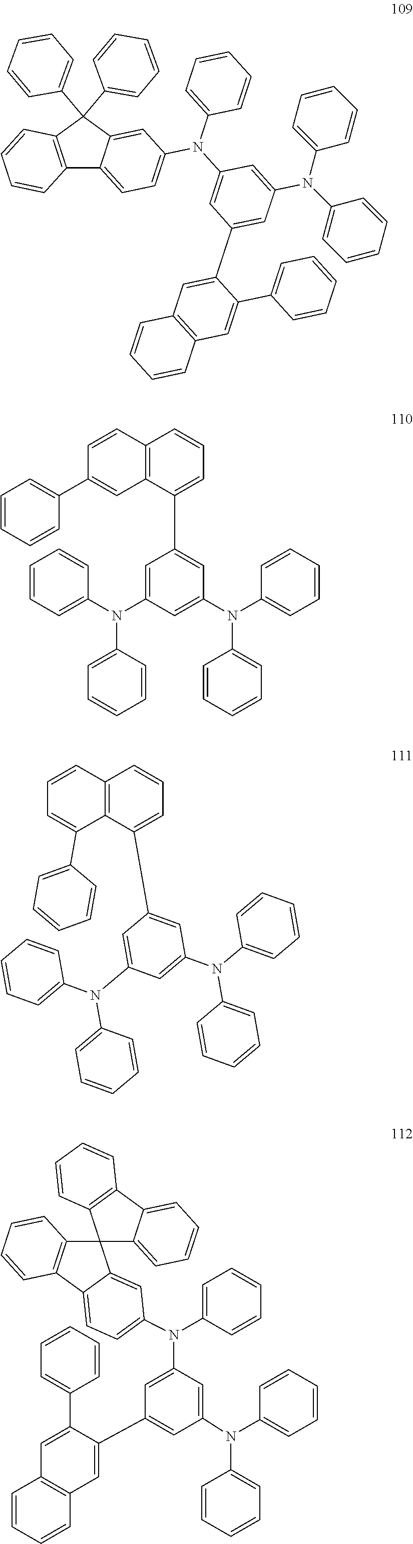 [SMILES: c1ccc(-c2cc3ccccc3cc2-c2cc(N(c3ccccc3)c3ccccc3)cc(N(c3ccccc3)c3ccc4c(c3)C(c3ccccc3)(c3ccccc3)c3ccccc3-4)c2)cc1 ; c1ccc(-c2ccc3cccc(-c4cc(N(c5ccccc5)c5ccccc5)cc(N(c5ccccc5)c5ccccc5)c4)c3c2)cc1 ; c1ccc(-c2cccc3cccc(-c4cc(N(c5ccccc5)c5ccccc5)cc(N(c5ccccc5)c5ccccc5)c4)c23)cc1 ; c1ccc(-c2cc3ccccc3cc2-c2cc(N(c3ccccc3)c3ccccc3)cc(N(c3ccccc3)c3ccc4c(c3)C3(c5ccccc5-c5ccccc53)c3ccccc3-4)c2)cc1]

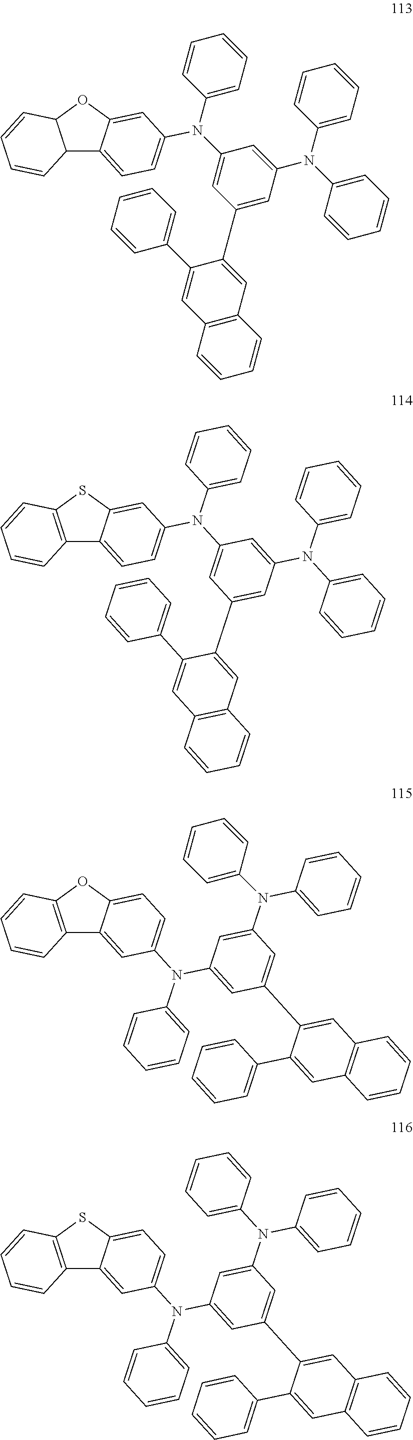 [SMILES: C1=CC2Oc3cc(N(c4ccccc4)c4cc(-c5cc6ccccc6cc5-c5ccccc5)cc(N(c5ccccc5)c5ccccc5)c4)ccc3C2C=C1 ; c1ccc(-c2cc3ccccc3cc2-c2cc(N(c3ccccc3)c3ccccc3)cc(N(c3ccccc3)c3ccc4c(c3)sc3ccccc34)c2)cc1 ; c1ccc(-c2cc3ccccc3cc2-c2cc(N(c3ccccc3)c3ccccc3)cc(N(c3ccccc3)c3ccc4oc5ccccc5c4c3)c2)cc1 ; c1ccc(-c2cc3ccccc3cc2-c2cc(N(c3ccccc3)c3ccccc3)cc(N(c3ccccc3)c3ccc4sc5ccccc5c4c3)c2)cc1]

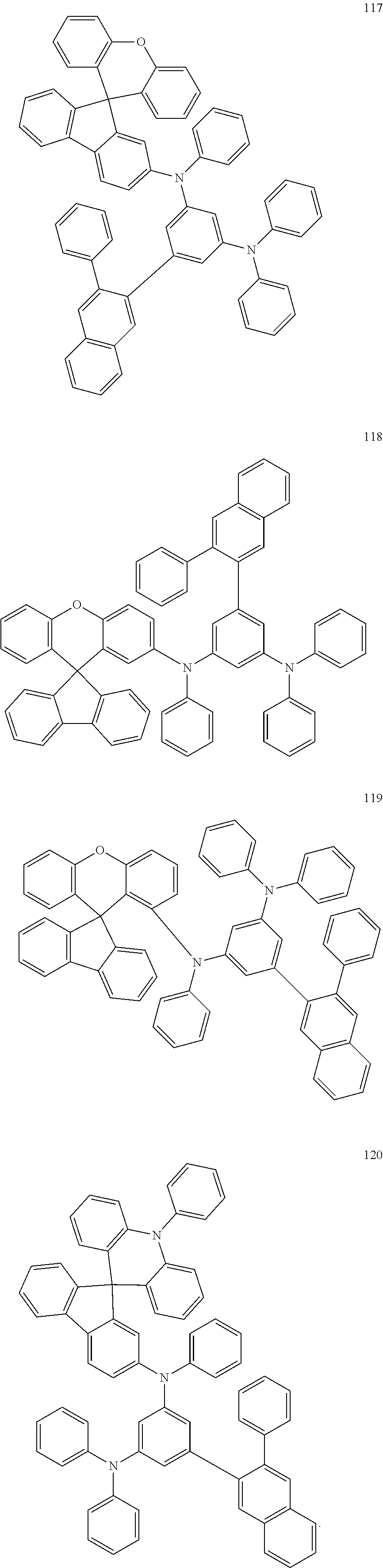 [SMILES: c1ccc(-c2cc3ccccc3cc2-c2cc(N(c3ccccc3)c3ccccc3)cc(N(c3ccccc3)c3ccc4c(c3)C3(c5ccccc5Oc5ccccc53)c3ccccc3-4)c2)cc1 ; c1ccc(-c2cc3ccccc3cc2-c2cc(N(c3ccccc3)c3ccccc3)cc(N(c3ccccc3)c3ccc4c(c3)C3(c5ccccc5O4)c4ccccc4-c4ccccc43)c2)cc1 ; c1ccc(-c2cc3ccccc3cc2-c2cc(N(c3ccccc3)c3ccccc3)cc(N(c3ccccc3)c3cccc4c3C3(c5ccccc5O4)c4ccccc4-c4ccccc43)c2)cc1 ; c1ccc(-c2cc3ccccc3cc2-c2cc(N(c3ccccc3)c3ccccc3)cc(N(c3ccccc3)c3ccc4c(c3)C3(c5ccccc5-4)c4ccccc4N(c4ccccc4)c4ccccc43)c2)cc1]

As the amine-containing compound represented by Formula 1 includes a naphthalene group substituted with a C₆-C₆₀ aryl group unsubstituted or substituted with at least one R₁₀ₐ, the amine-containing compound may have excellent charge transport ability. In addition, as the amine-containing compound represented by Formula 1 includes a diamine structure at a meta position (for example, having a diamine structure at carbon atoms located at meta positions of the phenyl group), the amine-containing compound may have a highest occupied molecular orbital (HOMO) energy level suitable for a hole transport layer.

Therefore, by using the amine-containing compound represented by Formula 1, a light-emitting device (e.g., an organic light-emitting device) having a low driving voltage, high luminescence efficiency, and long device lifespan characteristics may be realized.

Synthesis methods of the amine-containing compound represented by Formula 1 may be recognizable by one of ordinary skill in the art by referring to Synthesis Examples and/or Examples provided below.

According to another aspect, at least one amine-containing compound represented by Formula 1 may be used in a light-emitting device (e.g., an organic light-emitting device). Accordingly, provided is a light-emitting device including: a first electrode, a second electrode facing the first electrode; an interlayer arranged between the first electrode and the second electrode and including an emission layer; and the amine-containing compound represented by Formula 1.

According to some example embodiments, the first electrode may be an anode, and the second electrode may be a cathode,
the interlayer may further include a hole transport region located between the first electrode and the emission layer and an electron transport region located between the emission layer and the second electrode,
the hole transport region may include a hole injection layer, a hole transport layer, an emission auxiliary layer, an electron-blocking layer, or any combination thereof, and
the electron transport region may include a buffer layer, a hole-blocking layer, an electron control layer, an electron transport layer, an electron injection layer, or any combination thereof.

In some example embodiments, the amine-containing compound may be included between the first electrode and the second electrode of the light-emitting device. Accordingly, the amine-containing compound may be included in the interlayer of the light-emitting device, for example, in the hole transport region of the interlayer. That is, the hole transport region may include the amine-containing compound represented by Formula 1.

In some example embodiments, the hole transport region may include a hole injection layer, and the hole injection layer may include the amine-containing compound represented by Formula 1.

In some example embodiments, the hole transport region may include a hole transport layer, and the hole transport layer may include the amine-containing compound represented by Formula 1.

In some example embodiments, the emission layer in the interlayer of the light-emitting device may include a dopant and a host. The emission layer may emit red light, green light, blue light, and/or white light. For example, the emission layer may emit blue light. The blue light may have a maximum emission wavelength of, for example, about 400 nm to about 490 nm.

In one or more example embodiments, the blue light may have a maximum emission wavelength in a range of about 430 nm to about 480 nm, about 430 nm to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In some example embodiments, the emission layer of the interlayer of the light-emitting device may include a dopant and a host, and the host may include a first host compound and a second host compound. In this regard, the first host compound may be a hole-transporting host, and the second host compound may be an electron-transporting host.

In some example embodiments, the hole-transporting host may be a compound including a hole-transporting moiety, and the electron-transporting host may not only be a compound including an electron-transporting moiety but also a compound having bipolar characteristics.

The terms “hole-transporting host” and “electron-transporting host” may each be understood according to the relative difference between the hole mobility and electron mobility in the hole-transporting host and the electron-transporting host. For example, even when the electron-transporting host does not include an electron-transporting moiety, a bipolar compound exhibiting relatively higher electron mobility than the hole-transporting host may be also understood as the electron-transporting host.

In some example embodiments, the first host compound and the second host compound may form an exciplex.

In one or more example embodiments, the amine-containing compound may be included in the host. That is, the amine-containing compound may act as a host. For example, in some example embodiments, the amine-containing compound may act as the first host compound which is the hole-transporting host.

In some example embodiments, the emission layer in the interlayer of the light-emitting device may include a dopant and a host, and the dopant may include a phosphorescent dopant. The phosphorescent dopant may emit blue light.

In one or more example embodiments, the emission layer in the interlayer of the light-emitting device may include a dopant and a host, and the dopant may include a phosphorescent dopant and a delayed fluorescence material. The delayed fluorescence material may emit blue light.

In some example embodiments, the light-emitting device may further include a capping layer arranged outside the first electrode or outside the second electrode.

In some example embodiments, the light-emitting device may further include at least one of a first capping layer arranged outside the first electrode and a second capping layer arranged outside the second electrode, wherein at least one of the first capping layer and the second capping layer may include the amine-containing compound represented by Formula 1. More details for the first capping layer and/or second capping layer are the same as described in the specification.

In some example embodiments, the light-emitting device may further include a first capping layer arranged outside the first electrode. For example, the first capping layer may include the amine-containing compound represented by Formula 1.

In some example embodiments, the light-emitting device may further include a second capping layer arranged outside the second electrode. For example, the second capping layer may include the amine-containing compound represented by Formula 1.

In some example embodiments, the light-emitting device may further include a first capping layer arranged outside the first electrode and a second capping layer arranged outside the second electrode. For example, at least one of the first capping layer and the second capping layer may include the amine-containing compound represented by Formula 1.

The wording “(interlayer and/or capping layer) includes an amine-containing compound” as used herein may be understood as “(interlayer and/or capping layer) may include one kind of amine-containing compound represented by Formula 1 or two or more different kinds of amine-containing compounds, each represented by Formula 1”.

For example, the interlayer and/or capping layer may include Compound 1 only as the amine-containing compound. In some example embodiments, Compound 1 may be included in the hole transport region of the light-emitting device. For example, in some example embodiments, the amine-containing compound represented by Formula 1 may consist essentially Compound 1 and the interlayer and/or capping layer may consist essentially of Compound 1. Or, the interlayer may include, as the amine-containing compound, Compound 1 and Compound 2. In this regard, Compound 1 and Compound 2 may be present in an identical layer (for example, Compound 1 and Compound 2 may all be present in the hole transport region) or in different layers (for example, Compound 1 may be present in the hole transport region, and Compound 2 may be present in the electron transport region).

The term “interlayer” as used herein refers to a single layer and/or a plurality of layers between a first electrode and a second electrode of a light-emitting device.

In some example embodiments, the light-emitting device may be a tandem light-emitting device. Accordingly, a light-emitting device may include: a first electrode; a second electrode facing the first electrode; m emitting units stacked between the first electrode and the second electrode; and m−1 charge generation layers interposed between two neighboring emitting units of the m emitting units. In this regard, m may be an integer of 2 or more, m emitting units may each independently include an interlayer including an emission layer, and in at least one of the m emitting units, the interlayer may include the amine-containing compound represented by Formula 1.

The emitting unit herein is not particularly limited as long as the emitting unit has a function of emitting light. In some example embodiments, the emitting unit may include one or more emission layers. When necessary, the emitting unit may further include a layer other than the emission layer.

In some example embodiments, the m emitting units may each include a hole transport region, an emission layer, and an electron transport region which are sequentially arranged. The hole transport region, the emission layer, and the electron transport region may each be the same as described herein.

The charge generation layer may refer to a layer which acts as a cathode by generating electrons for one emitting unit of the two neighboring emitting units and acts as an anode by generating holes for the other one emitting unit of the two neighboring emitting units. The charge generation layer may not be directly connected to an electrode and separate adjacent emitting units. A light-emitting device including the m emitting units may include m−1 charge generation layers.

In some example embodiments, the charge generation layer may include an n-type charge generation layer and a p-type charge generation layer. The n-type refers to n-type semiconductor characteristics, that is, the characteristics of injecting or transporting electrons. The p-type refers to p-type semiconductor characteristics, that is, the characteristics of injecting or transporting holes. The n-type charge generation layer and the p-type charge generation layer may be in direct contact with each other to form an NP junction. By the NP junction, electrons and holes may be simultaneously generated between the n-type charge generation layer and the p-type charge generation layer. The generated electrons may be transferred to one of the two neighboring emitting units through the n-type charge generation layer. The generated holes may be transferred to the other one of the two neighboring emitting units through the p-type charge generation layer. In addition, because each of the charge generation layers include one n-type charge generation layer and one p-type charge generation layer, the light-emitting device including m−1 charge generation layers may each include m−1 n-type charge generation layers and m−1 p-type charge generation layers.

Another aspect provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In some example embodiments, the electronic apparatus may further include a color filter, a color conversion layer, a touch screen layer, a polarizing layer, or any combination thereof. For more details on the electronic apparatus, related descriptions provided herein may be referred to.

Another aspect of the disclosure provides electronic equipment including the light-emitting device. For example, the electronic equipment may be one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and a signboard. More details on the electronic equipment may be the same as described herein.

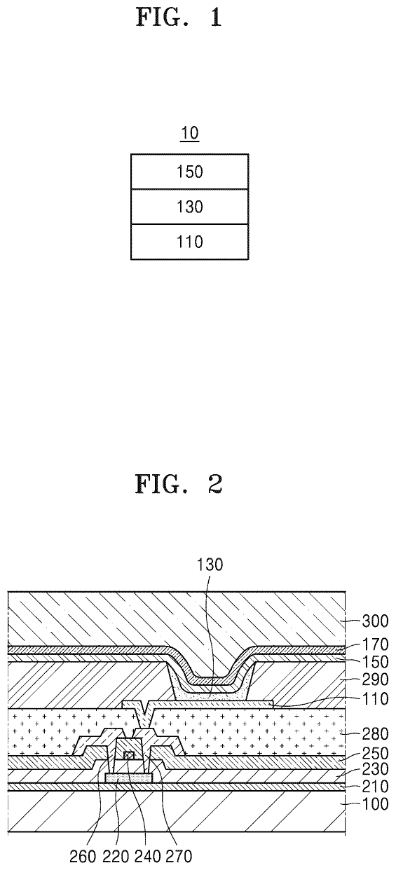

### [Description of FIG. 1]

FIG. 1 is a schematic cross-sectional view of a light-emitting device 10 according to some example embodiments. The light-emitting device 10 includes a first electrode 110, an interlayer 130, and a second electrode 150.

Hereinafter, the structure of the light-emitting device 10 according to some example embodiments and a method of manufacturing the light-emitting device 10 will be described with reference to FIG. 1.

### [First Electrode 110]

In FIG. 1, a substrate may be additionally located under the first electrode 110 or on the second electrode 150. As the substrate, a glass substrate or a plastic substrate may be used. In one or more embodiments, the substrate may be a flexible substrate, and may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), and/or any combination thereof. In one or more example embodiments, when the first electrode 110 is a semi-transmissive electrode or a reflective electrode, a material for forming the first electrode 110 may include magnesium (Mg), silver (Ag), aluminum (AI), aluminum-lithium (Al—Li), calcium (Ca), magnesium-indium (Mg—In), magnesium-silver (Mg—Ag), and/or any combination thereof.

The first electrode 110 may have a single-layered structure consisting of a single layer or a multi-layered structure including a plurality of layers. For example, the first electrode 110 may have a three-layered structure of ITO/Ag/ITO.

### [Interlayer 130]

The interlayer 130 may be located on the first electrode 110. The interlayer 130 may include an emission layer. In some example embodiments, the interlayer 130 includes a plurality of layers.

The interlayer 130 may further include a hole transport region located between the first electrode 110 and the emission layer, and an electron transport region located between the emission layer and the second electrode 150.

The interlayer 130 may further include, in addition to various organic materials, a metal-containing compound such as an organometallic compound, an inorganic material such as quantum dots, or the like.

In one or more example embodiments, the interlayer 130 may include, i) two or more emitting units sequentially stacked between the first electrode 110 and the second electrode 150, and ii) a charge generation layer located between the two or more emitting units. When the interlayer 130 includes emitting units and a charge generation layer as described above, the light-emitting device 10 may be a tandem light-emitting device.

### [Hole Transport Region in Interlayer 130]

The hole transport region may have: i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron-blocking layer, and/or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the layers of each structure being stacked sequentially from the first electrode 110.

The hole transport region may include a compound represented by Formula 201, a compound represented by Formula 202, or any combination thereof:

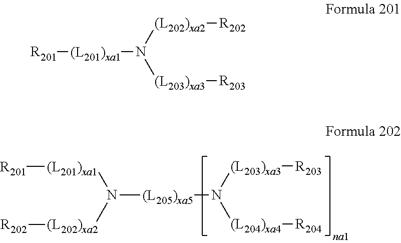

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)—*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
When xa1 is 0, *-(L₂₀₁)ₓₐ₁-*′ may be a single bond, when xa2 is 0, *-(L₂₀₂)ₓₐ₂-*′ may be a single bond, when xa3 is 0, *-(L₂₀₃)ₓₐ₃-*′ may be a single bond, and when xa4 is 0, *-(L₂₀₄)ₓₐ₄—*′ may be a single bond.
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group or the like) unsubstituted or substituted with at least one R₁₀ₐ (for example, Compound HT16 depicted below),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

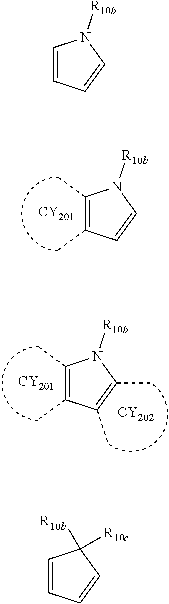

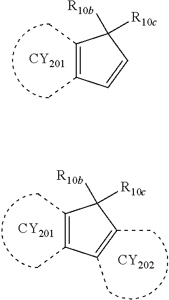

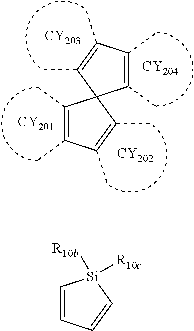

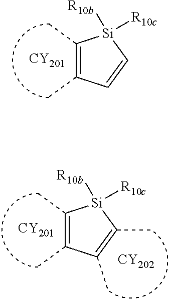

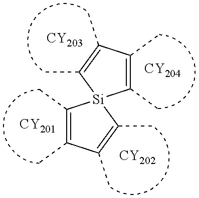

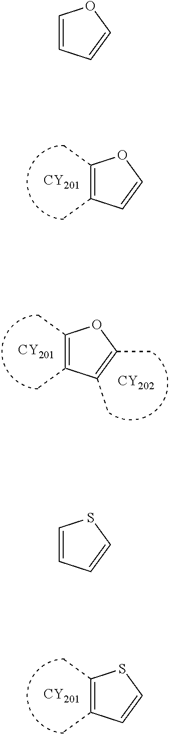

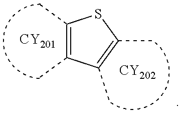

In Formulae CY201 to CY217, R_{10b} and R_{10c} may each be the same as described with respect to R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described above.

In some example embodiments, ring CY201 to ring CY204 in Formulae CY201 to CY217 may each independently be a benzene group, a naphthalene group, a phenanthrene group, or an anthracene group.

In one or more example embodiments, each of Formulae 201 and 202 may include at least one of groups represented by Formulae CY201 to CY203.

In one or more example embodiments, Formula 201 may include at least one of the groups represented by Formulae CY201 to CY203 and at least one of the groups represented by Formulae CY204 to CY217.

In one or more example embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more example embodiments, each of Formulae 201 and 202 may not include a group represented by one of Formulae CY201 to CY203.

In one or more example embodiments, each of Formulae 201 and 202 may not include a group represented by one of Formulae CY201 to CY203, and may include at least one of the groups represented by Formulae CY204 to CY217.

In one or more example embodiments, each of Formulae 201 and 202 may not include a group represented by one of Formulae CY201 to CY217.

In some example embodiments, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 9-(4-tert-Butylphenyl)-3,6-bis(triphenylsilyl)-9H-carbazole (CzSi), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or any combination thereof:

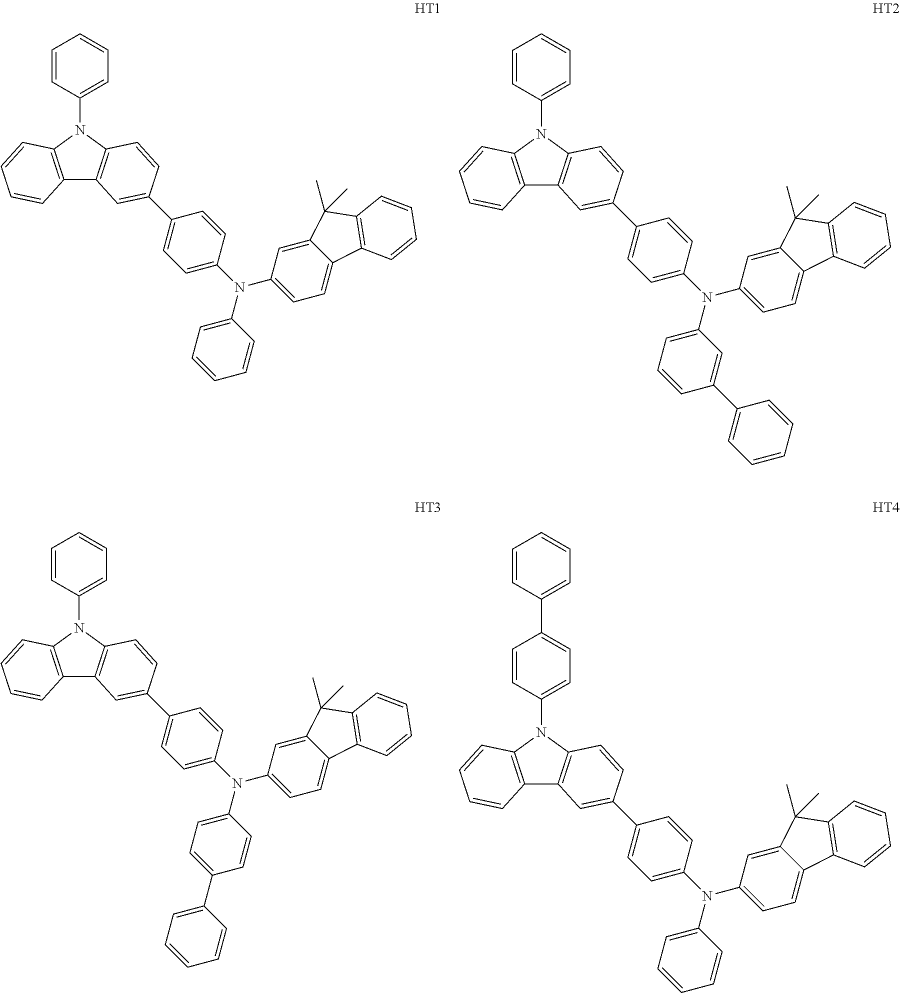 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

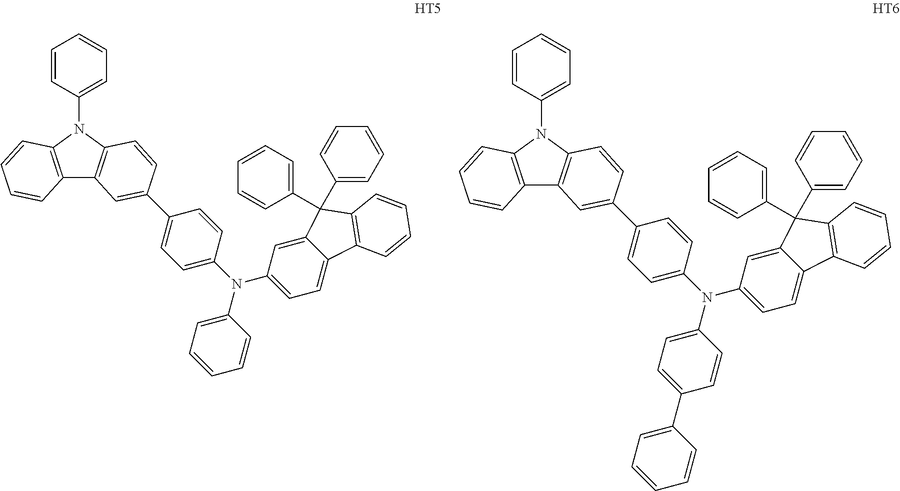 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

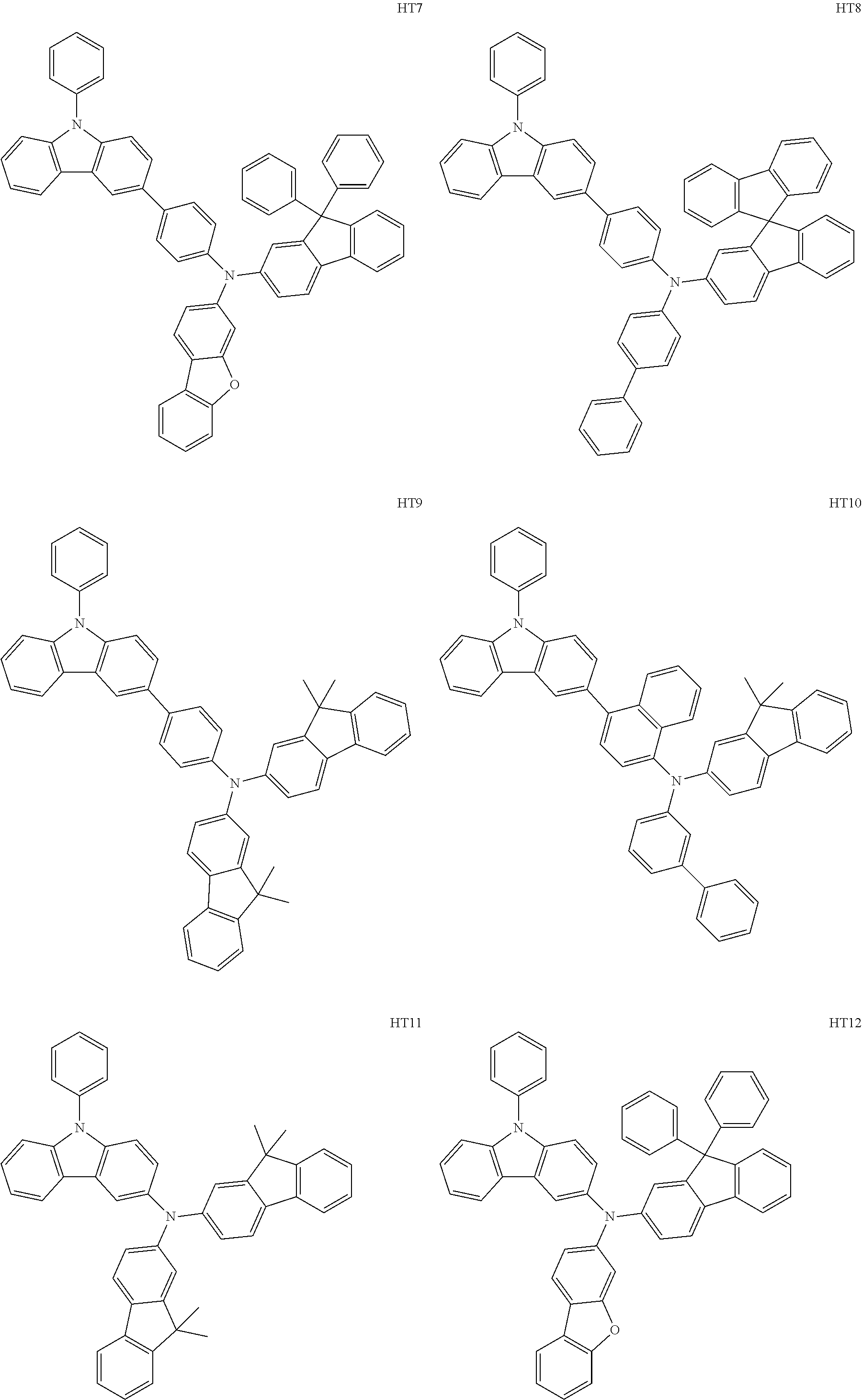

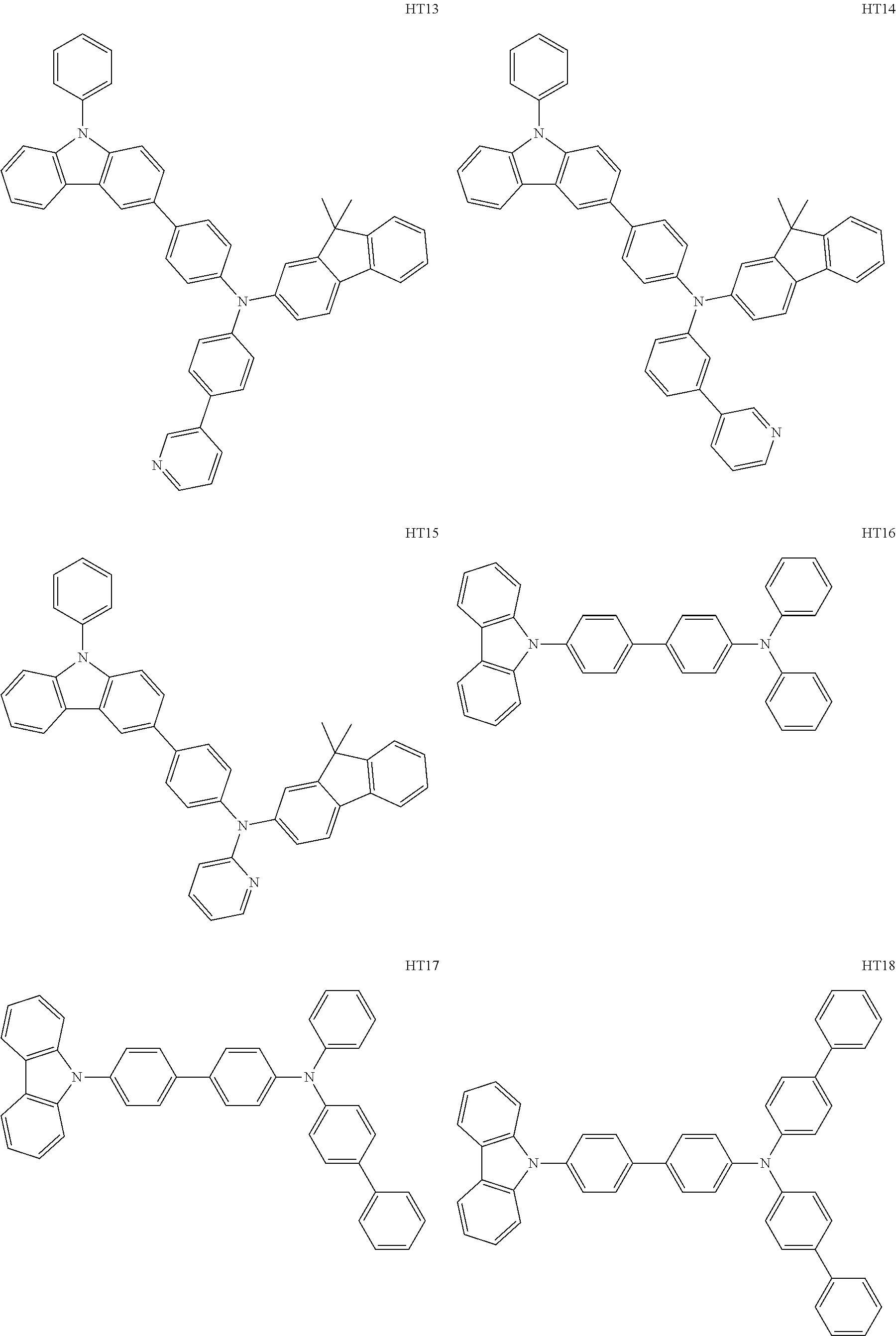

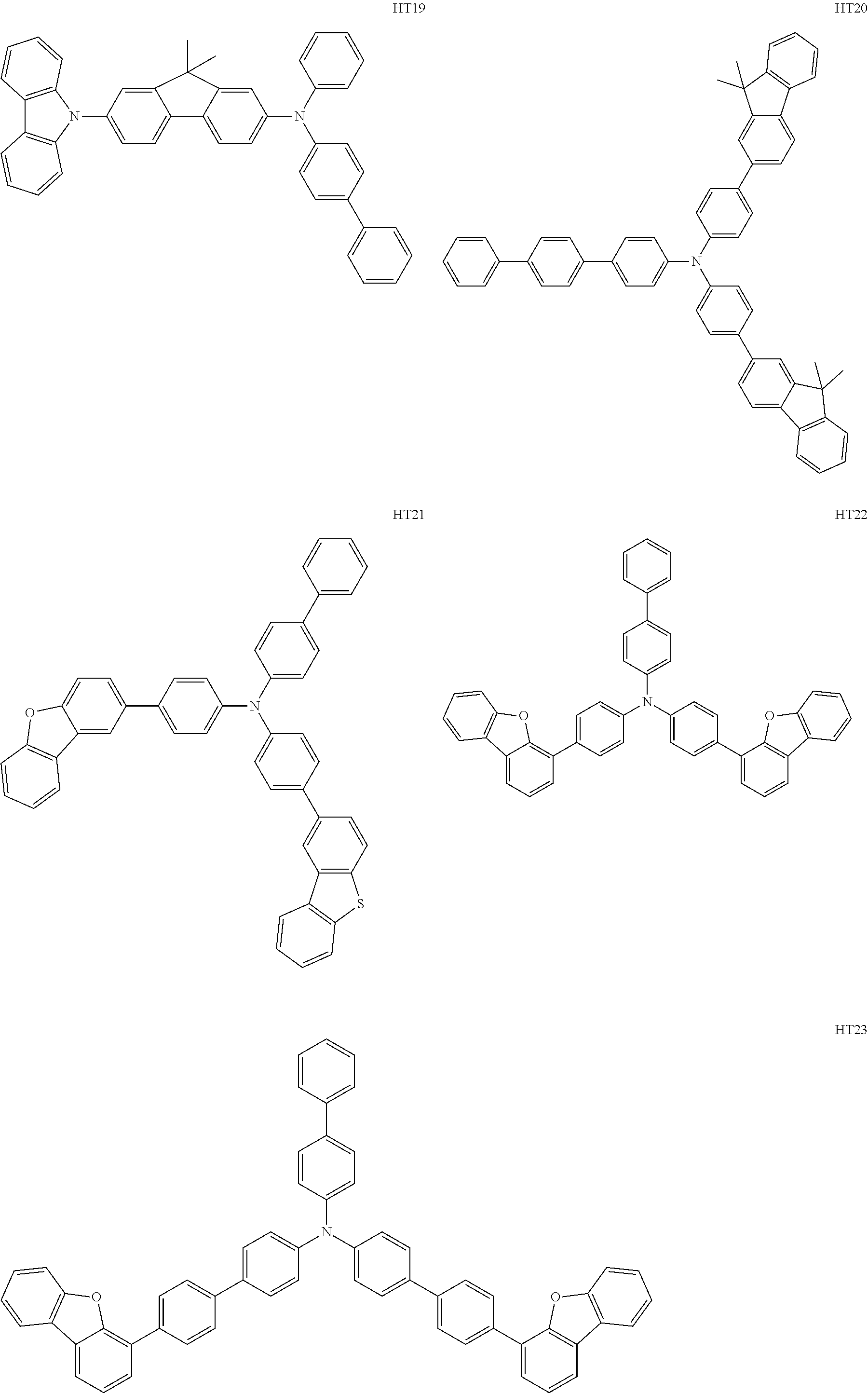

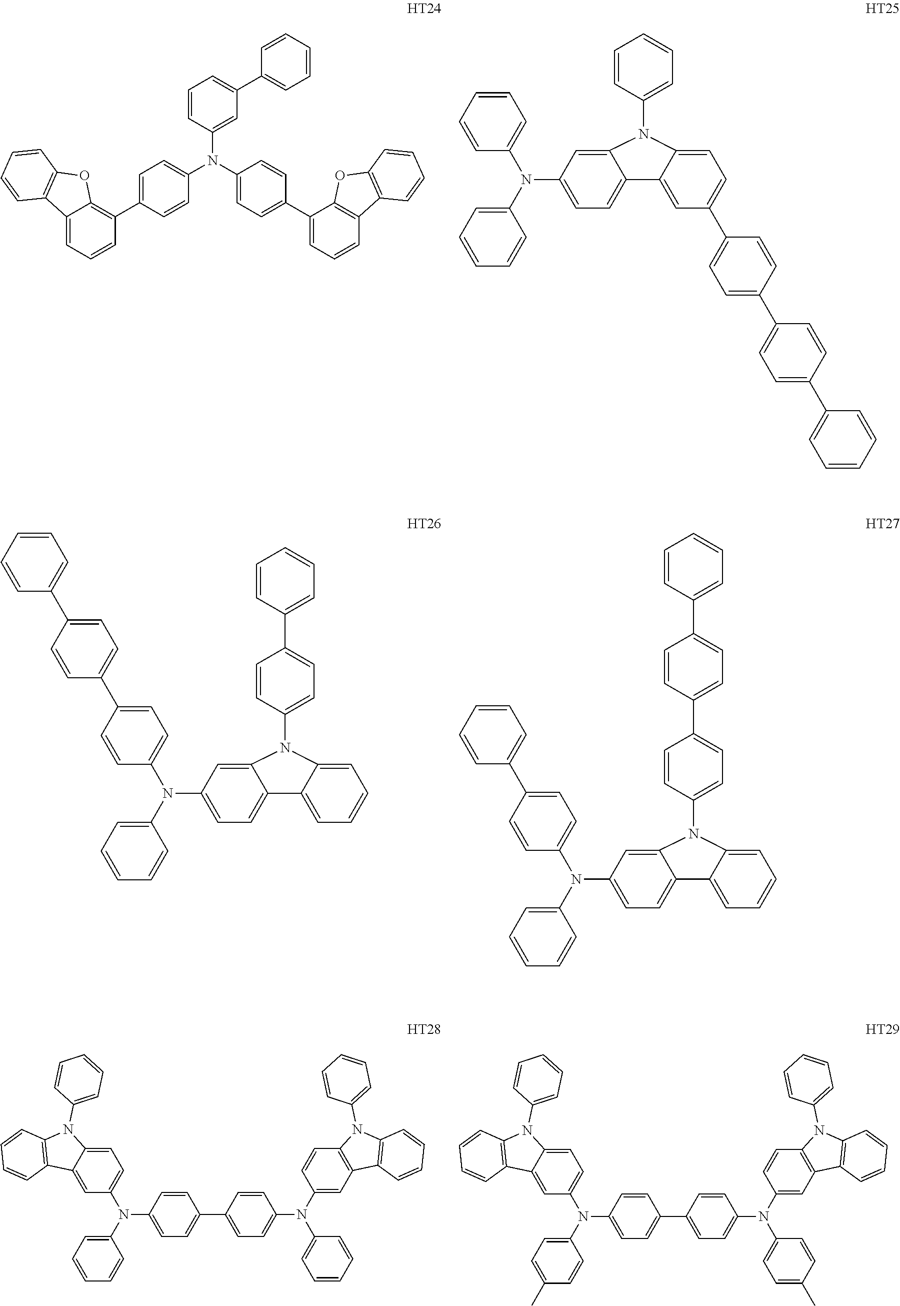

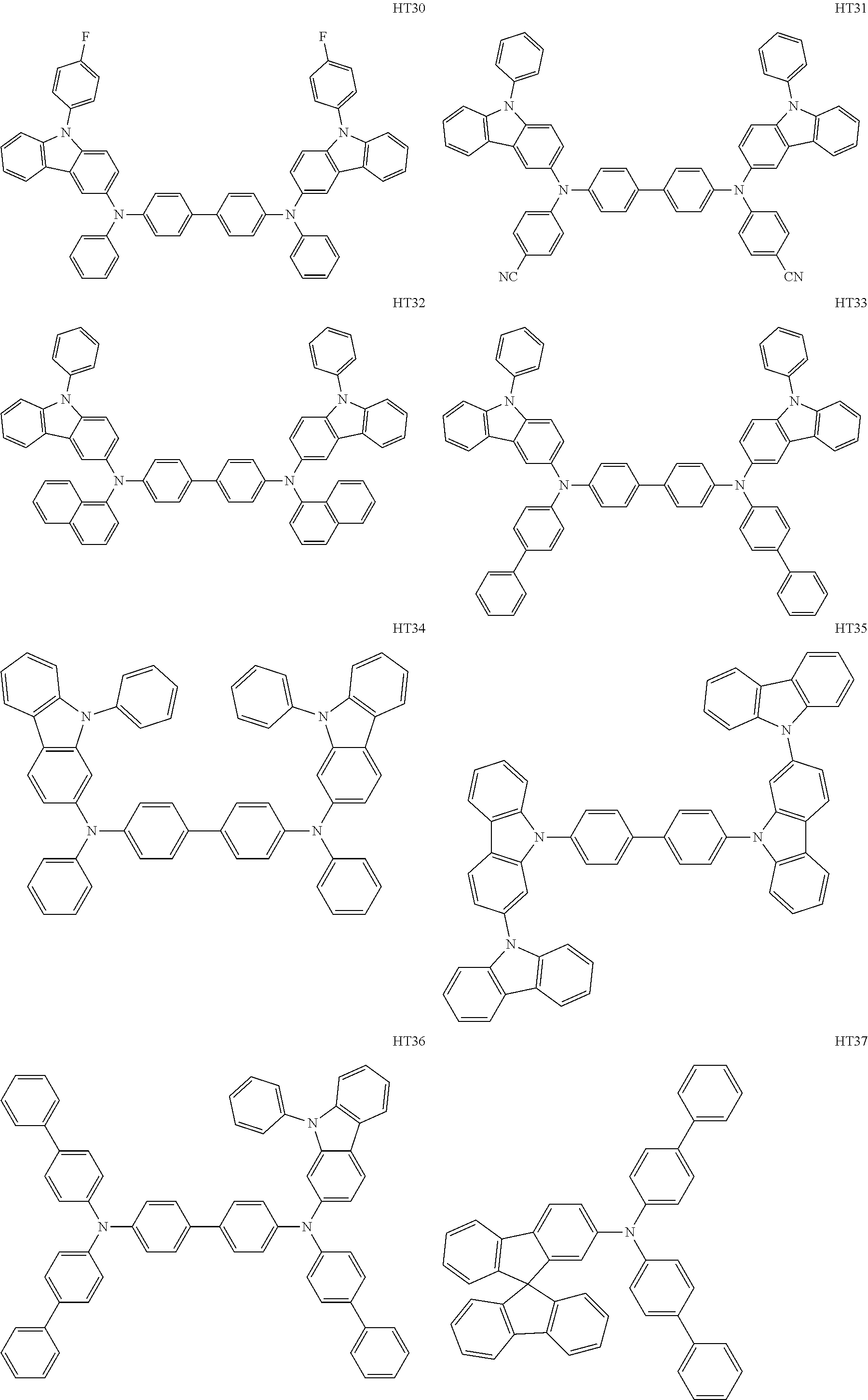

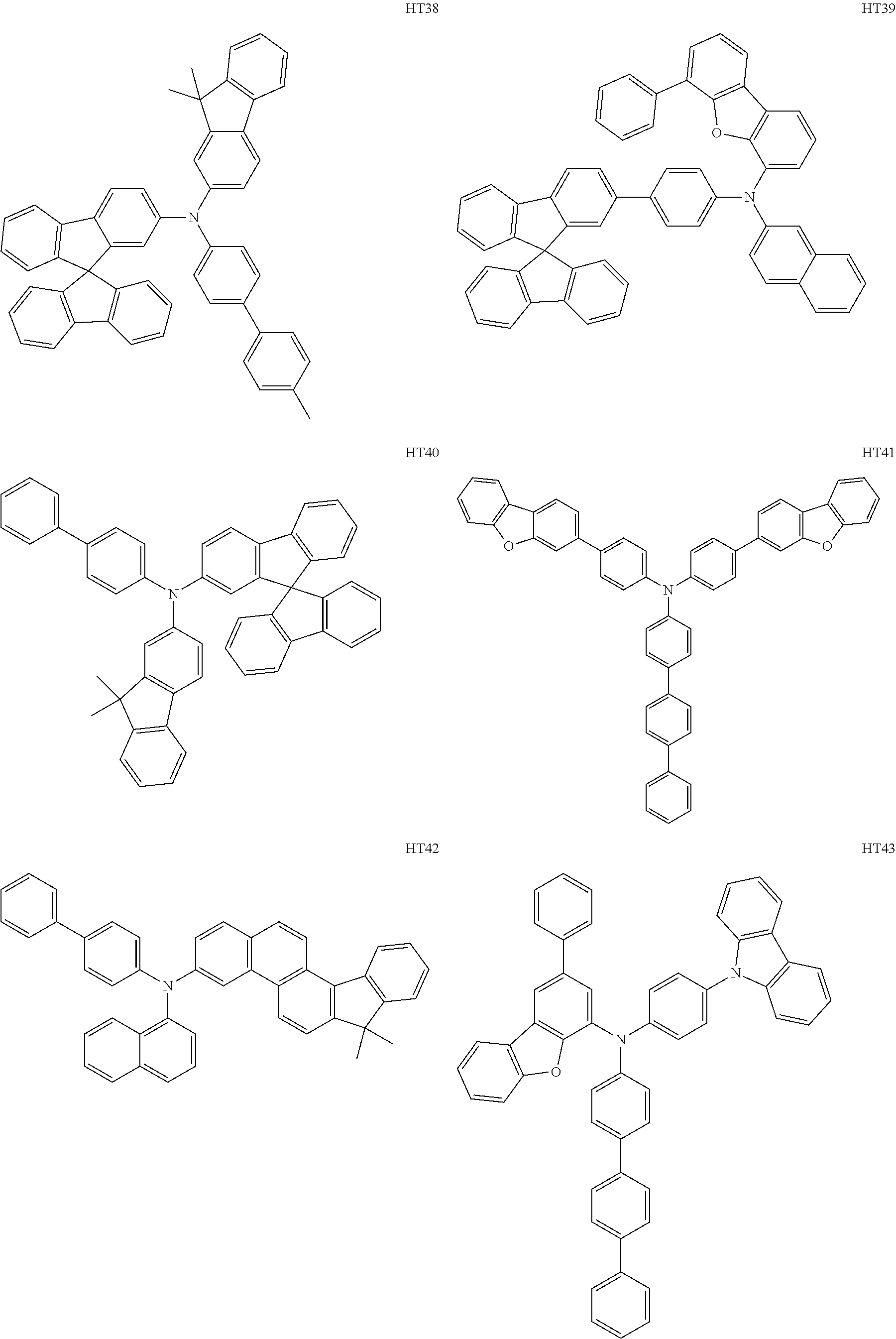

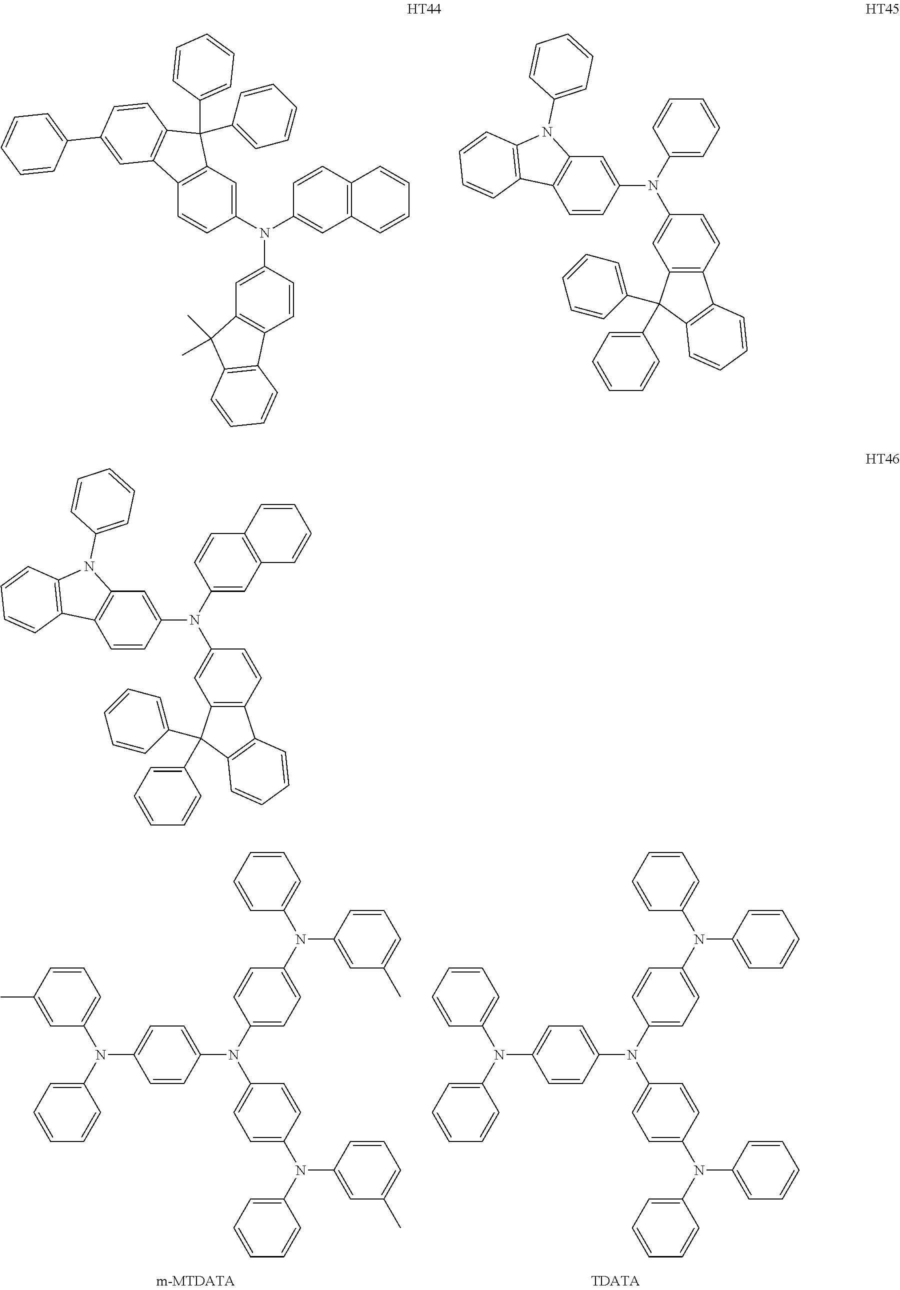

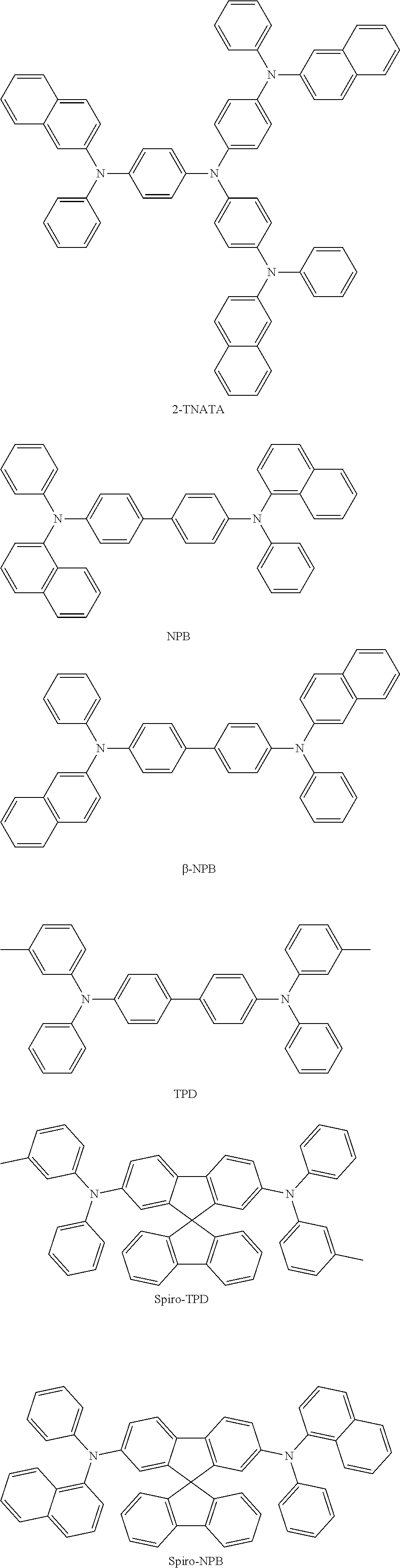

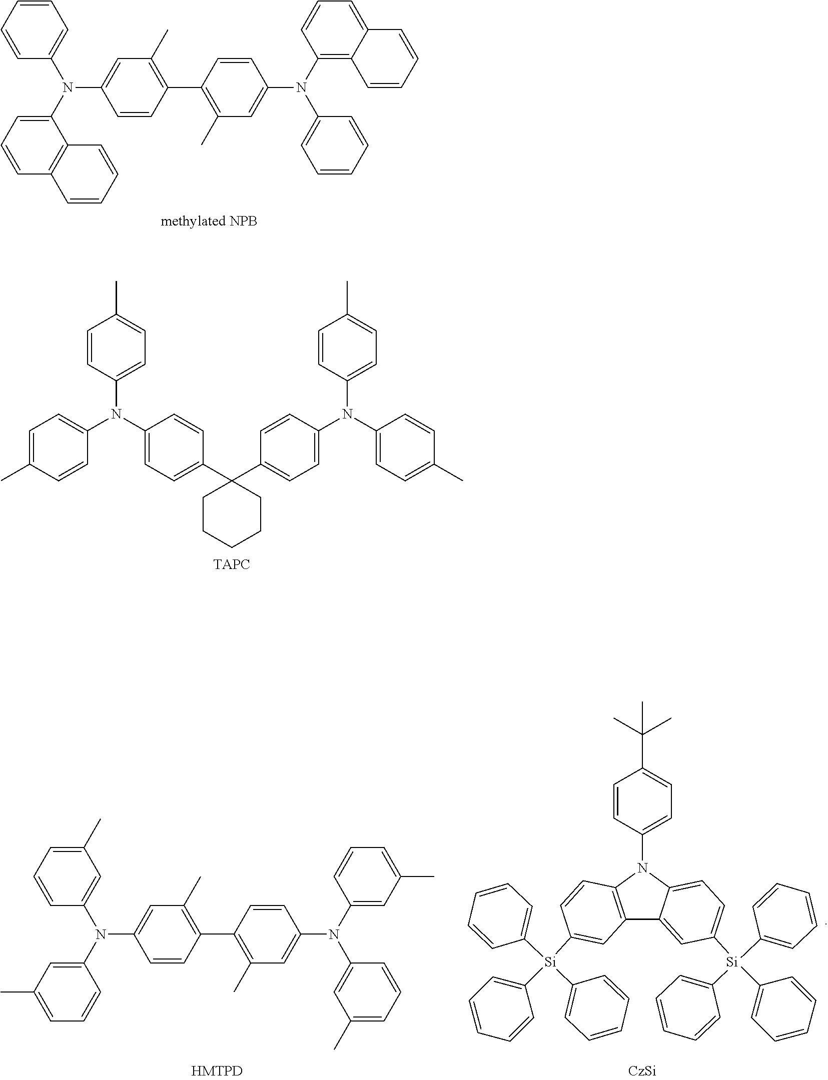

A thickness of the hole transport region may be in a range of about 50 Å to about 10,000 Å, for example, about 100 Å to about 4,000 Å. When the hole transport region includes a hole injection layer, a hole transport layer, and/or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole-transporting characteristics may be obtained without a substantial increase in driving voltage.

The emission auxiliary layer may increase light-emission efficiency by compensating for an optical resonance distance according to the wavelength of light emitted by an emission layer, and the electron-blocking layer may block the leakage of electrons from an emission layer to a hole transport region. Materials that may be included in the hole transport region may be included in the emission auxiliary layer and the electron-blocking layer.

[p-Dopant]

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

For example, the lowest unoccupied molecular orbital (LUMO) energy level of the p-dopant may be −3.5 eV or less.

In one or more embodiments, the p-dopant may include a quinone derivative, a cyano group-containing compound, a compound including element EL1 and element EL2, or any combination thereof.

Examples of the quinone derivative are TCNQ, F4-TCNQ, etc.

Examples of the cyano group-containing compound are HAT-CN, and a compound represented by Formula 221:

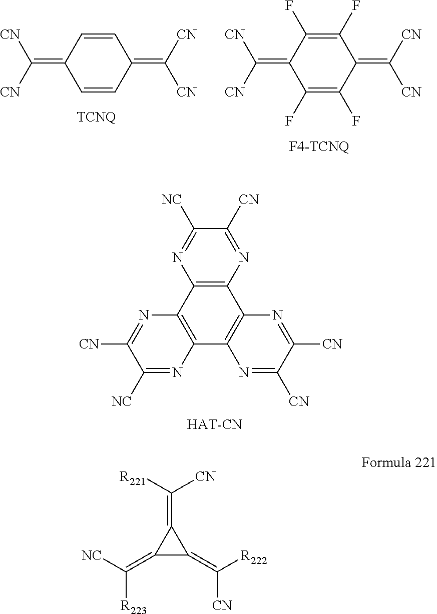

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with a cyano group; —F; —CI; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Examples of the metal are an alkali metal (for example, lithium (Li), sodium (Na), potassium (K), rubidium (Rb), cesium (Cs), etc.); alkaline earth metal (for example, beryllium (Be), magnesium (Mg), calcium (Ca), strontium (Sr), barium (Ba), etc.); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); post-transition metal (for example, zinc (Zn), indium (In), tin (Sn), etc.); and/or lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid are silicon (Si), antimony (Sb), and/or tellurium (Te).

Examples of the non-metal are oxygen (O) and/or halogens (for example, F, CI, Br, I, etc.).

Examples of the compound including element EL1 and element EL2 are metal oxide, metal halide (for example, metal fluoride, metal chloride, metal bromide, or metal iodide), metalloid halide (for example, metalloid fluoride, metalloid chloride, metalloid bromide, or metalloid iodide), metal telluride, or any combination thereof.

Examples of the metal oxide are tungsten oxide (for example, WO, W₂O₃, WO₂, WO₃, W₂O₅, etc.), vanadium oxide (for example, VO, V₂O₃, VO₂, V₂O₅, etc.), molybdenum oxide (for example, MoO, Mo₂O₃, MoO₂, MoO₃, Mo₂O₅, etc.), and rhenium oxide (for example, ReO₃, etc.).

Examples of the metal halide are alkali metal halide, alkaline earth metal halide, transition metal halide, post-transition metal halide, and/or lanthanide metal halide.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Examples of the alkaline earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgO₂, CaI₂, SrI₂, and/or BaI₂.

Examples of the transition metal halide are titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, CoI₂, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and/or gold halide (for example, AuF, AuCl, AuBr, Aul, etc.).

Examples of the post-transition metal halide are zinc halide (for example, ZnF₂, ZnCl₂, ZnBr₂, ZnI₂, etc.), indium halide (for example, InI₃, etc.), and/or tin halide (for example, SnI₂, etc.).

Examples of the lanthanide metal halide may include YbF, YbF₂, YbF₃, SmF₃, YbCl, YbCl₂, YbCl₃, SmCl₃, YbBr, YbBr₂, YbBr₃, SmBr3, Ybl, Ybl2, Ybl3, Sm13, and/or the like.

Examples of the metalloid halide are antimony halide (for example, SbCl₅ and the like) and/or the like.

Examples of the metal telluride are alkali metal telluride (for example, Li₂Te, Na₂Te, K₂Te, Rb₂Te, Cs₂Te, and the like), alkaline earth metal telluride (for example, BeTe, MgTe, CaTe, SrTe, BaTe, and the like), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and the like), post-transition metal telluride (for example, ZnTe, and the like), lanthanide metal telluride (for example, LaTe, CeTe, PrTe, NdTe, PmTe, EuTe, GdTe, TbTe, DyTe, HoTe, ErTe, TmTe, YbTe, LuTe, and the like), and/or the like.

### [Emission Layer in Interlayer 130]

When the light-emitting device 10 is a full-color light-emitting device, the emission layer may be patterned into a red emission layer, a green emission layer, and/or a blue emission layer, according to a sub-pixel. In one or more example embodiments, the emission layer may have a stacked structure of two or more layers of a red emission layer, a green emission layer, and a blue emission layer, in which the two or more layers contact each other or are separated from each other to emit white light. In one or more embodiments, the emission layer may include two or more materials of a red light-emitting material, a green light-emitting material, and a blue light-emitting material, in which the two or more materials are mixed with each other in a single layer to emit white light. For example, the emission layer may emit blue light.

In some example embodiments, the emission layer may include the amine-containing compound represented by Formula 1 as described in the specification.

The emission layer may include a host and a dopant.

In some example embodiments, the dopant include a phosphorescent dopant, a fluorescent dopant, or any combination thereof. The phosphorescent dopant, the fluorescent dopant, and the like that may be further included in the emission layer are each the same as described below.

The amount of the dopant in the emission layer may be from about 0.01 part by weight to about 15 parts by weight based on 100 parts by weight of the host.

In one or more example embodiments, the emission layer may include a quantum dot.

Meanwhile, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

### [Host]

The host may include, for example, a carbazole-containing compound, an anthracene-containing compound, or any combination thereof.

In some example embodiments, the host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5, when xb1 is 0 (L₃₀₁)_{xb1} may be a single bond,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each the same as described herein with respect to Q₁.

For example, when xb11 in Formula 301 is 2 or more, two or more of Ar₃₀₁(s) may be linked to each other via a single bond.

In one or more example embodiments, the host may include a compound represented by Formula 301-1, a compound represented by Formula 301-2, and/or any combination thereof:

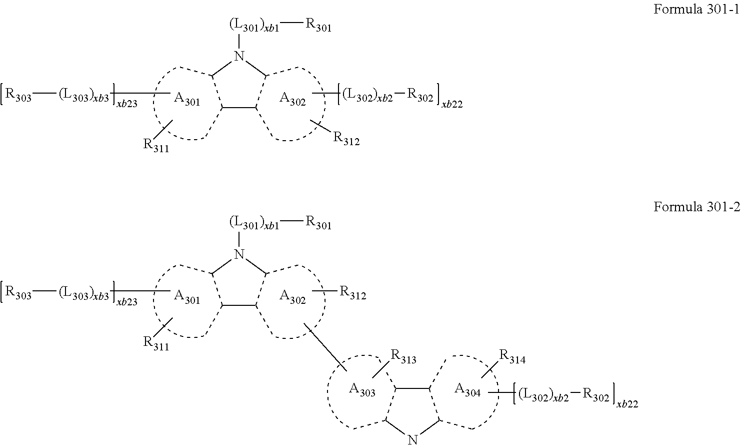

wherein, in Formulae 301-1 and 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described herein with respect to with L₃₀₁,
xb2 to xb4 may each independently be the same as described herein with respect to xb1, and
R₃₀₂ to R₃₀₅ and R₃₁₁ to R₃₁₄ may each be the same as described herein with respect to R₃₀₁.

In one or more example embodiments, the host may include an alkali earth metal complex, a post-transition metal complex, or any combination thereof. For example, the host may include a Be complex (for example, Compound H55), an Mg complex, a Zn complex, or any combination thereof.

In some example embodiments, the host may include: one of Compounds H1 to H128; 9,10-di(2-naphthyl)anthracene (ADN); 2-methyl-9,10-bis(naphthalen-2-yl)anthracene (MADN); 9,10-di(2-naphthyl)-2-t-butyl-anthracene (TBADN); 4,4′-bis(N-carbazolyl)-1,1′-biphenyl (CBP); 1,3-di-9-carbazolylbenzene (mCP); 1,3,5-tri(carbazol-9-yl)benzene (TCP); HTH; ETH and/or any combination thereof:

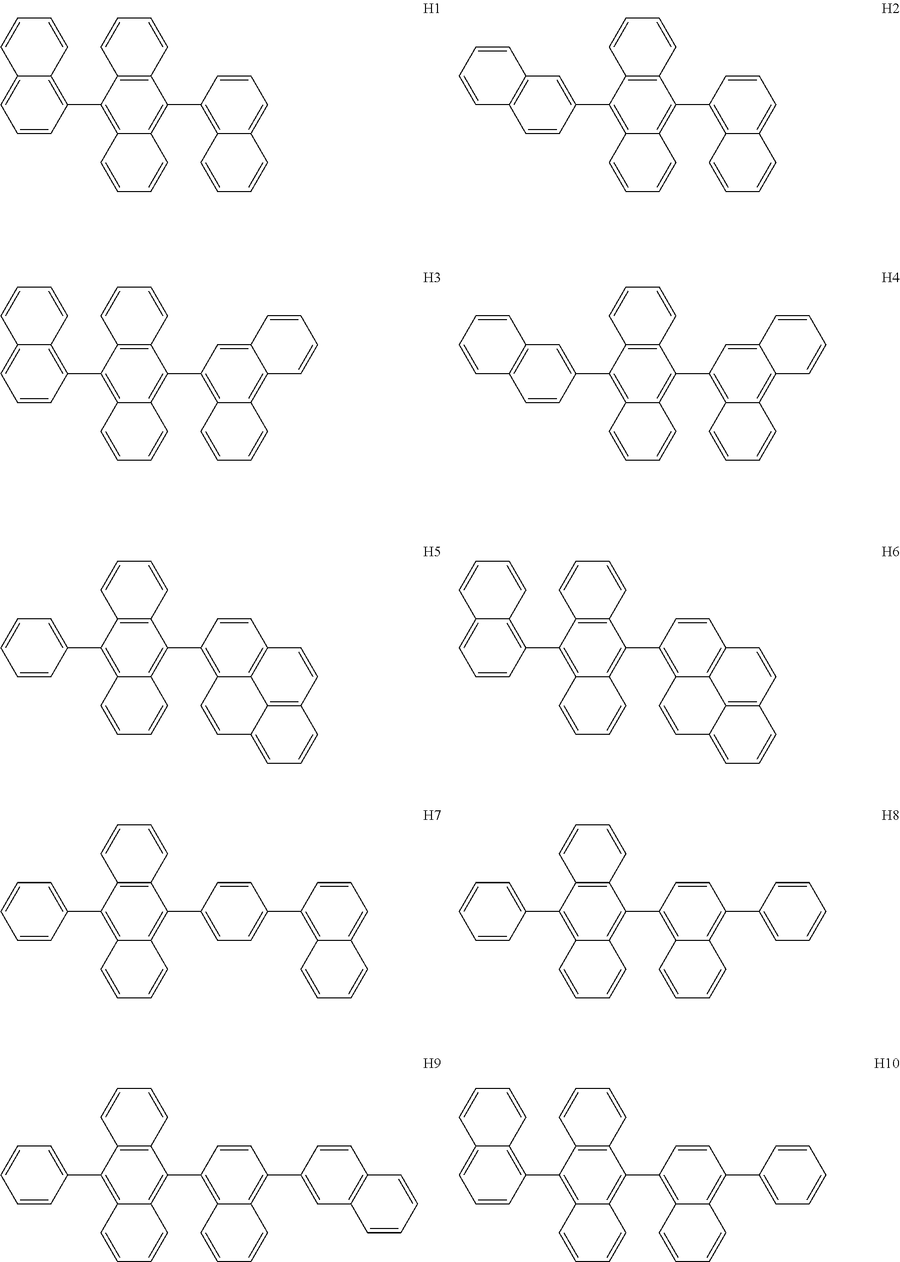

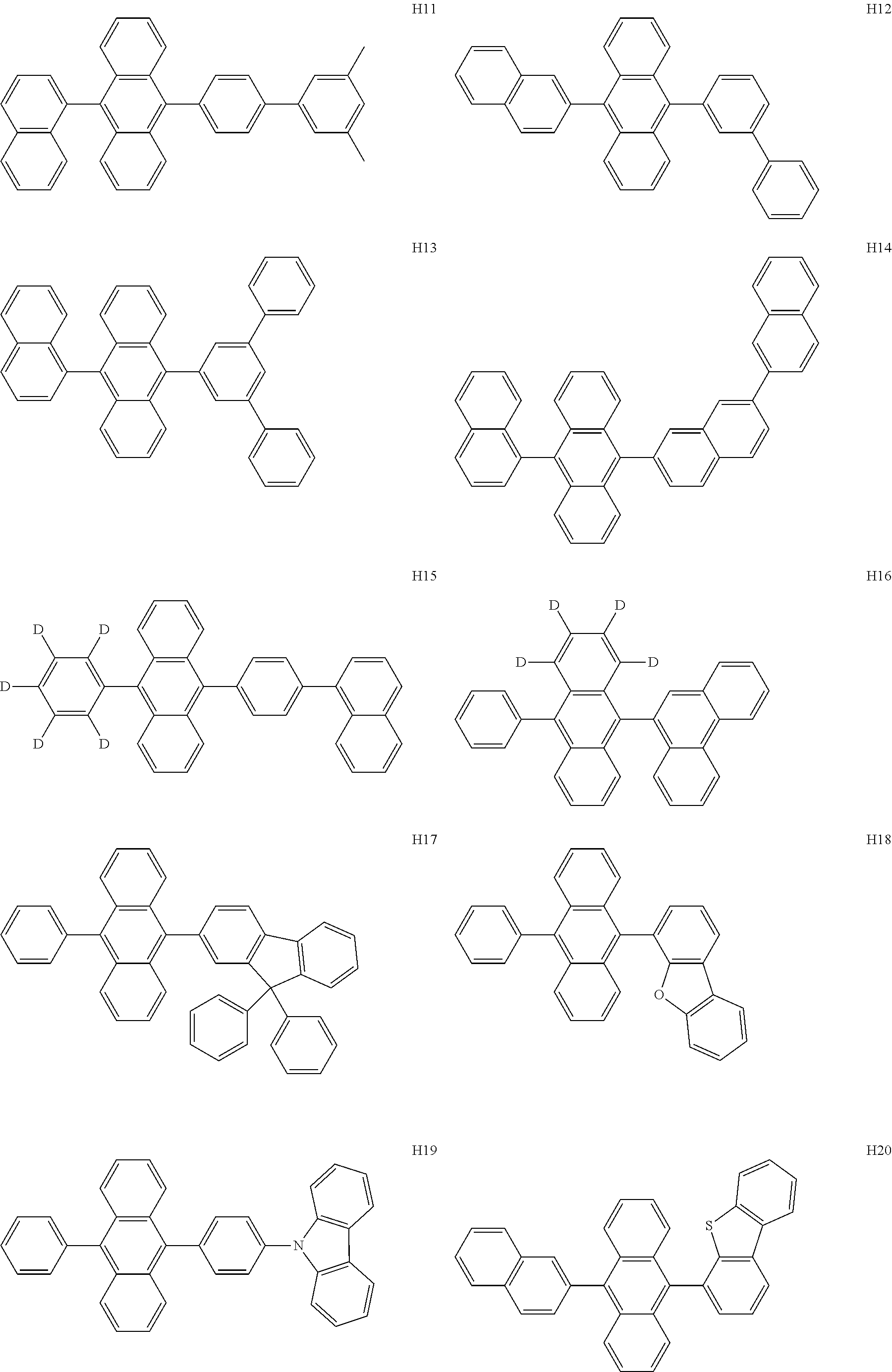

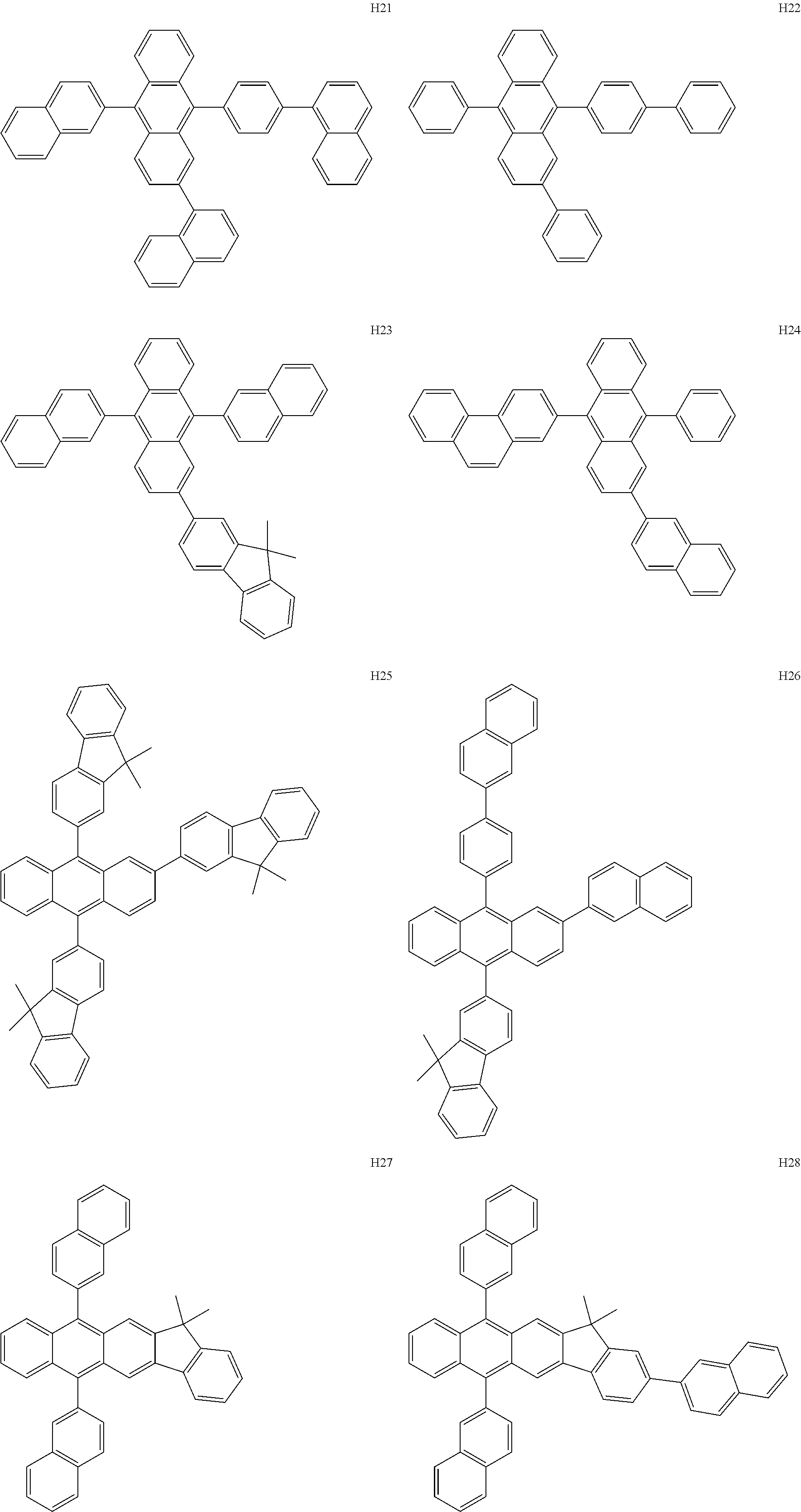

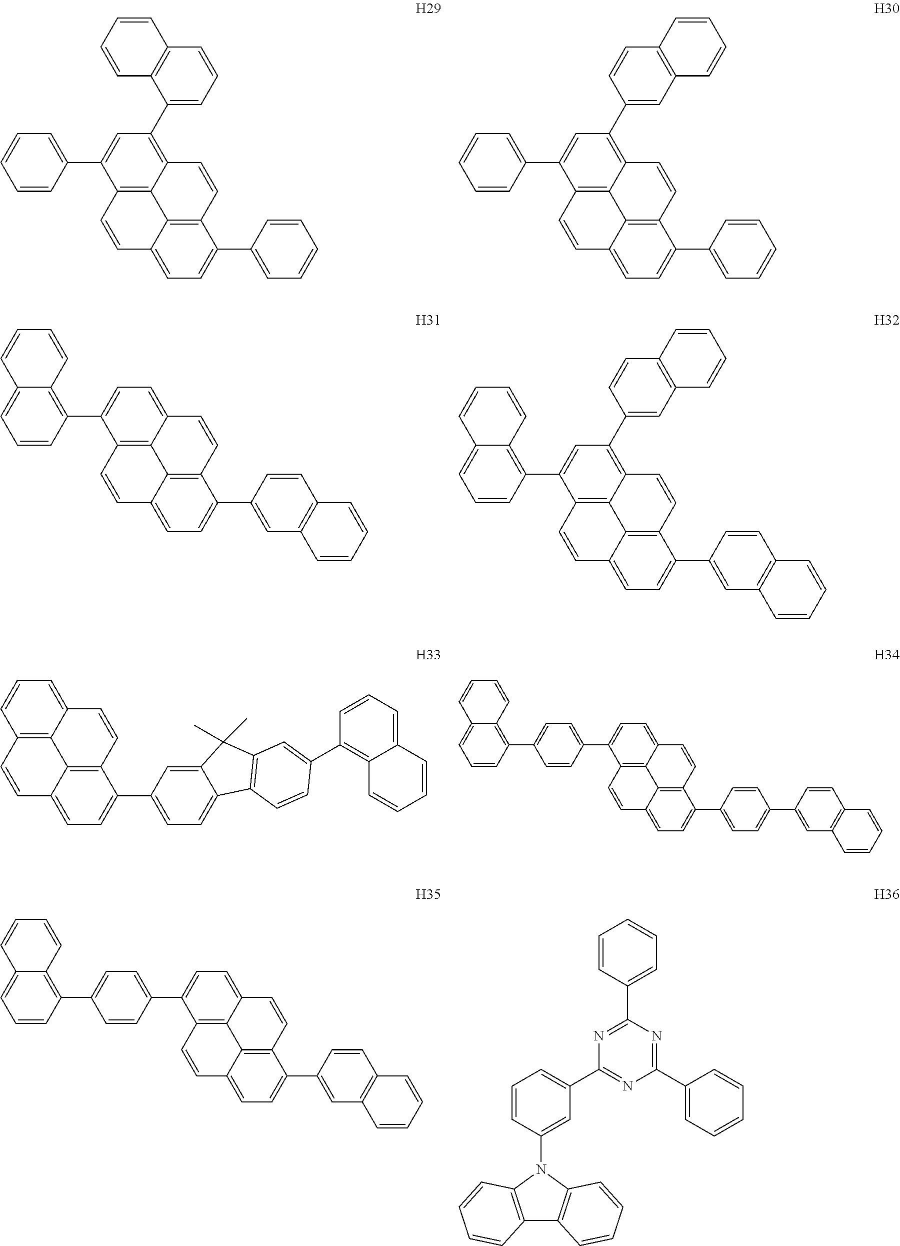

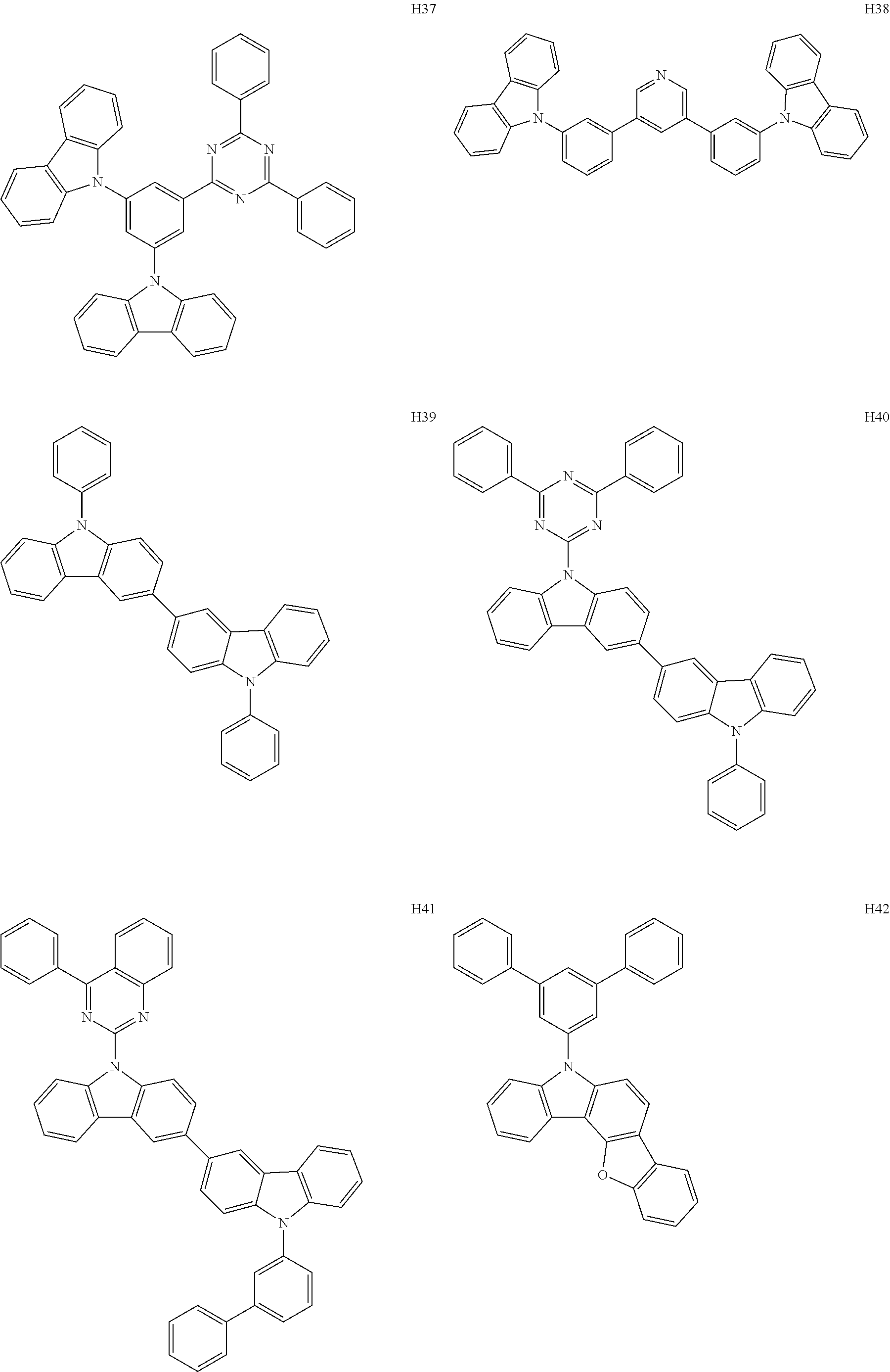 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

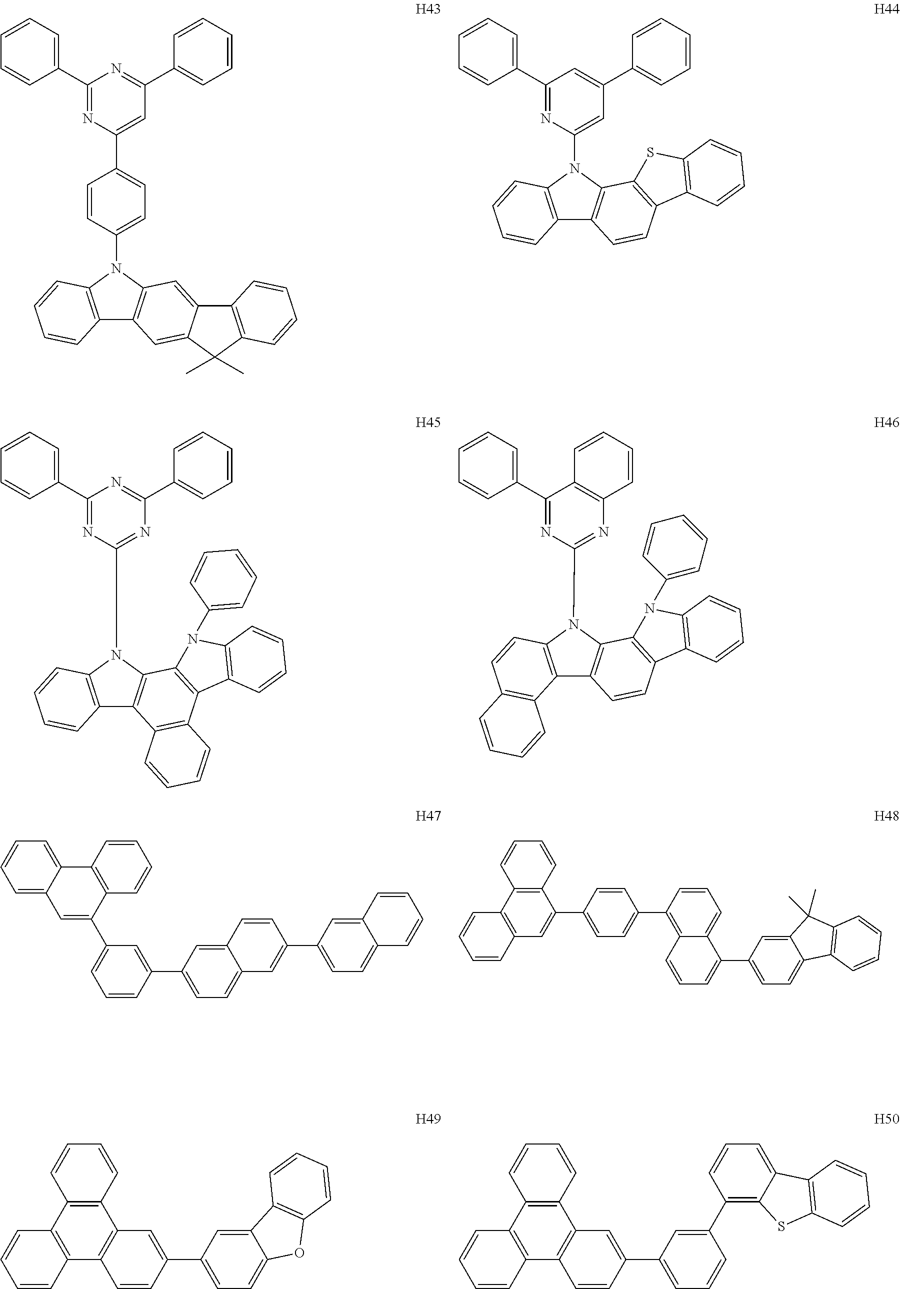

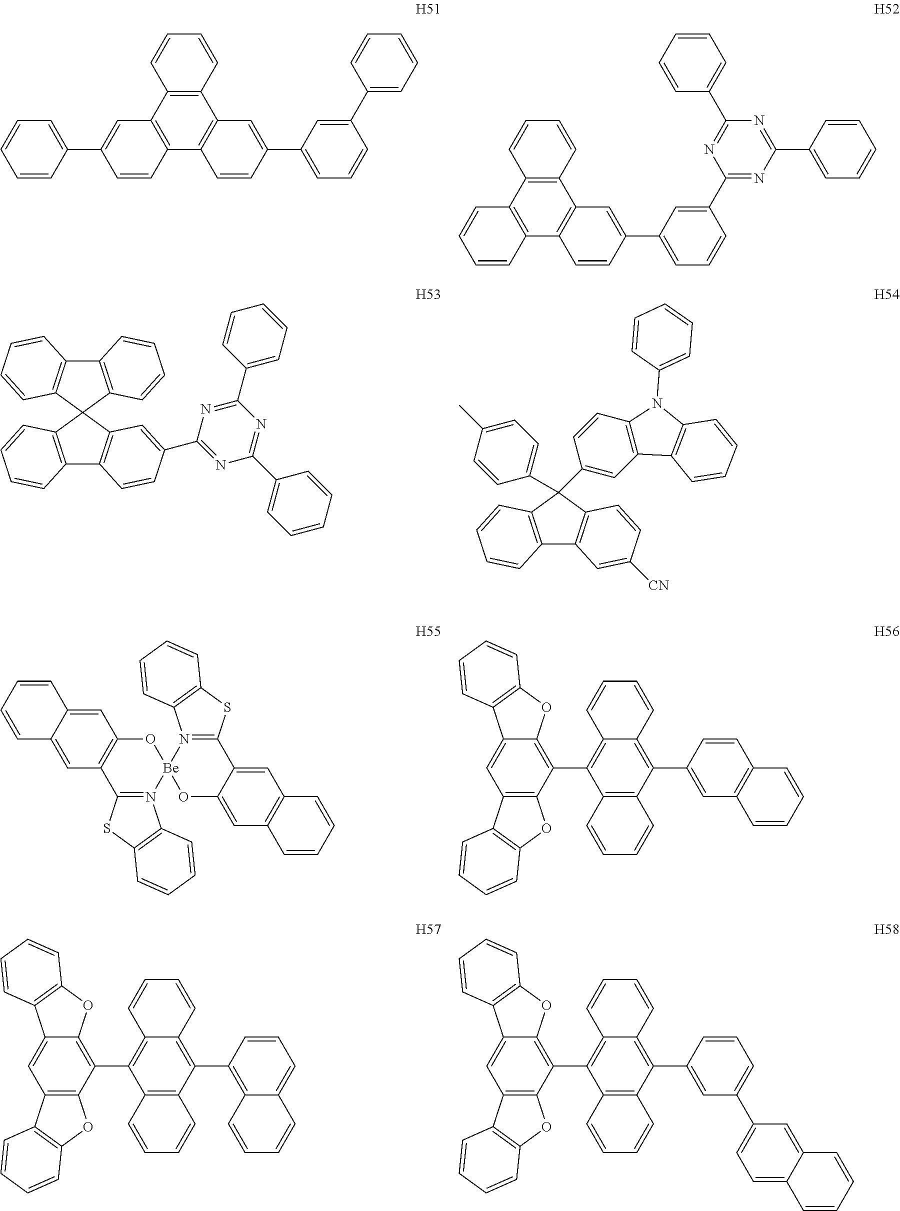

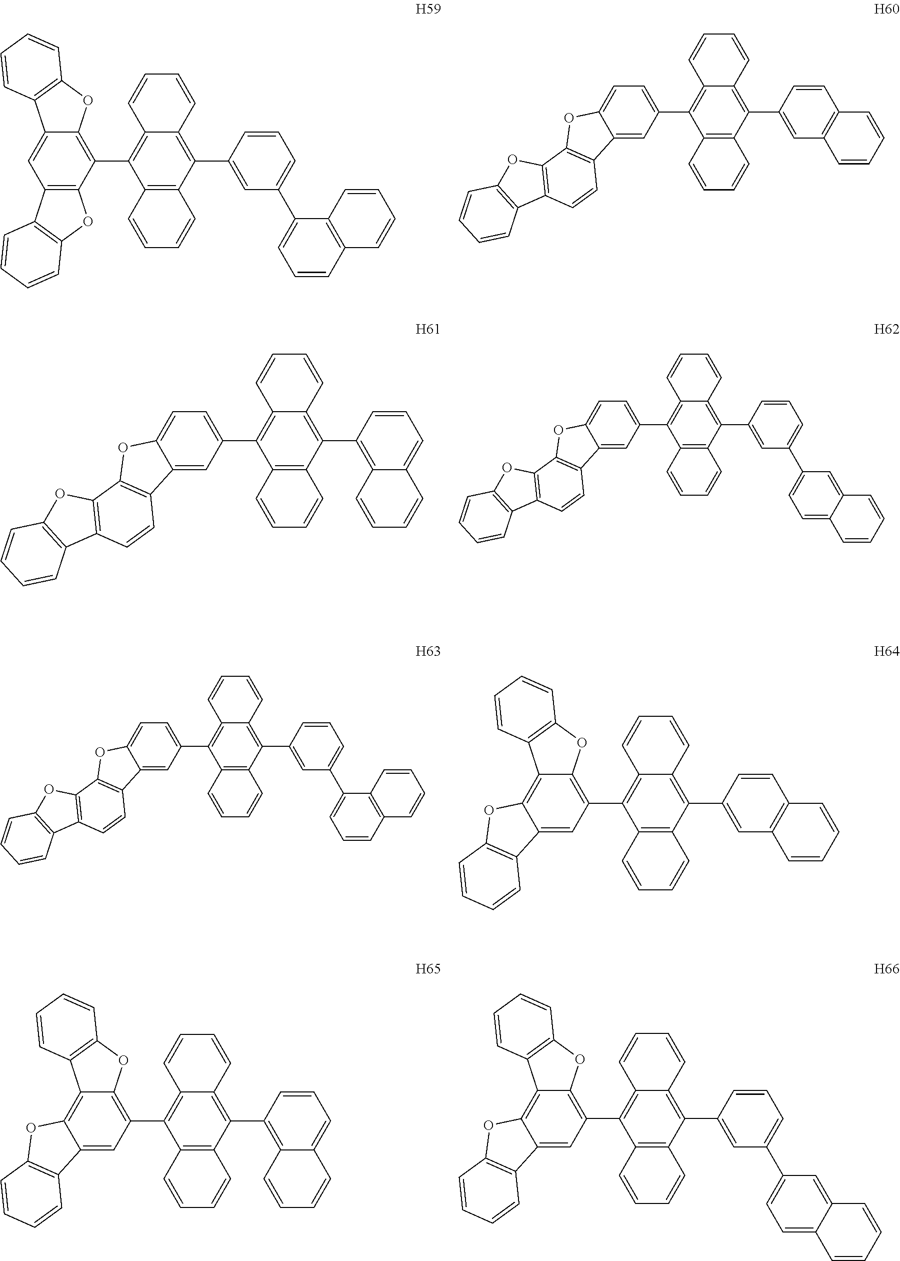

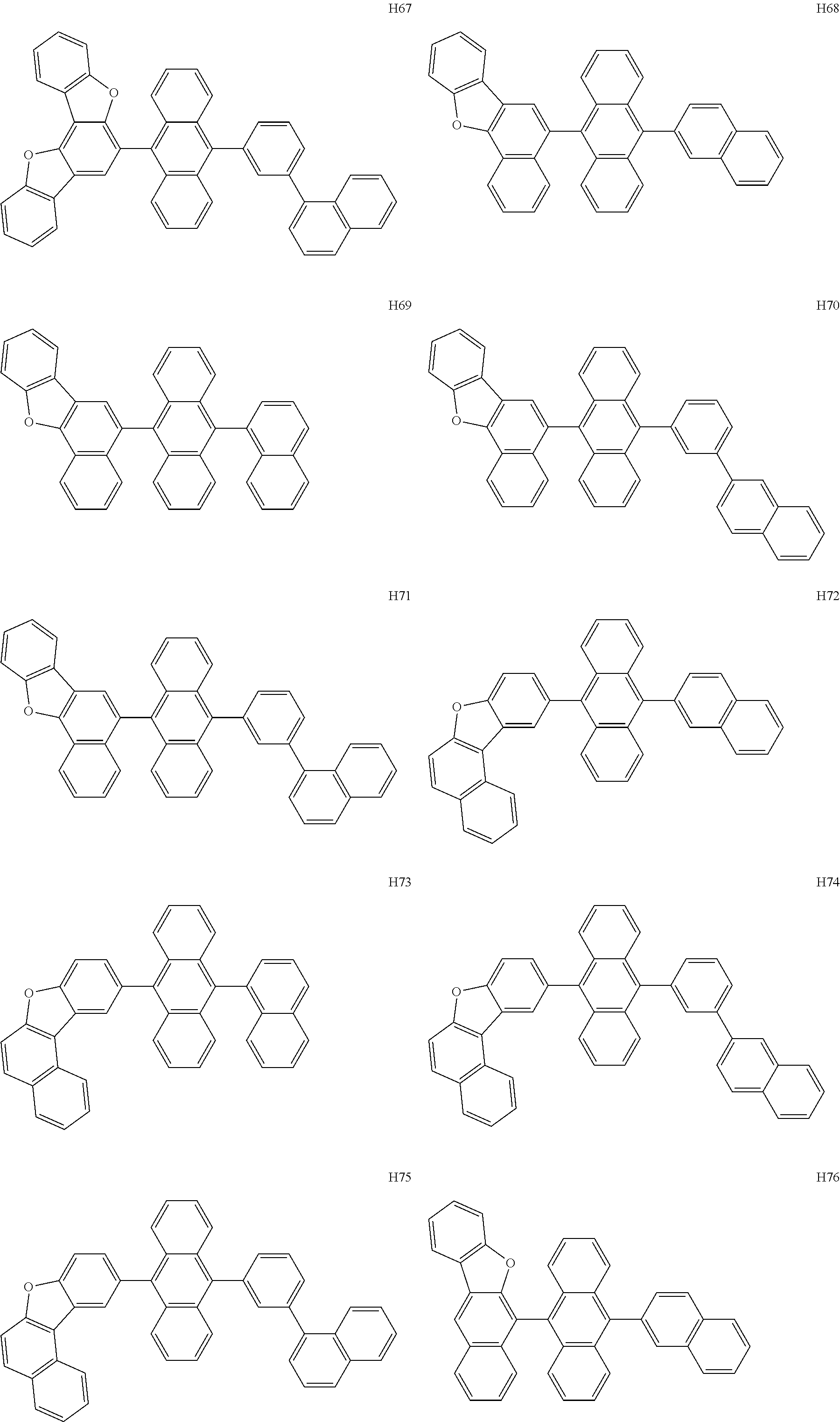

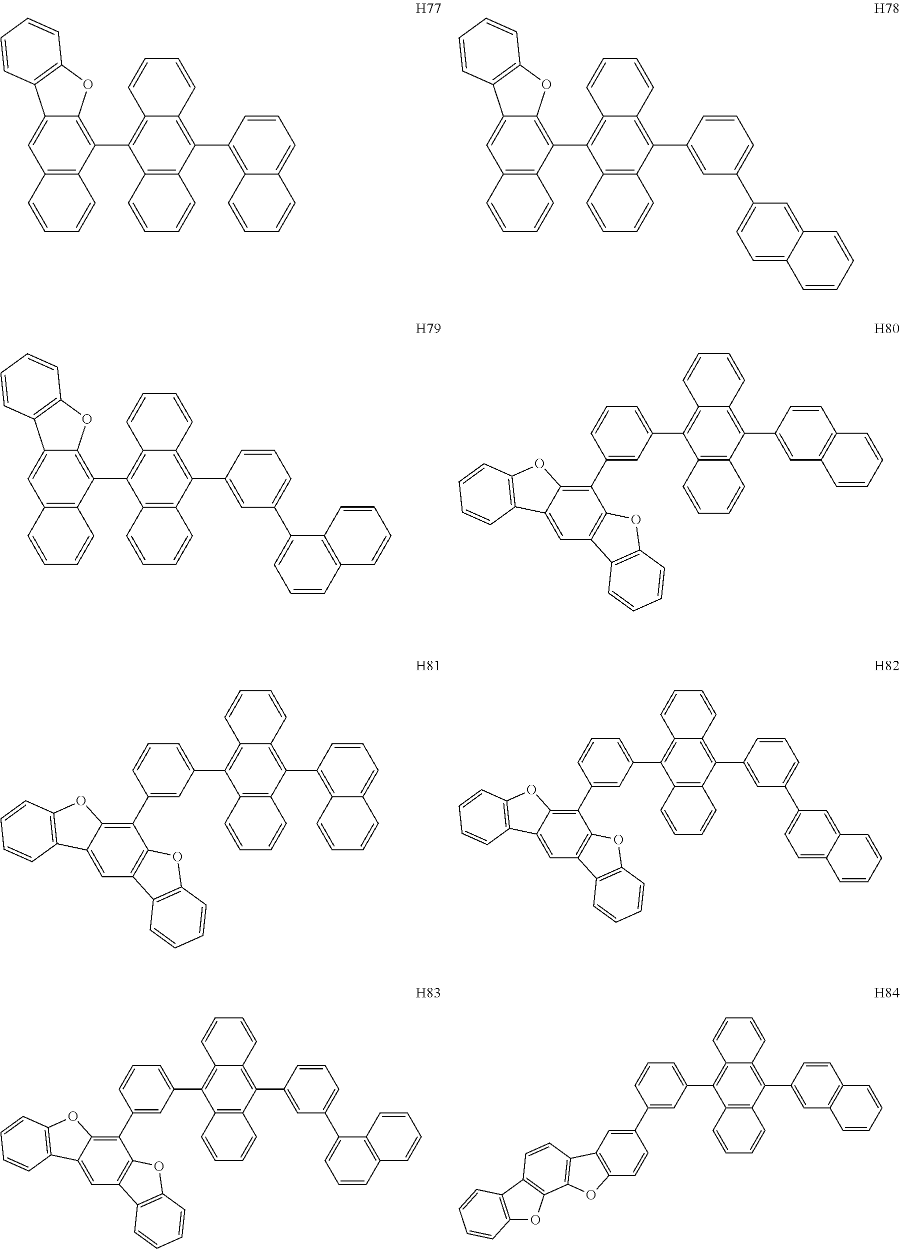

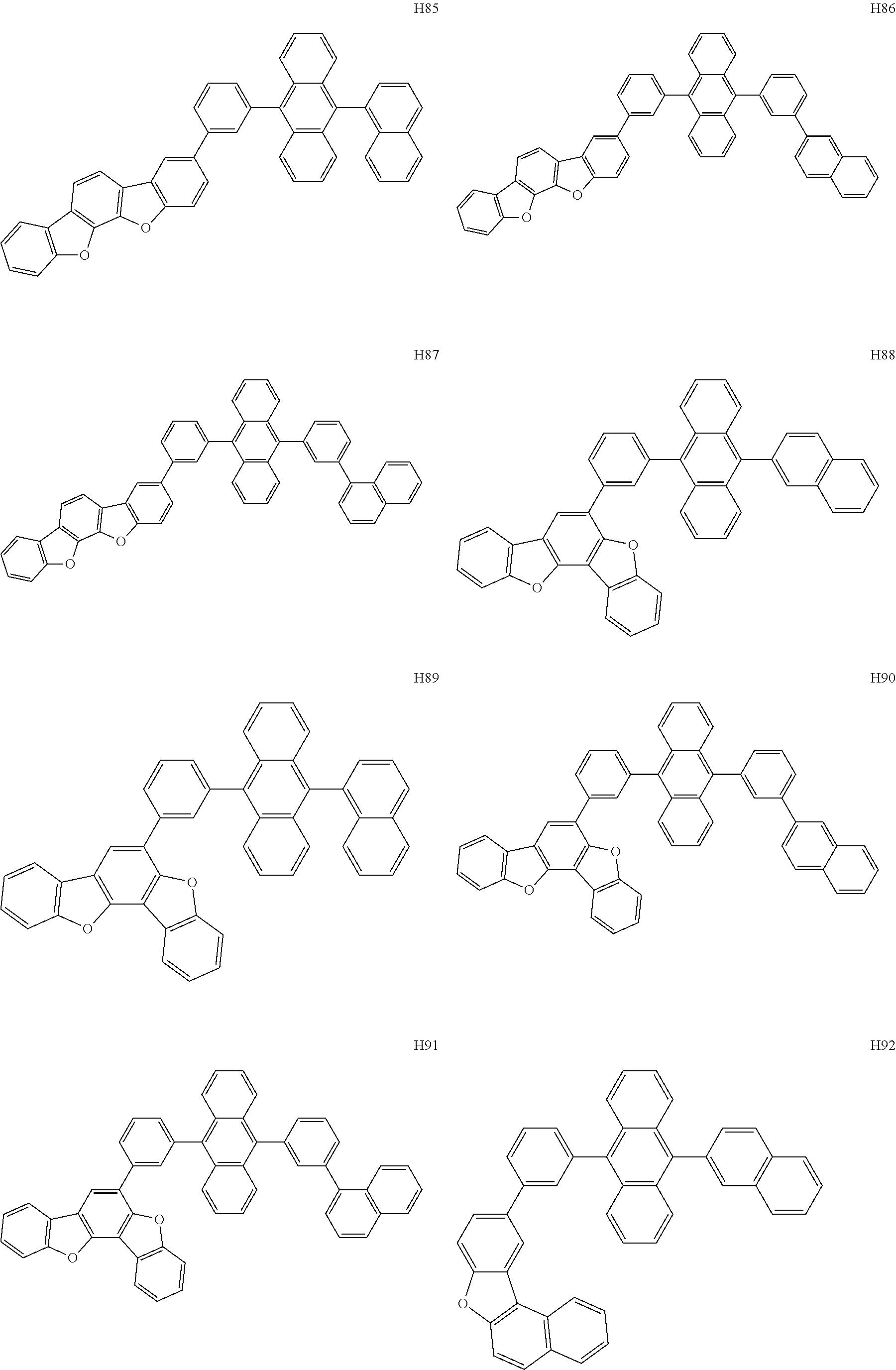

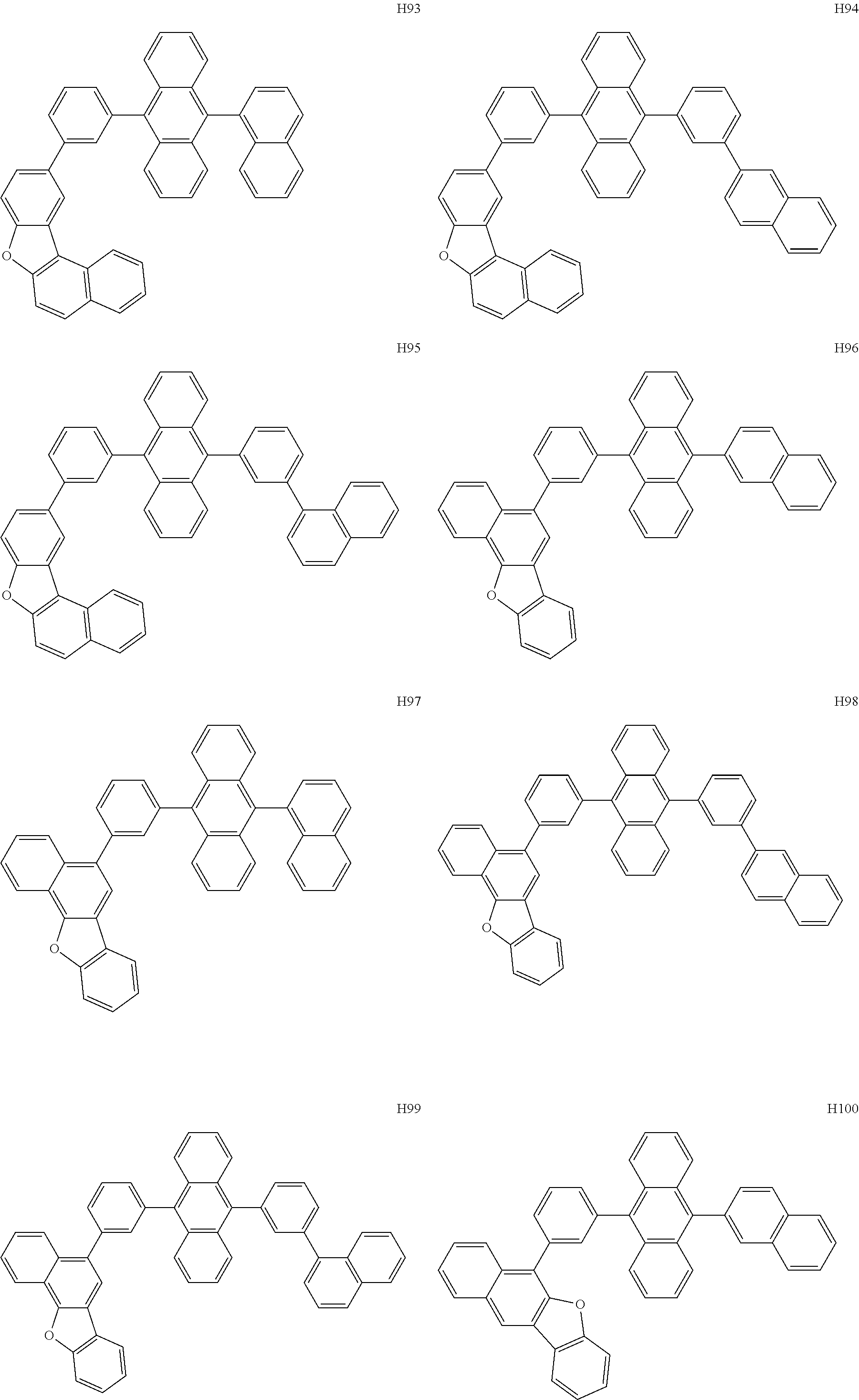

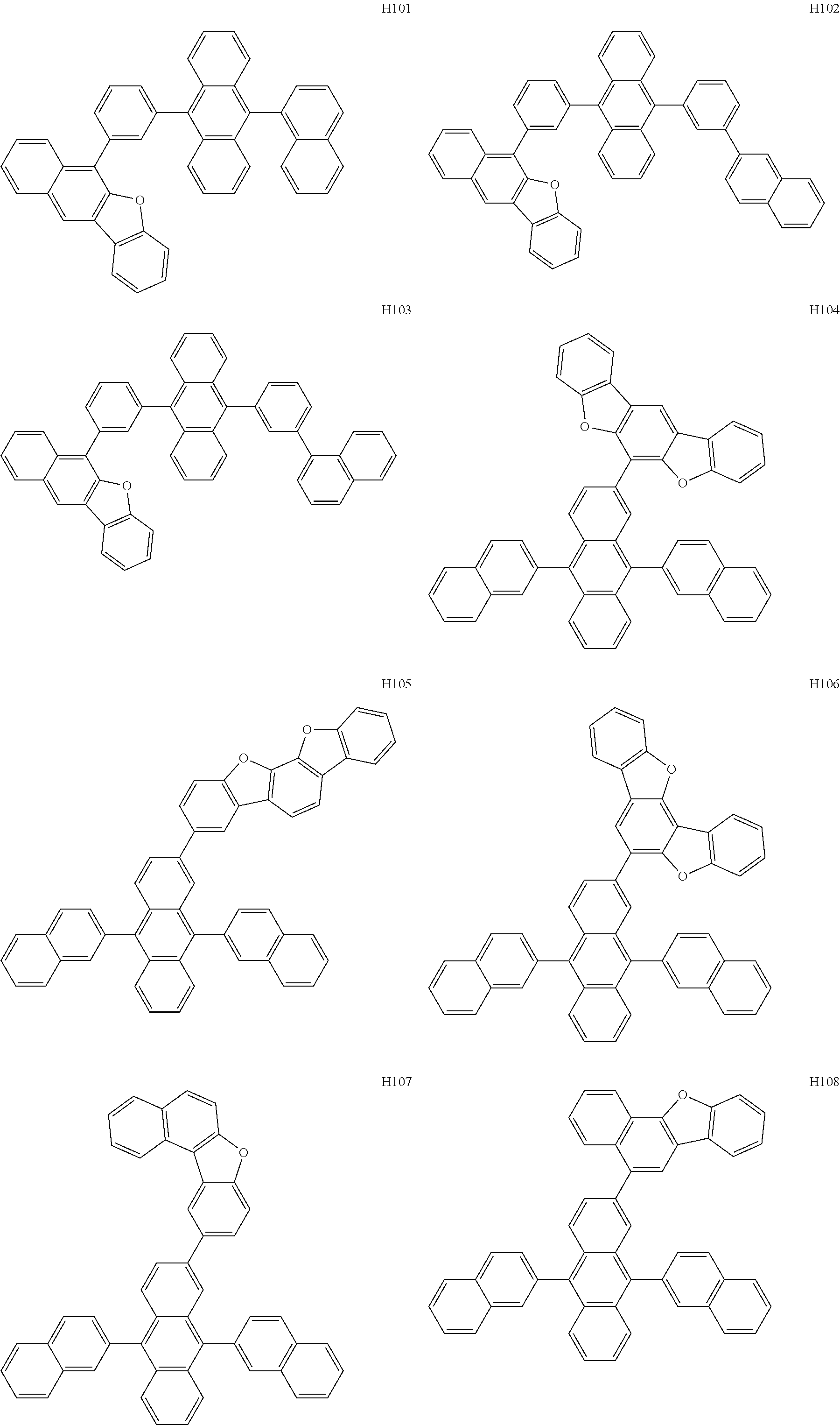

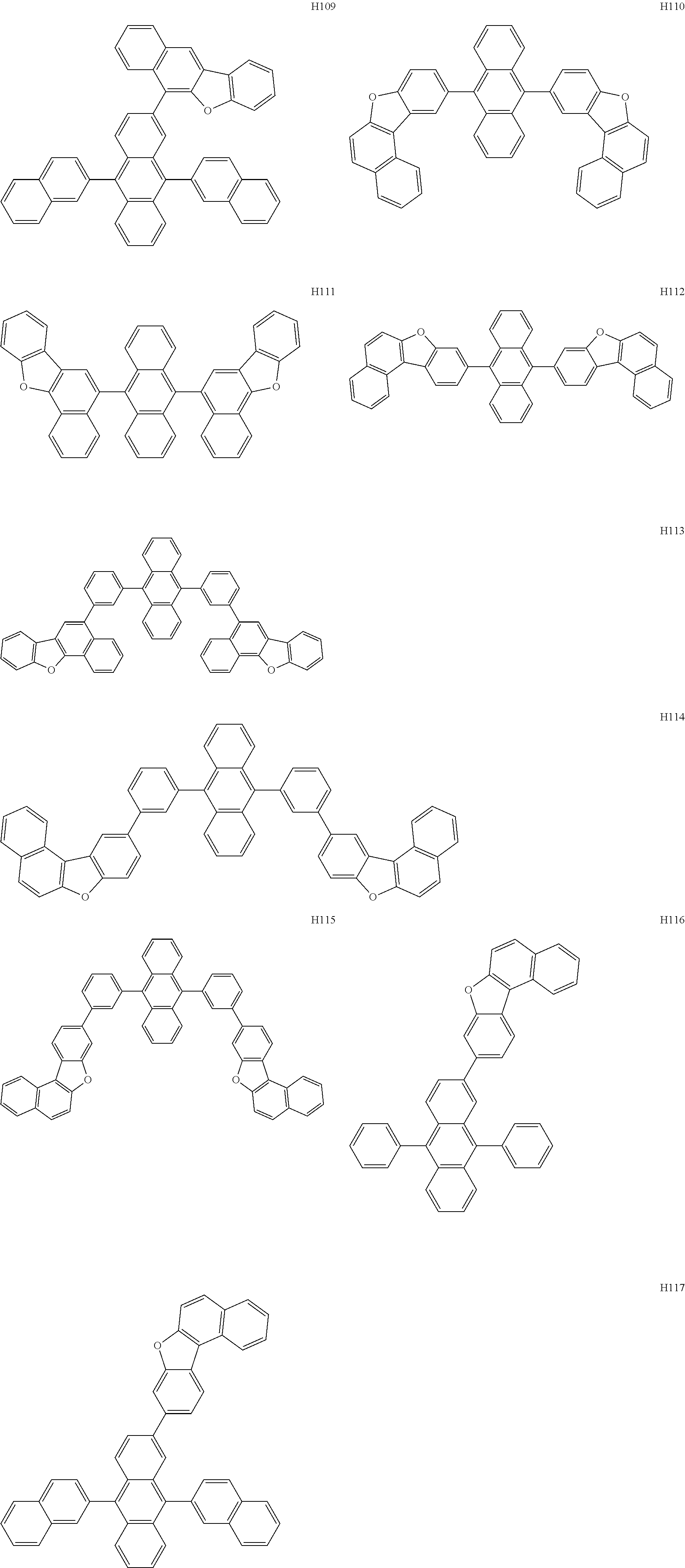

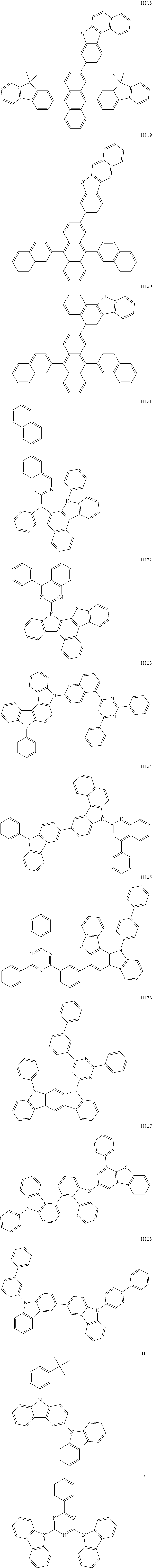

In some example embodiments, the host may include a first host compound and a second host compound.

In some example embodiments, the first host compound may be a hole-transporting host.

In some example embodiments, the second host compound may be an electron-transporting host.

In some example embodiments, the term “hole-transporting host” as used herein refers to a compound including a hole-transporting moiety.

In an embodiments, the term “electron-transporting host” as used herein refers to not only a compound including an electron-transporting moiety, but also a compound having bipolar properties.

The terms “hole-transporting host” and “electron-transporting host” may each be understood according to the relative difference between the hole mobility and electron mobility in the hole-transporting host and the electron-transporting host. For example, even when the electron transport host does not include an electron transport moiety, a bipolar compound exhibiting relatively higher electron mobility than the hole transport host may be understood as an electron transport host.

In some example embodiments, the hole-transporting host may be represented by one of Formulae 311-1 to 311-6, and the electron-transporting host may be represented by one of Formulae 312-1 to 312-4 and 313:

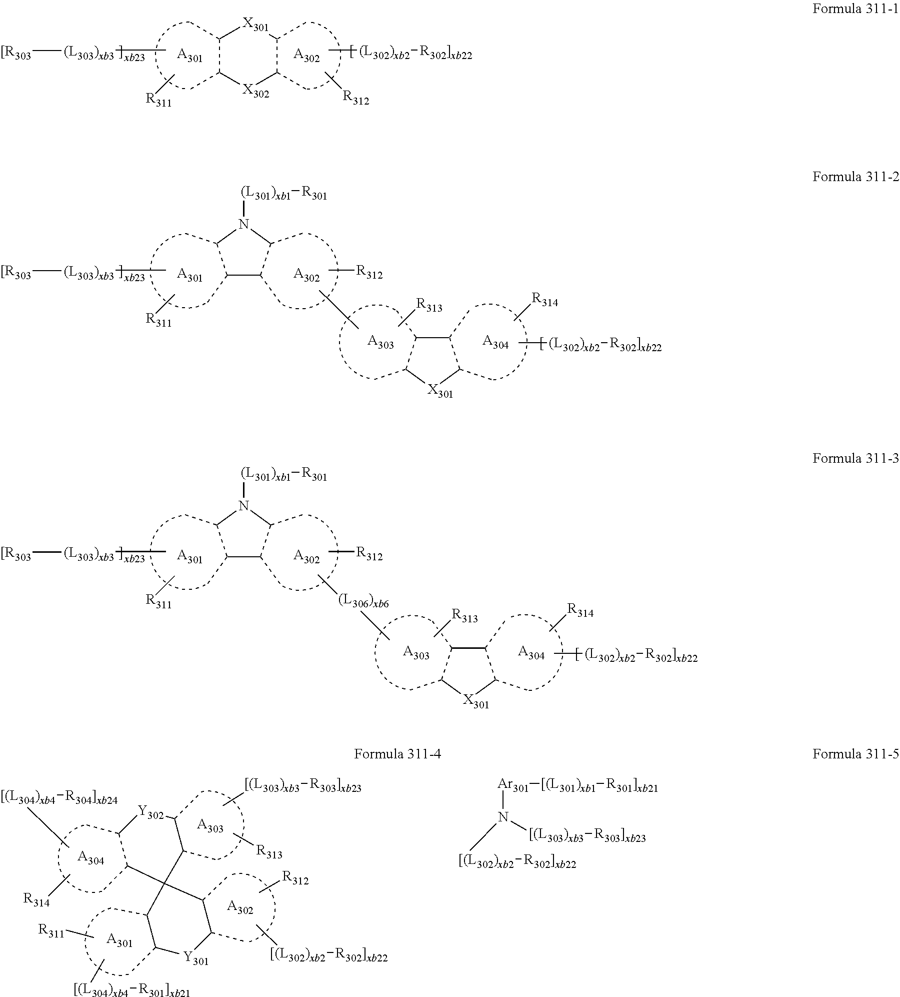

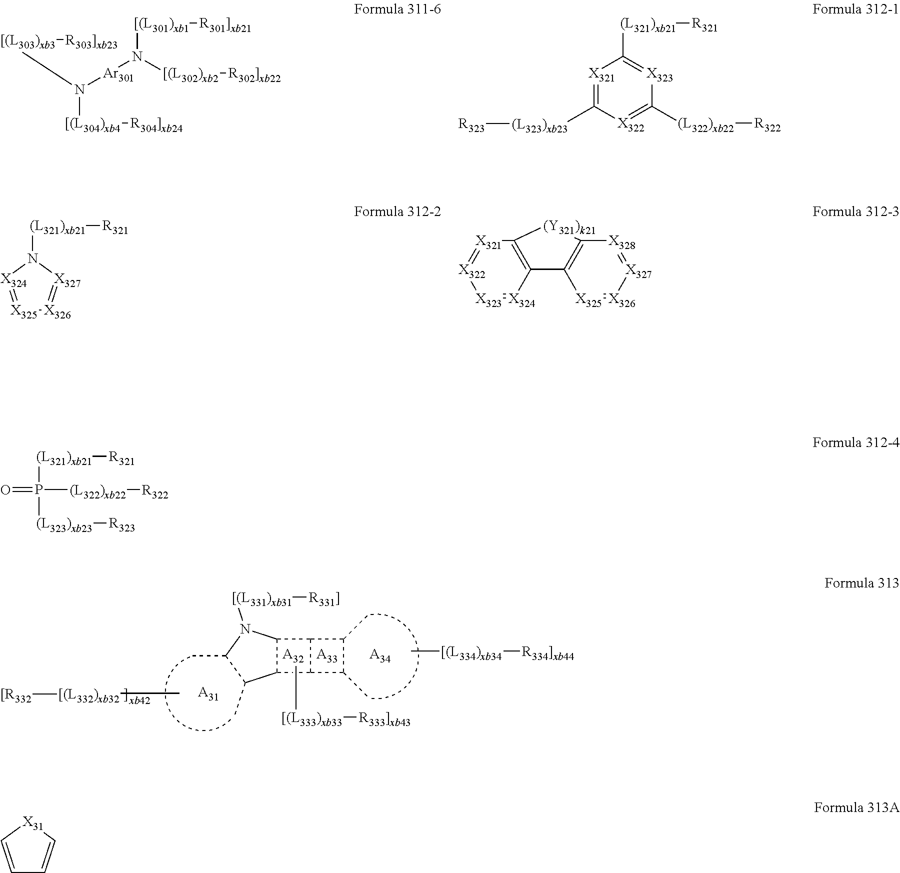

wherein, in Formulae 311-1 to 311-6, 312-1 to 312-4, 313, and 313A,
Ar₃₀₁ may be a C₃-C₆₀ carbocyclic group unsubstituted or substituted with at least one R₁₀ₐ or a C₁-C₆₀ heterocyclic group unsubstituted or substituted with at least one R₁₀ₐ,
Ar₃₀₁ to Ar₃₀₄ may each independently be a C₃-C₆₀ carbocyclic group or a C₁-C₆₀ heterocyclic group,
X₃₀₁ may be O, S, N[(L₃₀₄)_{xb4}-R₃₀₄], C[(L₃₀₄)_{xb4}-R₃₀₄][(L₃₀₅)_{xb5}-R₃₀₅], or Si[(L₃₀₄)_{xb4}-R₃₀₄][(L₃₀₅)_{xb5}-R₃₀₅],
X₃₀₂, Y₃₀₁, and Y₃₀₂ may each independently be a single bond, O, S, N[(L₃₀₅)_{xb5}-R₃₀₅], C[(L₃₀₄)_{xb4}-R₃₀₄][(L₃₀₅)_{xb5}-R₃₀₅], Si[(L₃₀₄)_{xb4}-R₃₀₄][(L₃₀₅)_{xb5}-R₃₀₅], or S(═O)₂,
xb1 to xb5 may respectively be 0, 1, 2, 3, 4, or 5, when xb1 is 0, *-(L₃₀₁)_{xb1}-*′ may be a single bond, when xb2 is 0, *-(L₃₀₂)_{xb2}-*′ may be a single bond, when xb3 is 0, *-(L₃₀₃)_{xb3}—*′ may be a single bond, when xb4 is 0, *-(L₃₀₄)_{xb4}-*′ may be a single bond, and when xb5 is 0, *-(L₃₀₅)_{xb5} may be a single bond,
xb6 may be 1, 2, 3, 4, or 5,
X₃₂₁ to X₃₂₈ may each independently be N or C[(L₃₂₄)_{xb24}-R_{324],}
Y₃₂₁ may be *—O—*′, *—S—*′, *—N[(L₃₂₅)_{xb25}-R₃₂₅]—*′, *—C[(L₃₂₅)_{xb25}-R₃₂₅][(L₃₂₆)_{xb26}-R₃₂₆]—*′, *—C[(L₃₂₅)_{xb25}-R₃₂₅]═C[(L₃₂₆)_{xb26}-R₃₂₆]*′, *—C[(L₃₂₅)_{xb25}-R₃₂₅]═N—*′, or *—N═C[(L₃₂₆)_{xb26}-R₃₂₆]—*′,
k21 may be 0, 1, or 2, wherein, when k21 is 0, Y321 may not be present,
xb21 to xb26 may each independently be 0, 1, 2, 3, 4, or 5,
A₃₁, A₃₂, and A₃₄ may each independently be a C₃-C₆₀ carbocyclic group or a C₁-C₃₀ heterocyclic group,
A₃₃ may be a group represented by Formula 313A,
X₃₁ may be N[(L₃₃₅)_{xb35}-(R₃₃₅)], O, S, Se, C[(L₃₃₅)_{xb35}-(R₃₃₅)][(L₃₃₆)_{xb36}-(R₃₃₆)], or Si[(L₃₃₅)_{xb35}-(R₃₃₅)][(L₃₃₆)_{xb36}-(R₃₃₆)],
xb31 to xb36 may each independently be 0, 1, 2, 3, 4, or 5, when xb31 is 0, *-(L₃₃₁)_{xb31}—*′ may be a single bond, when xb32 is 0, *-(L₃₃₂)_{xb32}-*′ may be a single bond, when xb33 is 0, *-(L₃₃₃)_{xb33}-*′ may be a single bond, when xb34 is 0, *-(L₃₃₄)_{xb34}-*′ may be a single bond, when xb35 is 0, *-(L₃₃₅)_{xb35}*′ may be a single bond, and when xb36 is a0, *-(L₃₃₆)_{xb36}-* may be a single bond.
xb42 to xb44 may each independently be 0, 1, 2, 3, 4, 5, 6, 7, 8, 9, or 10,
L₃₀₁ to L₃₀₆, L₃₂₁ to L₃₂₆, and L₃₃₁ to L₃₃₆ may each independently be a single bond, a C₂-C₂₀ alkylene group unsubstituted or substituted with at least one R₁₀ₐ, a C₂-C₂₀ alkenylene group unsubstituted or substituted with at least one R₁₀ₐ, a C₁-C₂₀ alkynylene group unsubstituted or substituted with at least one R₁₀ₐ, a C₃-C₁₀ cycloalkylene group unsubstituted or substituted with at least one R₁₀ₐ, a C₁-C₁₀ heterocycloalkylene group unsubstituted or substituted with at least one R₁₀ₐ, a C₃-C₁₀ cycloalkenylene group unsubstituted or substituted with at least one R₁₀ₐ, a C₁-C₁₀ heterocycloalkenylene group unsubstituted or substituted with at least one R₁₀ₐ, a C₆-C₆₀ arylene group unsubstituted or substituted with at least one R₁₀ₐ, a C₁-C₆₀ heteroarylene group unsubstituted or substituted with at least one R₁₀ₐ, a divalent non-aromatic condensed polycyclic group unsubstituted or substituted with at least one R₁₀ₐ, or a divalent non-aromatic condensed heteropolycyclic group unsubstituted or substituted with at least one R₁₀ₐ,
R₃₀₁ to R₃₀₅, R₃₁₁ to R₃₁₄, R₃₂₁ to R₃₂₆, and R₃₃₁ to R₃₃₆ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
two or more substituents of R₃₂₁ to R₃₂₆ are optionally bonded together to form a C₃-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇—C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be: hydrogen; deuterium; —F; —CI; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof.

In some example embodiments, the first host compound and the second host compound may form an exciplex.

### [Phosphorescent Dopant]

In one or more example embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, and/or any combination thereof.

The phosphorescent dopant may be electrically neutral.

For example, the phosphorescent dopant may include an organometallic compound represented by Formula 401:

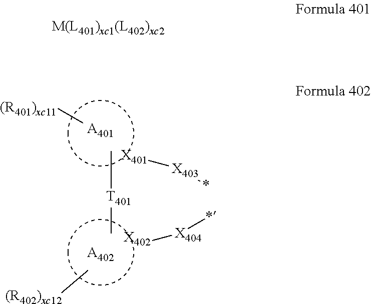

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein when xc1 is two or more, two or more of L₄₀₁(s) may be identical to or different from each other,
L₄₀₂ may be an organic ligand, and xc2 may be 0, 1, 2, 3, or 4, and when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)—*′, *—C(Q₄₁₁)(Q₄₁₂)-*′, *—C(Q₄₁₁)═C(Q₄₁₂)*′, *—C(Q₄₁₁)═*′, or *═C═*′,
X₄₀₃ and X₄₀₄ may each independently be a chemical bond (for example, a covalent bond or a coordination bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ may each be the same as described herein with respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be the same as described herein with respect to Q₁,
xc11 and xc12 may each independently be an integer from 0 to 10, and
* and *′ in Formula 402 each indicates a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more example embodiments, when xc1 in Formula 401 is 2 or more, two ring A₄₀₁(s) in two or more of L₄₀₁(s) may be optionally linked to each other via T₄₀₂, which is a linking group, or two ring A₄₀₂(s) may be optionally linked to each other via T₄₀₃, which is a linking group (see Compounds PD1 to PD4 and PD7). T₄₀₂ and T₄₀₃ may each be the same as described herein with respect to T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus-containing group (for example, a phosphine group, a phosphite group, etc.), or any combination thereof.

The phosphorescent dopant may include, for example, one of Compounds PD1 to PD40 or any combination thereof:

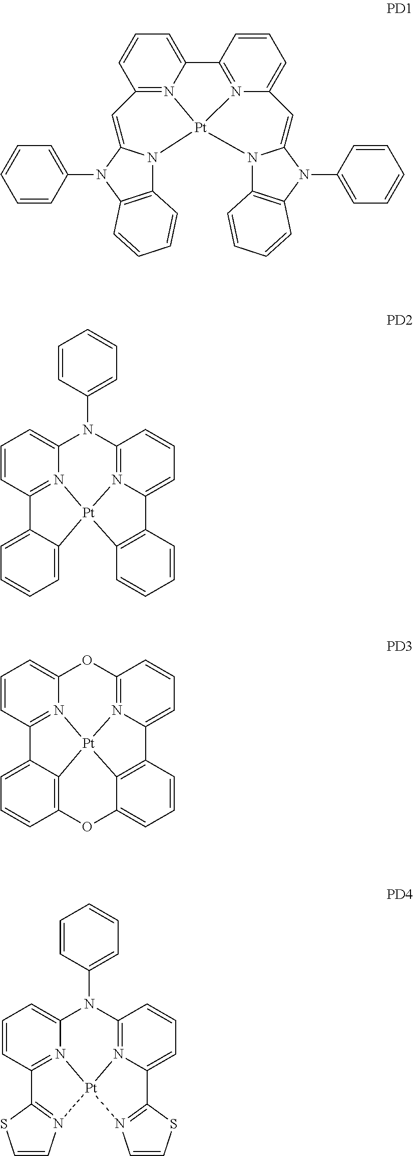

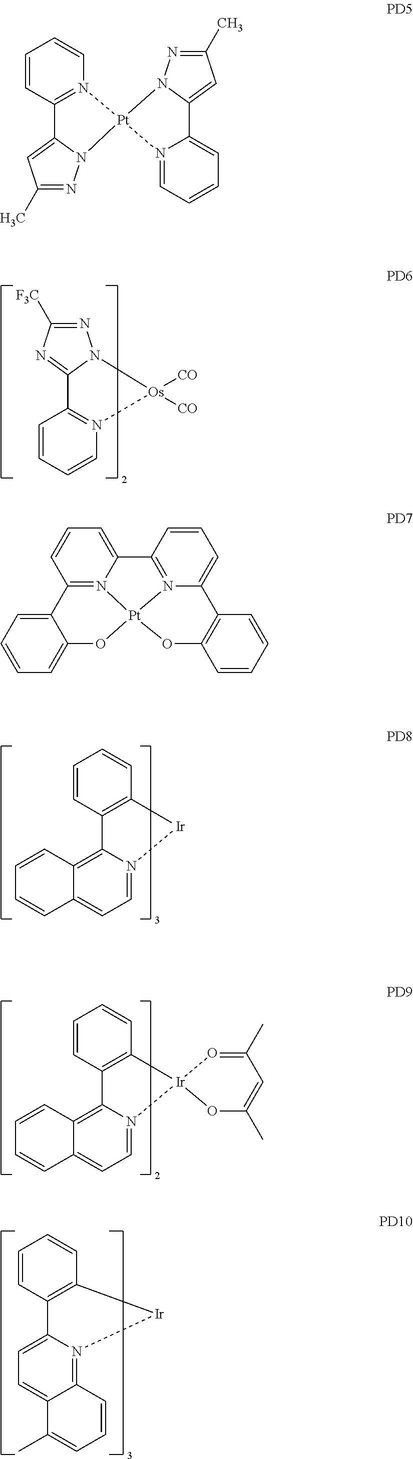

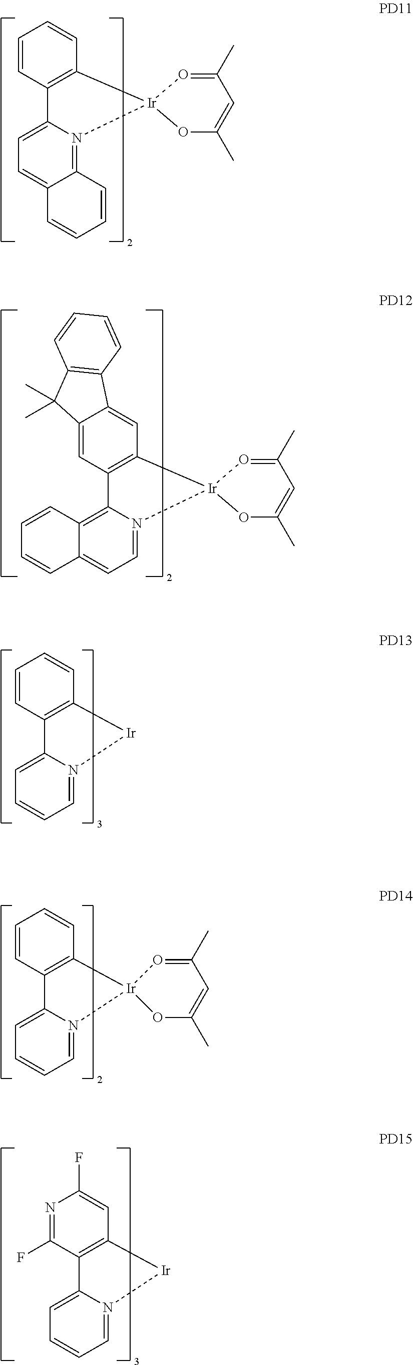

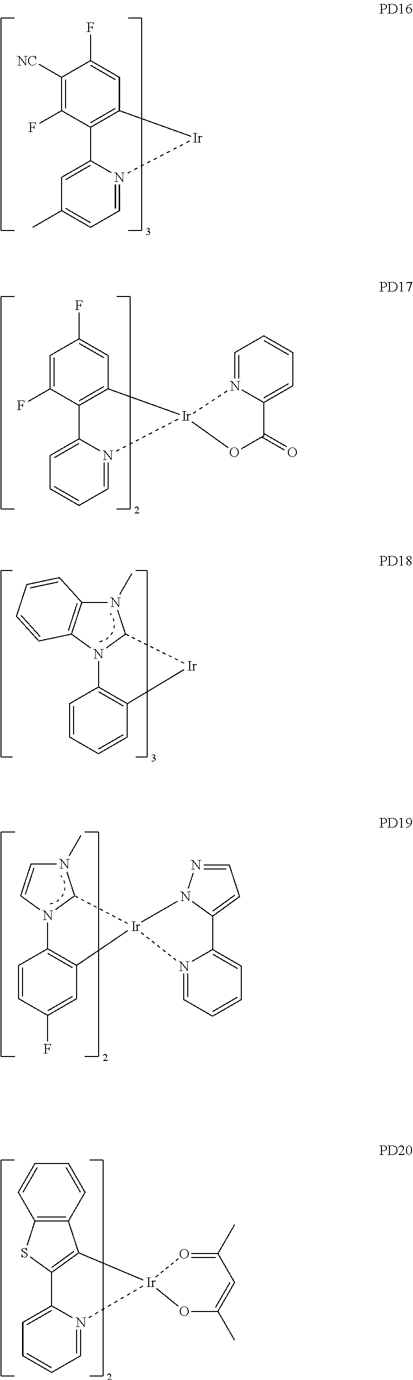

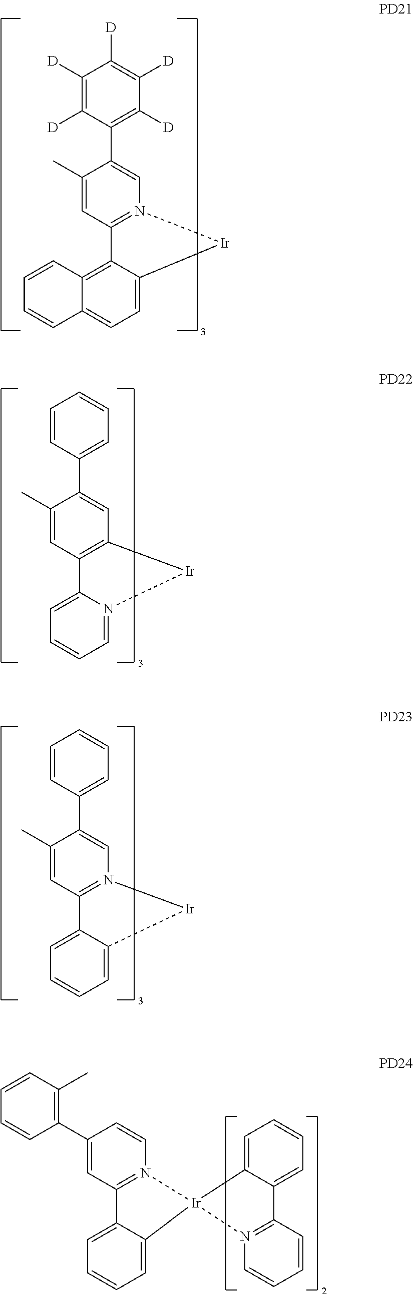

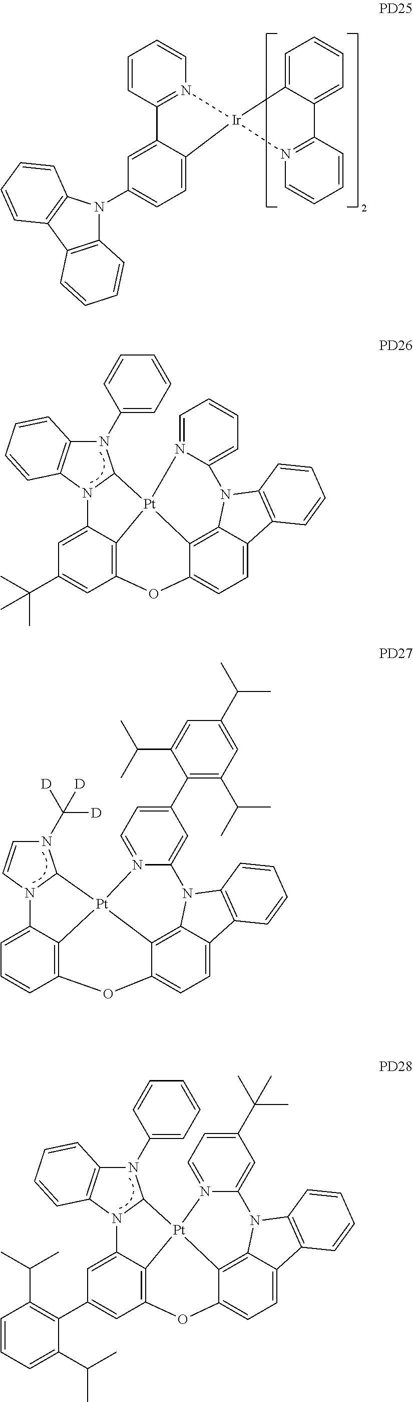

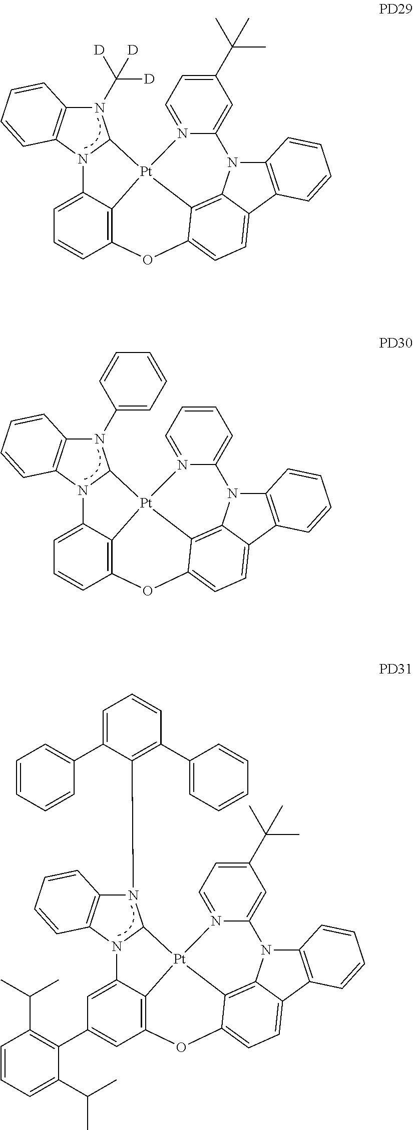

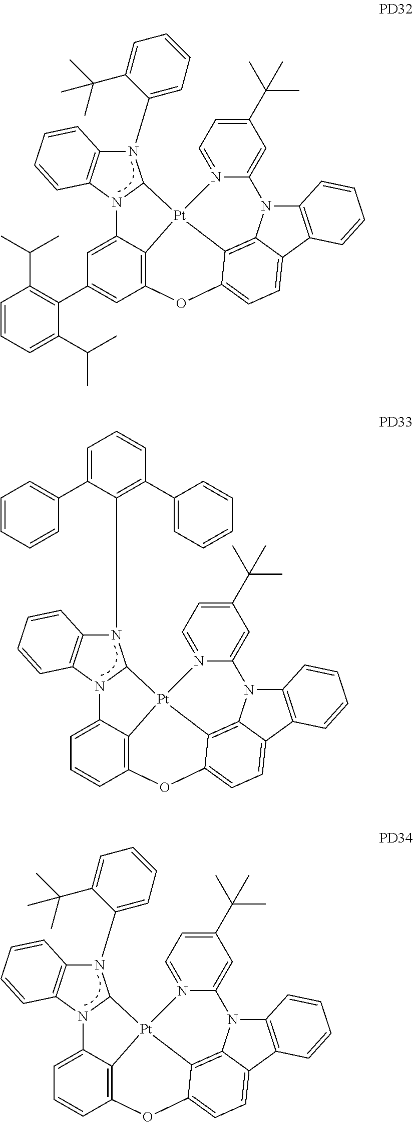

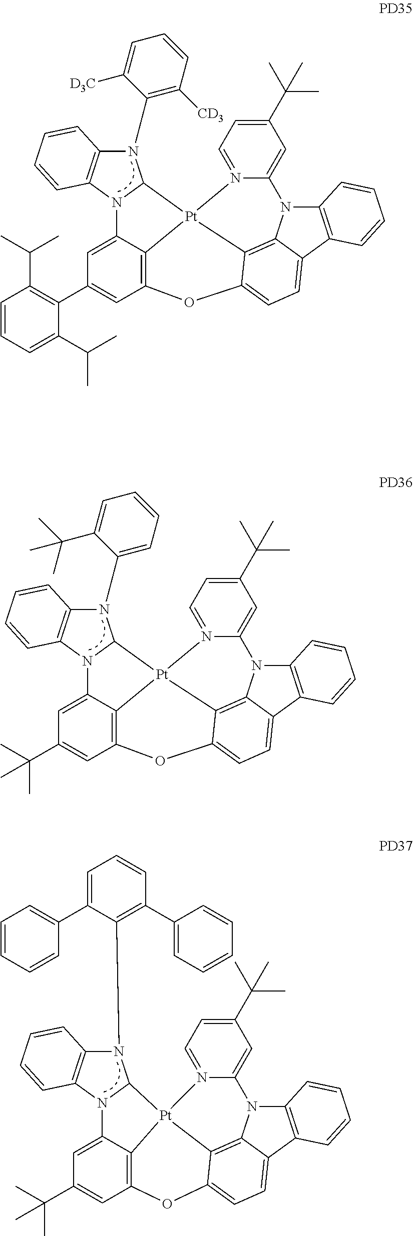

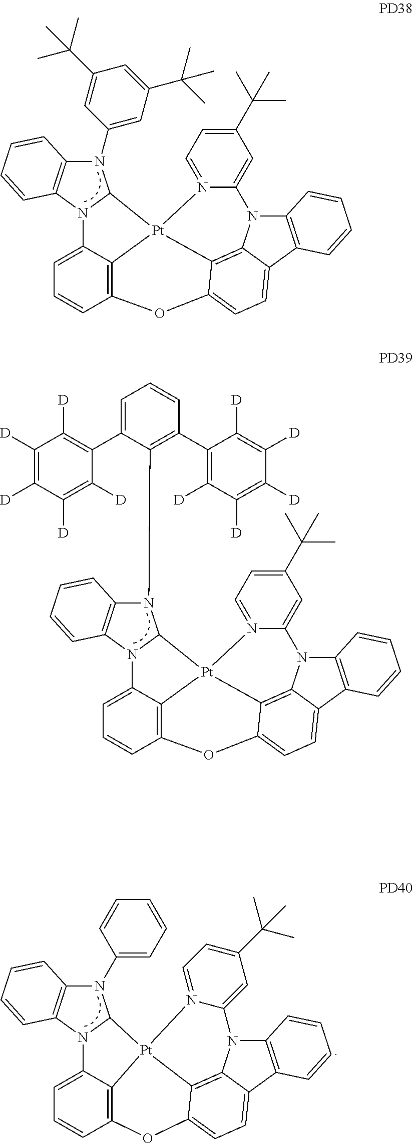

### [Fluorescent Dopant]

The fluorescent dopant may include an amine group-containing compound, a styryl group-containing compound, or any combination thereof.

For example, the fluorescent dopant may include a compound represented by Formula 501:

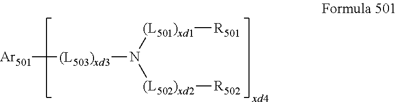

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, when xd1 is 0, (L₅₀₁)_{xd1} is a single bond, when xd2 is 0, (L₅₀₂)_{xd2} is a single bond, and when xd3 is 0, (L₅₀₃)ₓₐ₃ is a single bond, and
xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In one or more example embodiments, xd4 in Formula 501 may be 2.

For example, the fluorescent dopant may include: one of Compounds FD1 to FD36; DPVBi; DPAVBi; or any combination thereof:

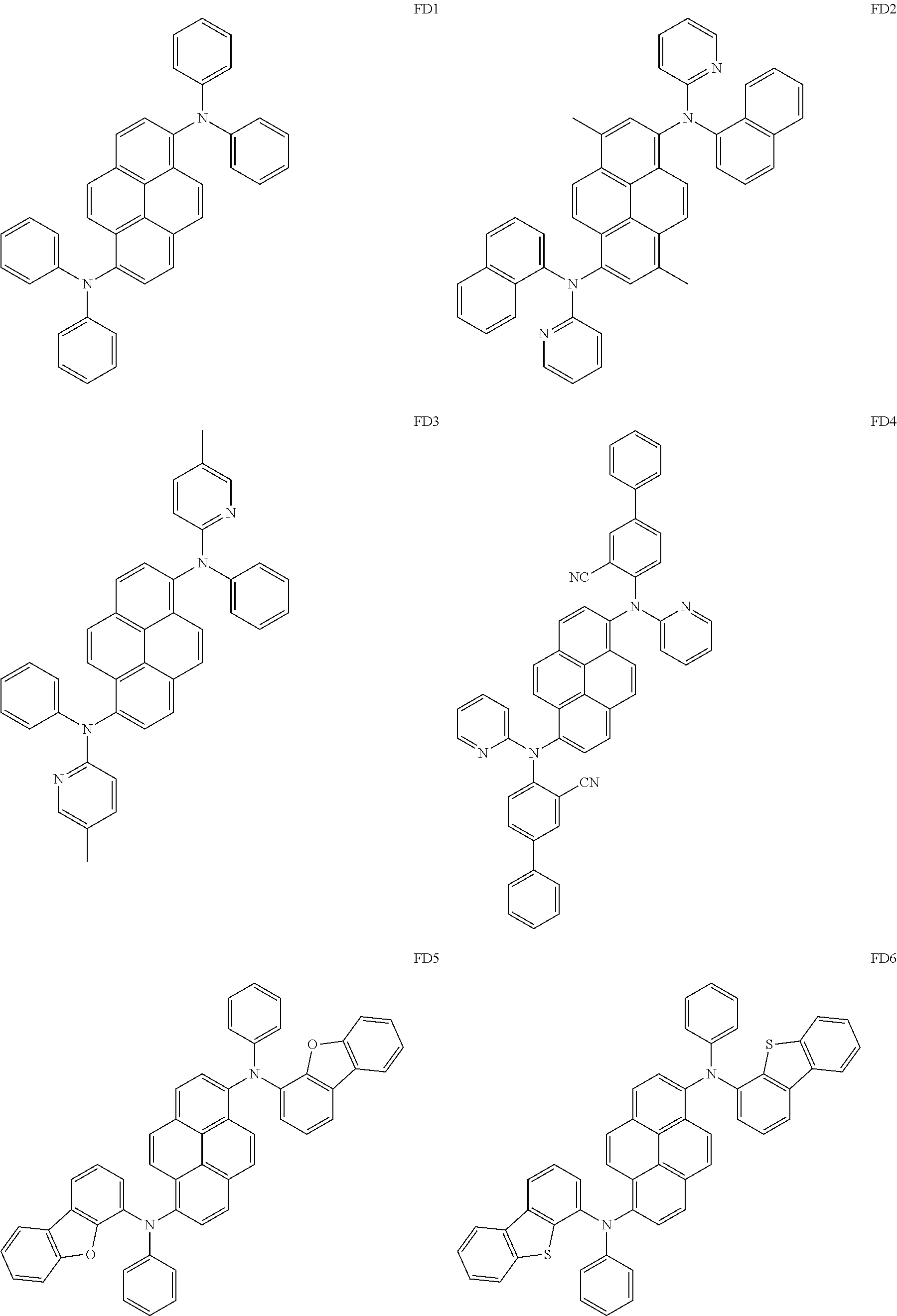

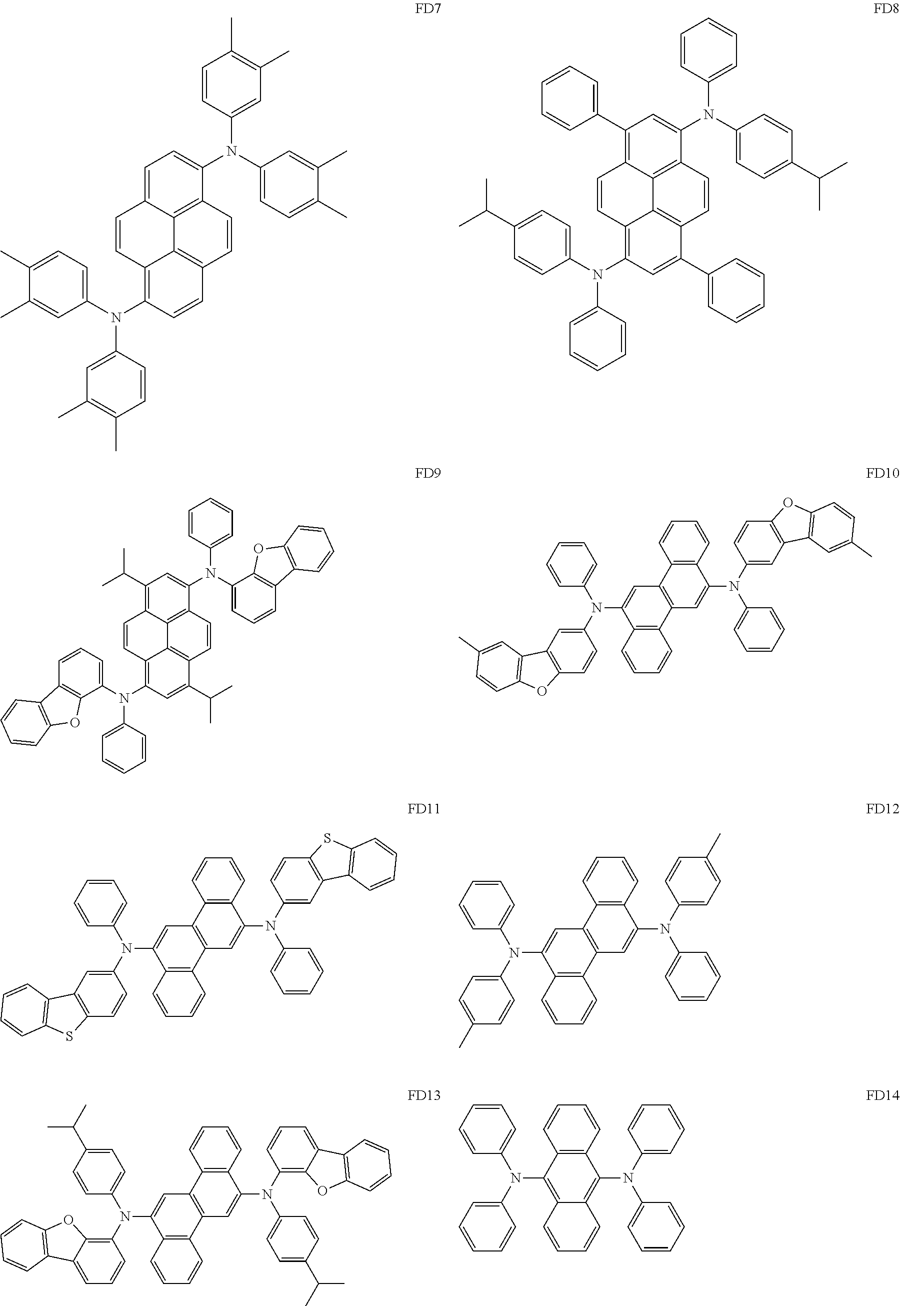

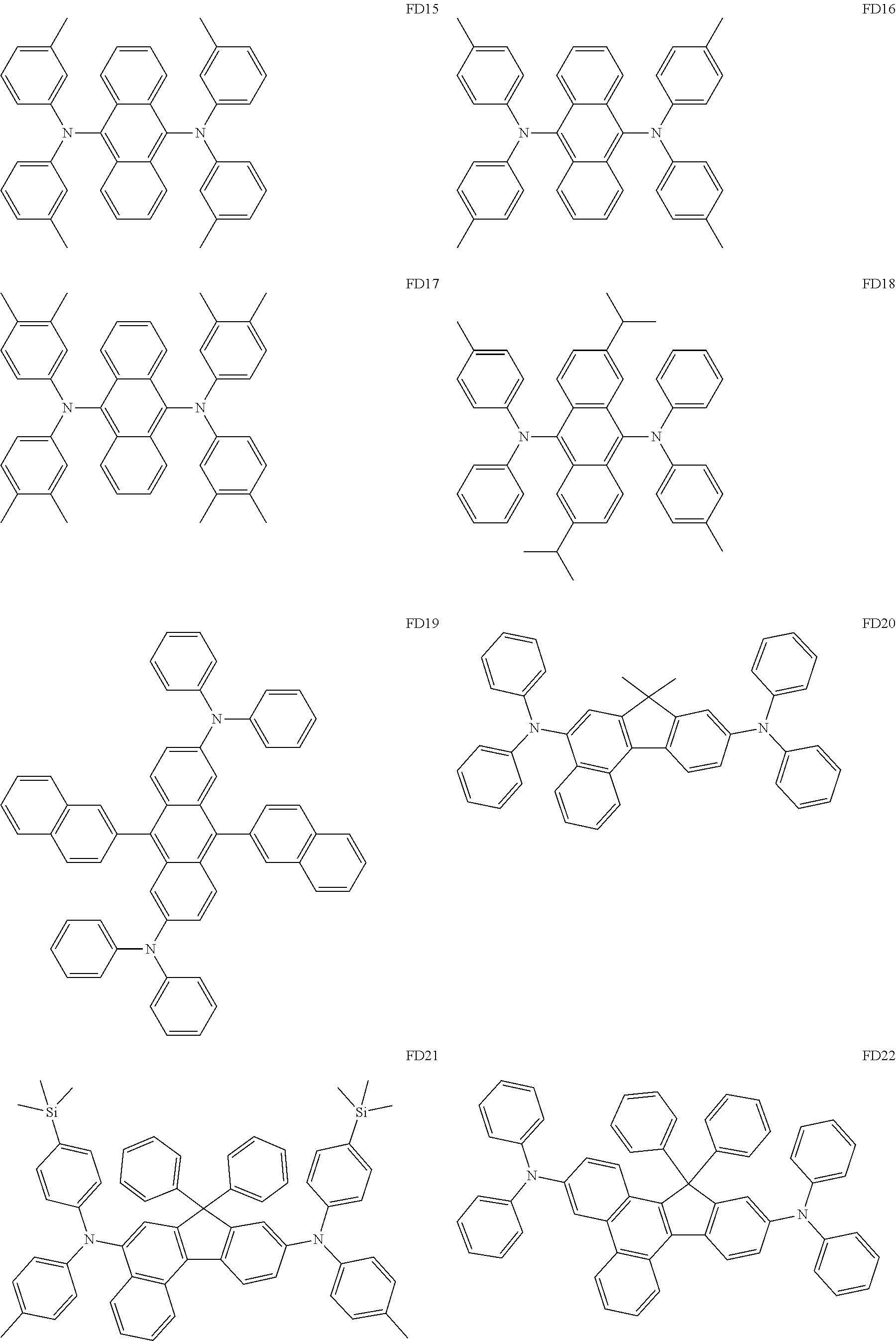

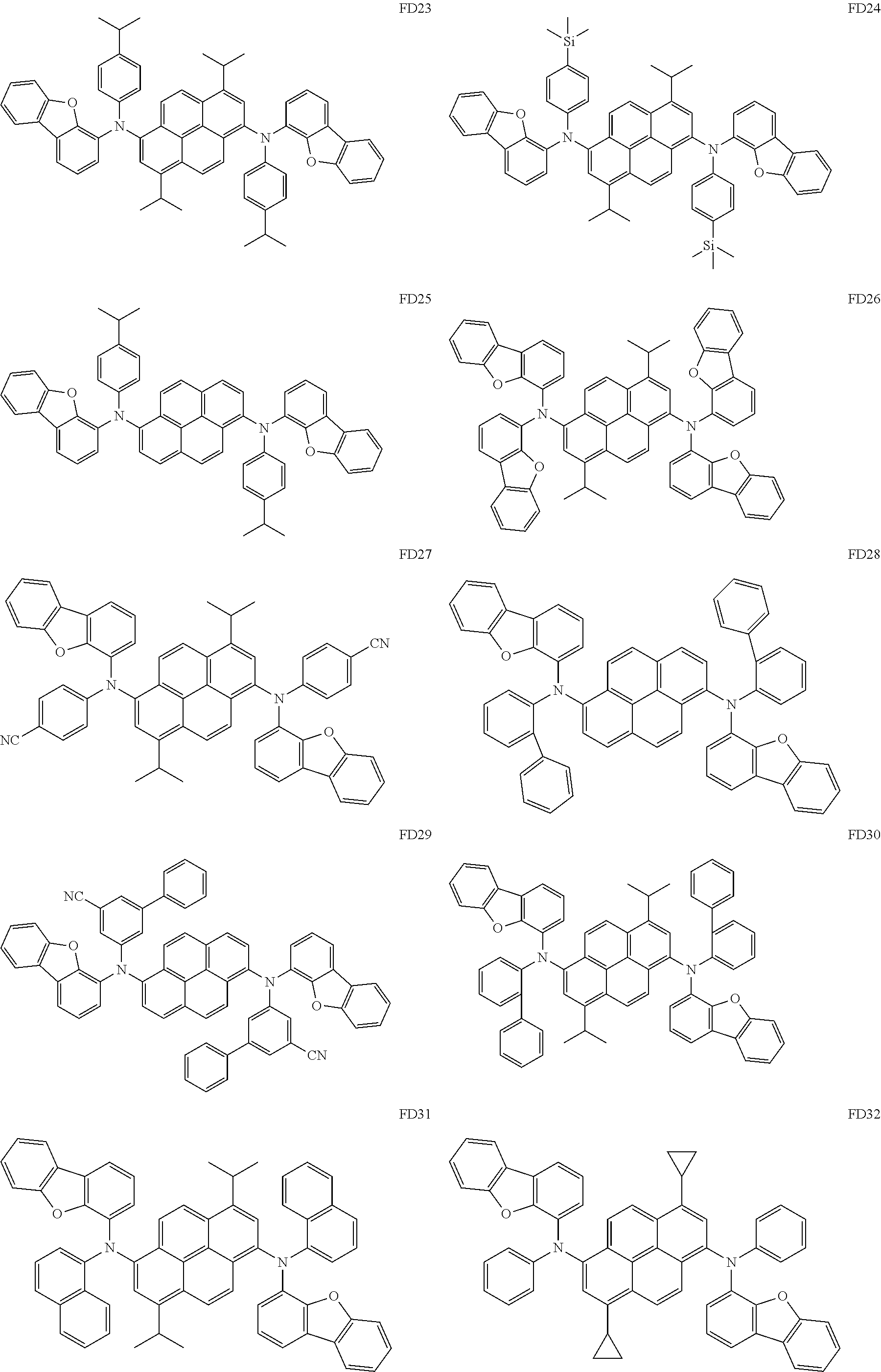

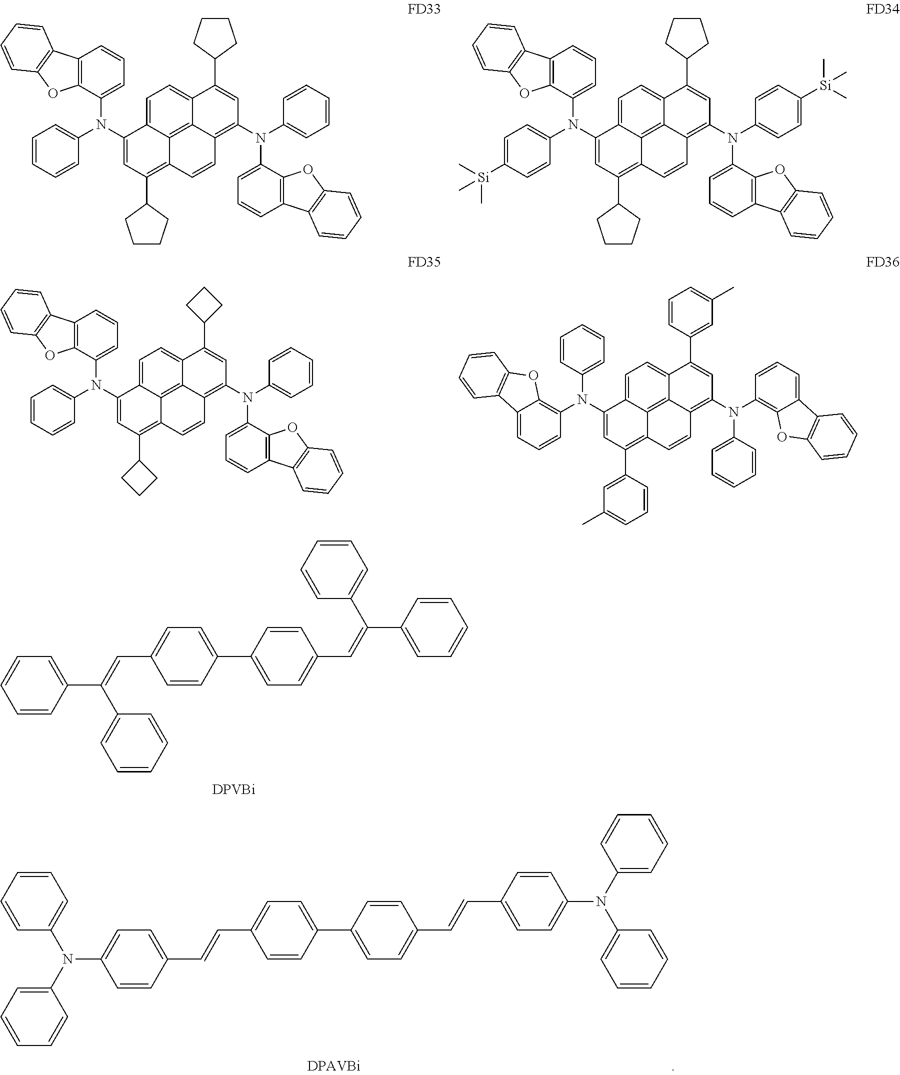

### [Delayed Fluorescence Material]

The emission layer may further include a delayed fluorescence material.

In some example embodiments, the delayed fluorescence material may be selected from compounds capable of emitting delayed fluorescent light based on a delayed fluorescence emission mechanism.

The delayed fluorescence material included in the emission layer may act as a host or a dopant depending on the type of other materials included in the emission layer.

In one or more example embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescent material and the singlet energy level (eV) of the delayed fluorescent material satisfies the above-described range, up-conversion from the triplet state to the singlet state of the delayed fluorescent materials may effectively occur, and thus, the luminescence efficiency of the organic light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed while sharing boron (B).

Examples of the delayed fluorescence material may include at least one of Compounds DF1 to DF14 and t-DABNA:

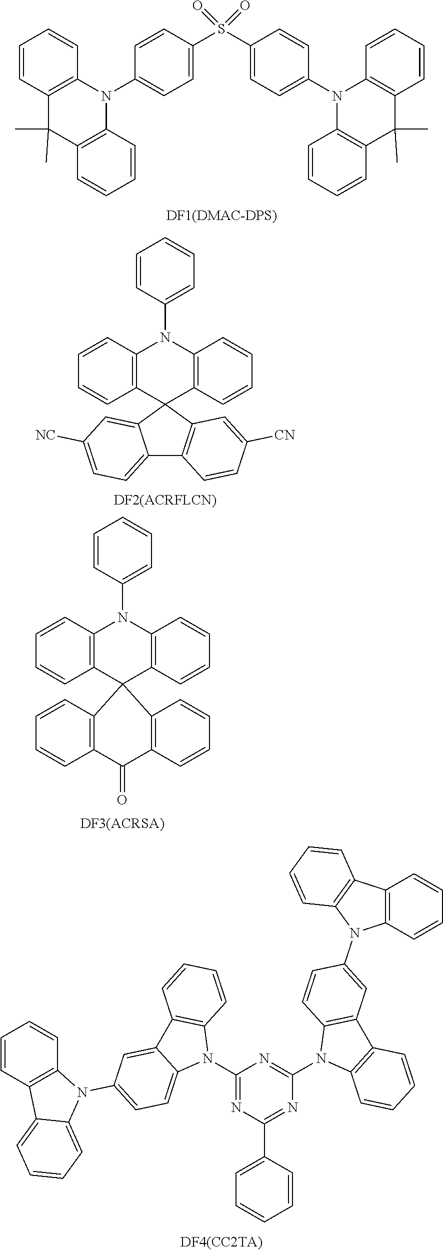

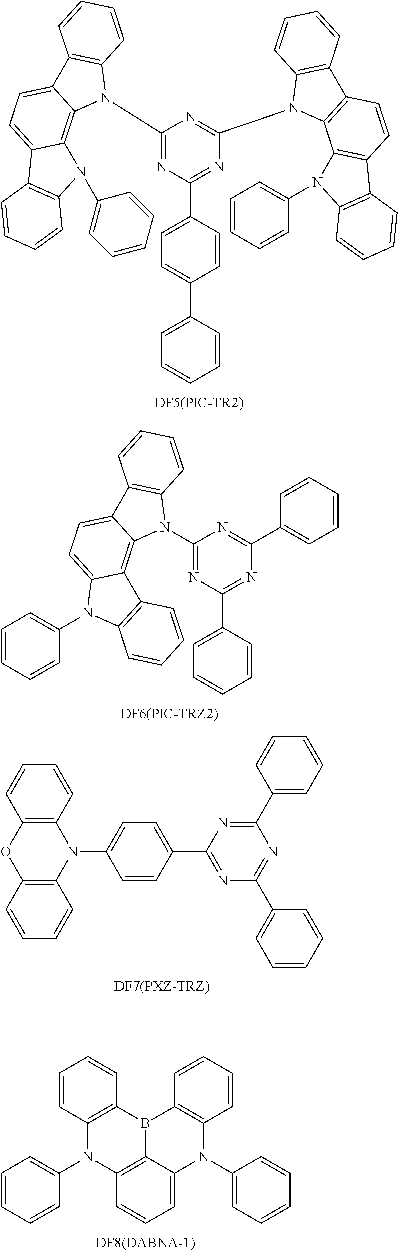 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1 ; c1ccc(N2c3ccccc3B3c4ccccc4N(c4ccccc4)c4cccc2c43)cc1]

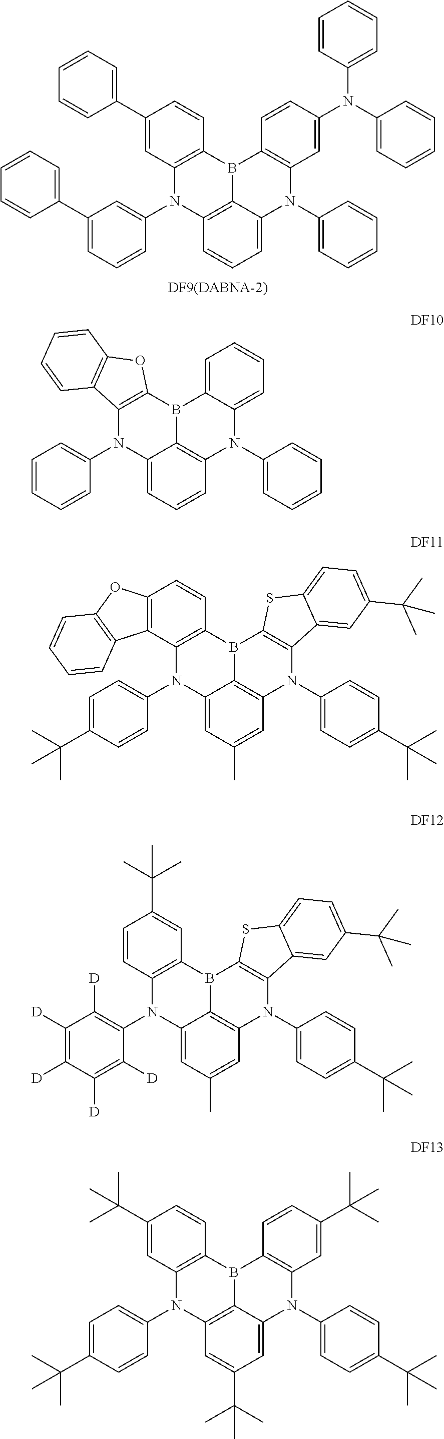

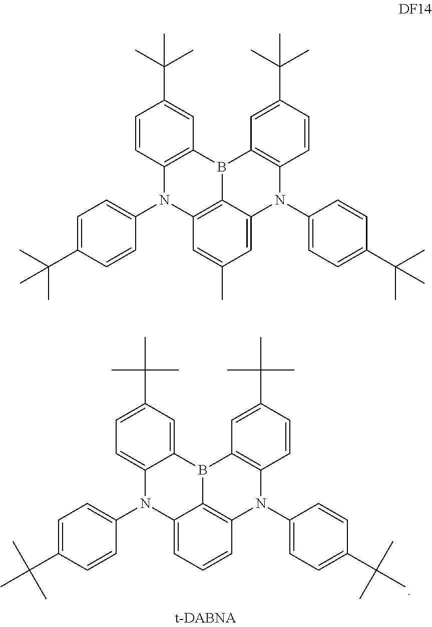

### [Quantum Dot]

The emission layer may include a quantum dot.

The term “quantum dot” as used herein refers to a crystal of a semiconductor compound. Quantum dots may emit light of various emission wavelengths according to the size of the crystal. Quantum dots may emit light of various emission wavelengths by adjusting the ratio of elements in the quantum dot compound.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the quantum dot particle crystals grow, the organic solvent may naturally act as a dispersant coordinated on the surface of the quantum dot particle crystals, and may control the growth of the crystals. Accordingly, in the wet chemical process, the growth of quantum dot particle crystals may be controlled through a process which costs lower and is easier than a vapor deposition method, such as metal organic chemical vapor deposition (MOCVD) or molecular beam epitaxy (MBE).

The quantum dot may include a Group III-VI semiconductor compound; a Group II-VI semiconductor compound; a Group III-V semiconductor compound; a Group I-III-VI semiconductor compound; a Group IV-VI semiconductor compound; a Group IV element or compound; or any combination thereof.

Examples of the Group II-VI semiconductor compound are a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound are a binary compound, such as GaN, GaP, GaAs, GaSb, AlN, AIP, AlAs, AlSb, InN, InP, InAs, InSb, or the like; a ternary compound, such as GaNP, GaNAs, GaNSb, GaPAs, GaPSb, AlNP, AlNAs, AlNSb, AIPAs, AlPSb, InGaP, InNP, InAlP, InNAs, InNSb, InPAs, InPSb, or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, or the like; and/or any combination thereof. Meanwhile, the Group III-V semiconductor compound may further include a Group II element. Examples of the Group III-V semiconductor compound further including a Group II element are InZnP, InGaZnP, InAlZnP, etc.

Examples of the Group III-VI semiconductor compound may include: a binary compound, such as GaS, Ga₂S₃, GaSe, Ga₂Se₃, GaTe, InS, InSe, In₂Se₃, or InTe; a ternary compound, such as InGaS₃ or InGaSe₃; and/or any combination thereof.

Examples of the Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, or the like; a quaternary compound, such as AgInGaS, AgInGaS₂, AgInGaSe, AgInGaSe₂, CuInGaS, CuInGaS₂, or the like; or any combination thereof.

Examples of the Group IV-VI semiconductor compound are: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; and/or any combination thereof.

The Group IV element or compound may include: a single element material, such as Si or Ge; a binary compound, such as SiC or SiGe; and/or any combination thereof.

Each element included in a multi-element compound such as the binary compound, the ternary compound, and the quaternary compound may be present at a uniform concentration or non-uniform concentration in a particle. The above formulae refer to the types of elements included in each compound, and the element ratios in these compounds may be different from each other. For example, AgInGaS2 may indicate AgInₓGa₁₋ₓS₂ (where x is a real number satisfying 0<x<1).

Meanwhile, the quantum dot may have a single structure in which the concentration of each element in the quantum dot is uniform, or a core-shell dual structure. For example, the material included in the core and the material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot may be an oxide of metal, or non-metal, a semiconductor compound, and any combination thereof. Examples of the oxide of metal or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and any combination thereof. Examples of the semiconductor compound are, as described herein, Group III-VI semiconductor compounds; Group II-VI semiconductor compounds; Group III-V semiconductor compounds; Group I-III-VI semiconductor compounds; Group IV-VI semiconductor compounds; and any combination thereof. For example, the semiconductor compound may include CdS, CdSe, CdTe, ZnS, ZnSe, ZnTe, ZnSeS, ZnSTe, GaAs, GaP, GaS, GaSe, AgGaS, AgGaS₂, GaSb, HgS, HgSe, HgTe, InAs, InP, InGaP, InSb, AlAs, AIP, AlSb, or any combination thereof.

Each element included in a multi-element compound, such as the binary compound and the ternary compound, may be present at a uniform concentration or non-uniform concentration in a particle. The above formulae refer to the types of elements included in each compound, and the element ratios in these compounds may be different from each other.

A full width at half maximum (FWHM) of the emission wavelength spectrum of the quantum dot may be about 45 nm or less, for example, about 40 nm or less, for example, about 30 nm or less, and within these ranges, color purity or color reproducibility may be increased. In addition, since the light emitted through the quantum dot is emitted in all directions, the wide viewing angle may be improved.

In addition, the quantum dot may be in the form of a spherical nanoparticle, a pyramidal nanoparticle, a multi-arm nanoparticle, a cubic nanoparticle, a nanotube, a nanowire, a nanofiber, or a nanoplate.

By adjusting the size of the quantum dots, the energy band gap may be adjusted, and thus, light of various wavelengths may be obtained in a quantum dot emission layer. Thus, by using quantum dots as described above (by using quantum dots of different sizes or by varying the ratio of elements in a quantum dot compound), a light-emitting device that emits light of various wavelengths may be realized. In one or more embodiments, the size of the quantum dots or the ratio of elements in the quantum dot compound may be selected so that red light, green light, and/or blue light can be emitted.

In one or more embodiments, the quantum dots may be configured to emit white light by combination of light of various colors.

### [Electron Transport Region in Interlayer 130]

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron-transporting region may include a buffer layer, a hole-blocking layer, an electron control layer, an electron-transporting layer, an electron injection layer, and/or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the constituting layers of each structure being sequentially stacked from an emission layer.

In some example embodiments, the electron transport region (for example, the buffer layer, the hole-blocking layer, the electron control layer, or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5, when xe1 is 0, *-(L₆₀₁)ₓₑ₁-* is a single bond,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described herein with respect to Q₁,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In some example embodiments, Ar₆₀₁ in Formula 601 may be an anthracene group unsubstituted or substituted with at least one R₁₀ₐ.

In some example embodiments, the electron transport region may include a compound represented by Formula 601-1:

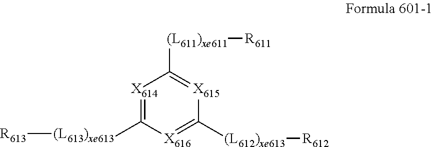

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each be the same as described herein with respect to L₆₀₁,
xe611 to xe613 may each be the same as described herein with respect to xe1,
R₆₁₁ to R₆₁₃ may each be the same as described herein with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alg3, BAlq, TAZ, NTAZ, TSPO1, TPBI, or any combination thereof:

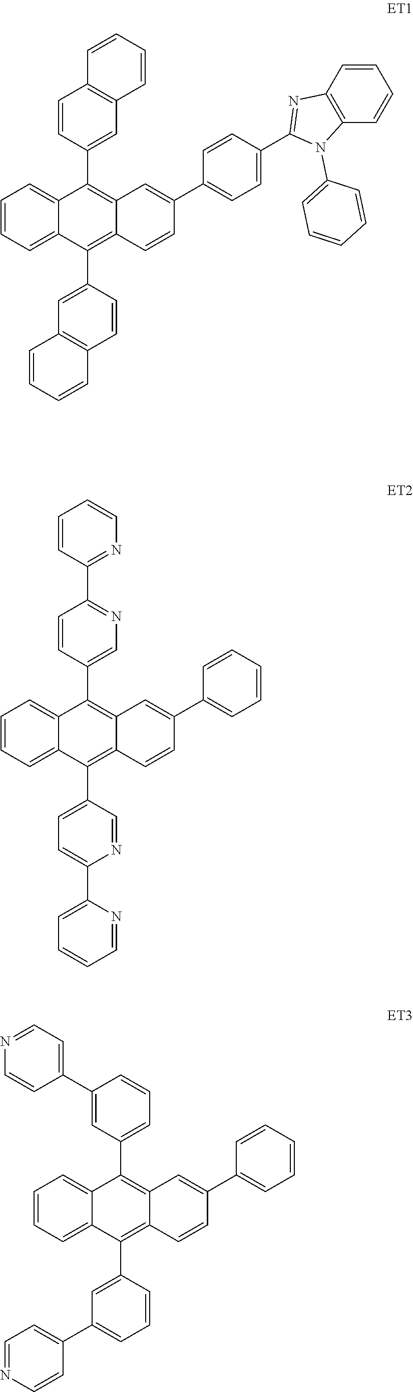 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

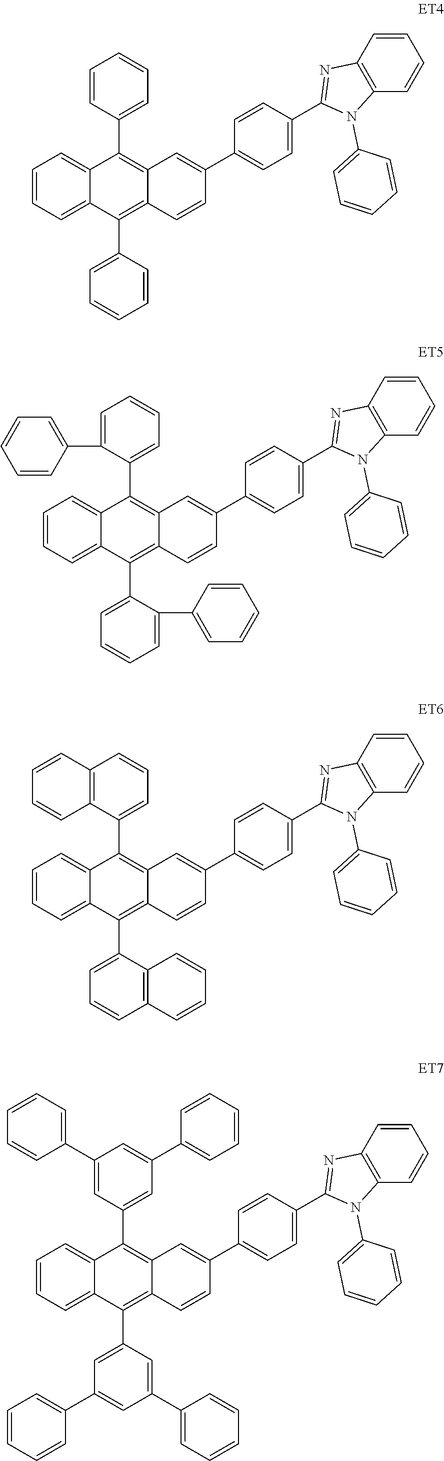 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

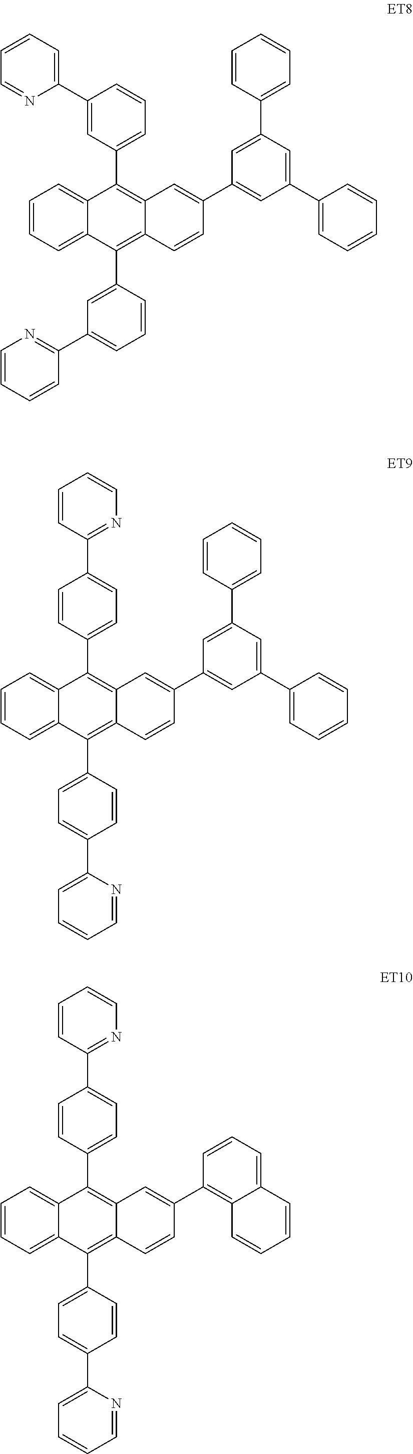 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

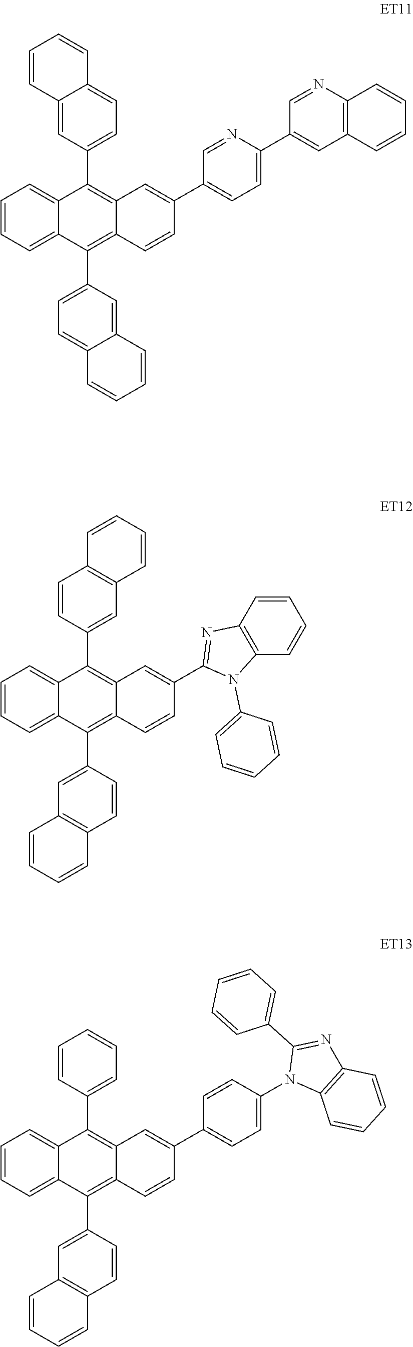 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

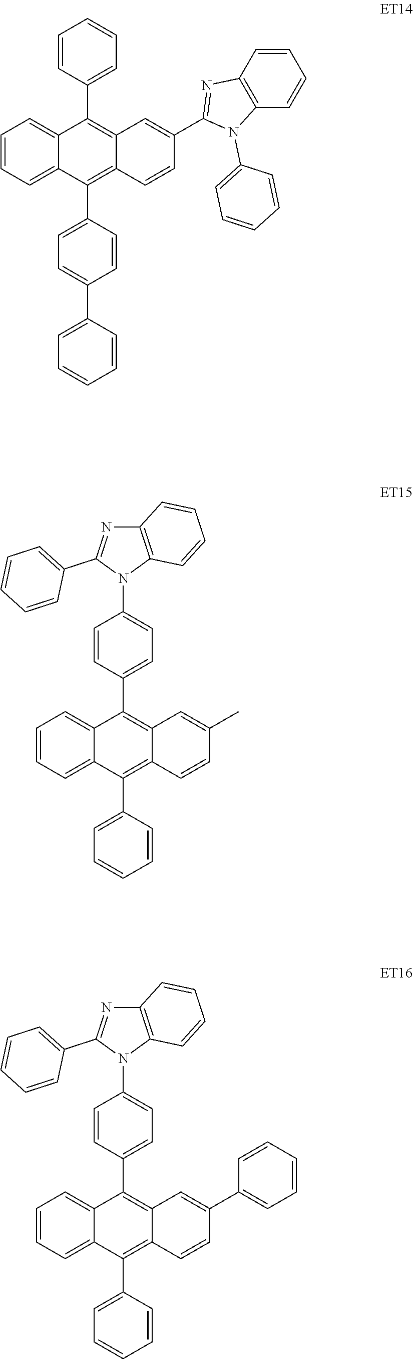 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

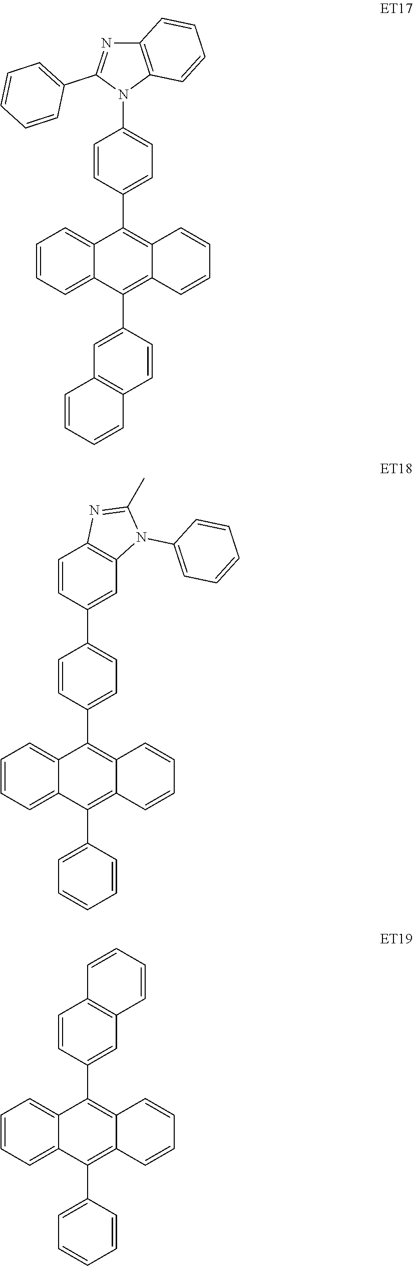

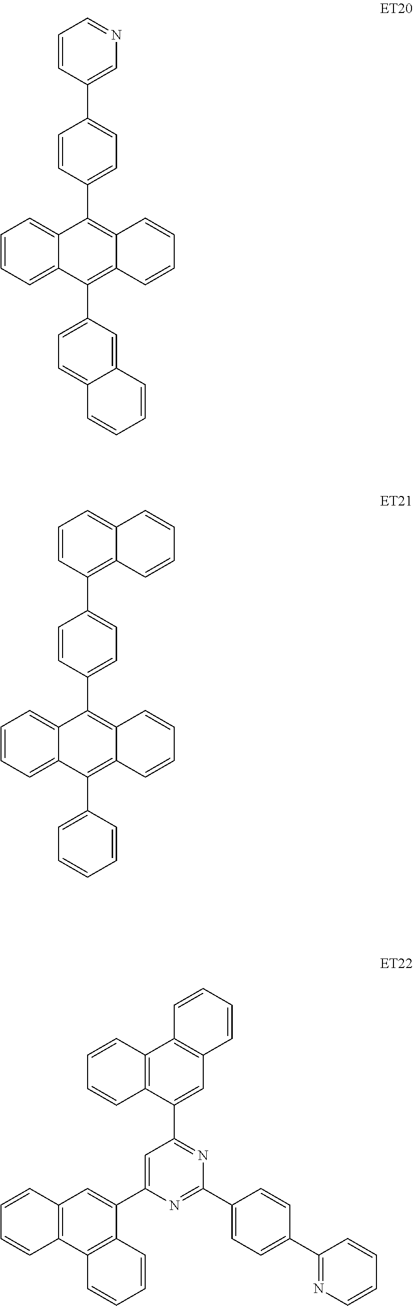

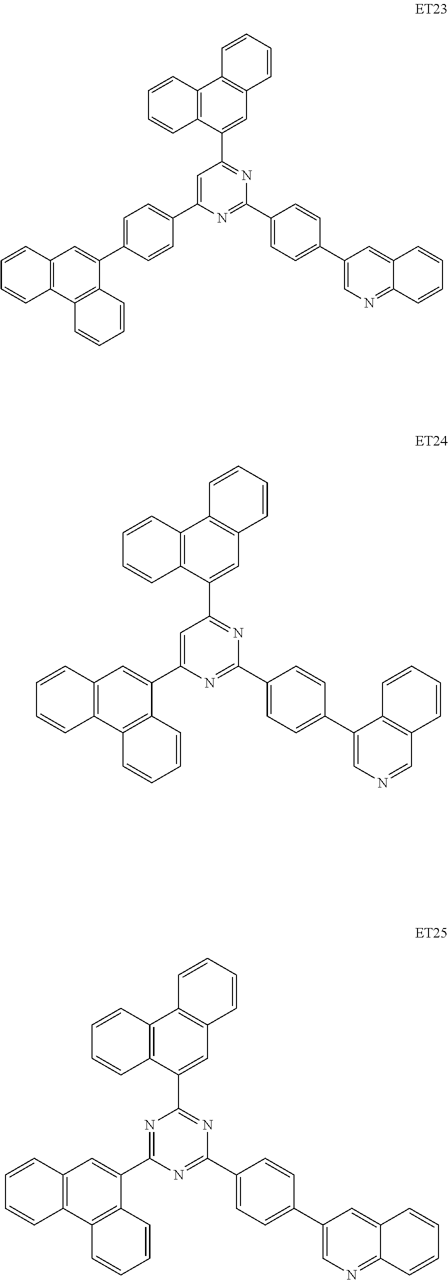 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)cncc2c1 ; c1ccc2ncc(-c3ccc(-c4nc(-c5cc6ccccc6c6ccccc56)nc(-c5cc6ccccc6c6ccccc56)n4)cc3)cc2c1]

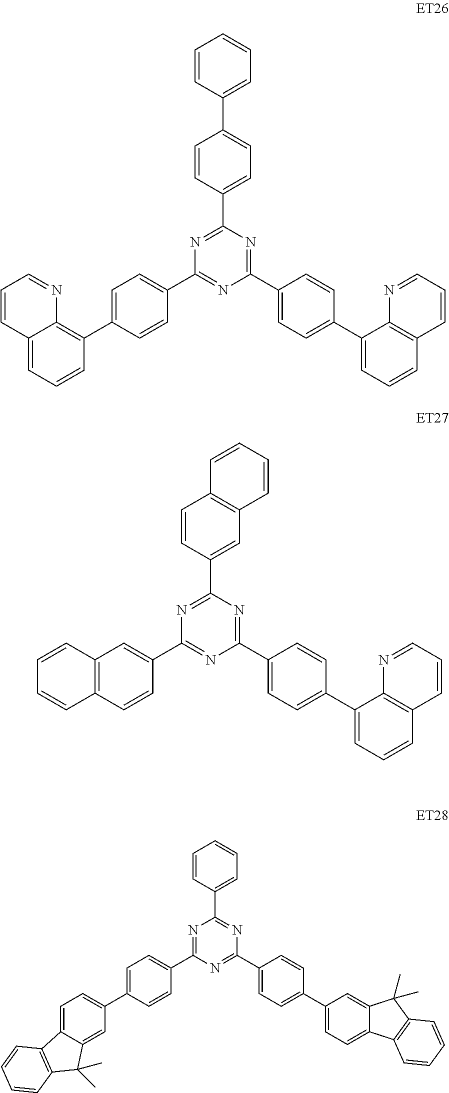 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

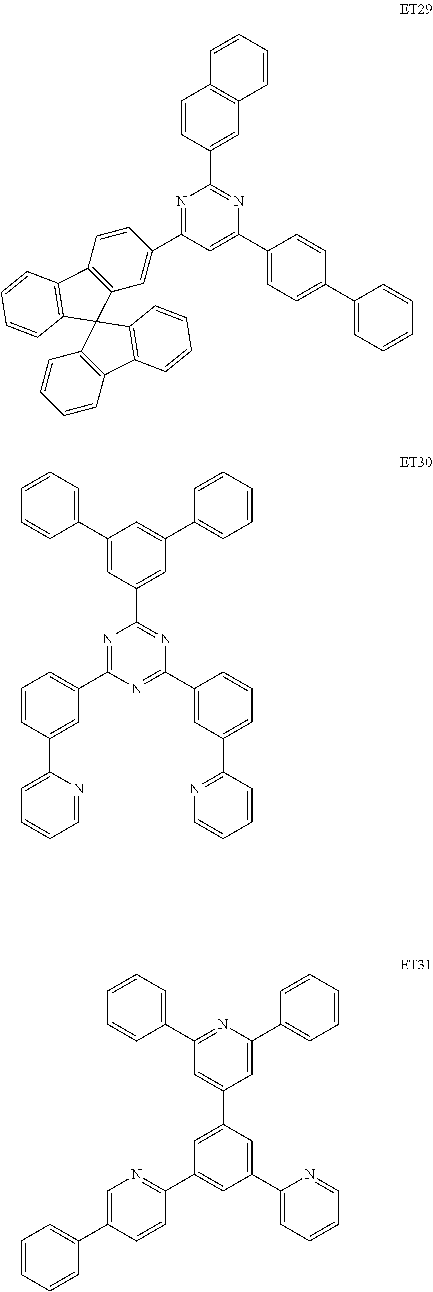 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

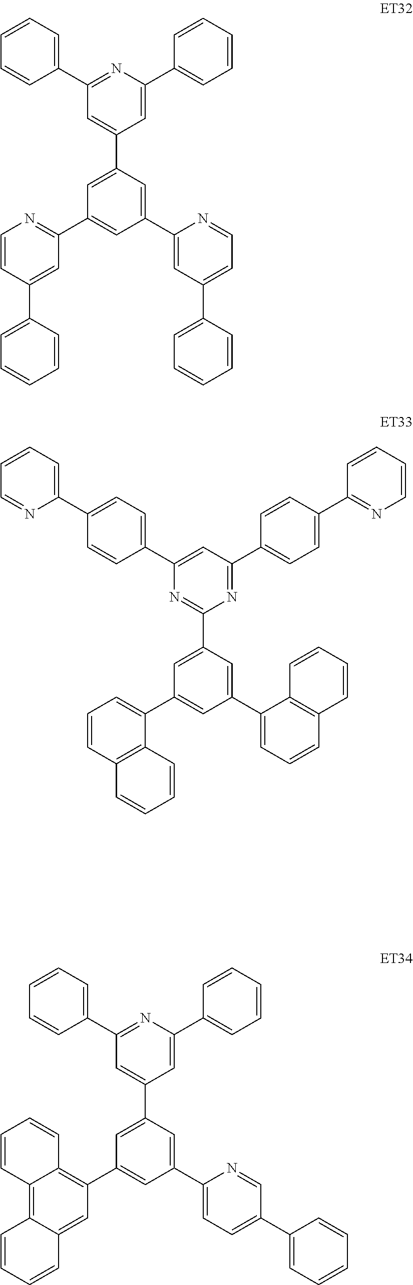 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

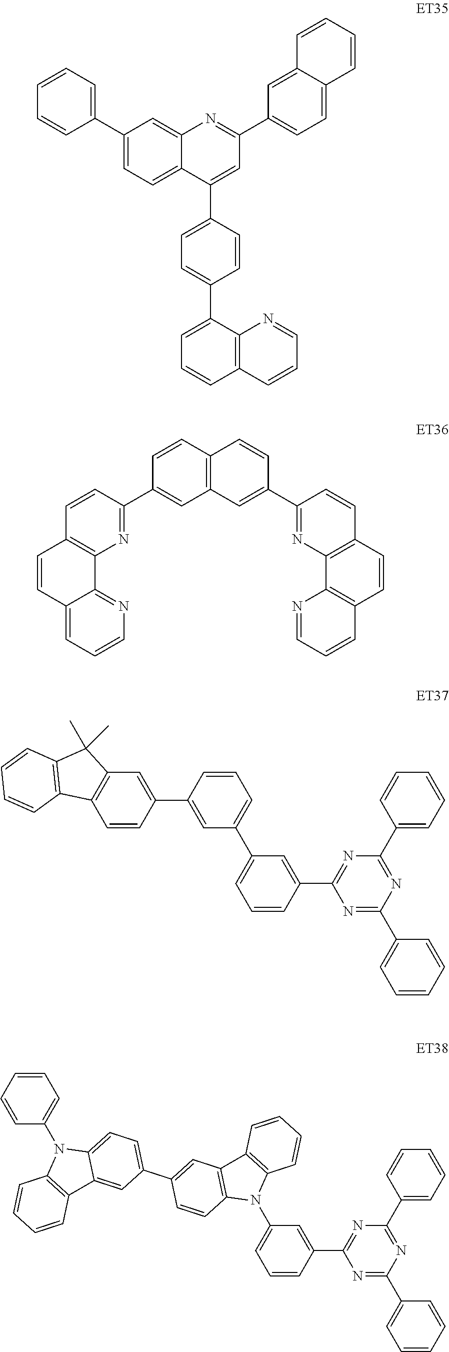 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

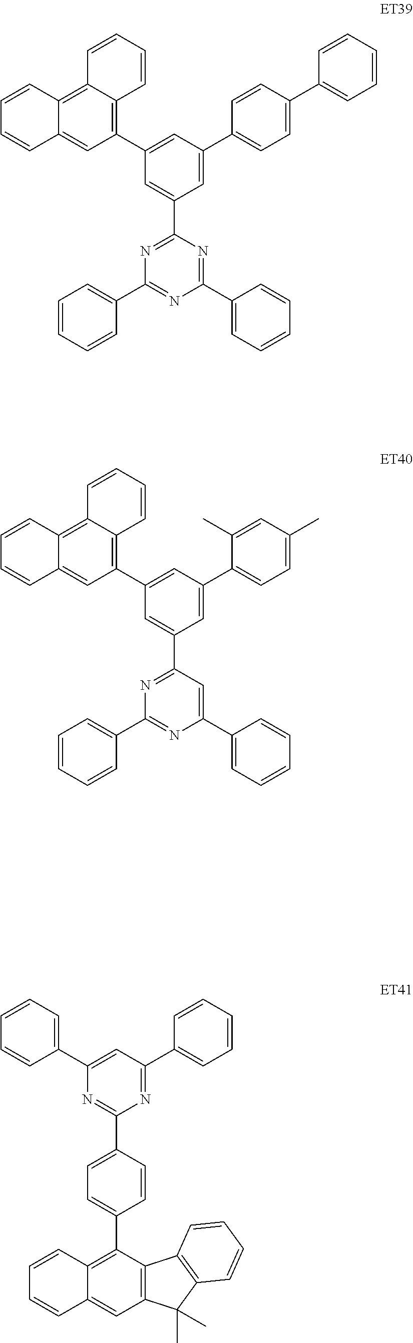 [SMILES: c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1]

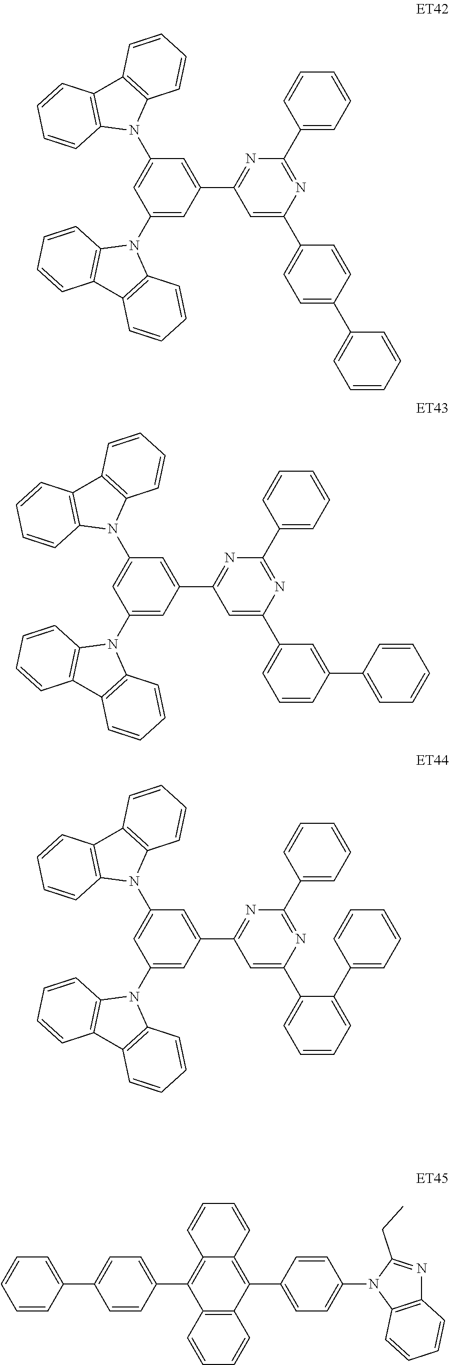 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

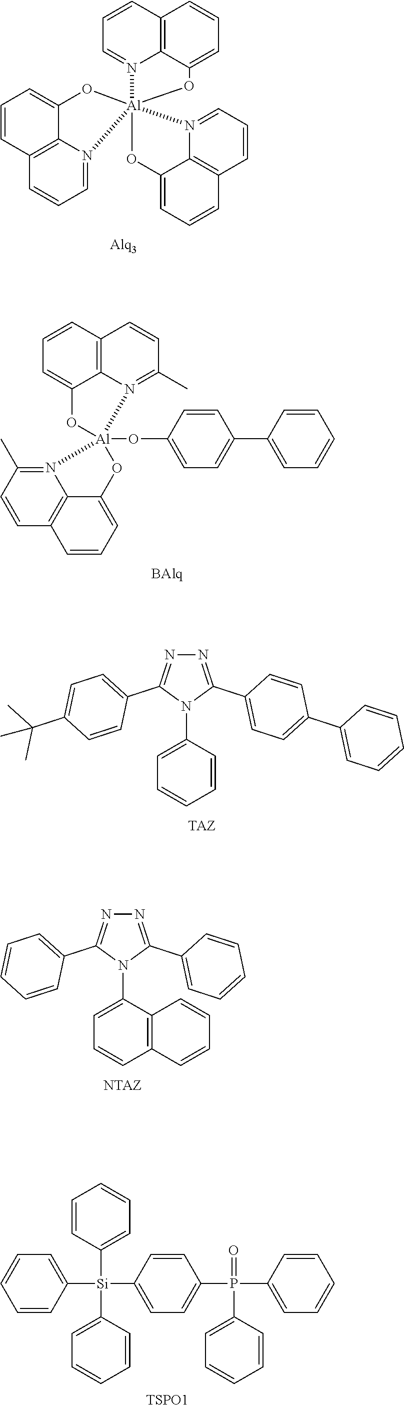

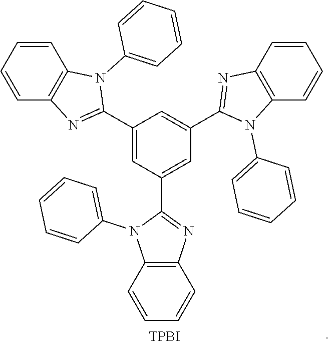

A thickness of the electron transport region may be from about 100 Å to about 5,000 Å, for example, about 160 Å to about 4,000 Å. When the electron transport region includes a buffer layer, a hole-blocking layer, an electron control layer, an electron transport layer, or any combination thereof, the thickness of the buffer layer, the hole-blocking layer, or the electron control layer may each independently be from about 20 Å to about 1,000 Å, for example, about 30 Å to about 300A, and the thickness of the electron transport layer may be from about 100 Å to about 1,000 Å, for example, about 150 Å to about 500 Å. When the thickness of the buffer layer, the hole-blocking layer, the electron control layer, the electron transport layer, and/or the electron transport region are within these ranges, satisfactory electron-transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

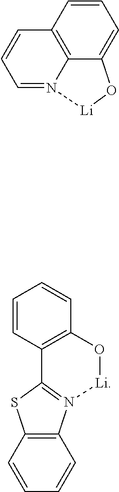

The electron transport region may include an electron injection layer that facilitates the injection of electrons from the second electrode 150. The electron injection layer may contact, for example directly contact, the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, or iodides), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, Lil, Nal, Csl, or KI; or any combination thereof. The alkaline earth metal-containing compound may include an alkaline earth metal oxide, such as BaO, SrO, CaO, BaxSri-xO (wherein x is a real number satisfying the condition of 0<x<1), BaxCai-xO (wherein x is a real number satisfying the condition of 0<x<1), or the like. The rare earth metal-containing compound may include YbF3, ScF3, Sc203, Y₂O₃, Ce203, GdF3, TbF3, Ybl3, ScI3, Tbl3, or any combination thereof. In one or more embodiments, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may consist of: i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

### [Second Electrode 150]

The second electrode 150 may be located on the interlayer 130 having a structure as described above. The second electrode 150 may be a cathode, which is an electron injection electrode, and as the material for the second electrode 150, a metal, an alloy, an electrically conductive compound, or any combination thereof, each having a low-work function, may be used.

The second electrode 150 may include lithium (Li), sodium (Na), silver (Ag), magnesium (Mg), aluminum (AI), silver-lithium (Ag—Li), silver-sodium (Ag—Na), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### [Capping Layer]

A first capping layer may be located outside the first electrode 110 (e.g., the first electrode 110 may be located between the first capping layer and the interlayer 130), and/or a second capping layer may be located outside the second electrode 150 (e.g., the second electrode 150 may be located between the second capping later and the interlayer). In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in an emission layer of the interlayer 130 of the light-emitting device 10 may be extracted toward the outside through the first electrode 110 which is a semi-transmissive electrode or a transmissive electrode, and the first capping layer. Light generated in an emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at 470 nm). In some example embodiments, each of the first capping layer and the second capping layer may include a material having a refractive index of 1.8 or more. In some example embodiments, each of the first capping layer and the second capping layer may include a material having a refractive index of 2.0 or more.

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include carbocyclic compounds, heterocyclic compounds, amine group-containing compounds, porphine derivatives, phthalocyanine derivatives, a naphthalocyanine derivatives, alkali metal complexes, alkaline earth metal complexes, and/or any combination thereof. Optionally, the carbocyclic compound, the heterocyclic compound, and the amine group-containing compound may be substituted with a substituent including O, N, S, Se, Si, F, Cl, Br, I, and/or any combination thereof. In one or more example embodiments, at least one of the first capping layer and the second capping layer may each independently include an amine group-containing compound.

For example, at least one of the first capping layer and the second capping layer may each independently include a compound represented by Formula 201, a compound represented by Formula 202, and/or any combination thereof.

In one or more example embodiments, at least one of the first capping layer and the second capping layer may each independently include one of Compounds HT28 to HT33, one of Compounds CP1 to CP6, β-NPB, and/or any combination thereof:

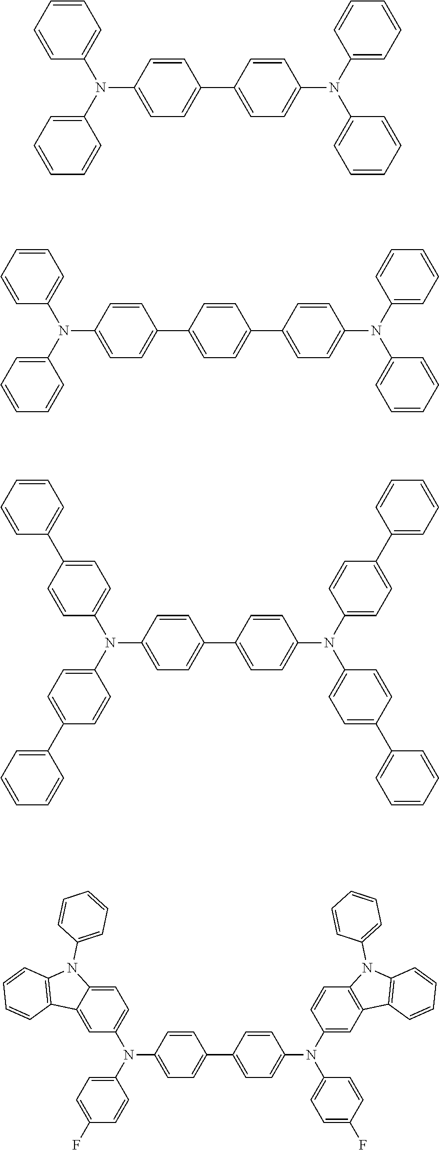 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

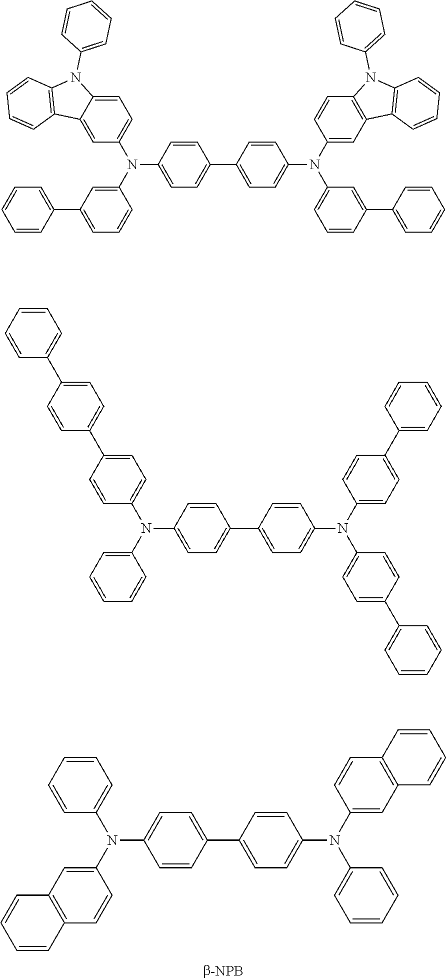 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### [Film]

The amine-containing compound represented by Formula 1 may be included in various films.

Thus, another aspect of the disclosure provides a film including the amine-containing compound represented by Formula 1. The film may be, for example, an optical member (or a light control means) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like) or a protective member (for example, an insulating layer, a dielectric layer, or the like).

### [Electronic Apparatus]

The light-emitting device may be included in various electronic apparatuses. For example, the electronic apparatus including the light-emitting device may be a light-emitting apparatus, an authentication apparatus, or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be located in at least one direction in which light emitted from the light-emitting device travels. For example, the light emitted from the light-emitting device may be blue light or white light. For details on the light-emitting device, related description provided above may be referred to. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining layer may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the plurality of color filter areas (or the plurality of color conversion areas) may include quantum dots. In particular, the first area may include a red quantum dot, the second area may include a green quantum dot, and the third area may not include a quantum dot. For details on the quantum dot, related descriptions provided herein may be referred to. The first area, the second area, and/or the third area may each include a scatter.

For example, the light-emitting device may emit first light, the first area may absorb the first light to emit first-first color light, the second area may absorb the first light to emit second-first color light, and the third area may absorb the first light to emit third-first color light. In this regard, the first-first color light, the second-first color light, and the third-first color light may have different maximum emission wavelengths. In particular, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, or the like.

The activation layer may include crystalline silicon, amorphous silicon, an organic semiconductor, an oxide semiconductor, or the like.

The electronic apparatus may further include a sealing portion for sealing the light-emitting device. The sealing portion may be located between the color filter and/or the color conversion layer and the light-emitting device. The sealing portion allows light from the light-emitting device to be extracted to the outside, and simultaneously prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally located on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus (for example, a light-emitting apparatus) may be applied to various electronic equipment. In some example embodiments, the electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement apparatuses, pulse wave measurement apparatuses, electrocardiogram displays, ultrasonic diagnostic apparatuses, or endoscope apparatuses), fish finders, various measuring instruments, meters (for example, meters for a vehicle, an aircraft, and a vessel), projectors, and the like.

### [Electronic Equipment]

The electronic apparatus may be applied to various electronic equipment. Accordingly, the light-emitting device may be included in various electronic equipment.

In some example embodiments, the light-emitting apparatus may be applied to various electronic equipment. The electronic equipment according to some example embodiments may include the light-emitting apparatus described above and may further include, in addition to the light-emitting apparatus, a module or an apparatus which has an additional function.

For example, the electronic equipment including the light-emitting device may be one of a flat panel display, a curved display, a computer monitor, a medical monitor, a TV, a billboard, indoor or outdoor illuminations and/or signal light, a head-up display, a fully or partially transparent display, a flexible display, a rollable display, a foldable display, a stretchable display, a laser printer, a phone, a cell phone, a tablet, a phablet, a personal digital assistant (PDA), a wearable device, a laptop computer, a digital camera, a camcorder, a viewfinder, a micro display, a 3D display, a virtual reality display, an augmented reality display, a vehicle, a video wall including multiple displays tiled together, a theater screen, a stadium screen, a phototherapy device, and a signboard.

The light-emitting device may have excellent effects in terms of luminescence efficiency long lifespan, and thus the electronic equipment including the light-emitting device may have characteristics, such as high luminance, high resolution, and low power consumption.

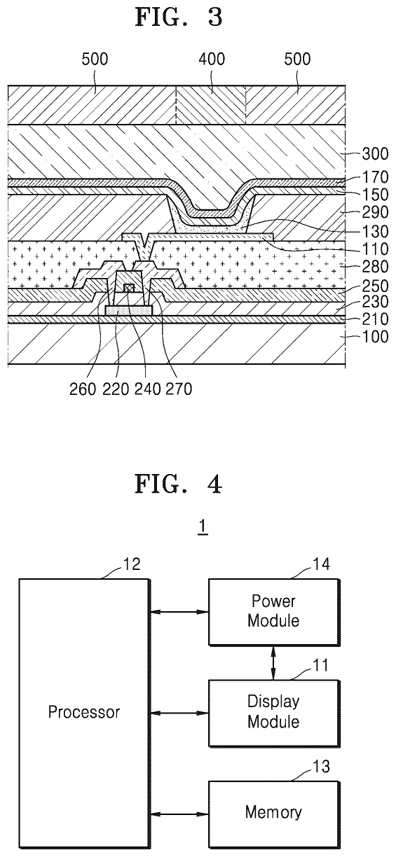

### [Description of FIGS. 2 and 3]

FIG. 2 is a cross-sectional view of an electronic apparatus according to some example embodiments (e.g., a light-emitting apparatus).

The light-emitting apparatus of FIG. 2 includes a substrate 100, a thin-film transistor (TFT), a light-emitting device, and a sealing portion 300 that seals the light-emitting device.

The substrate 100 may be a flexible substrate, a glass substrate, or a metal substrate. A buffer layer 210 may be located on the substrate 100. The buffer layer 210 may prevent penetration of impurities through the substrate 100 and may provide a flat surface on the substrate 100.

A TFT may be located on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be located on the activation layer 220, and the gate electrode 240 may be located on the gate insulating film 230.

An interlayer insulating film 250 may be located on the gate electrode 240. The interlayer insulating film 250 may be placed between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be located on the interlayer insulating film 250. The interlayer insulating film 250 and the gate insulating film 230 may be formed to expose the source region and the drain region of the activation layer 220, and the source electrode 260 and the drain electrode 270 may be located in contact with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be located on the passivation layer 280. The passivation layer 280 may be located to expose a portion of the drain electrode 270, not fully covering the drain electrode 270, and the first electrode 110 may be located to be connected to the exposed portion of the drain electrode 270.

A pixel defining layer 290 including an insulating material may be located on the first electrode 110. The pixel defining layer 290 may expose a certain region of the first electrode 110, and an interlayer 130 may be formed in the exposed region of the first electrode 110. The pixel defining layer 290 may be a polyimide or polyacrylic organic film.

Although not shown in FIG. 2, at least some layers of the interlayer 130 may extend beyond the upper portion of the pixel defining layer 290 to be located in the form of a common layer.

The second electrode 150 may be located on the interlayer 130, and a capping layer 170 may be additionally formed on the second electrode 150. The capping layer 170 may be formed to cover the second electrode 150.

The sealing portion 300 may be located on the capping layer 170. The sealing portion 300 may be located on a light-emitting device to protect the light-emitting device from moisture or oxygen. The sealing portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic-based resin (for example, polymethyl methacrylate, polyacrylic acid, or the like), an epoxy-based resin (for example, aliphatic glycidyl ether (AGE), or the like), or any combination thereof; or any combination of the inorganic films and the organic films.

FIG. 3 shows a cross-sectional view showing a light-emitting apparatus according to some example embodiments of the disclosure.

The light-emitting apparatus of FIG. 3 is the same as the light-emitting apparatus of FIG. 2, except that a light-shielding pattern 500 and a functional region 400 are additionally located on the sealing portion 300. The functional region 400 may be i) a color filter area, ii) a color conversion area, or iii) a combination of the color filter area and the color conversion area. In some example embodiments, the light-emitting device included in the light-emitting apparatus of FIG. 3 may be a tandem light-emitting device.

### [Description of FIG. 4]

FIG. 4 is a block diagram of electronic equipment 1 according to some example embodiments. Referring to FIG. 4, the electronic equipment 1 according to some example embodiments may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), and a controller.

The memory 13 may store data information required for operations of the processor 12 or the display module 11. When the processor 12 executes an application stored in the memory 13, an image data signal and/or an input control signal may be transmitted to the display module 11, and the display module 11 may process the provided signal and output image information on a display.

The power module 14 may include a power supply module such as a power adapter, a battery device, etc., and a power conversion module configured to convert power supplied by the power supply module and generate power required for operations of the electronic equipment 1.

At least one of the components of the electronic equipment 1 may be included in the light-emitting apparatus according to one or more example embodiments described above. In addition, some of individual modules included in a single module on a functional basis may be included in a light-emitting apparatus, and the other may be provided separately from the light-emitting apparatus. For example, the light-emitting apparatus may include the display module, and the processor 12, the memory 13, and the power module 14 may be provided as an apparatus other than the light-emitting apparatus in the electronic equipment 1.

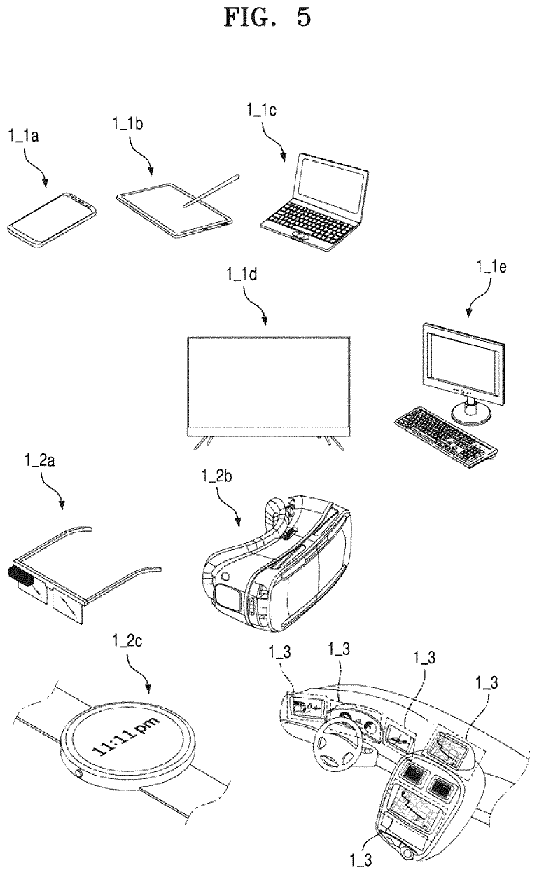

### [Description of FIG. 5]

FIG. 5 is schematic views of electronic equipment according to one or more embodiments.

Referring to FIG. 5, various electronic equipment to which the electronic apparatus (for example, the light-emitting apparatus) is applied may include not only electronic equipment for image display, such as a smartphone 1_1a, a tablet personal computer (PC) 1_1b, a laptop 1_1c, a television (TV) 1_1d, a desk monitor 1_1e, etc., but also wearable electronic equipment including a display module, such as smart glasses 1_2a, a head mount display 1_2b, a smart watch 1_2c, etc., electronic equipment for vehicles 1_3 including a display module, such as a room mirror display, a dashboard of a vehicle, a center fascia, a center information display (CID) arranged on a center fascia, etc.

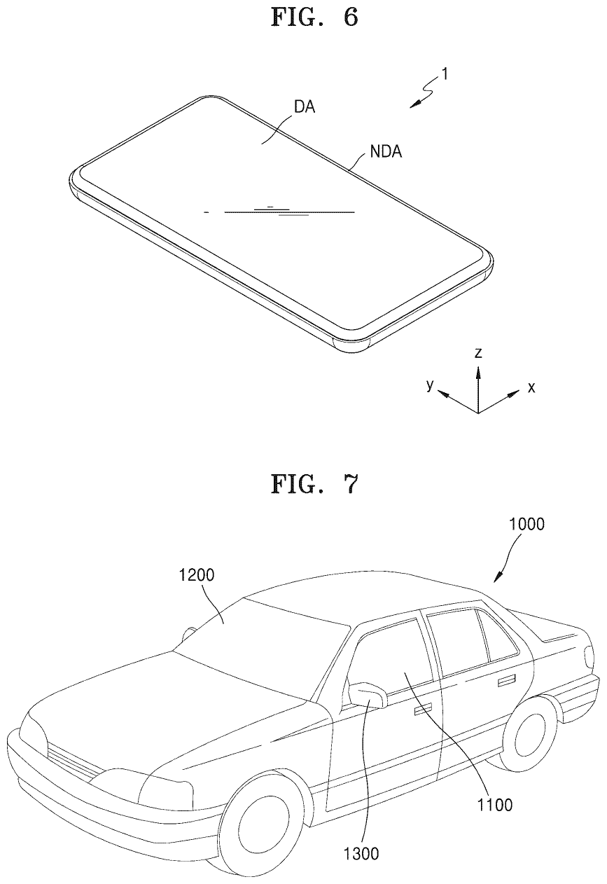

### [Description of FIG. 6]

FIG. 6 is a schematic perspective view of the electronic equipment 1 including the light-emitting device according to some example embodiments. The electronic equipment 1 may be, as a device apparatus, that displays a moving image or still image, a portable electronic equipment, such as a mobile phone, a smart phone, a tablet personal computer (PC), a mobile communication terminal, an electronic notebook, an electronic book, a portable multimedia player (PMP), a navigation, or an ultra mobile PC (UMPC) as well as various products, such as a television, a laptop, a monitor, a billboard, or an Internet of things (IOT) device. The electronic equipment 1 may be such a product above or a part thereof. In addition, the electronic equipment 1 may be a wearable device, such as a smart watch, a watch phone, a glasses-type display, or a head mounted display (HMD), or a part of the wearable device. However, the example embodiments of the disclosure are not limited thereto. For example, the electronic equipment 1 may be a center information display (CID) on an instrument panel and a center fascia or dashboard of a vehicle, a room mirror display instead of a side mirror of a vehicle, an entertainment display for a rear seat of a vehicle, a display placed on the back of a front seat, a head up display (HUD) installed in front of a vehicle or projected on a front window glass, or a computer generated hologram augmented reality head up display (CGH AR HUD). FIG. 6 illustrates some example embodiments in which the electronic equipment 1 is a smartphone for convenience of explanation.

The electronic equipment 1 may include a display area DA and a non-display area NDA outside the display area DA. A display device may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display elements arranged on the display area DA may be arranged. On the non-display area NDA, a pad, which is an area to which an electronic element or a printing circuit board may be electrically connected, may be arranged.

In the electronic equipment 1, a length in the x-axis direction and a length in the y-axis direction may be different from each other. For example, as shown in FIG. 6, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

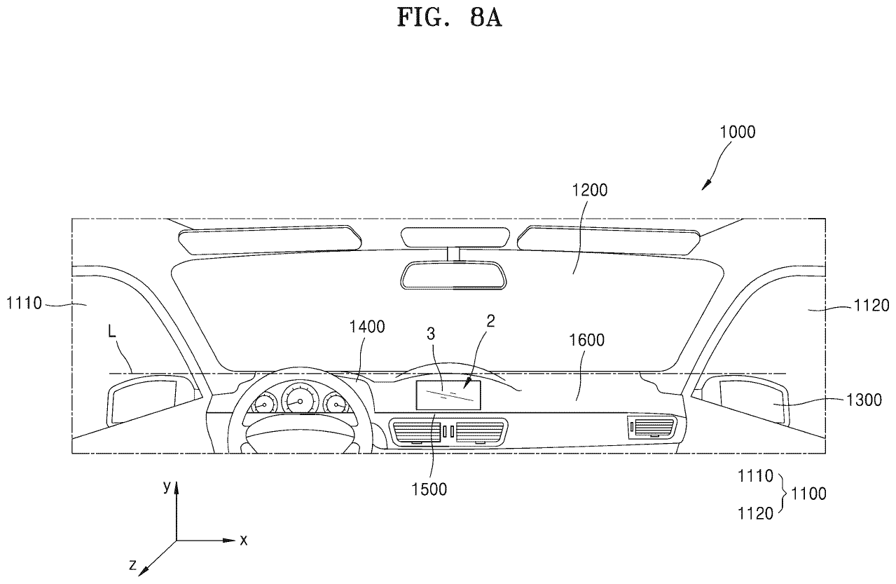

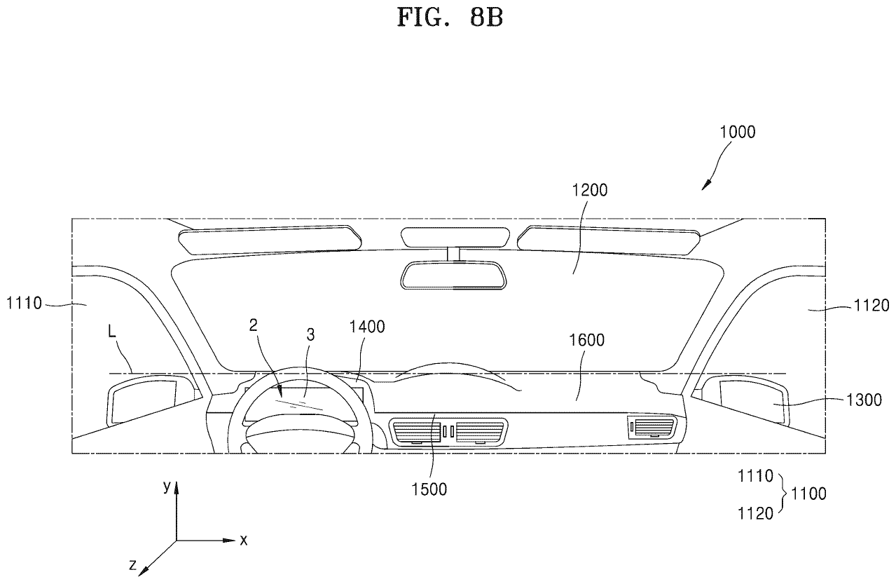

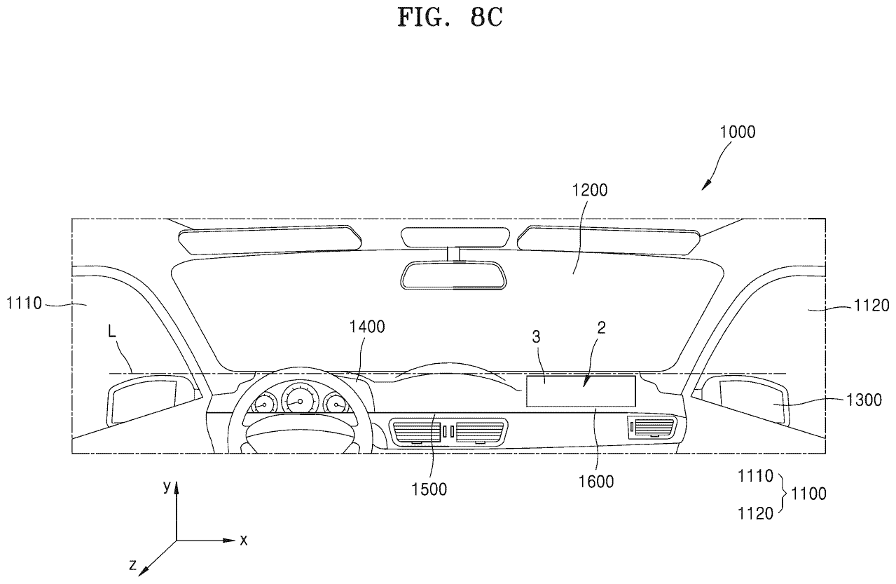

### [Description of FIG. 7 and FIGS. 8A to 8C]

FIG. 7 is a diagram illustrating the exterior of a vehicle 1000 as electronic equipment including a light-emitting device according to some example embodiments. FIGS. 8A to 8C are each a schematic view of the interior of the vehicle 1000 according to one or more example embodiments.

Referring to FIGS. 7, 8A, 8B, and 8C, the vehicle 1000 may refer to various apparatuses for moving a subject object to be transported, such as a human, an object, or an animal, from a departure point to a destination. The vehicle 1000 may include a vehicle traveling on a road or track, a vessel moving over a sea or river, an airplane flying in the sky using the action of air, and the like.

The vehicle 1000 may travel on a road or a track. The vehicle 1000 may move in a predetermined direction according to the rotation of at least one wheel. For example, the vehicle 1000 may include a three-wheeled or four-wheeled vehicle, a construction machine, a two-wheeled vehicle, a prime mover device, a bicycle, and a train running on a track.

The vehicle 1000 may include a body having an interior and an exterior, and a chassis in which mechanical apparatuses necessary for driving are installed as other parts except for the body. The exterior of the body of the vehicle may include a front panel, a bonnet, a roof panel, a rear panel, a trunk, a pillar provided at a boundary between doors, and the like. The chassis of the vehicle 1000 may include a power generating device, a power transmitting device, a driving device, a steering device, a braking device, a suspension device, a transmission device, a fuel device, front and rear wheels, left and right wheels, and the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In some example embodiments, the side window glass 1100 may be installed on a door of the vehicle 1000. A plurality of side window glasses 1100 may be provided and may face each other. In some example embodiments, the side window glass 1100 may include a first side window glass 1110 and a second side window glass 1120. In some example embodiments, the first side window glass 1110 may be arranged adjacent to the cluster 1400. The second side window glass 1120 may be arranged adjacent to the passenger seat dashboard 1600.

In some example embodiments, the side window glasses 1100 may be spaced apart from each other in the x-direction or the −x-direction. For example, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the body of the vehicle. In some example embodiments, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of a steering wheel. The cluster 1400 may include a tachometer, a speedometer, a coolant thermometer, a fuel gauge, a turn signal indicator, a high beam indicator, a warning light, a seat belt warning light, an odometer, an tachograph, an automatic shift selector indicator lamp, a door open warning light, an engine oil warning light, and/or a low fuel warning light.

The center fascia 1500 may include a control panel on which a plurality of buttons for adjusting an audio device, an air conditioning device, and a seat heater are disposed.

The center fascia 1500 may be arranged on one side of the cluster 1400.

A passenger seat dashboard 1600 may be spaced apart from the cluster 1400, and the center fascia 1500 may be arranged between the cluster 1400 and the passenger seat dashboard 1600. In some example embodiments, the cluster 1400 may be arranged to correspond to a driver seat (not shown), and the passenger seat dashboard 1600 may be disposed to correspond to a passenger seat (not shown). In some example embodiments, the cluster 1400 may be adjacent to the first side window glass 1110, and the passenger seat dashboard 1600 may be adjacent to the second side window glass 1120.

In some example embodiments, the display apparatus 2 may include a display panel 3, and the display panel 3 may display an image. The display apparatus 2 may be arranged inside the vehicle 1000. In some example embodiments, the display apparatus 2 may be arranged between the side window glasses 1100 facing each other. The display apparatus 2 may be arranged on at least one of the cluster 1400, the center fascia 1500, and the passenger seat dashboard 1600.

The display apparatus 2 may include an organic light-emitting display device, an inorganic electroluminescent (EL) display device, a quantum dot display device, and the like. Hereinafter, as the display apparatus 2 according to some example embodiments of the disclosure, an organic light-emitting display device including the light-emitting device according to the disclosure will be described as an example, but various types of display devices as described above may be used in example embodiments of the disclosure.

Referring to FIG. 8A, the display apparatus 2 may be arranged on the center fascia 1500. In some example embodiments, the display apparatus 2 may display navigation information. In some example embodiments, the display apparatus 2 may display information regarding audio settings, video setting, or vehicle settings.

Referring to FIG. 8B, the display apparatus 2 may be arranged on the cluster 1400. When the display apparatus 2 is arranged on the cluster 1400, the cluster 1400 may display driving information and the like through the display apparatus 2. That is, the cluster 1400 may digitally implement driving information and the like. The cluster 1400 may digitally display vehicle information and driving information as images. For example, a needle and a gauge of a tachometer and various warning light icons may be displayed by a digital signal.

Referring to FIG. 8C, the display apparatus 2 may be arranged on the passenger seat dashboard 1600. The display apparatus 2 may be embedded in the passenger seat dashboard 1600 or arranged on the passenger seat dashboard 1600. In some example embodiments, the display apparatus 2 arranged on the passenger seat dashboard 1600 may display an image related to information displayed on the cluster 1400 and/or information displayed on the center fascia 1500. In one or more example embodiments, the display apparatus 2 arranged on the passenger seat dashboard 1600 may display information different from information displayed on the cluster 1400 and/or information displayed on the center fascia 1500.

As described herein, any devices, systems, modules, portions, units, controllers, circuits, and/or portions thereof according to any of the example embodiments, and/or any portions thereof (including, without limitation, all devices and elements therein, each recited separately in a list, any portion thereof, or the like) may include, may be included in, and/or may be implemented by one or more instances of processing circuitry such as hardware including logic circuits; a hardware/software combination such as a processor executing software; or a combination thereof. For example, the processing circuitry more specifically may include, but is not limited to, a central processing unit (CPU), an arithmetic logic unit (ALU), a graphics processing unit (GPU), an application processor (AP), a digital signal processor (DSP), a microcomputer, a field programmable gate array (FPGA), and programmable logic unit, a microprocessor, application-specific integrated circuit (ASIC), a neural network processing unit (NPU), an Electronic Control Unit (ECU), an Image Signal Processor (ISP), and the like. In some example embodiments, the processing circuitry may include a non-transitory computer readable storage device (e.g., a memory), for example a solid state drive (SSD), storing a program of instructions, and a processor (e.g., CPU) configured to execute the program of instructions to implement the functionality and/or methods performed by some or all of any devices, systems, modules, portions, units, controllers, circuits, and/or portions thereof according to any of the example embodiments.

### [Manufacturing Method]

Respective layers constituting the hole transport region, the emission layer, and respective layers constituting the electron transport region may be formed in a certain region by using one or more suitable methods selected from vacuum deposition, spin coating, casting, Langmuir-Blodgett (LB) deposition, ink-jet printing, laser-printing, and laser-induced thermal imaging.

When respective layers constituting the hole transport region, an emission layer, and respective layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

As used herein, “Cₓ-C_{y}” or “Cₓ to C_{y}” refers that a number (e.g., quantity) of carbons included in a corresponding group is x to y, wherein x and y may each be any natural number. For example, “C₁-C₆” and “C₁ to C₆” means that a number of carbons included the corresponding group is 1 to 6, and “C₆-C₂₀” and “C₆ to C₂₀” means that a number of carbons included in the corresponding group is 6 to 20.

The term “monovalent hydrocarbon group” as used herein may refer to a monovalent moiety derived from an organic compound including carbon and hydrogen, or a derivative thereof, and the monovalent hydrocarbon group may include, for example, a linear or branched alkyl group (for example, a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (a cycloalkyl group) (for example, a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group; a monovalent unsaturated aliphatic hydrocarbon group (an alkenyl group, and an alkynyl group) (for example, an allyl group); a monovalent unsaturated cycloaliphatic hydrocarbon group (a cycloalkenyl group) (for example, 3-cyclohexenyl); an aryl group (for example, a phenyl group, a 1-naphthyl group, and a 2-naphthyl group); an arylalkyl group (for example, a benzyl group, and a diphenylmethyl group); a monovalent hydrocarbon group containing one or more heteroatoms (for example, a tetrahydrofuranyl group, a methoxymethyl group, a ethoxymethyl group, a methylthiomethyl group, an acetamidemethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group), or any combination thereof. In addition, in these groups, some hydrogen atoms may be substituted with a moiety containing one or more heteroatoms such as oxygen, sulfur, nitrogen, or a halogen atom, or some carbon atoms may be replaced by a moiety containing one or more heteroatoms such as oxygen, sulfur, or nitrogen, and thus, these groups may contain a hydroxyl group, a cyano group, a carbonyl group, a carboxyl group, an ether bond, an ester bond, a sulfonic acid ester bond, a carbonate group, a lactone ring, a sultone ring, a carboxylic acid anhydride moiety, or a haloalkyl moiety.

The term “divalent hydrocarbon group” as used herein refers to a divalent moiety, in which any one hydrogen of the monovalent hydrocarbon group is replaced by a bonding site with an adjacent atom. The divalent hydrocarbon group may include, for example, a linear or branched alkylene group, a cycloalkylene group, an alkenylene group, an alkynylene group, a cycloalkylene group, an arylene group, groups in which some carbon atoms of the aforementioned groups are replaced by heteroatoms, and the like.

The term “alkylthio group” as used herein refers to a monovalent group having a formula of —SA₁₀₁, wherein A₁₀₁ is an alkyl group.

As used herein, the term “arylene group” refers to a divalent group having a carbocyclic aromatic system.

The term “heteroaryloxy group” as used herein refers to a monovalent group having a formula of —OA₁₀₉, wherein A₁₀₉ is a heteroaryl group.

The term “heteroarylthio group” refers to a monovalent group having a chemical formula of —SA₁₀₅, wherein A₁₀₅ is a heteroaryl group.

The term “arylalkyl group” refers to a group in which a monovalent group having a carbocyclic aromatic system is substituted for an alkyl group, and specific examples thereof include a benzyl group, a diphenylmethyl group, and the like.

The term “heteroarylalkyl group” refers to a group in which a monovalent group having a heterocyclic aromatic system is substituted for an alkyl group.

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the C₁-C₆₀ heterocyclic group has 3 to 61 ring-forming atoms.

The “cyclic group” as used herein may include the C₃-C₆₀ carbocyclic group, and the C₁-C₆₀ heterocyclic group.

The term “π electron-rich C₃-C₆₀ cyclic group” as used herein refers to a cyclic group that has three to sixty carbon atoms and does not include *—N═*′ as a ring-forming moiety, and the term “π electron-deficient nitrogen-containing C₁-C₆₀ cyclic group” as used herein refers to a heterocyclic group that has one to sixty carbon atoms and includes *—N═*′ as a ring-forming moiety.

For example, the C₃-C₆₀ carbocyclic group may be i) group T1 or ii) a condensed cyclic group in which two or more groups T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) group T2, ii) a condensed cyclic group in which two or more groups T2 are condensed with each other, or iii) a condensed cyclic group in which at least one group T2 and at least one group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the π electron-rich C₃-C₆₀ cyclic group may be i) group T1, ii) a condensed cyclic group in which two or more groups T1 are condensed with each other, iii) group T3, iv) a condensed cyclic group in which two or more groups T3 are condensed with each other, or v) a condensed cyclic group in which at least one group T3 and at least one group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),
the π electron-deficient nitrogen-containing C₁-C₆₀ cyclic group may be i) group T4, ii) a condensed cyclic group in which two or more groups T4 are condensed with each other, iii) a condensed cyclic group in which at least one group T4 and at least one group T1 are condensed with each other, iv) a condensed cyclic group in which at least one group T4 and at least one group T3 are condensed with each other, or v) a condensed cyclic group in which at least one group T4, at least one group T1, and at least one group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),
the group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,
the group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the group T3 may be a furan group, a thiophene group, a 1H-pyrrole group, a silole group, or a borole group, and
the group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (for example, a divalent group, a trivalent group, a tetravalent group, etc.) according to the structure of a formula for which the corresponding term is used. For example, the “benzene group” may be a benzo group, a phenyl group, a phenylene group, or the like, which may be easily understood by one of ordinary skill in the art according to the structure of a formula including the “benzene group.”

In some example embodiments, examples of the monovalent C₃-C₆₀ carbocyclic group and the monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has one to sixty carbon atoms, and specific examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof are an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group, a propynyl group, and the like. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group of 1 to 10 carbon atoms, further including, in addition to carbon atoms, at least one heteroatom, as ring-forming atoms, and specific examples are a 1,2,3,4-oxatriazolidinyl group, a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term C₃-C₁₀ cycloalkenyl group used herein refers to a monovalent cyclic group that has three to ten carbon atoms and at least one carbon-carbon double bond in the ring thereof and no aromaticity, and specific examples thereof are a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group of 1 to 10 carbon atoms, further including, in addition to carbon atoms, at least one heteroatom, as ring-forming atoms, and having at least one double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of 6 to 60 carbon atoms, and the term “C₆-C₆₀ arylene group” as used herein refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as used herein refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed to each other, further including, in addition to a carbon atom, at least one heteroatom as a ring-forming atom, and having no aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, and a benzothienodibenzothiophenyl group.

The term “divalent non-aromatic condensed heteropolycyclic group” as used herein refers to a divalent group having the same structure as the monovalent non-aromatic condensed heteropolycyclic group described above.

The term “C₆-C₆₀ aryloxy group” as used herein indicates —OA₁₀₂ (wherein A₁₀₂ is a C₆-C₆₀ aryl group), and the term “C₆-C₆₀ arylthio group” as used herein indicates —SA₁₀₃ (wherein A₁₀₃ is a C₆-C₆₀ aryl group).

The term “C₇-C₆₀ aryl alkyl group” used herein refers to -A₁₀₄A₁₀₅ (where A₁₀₄ may be a C₁-C₅₄ alkylene group, and A₁₀₅ may be a C₆-C₅₉ aryl group), and the term C₂-C₆₀ heteroaryl alkyl group” used herein refers to -A₁₀₆A₁₀₇ (where A₁₀₆ may be a C₁-C₅₉ alkylene group, and A₁₀₇ may be a C₁-C₅₉ heteroaryl group).

The term “R₁₀ₐ” as used herein refers to:
deuterium, —F, —CI, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).
Q₁ to Q₃, Q₁₁ to Q₁₃, Q₂₁ to Q₂₃, and Q₃₁ to Q₃₃ as used herein may each independently be: hydrogen; deuterium; —F; —CI; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof.

The term “heteroatom” as used herein refers to any atom other than a carbon atom and a hydrogen atom. Examples of the heteroatom are O, S, N, P, Si, B, Ge, Se, and any combinations thereof.

The term “transition metal” used herein includes hafnium (Hf), tantalum (Ta), tungsten (W), rhenium (Re), osmium (Os), iridium (Ir), platinum (Pt), gold (Au), and the like.

“Ph” as used herein refers to a phenyl group, “Me” as used herein refers to a methyl group, “Et” as used herein refers to an ethyl group, “tert-Bu” or “Bu^{t}” as used herein refers to a tert-butyl group, and “OMe” as used herein refers to a methoxy group.

The term “biphenyl group” as used herein refers to “a phenyl group substituted with a phenyl group.” In other words, the “biphenyl group” is a substituted phenyl group having a C₆-C₆₀ aryl group as a substituent.

The term “terphenyl group” as used herein refers to “a phenyl group substituted with a biphenyl group”. In other words, the “terphenyl group” is a substituted phenyl group having, as a substituent, a C₆-C₆₀ aryl group substituted with a C₆-C₆₀ aryl group.
* and *′ as used herein, unless defined otherwise, each refer to a binding site to a neighboring atom in a corresponding formula or moiety.

In the specification, the terms “x-axis”, “y-axis”, and “z-axis” are not limited to three axes in an orthogonal coordinate system, and may be interpreted in a broader sense than the aforementioned three axes in an orthogonal coordinate system. For example, the x-axis, y-axis, and z-axis may describe axes that are orthogonal to each other, or may describe axes that are in different directions that are not orthogonal to each other

### EXAMPLES

### Synthesis Example 1: Synthesis of Compound 1

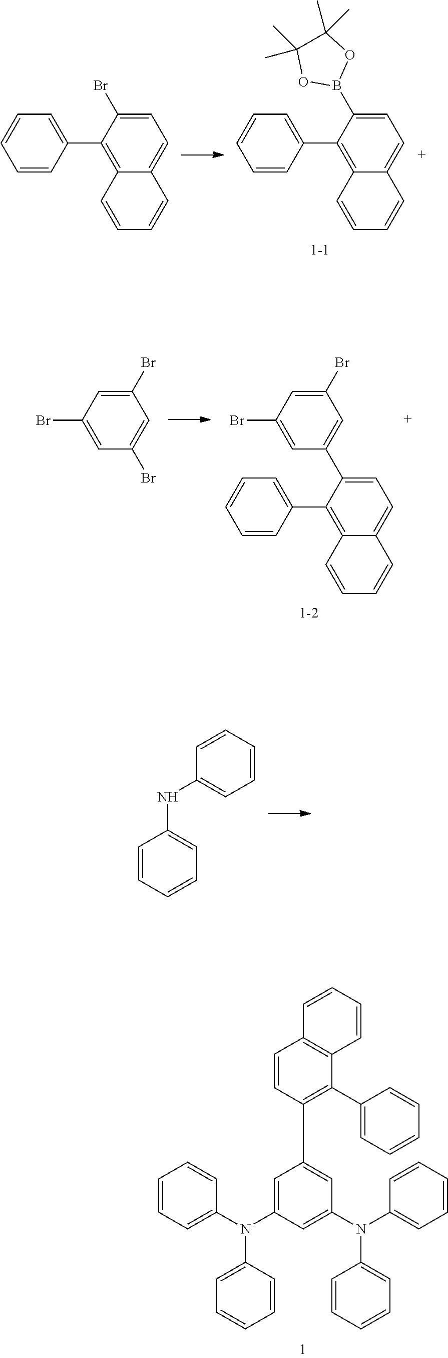 [SMILES: Brc1ccc2ccccc2c1-c1ccccc1 ; CC1(C)OB(c2ccc3ccccc3c2-c2ccccc2)OC1(C)C ; Brc1cc(Br)cc(Br)c1 ; Brc1cc(Br)cc(-c2ccc3ccccc3c2-c2ccccc2)c1 ; c1ccc(Nc2ccccc2)cc1 ; c1ccc(-c2c(-c3cc(N(c4ccccc4)c4ccccc4)cc(N(c4ccccc4)c4ccccc4)c3)ccc3ccccc23)cc1]

### Synthesis of Intermediate 1-1

2.83 g (10 mmol) of 2-bromo-1-phenylnaphthalene was dissolved in 30 ml of THF, and 5 ml of n-BuLi (2.5 M in hexane) was added thereto at −78° C. After 1 hour, 4.08 ml (40 mmol) of 2-isopropoxy-4,4,5,5-tetramethyl-1,3,2-dioxaborolane was added thereto at the same temperature. The reaction solution was stirred at room temperature for 5 hours, and water was added thereto. Then, the resultant mixture was washed with 30 ml of diethyl ether three times. The washed diethyl ether layer was dried using MgSO₄ and then dried under reduced pressure to obtain a product, which was then separated and purified by silica gel column chromatography, to thereby obtain 2.48 g (yield: 75%) of Intermediate 1-1 as a white solid. The resulting compound was identified by LC-MS. C₂₂H₂₃BO₂: M⁺ 330.2

### Synthesis of Intermediate 1-2

3.30 g (10.0 mmol) of Intermediate 1-1, 3.78 g (12.0 mmol) of 1,3,5-tribromobenzene, 0.58 g (0.5 mmol) of Pd(PPh₃)₄, and 4.14 g (30.0 mmol) of K₂CO₃ were dissolved in 60 ml of a mixed solution of THF/H₂O (2:1 by volume) and then stirred for 16 hours at 80° C. The reaction solution was cooled to room temperature, and then an extraction process was performed thereon three times by using 60 ml of water and 60 ml of diethyl ether. The resulting organic layer was dried by using MgSO₄. A solvent was then removed therefrom by evaporation. The resulting residue was separated and purified through silica gel chromatography to thereby obtain 3.07 g of Intermediate 1-2 (yield: 70%). The resulting compound was identified by LC-MS. C₂₂H₁₄Br₂: M⁺ 438.1

### Synthesis of Compound 1

4.38 g (10 mmol) of Intermediate 1-2, 3.72 g (22 mmol) of diphenylamine, 0.92 g (1 mmol) of tris(dibenzylideneacetone)dipalladium(0) (Pd₂dba₃), 0.48 g (2 mmol) of P(t-Bu)3, and 5.76 g (60 mmol) of sodium tert-butoxide were dissolved in 60 ml of toluene, followed by stirred at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 4.31 g (yield: 70%) of Compound 1. The resulting compound was identified by LC-MS. C₄₆H₃₄N₂: M⁺ 614.7

### Synthesis Example 2: Synthesis of Compound 6

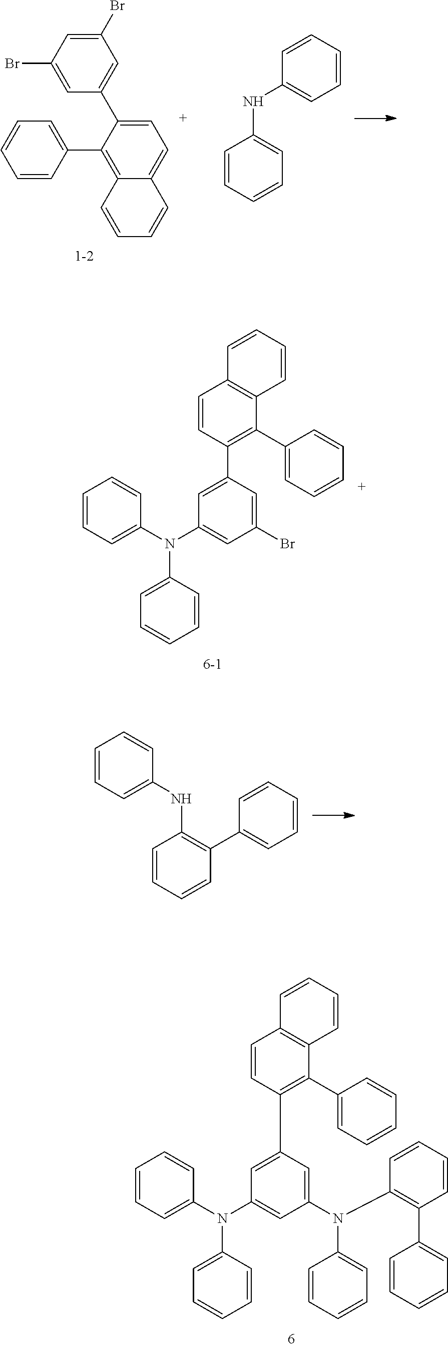 [SMILES: Brc1cc(Br)cc(-c2ccc3ccccc3c2-c2ccccc2)c1 ; c1ccc(Nc2ccccc2)cc1 ; Brc1cc(-c2ccc3ccccc3c2-c2ccccc2)cc(N(c2ccccc2)c2ccccc2)c1 ; c1ccc(Nc2ccccc2-c2ccccc2)cc1 ; c1ccc(-c2ccccc2N(c2ccccc2)c2cc(-c3ccc4ccccc4c3-c3ccccc3)cc(N(c3ccccc3)c3ccccc3)c2)cc1]

### Synthesis of Intermediate 6-1

4.38 g (10 mmol) of Intermediate 1-2, 1.69 g (10 mmol) of diphenylamine, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene, followed by stirring at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 3.68 g (yield: 70%) of Intermediate 6-1. The resulting compound was identified by LC-MS. C₃₄H₂₄BrN: M⁺ 526.4

### Synthesis of Compound 6

5.26 g (10 mmol) of Intermediate 6-1, 1.69 g (10 mmol) of N-phenyl-[1,1′-biphenyl]-2-amine, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene and then stirred at 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 4.83 g (yield: 70%) of Compound 6. The resulting compound was identified by LC-MS. C₅₂H₃₈N₂: M⁺ 690.3

### Synthesis Example 3: Synthesis of Compound 7

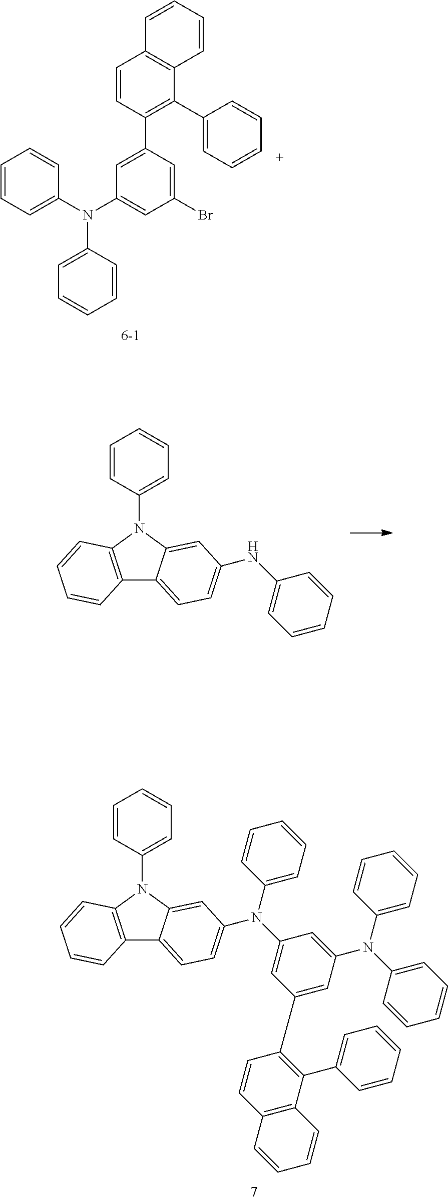 [SMILES: Brc1cc(-c2ccc3ccccc3c2-c2ccccc2)cc(N(c2ccccc2)c2ccccc2)c1 ; c1ccc(Nc2ccc3c4ccccc4n(-c4ccccc4)c3c2)cc1 ; c1ccc(-c2c(-c3cc(N(c4ccccc4)c4ccccc4)cc(N(c4ccccc4)c4ccc5c6ccccc6n(-c6ccccc6)c5c4)c3)ccc3ccccc23)cc1]

Compound 7 was synthesized in the same manner as in the synthesis of Compound 6, except that N,9-diphenyl-9H-carbazol-2-amine was used instead of N-phenyl-[1,1′-biphenyl]-2-amine. The resulting compound was identified by LC-MS. C₅₈H₄₁N₃: M+ 779.9

### Synthesis Example 4: Synthesis of Compound 23

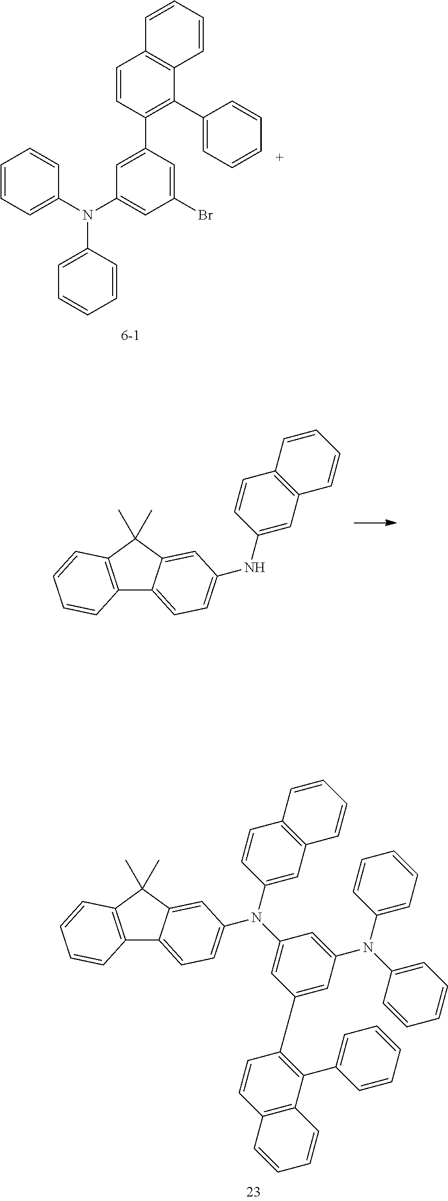 [SMILES: Brc1cc(-c2ccc3ccccc3c2-c2ccccc2)cc(N(c2ccccc2)c2ccccc2)c1 ; CC1(C)c2ccccc2-c2ccc(Nc3ccc4ccccc4c3)cc21 ; CC1(C)c2ccccc2-c2ccc(N(c3cc(-c4ccc5ccccc5c4-c4ccccc4)cc(N(c4ccccc4)c4ccccc4)c3)c3ccc4ccccc4c3)cc21]

Compound 23 was synthesized in the same manner as in the synthesis of Compound 6, except that 9,9-dimethyl-N-(naphthalen-2-yl)-9H-fluoren-2-amine was used instead of N-phenyl-[1,1′-biphenyl]-2-amine. The resulting compound was identified by LC-MS. C₅₉H₄₄N₂: M⁺ 781.0

### Synthesis Example 5: Synthesis of Compound 67

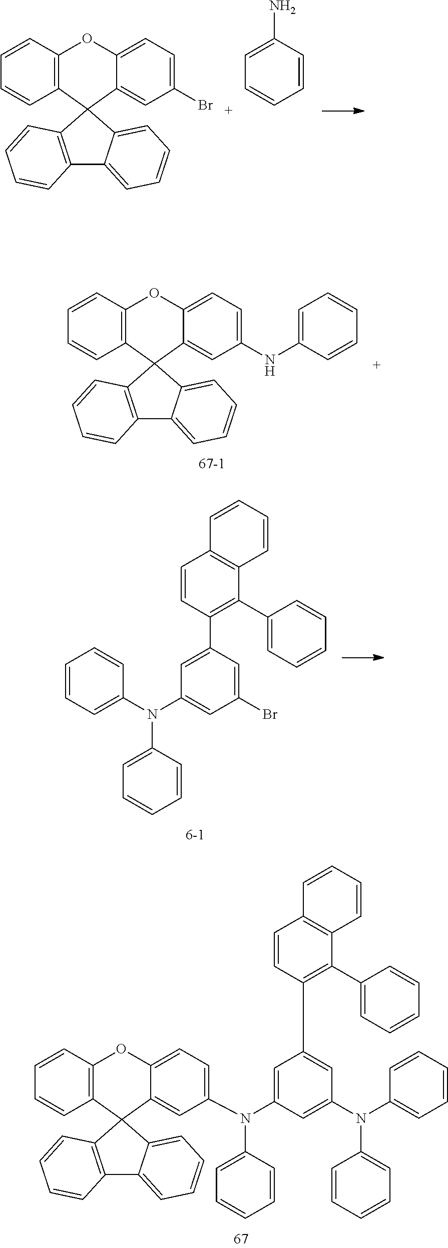

### Synthesis of Intermediate 67-1

4.11 g (10 mmol) of 2′-bromospiro[fluorene-9,9′-xanthene], 1.12 g (12 mmol) of aniline, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene and then stirred at 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 2.97 g (yield: 70%) of Intermediate 67-1. The resulting compound was identified by LC-MS. C₃₁H₂₁ NO: M⁺ 423.5

### Synthesis of Compound 67

4.24 g (10 mmol) of Intermediate 67-1, 5.26 g (10 mmol) of Intermediate 6-1, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene, followed by stirring at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 6.08 g (yield: 70%) of Compound 67. The resulting compound was identified by LC-MS. C₆₅H₄₄N₂O: M⁺ 869.0

### Synthesis Example 6: Synthesis of Compound 85

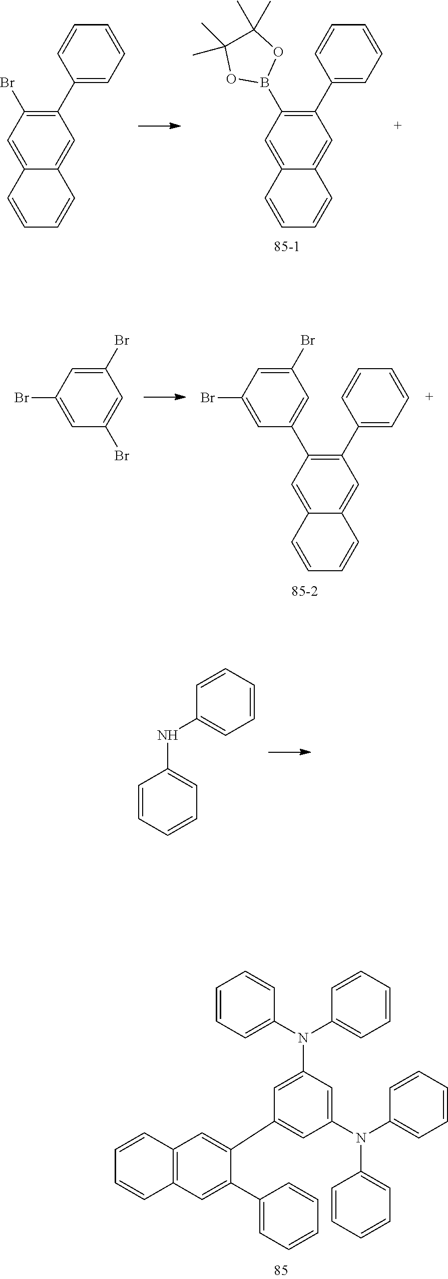 [SMILES: Brc1cc2ccccc2cc1-c1ccccc1 ; CC1(C)OB(c2cc3ccccc3cc2-c2ccccc2)OC1(C)C ; Brc1cc(Br)cc(Br)c1 ; Brc1cc(Br)cc(-c2cc3ccccc3cc2-c2ccccc2)c1 ; c1ccc(Nc2ccccc2)cc1 ; c1ccc(-c2cc3ccccc3cc2-c2cc(N(c3ccccc3)c3ccccc3)cc(N(c3ccccc3)c3ccccc3)c2)cc1]

### Synthesis of Intermediate 85-1

2.83 g (10 mmol) of 2-bromo-3-phenylnaphthalene was dissolved in 30 ml of THF, and 5 ml of n-BuLi (2.5 M in hexane) was added thereto at −78° C. After 1 hour, 4.08 ml (40 mmol) of 2-isopropoxy-4,4,5,5-tetramethyl-1,3,2-dioxaborolane was added thereto at the same temperature. The reaction solution was stirred at room temperature for 5 hours, and water was added thereto. Then, the resultant mixture was washed with 30 ml of diethyl ether three times. The washed diethyl ether layer was dried using MgSO₄ and then dried under reduced pressure to obtain a product, which was then separated and purified by silica gel column chromatography, to thereby obtain 2.31 g (yield: 70%) of Intermediate 85-1 as a white solid. The resulting compound was identified by LC-MS. C₂₂H₂₃BO₂: M⁺ 330.2

### Synthesis of Intermediate 85-2

3.30 g (10.0 mmol) of Intermediate 85-1, 3.78 g (12.0 mmol) of 1,3,5-tribromobenzene, 0.58 g (0.5 mmol) of Pd(PPh₃)₄, and 4.14 g (30.0 mmol) of K₂CO₃ were dissolved in 60 ml of a mixed solution of THF/H₂O (2:1 by volume) and then stirred for 16 hours at 80° C. The reaction solution was cooled at room temperature, followed by three times of an extraction process using 60 ml of water and 60 ml of diethyl ether. The resulting organic layer was dried by using MgSO₄. A solvent was then removed therefrom by evaporation. The resulting residue was separated and purified through silica gel chromatography to thereby obtain 3.15 g of Intermediate 85-2 (yield: 72%). The resulting compound was identified by LC-MS. C₂₂H₁₄Br₂: M⁺ 438.1

### Synthesis of Compound 85

4.38 g (10 mmol) of Intermediate 85-2, 3.72 g (22 mmol) of diphenylamine, 0.92 g (1 mmol) of tris(dibenzylideneacetone)dipalladium(0) (Pd₂dba₃), 0.48 g (2 mmol) of P(t-Bu)3, and 5.76 g (60 mmol) of sodium tert-butoxide were dissolved in 60 ml of toluene, followed by stirred at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 4.31 g (yield: 70%) of Compound 85. The resulting compound was identified by LC-MS. C₄₆H₃₄N₂: M⁺ 614.7

### Synthesis Example 7: Synthesis of Compound 97

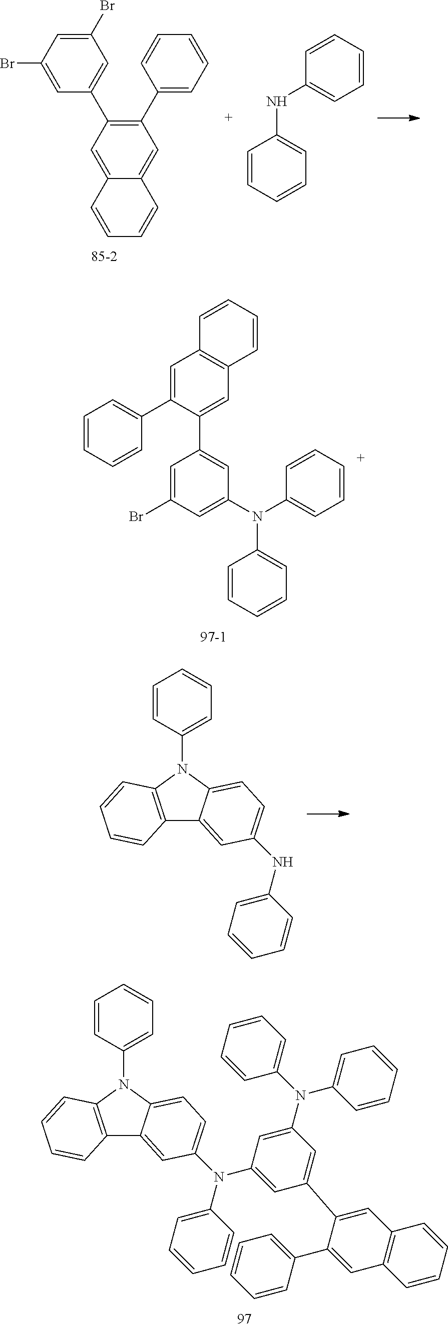 [SMILES: Brc1cc(Br)cc(-c2cc3ccccc3cc2-c2ccccc2)c1 ; c1ccc(Nc2ccccc2)cc1 ; Brc1cc(-c2cc3ccccc3cc2-c2ccccc2)cc(N(c2ccccc2)c2ccccc2)c1 ; c1ccc(Nc2ccc3c(c2)c2ccccc2n3-c2ccccc2)cc1 ; c1ccc(-c2cc3ccccc3cc2-c2cc(N(c3ccccc3)c3ccccc3)cc(N(c3ccccc3)c3ccc4c(c3)c3ccccc3n4-c3ccccc3)c2)cc1]

### Synthesis of Intermediate 97-1

4.38 g (10 mmol) of Intermediate 85-2, 1.69 g (10 mmol) of diphenylamine, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene, followed by stirring at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 2.89 g (yield: 55%) of Intermediate 97-1. The resulting compound was identified by LC-MS. C₃₄H₂₄BrN: M⁺ 526.4

### Synthesis of Compound 97

4.38 g (10 mmol) of Intermediate 97-1, 3.34 g (10 mmol) of N,9-diphenyl-9H-carbazol-3-amine, 0.46 g (0.5 mmol) of tris(dibenzylideneacetone)dipalladium(0) (Pd₂dba₃), 0.24 g (1 mmol) of P(t-Bu)3, and 2.88 g (30 mmol) of sodium tert-butoxide were dissolved in 60 ml of toluene and then stirred at 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 5.45 g (yield: 70%) of Compound 97. The resulting compound was identified by LC-MS. C₅₈H₄₁N₃: M⁺ 779.3

### Synthesis Example 8: Synthesis of Compound 111

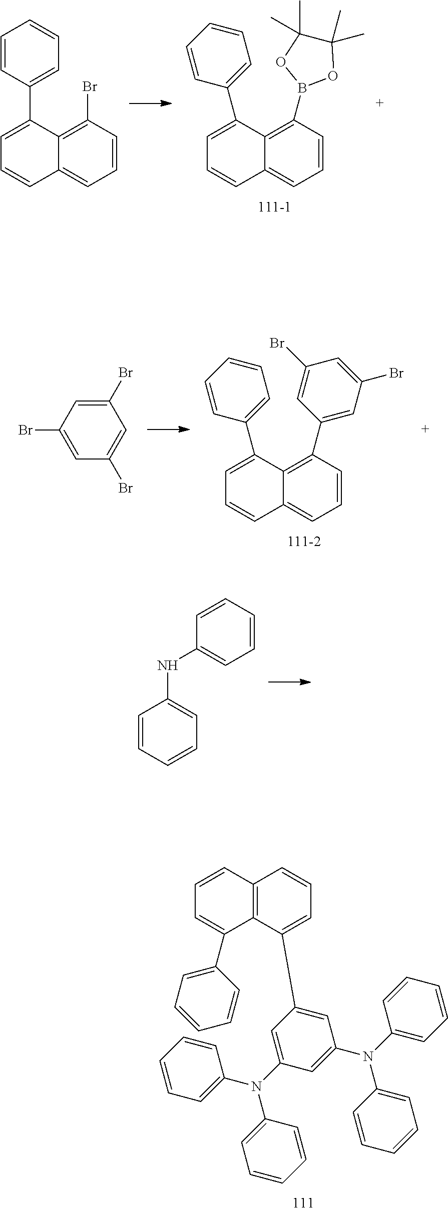 [SMILES: Brc1cccc2cccc(-c3ccccc3)c12 ; CC1(C)OB(c2cccc3cccc(-c4ccccc4)c23)OC1(C)C ; Brc1cc(Br)cc(Br)c1 ; Brc1cc(Br)cc(-c2cccc3cccc(-c4ccccc4)c23)c1 ; c1ccc(Nc2ccccc2)cc1 ; c1ccc(-c2cccc3cccc(-c4cc(N(c5ccccc5)c5ccccc5)cc(N(c5ccccc5)c5ccccc5)c4)c23)cc1]

### Synthesis of Intermediate 111-1

2.83 g (10 mmol) of 1-bromo-8-phenylnaphthalene was dissolved in 30 ml of THF, and 5 ml of n-BuLi (2.5 M in hexane) was added thereto at −78° C. After 1 hour, 4.08 ml (40 mmol) of 2-isopropoxy-4,4,5,5-tetramethyl-1,3,2-dioxaborolane was added thereto at the same temperature. The reaction solution was stirred at room temperature for 5 hours, and water was added thereto. Then, the resultant mixture was washed with 30 ml of diethyl ether three times. The washed diethyl ether layer was dried using MgSO₄ and then dried under reduced pressure to obtain a product, which was then separated and purified by silica gel column chromatography, to thereby obtain 1.65 g (yield: 50%) of Intermediate 111-1 as a white solid. The resulting compound was identified by LC-MS. C₂₂H₂₃BO₂: M⁺ 330.2

### Synthesis of Intermediate 111-2

3.30 g (10.0 mmol) of Intermediate 111-1, 3.78 g (12.0 mmol) of 1,3,5-tribromobenzene, 0.58 g (0.5 mmol) of Pd(PPh₃)₄, and 4.14 g (30.0 mmol) of K₂CO₃ were dissolved in 60 ml of a mixed solution of THF/H₂O (2:1 by volume) and then stirred for 16 hours at 80° C. The reaction solution was cooled to room temperature, and then an extraction process was performed thereon three times by using 60 ml of water and 60 ml of diethyl ether. The resulting organic layer was dried by using MgSO₄. A solvent was then removed therefrom by evaporation. The resulting residue was separated and purified through silica gel chromatography to thereby obtain 2.23 g of Intermediate 111-2 (yield: 51%). The resulting compound was identified by LC-MS. C₂₂H₁₄Br₂: M⁺ 438.1

### Synthesis of Compound 111

4.38 g (10 mmol) of Intermediate 111-2, 3.72 g (22 mmol) of diphenylamine, 0.92 g (1 mmol) of tris(dibenzylideneacetone)dipalladium(0) (Pd₂dba₃), 0.48 g (2 mmol) of P(t-Bu)3, and 5.76 g (60 mmol) of sodium tert-butoxide were dissolved in 60 ml of toluene, followed by stirred at a temperature of 80° C. for 3 hours. The reaction solution was cooled to room temperature, and then, 40 ml of water was added thereto, followed by three times of extraction process using 50 ml of ethyl ether. The collected organic layer was dried using MgSO₄, and the residue obtained by evaporating the solvent was separated and purified by silica gel chromatography, to thereby obtain 4.31 g (yield: 70%) of Compound 111. The resulting compound was identified by LC-MS. C₄₆H₃₄N₂: M⁺ 614.7

### Comparative Example 1

As an anode, a glass substrate (product of Corning Inc.) with a 15 Ω/cm² (1,200 Å) ITO electrode formed thereon was cut to a size of 50 mm×50 mm×0.7 mm, sonicated using isopropyl alcohol and pure water each for 5 minutes, and then cleaned by irradiation of ultraviolet rays and exposure of ozone thereto for 30 minutes. Then, the resultant glass substrate was mounted on a vacuum deposition apparatus.

NPB was deposited on the anode to form a hole injection layer having a thickness of 300 Å. HT3 was then deposited on the hole injection layer to form a hole transport layer having a thickness of 200 Å. CzSi was deposited on the hole transport layer to form an emission auxiliary layer having a thickness of 100 Å.

HTH, ETH, PD40, and t-DABNA were co-deposited on the emission auxiliary layer at a weight ratio of 42:42:15:1 to form an emission layer having a thickness of 200 Å. In this regard, HTH and ETH were a host, PD40 was a phosphorescent dopant (sensitizer), and t-DABNA was a delayed fluorescence material.

TSPO1 was deposited on the emission layer to form a hole-blocking layer having a thickness of 200 Å, and then, TPBI was deposited on the hole-blocking layer to form an electron transport layer having a thickness of 300 Å. LiF was deposited on the electron transport layer to form an electron injection layer having a thickness of 10 Å, and Al was deposited on the electron injection layer to form a cathode having a thickness of 3,000 Å, thereby manufacturing a light-emitting device.

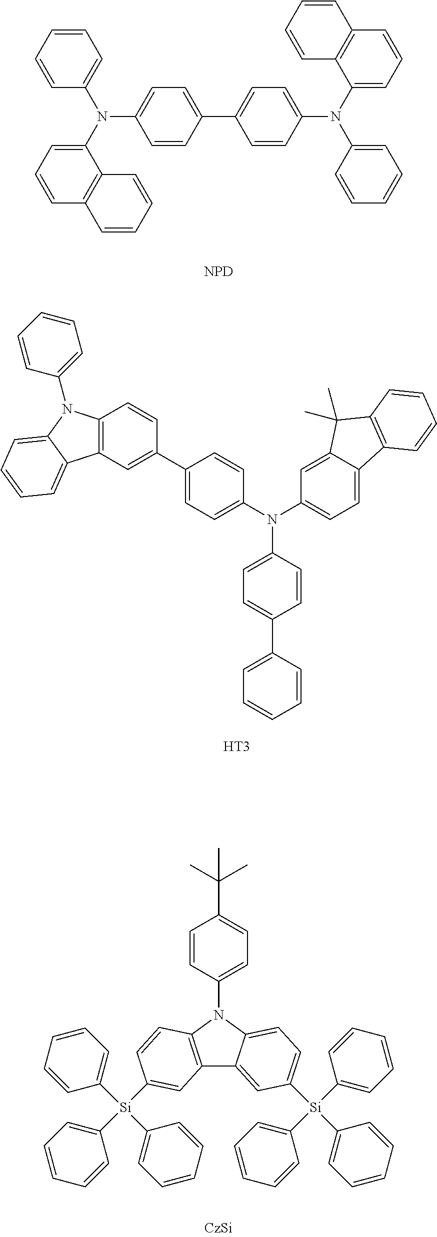 [SMILES: c1ccc(N(c2ccc(-c3ccc(N(c4ccccc4)c4cccc5ccccc45)cc3)cc2)c2cccc3ccccc23)cc1 ; "NPD" ; CC1(C)c2ccccc2-c2ccc(N(c3ccc(-c4ccccc4)cc3)c3ccc(-c4ccc5c(c4)c4ccccc4n5-c4ccccc4)cc3)cc21 ; "HT3" ; CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1]

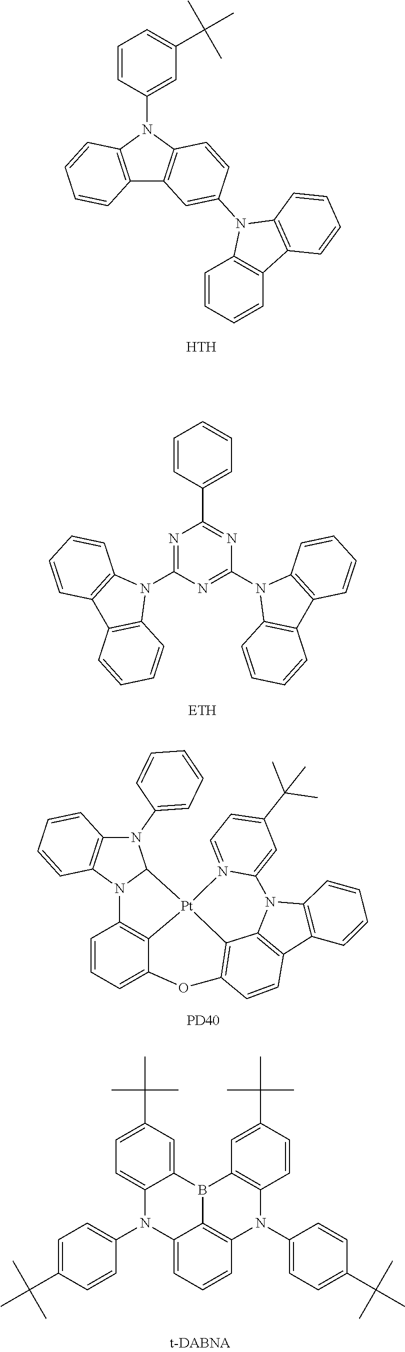

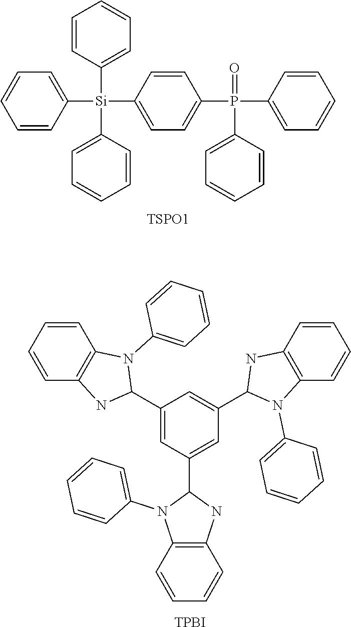

### Examples 1 to 8 and Comparative Examples 2 to 6

Light-emitting devices were manufactured in the same manner as in Comparative Example 1, except that the hole transport layer material was changed as shown in Table 1.

### Evaluation Example

The driving voltage (V), luminexcence efficiency (cd/A), maximum emission wavelength (nm), and device lifespan (hr) of the light-emitting devices of Examples 1 to 8 and Comparative Examples 1 to 6 were measured by using V7000 OLED IVL Test System (Polaronix) at a luminance of 1,000 cd/m², and the results thereof are shown in Table 1. In Table 1, the device lifespan (T95) is a measure result of the time (hr) taken until the luminance declines to 95% of the initial luminance.

TABLE 1

| | Hole | Exciplex | | | | | Maximum | |
|---|---|---|---|---|---|---|---|---|
| | transport | Host | Phosphorescent | Boron | Driving | Luminescence | Emission | Device |
| | layer | (HT:ET = | Dopant | Dopant | voltage | efficiency | wavelength | Lifespan |
| | material | 5:5) | (Sensitizer) | (t-DABNA1) | (V) | (cd/A) | (nm) | (T₉₅, hr) |
| Example1 | Compound 1 | HTH/ETH | PD40 | t-DABNA | 4.8 | 20.2 | 463 | 198 |
| Example 2 | Compound 6 | HTH/ETH | PD40 | t-DABNA | 4.5 | 20.1 | 463 | 208 |
| Example 3 | Compound 7 | HTH/ETH | PD40 | t-DABNA | 4.6 | 20.1 | 463 | 192 |
| Example 4 | Compound 23 | HTH/ETH | PD40 | t-DABNA | 4.5 | 19.9 | 463 | 185 |
| Example 5 | Compound 67 | HTH/ETH | PD40 | t-DABNA | 4.5 | 20.2 | 463 | 209 |
| Example 6 | Compound 85 | HTH/ETH | PD40 | t-DABNA | 4.4 | 21.3 | 463 | 191 |
| Example 7 | Compound 97 | HTH/ETH | PD40 | t-DABNA | 4.4 | 21.5 | 463 | 240 |
| Example 8 | Compound 111 | HTH/ETH | PD40 | t-DABNA | 4.6 | 21.1 | 463 | 260 |
| Comparative | HT3 | HTH/ETH | PD40 | t-DABNA | 5.6 | 18.8 | 463 | 95 |
| Example 1 | | | | | | | | |
| Comparative | Compound C1 | HTH/ETH | PD40 | t-DABNA | 5.3 | 18.6 | 463 | 155 |
| Example 2 | | | | | | | | |
| Comparative | Compound C2 | HTH/ETH | PD40 | t-DABNA | 5.2 | 18.9 | 463 | 165 |
| Example 3 | | | | | | | | |
| Comparative | Compound C3 | HTH/ETH | PD40 | t-DABNA | 5.5 | 18.0 | 463 | 120 |
| Example 4 | | | | | | | | |
| Comparative | Compound C4 | HTH/ETH | PD40 | t-DABNA | 6.6 | 15.5 | 463 | 15 |
| Example 5 | | | | | | | | |
| Comparative | Compound C5 | HTH/ETH | PD40 | t-DABNA | 5.2 | 18.5 | 463 | 105 |
| Example 6 | | | | | | | | |

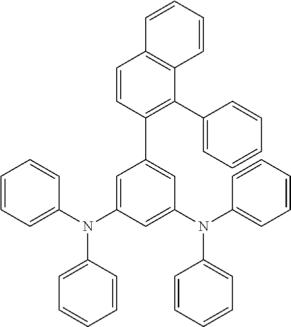 [SMILES: c1ccc(-c2c(-c3cc(N(c4ccccc4)c4ccccc4)cc(N(c4ccccc4)c4ccccc4)c3)ccc3ccccc23)cc1]

1 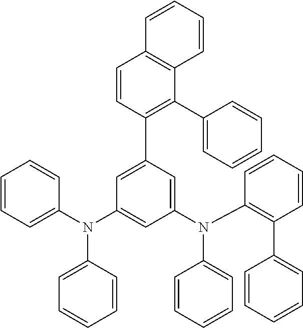 [SMILES: c1ccc(-c2ccccc2N(c2ccccc2)c2cc(-c3ccc4ccccc4c3-c3ccccc3)cc(N(c3ccccc3)c3ccccc3)c2)cc1]
6 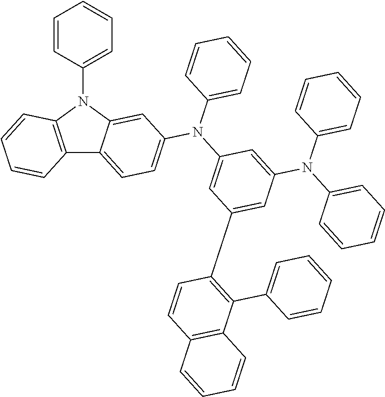 [SMILES: c1ccc(-c2c(-c3cc(N(c4ccccc4)c4ccccc4)cc(N(c4ccccc4)c4ccc5c6ccccc6n(-c6ccccc6)c5c4)c3)ccc3ccccc23)cc1]
7 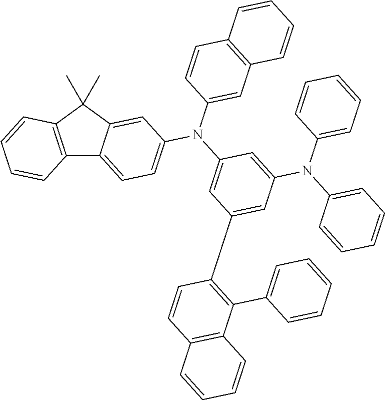 [SMILES: CC1(C)c2ccccc2-c2ccc(N(c3cc(Cc4ccc5ccccc5c4-c4ccccc4)cc(N(c4ccccc4)c4ccccc4)c3)c3ccc4ccccc4c3)cc21]
23 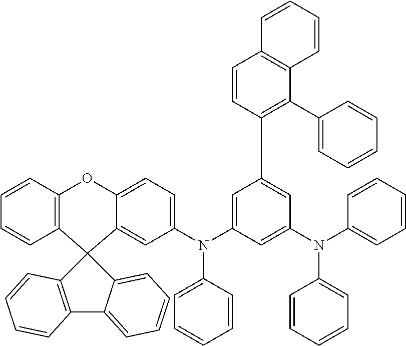 [SMILES: c1ccc(-c2c(-c3cc(N(c4ccccc4)c4ccccc4)cc(N(c4ccccc4)c4ccc5c(c4)C4(c6ccccc6O5)c5ccccc5-c5ccccc54)c3)ccc3ccccc23)cc1]
67 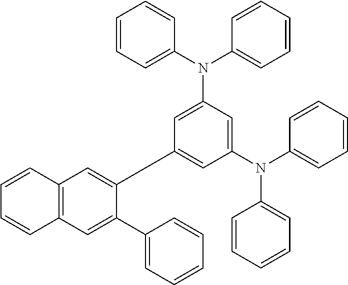 [SMILES: c1ccc(-c2cc3ccccc3cc2-c2cc(N(c3ccccc3)c3ccccc3)cc(N(c3ccccc3)c3ccccc3)c2)cc1]
85 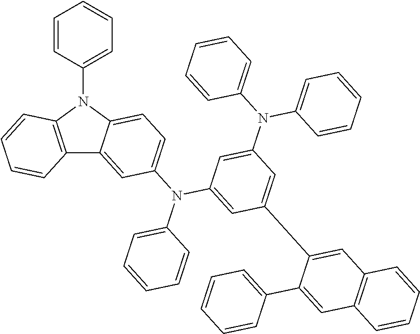 [SMILES: c1ccc(-c2cc3ccccc3cc2Cc2cc(N(c3ccccc3)c3ccccc3)cc(N(c3ccccc3)c3ccc4c(c3)c3ccccc3n4-c3ccccc3)c2)cc1]
97 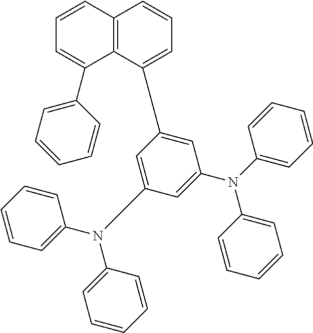 [SMILES: c1ccc(-c2cccc3cccc(Cc4cc(CN(c5ccccc5)c5ccccc5)cc(N(c5ccccc5)c5ccccc5)c4)c23)cc1]
111 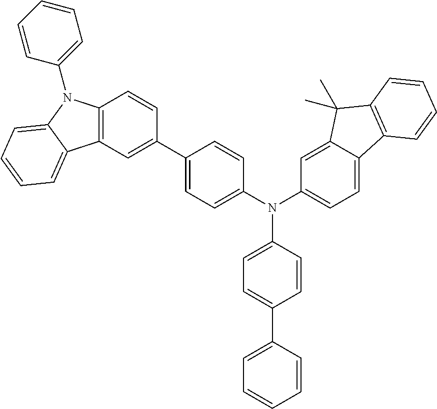 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]
HT3 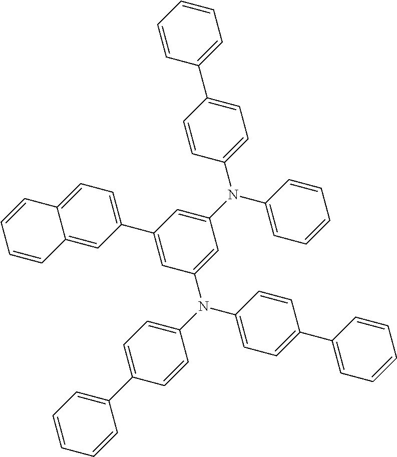 [SMILES: c1ccc(-c2ccc(N(c3ccccc3)c3cc(-c4ccc5ccccc5c4)cc(N(c4ccc(-c5ccccc5)cc4)c4ccc(-c5ccccc5)cc4)c3)cc2)cc1]
C1 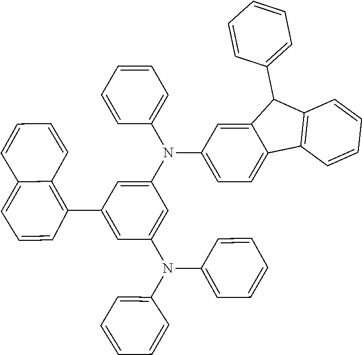 [SMILES: c1ccc(C2c3ccccc3-c3ccc(N(c4ccccc4)c4cc(-c5cccc6ccccc56)cc(N(c5ccccc5)c5ccccc5)c4)cc32)cc1]
C2 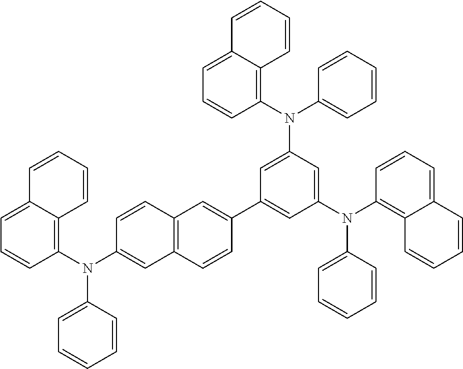 [SMILES: c1ccc(N(c2cc(-c3ccc4cc(N(c5ccccc5)c5cccc6ccccc56)ccc4c3)cc(N(c3ccccc3)c3cccc4ccccc34)c2)c2cccc3ccccc23)cc1]
C4 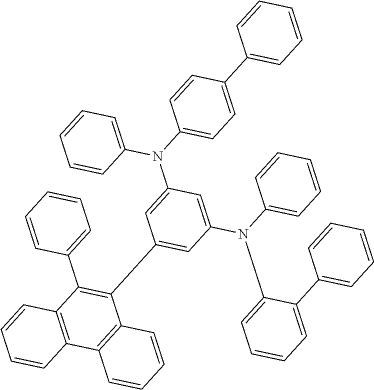 [SMILES: c1ccc(-c2ccc(N(c3ccccc3)c3cc(Cc4c(-c5ccccc5)c5ccccc5c5ccccc45)cc(N(c4ccccc4)c4ccccc4-c4ccccc4)c3)cc2)cc1]
C5

From Table 1, it was confirmed that the light-emitting devices according to Examples 1 to 8 had excellent driving voltage, luminescence efficiency, and device lifespan characteristics compared to those of the light-emitting devices according to Comparative Examples 1 to 6.

By using the amine-containing compound having excellent charge transport ability, a light-emitting device having characteristics of low driving voltage, high luminescence efficiency, and long device lifespan may be manufactured. In addition, high-quality electronic apparatus and electronic equipment may be manufactured by using the light-emitting device.

It should be understood that example embodiments described herein should be considered in a descriptive sense only and not for purposes of limitation. Descriptions of features or aspects within each example embodiment should typically be considered as available for other similar features or aspects in other example embodiments. While one or more example embodiments have been described with reference to the figures, it will be understood by those of ordinary skill in the art that various changes in form and details may be made therein without departing from the spirit and scope as defined by the following claims.

What is claimed is:

1. A light-emitting device comprising:
a first electrode;
a second electrode facing the first electrode;
an interlayer between the first electrode and the second electrode, the interlayer including an emission layer; and
an amine-containing compound represented by Formula 1: 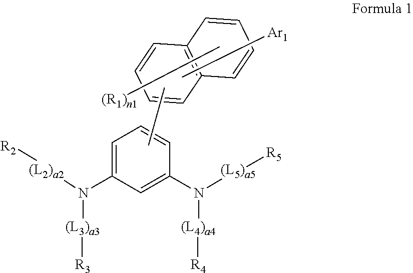
wherein, in Formula 1,
Ar₁ is a C₆-C₆₀ aryl group unsubstituted or substituted with at least one R₁₀ₐ,
L₂ to L₅ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a2 to a5 are each independently 0, 1, 2, or 3,
when a2 is 0, *-(L₂)ₐ₂-*′ is a single bond, when a3 is 0, *-(L₃)ₐ₃-*′ is a single bond, when a4 is 0, *-(L₄)ₐ₄-*′ is a single bond, and when a5 is 0, *-(L₅)ₐ₅-*′ is a single bond,
n1 is 0, 1, 2, 3, 4, 5, or 6,
R₁ to R₅ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆a aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)2(Q₁), —P(═O)(Q₁)(Q₂), or —P(═S)(Q₁)(Q₂),
neighboring two or more of R₂ to R₅ are optionally bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆a alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)2(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-Ceo arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)2(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)2(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof, and
* and *′ each indicate a binding site to a neighboring atom.

2. The light-emitting device of claim 1, wherein
the first electrode is an anode,
the second electrode is a cathode,
the interlayer further comprises a hole transport region arranged between the first electrode and the emission layer and an electron transport region arranged between the emission layer and the second electrode,
the hole transport region comprises a hole injection layer, a hole transport layer, an emission auxiliary layer, an electron-blocking layer, or any combination thereof, and
the electron transport region comprises a buffer layer, a hole-blocking layer, an electron control layer, an electron transport layer, an electron injection layer, or a combination thereof.

3. The light-emitting device of claim 2, wherein the hole transport region comprises the amine-containing compound represented by Formula 1.

4. The light-emitting device of claim 2, wherein
the hole transport region comprises the hole transport layer, and
the hole transport layer comprises the amine-containing compound represented by Formula 1.

5. The light-emitting device of claim 1, wherein the emission layer emits blue light.

6. The light-emitting device of claim 1, further comprising:
at least one of a first capping layer outside the first electrode and a second capping layer outside the second electrode,
wherein at least one of the first capping layer and the second capping layer comprises the amine-containing compound represented by Formula 1.

7. A light-emitting device comprising:
a first electrode;
a second electrode facing the first electrode;
m emitting units stacked between the first electrode and the second electrode; and
m−1 charge generation layers each arranged between two neighboring emitting units from among the m emitting units,
wherein m is an integer of 2 or more,
the m emitting units each independently comprise an interlayer comprising an emission layer, and
in at least one of the m emitting units, the interlayer comprises an amine-containing compound represented by Formula 1: 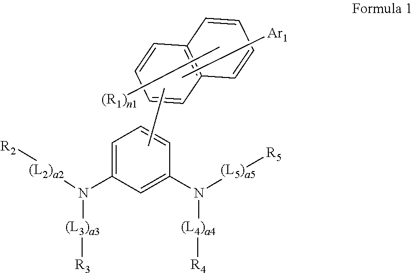
wherein, in Formula 1,
Ar is a C₆-C₆₀ aryl group unsubstituted or substituted with at least one R₁₀ₐ,
L₂ to L₅ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a2 to a5 are each independently 0, 1, 2, or 3,
when a2 is 0, *-(L₂)ₐ₂-*′ is a single bond, when a3 is 0, *-(L₃)ₐ₃-*′ is a single bond, when a4 is 0, *-(L₄)ₐ₄-*′ is a single bond, and when a5 is 0, *-(L₅)ₐ₅-*′ is a single bond,
n1 is 0, 1, 2, 3, 4, 5, or 6,
R₁ to R₅ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)2(Q₁), —P(═O)(Q₁)(Q₂), or —P(═S)(Q₁)(Q₂),
neighboring two or more of R₂ to R₅ are optionally bonded to each other to form a C₅-C₆₀ carbocyclic group unsubstituted or substituted with at least one R₁₀ₐ or a C₁-C₆₀ heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-Ceo arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)2(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)2(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof, * and *′ each indicate a binding site to a neighboring atom.

8. An electronic apparatus comprising the light-emitting device of claim 1.

9. The electronic apparatus of claim 8, further comprising:
a color filter, a color conversion layer, a touch screen layer, a polarizing layer, or any combination thereof.

10. Electronic equipment comprising the light-emitting device of claim 1.

11. The electronic equipment of claim 10, wherein the electronic equipment is one of a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, and a signboard.

12. An amine-containing compound represented by Formula 1: 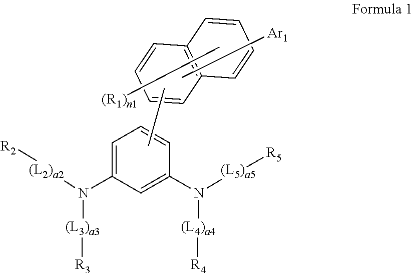
wherein, in Formula 1,
Ar₁ is a C₆-C₆₀ aryl group unsubstituted or substituted with at least one R₁₀ₐ,
L₂ to L₅ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a2 to a5 are each independently 0, 1, 2, or 3,
when a2 is 0, *-(L₂)ₐ₂-*′ is a single bond, when a3 is 0, *-(L₃)ₐ₃-*′ is a single bond, when a4 is 0, *-(L₄)ₐ₄-*′ is a single bond, and when a5 is 0, *-(L₅)ₐ₅-*′ is a single bond,
n1 is 0, 1, 2, 3, 4, 5, or 6,
R₁ to R₅ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆a aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)2(Q₁), —P(═O)(Q₁)(Q₂), or —P(═S)(Q₁)(Q₂),
neighboring two or more of R₂ to R₅ are optionally bonded to each other to form a C₅-C₆₀ carbocyclic group unsubstituted or substituted with at least one R₁₀ₐ or a C₁-C₆a heterocyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆a alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)2(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₁-C₆₀ heteroaryloxy group, a C₁-C₆₀ heteroarylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)2(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)2(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, a pyridinyl group, a pyrimidinyl group, a pyridazinyl group, a pyrazinyl group, a triazinyl group, or any combination thereof, and
* and *′ each indicate a binding site to a neighboring atom.

13. The amine-containing compound of claim 12, wherein a group represented by 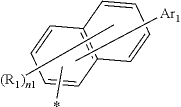
in Formula 1 is a group represented by one of Formulae 1A to 1N: 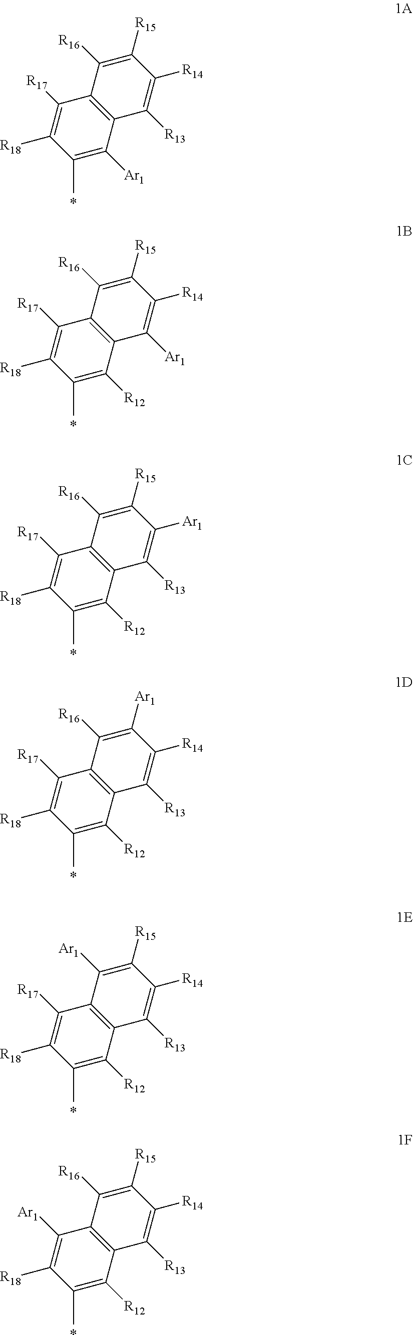 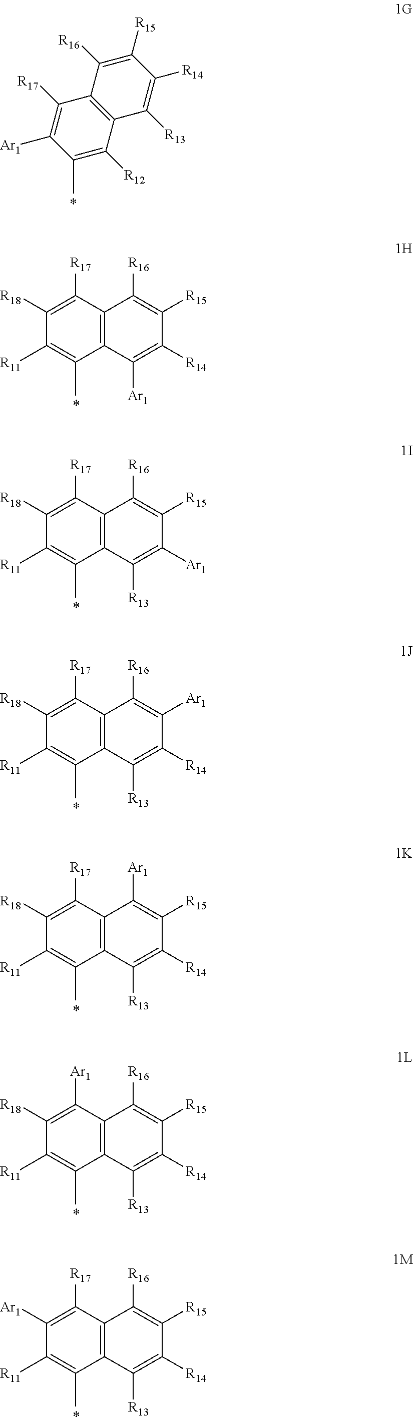 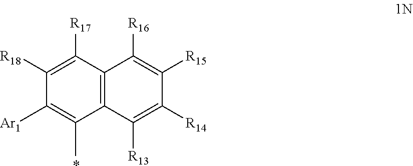
wherein, in Formulae 1A to 1N,
Ar₁ is as described in claim 12,
R₁₁ to R₁₈ are each as described in connection with R₁ in claim 12, and
* indicates a binding site to a neighboring atom.

14. The amine-containing compound of claim 12, wherein Ar₁ is a phenyl group unsubstituted or substituted with at least one R₁₀ₐ.

15. The amine-containing compound of claim 12, wherein R₁ is:
hydrogen, deuterium, —F, a C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a C₁-C₁₀ alkyl group, a cyclopentyl group, a cyclohexyl group, a cycloheptyl group, a cyclopentenyl group, a cyclohexenyl group, a cycloheptenyl group, or a combination thereof;
a cyclopentyl group, a cyclohexyl group, a cycloheptyl group, a cyclopentenyl group, a cyclohexenyl group, or a cycloheptenyl group, each unsubstituted or substituted with deuterium, —F, —CD₃, —CD₂H, —CDH₂, —CF₃, —CF₂H, —CFH₂, a C₁-C₂₀ alkyl group, a C₁-C₂₀ alkoxy group, a cyclopentyl group, a cyclohexyl group, a cycloheptyl group, a cyclopentenyl group, a cyclohexenyl group, a cycloheptenyl group, —Si(Q₃₁)(Q₃₂)(Q₃₃), —N(Q₃₁)(Q₃₂), or any combination thereof; or
—Si(Q₁)(Q₂)(Q₃) or —N(Q₁)(Q₂).

16. The amine-containing compound of claim 12, wherein R₂ to R₅ are each independently
a C₆-C₆₀ aryl group unsubstituted or substituted with at least one R₁₀ₐ,
a C₁-C₆₀ heteroaryl group unsubstituted or substituted with at least one R₁₀ₐ,
a monovalent non-aromatic condensed polycyclic group unsubstituted or substituted with at least one R₁₀ₐ, or
a monovalent non-aromatic condensed heteropolycyclic group unsubstituted or substituted with at least one R₁₀ₐ.

17. The amine-containing compound of claim 12, wherein R₂ to R₅ are each independently a group represented by one of Formulae 2-1 to 2-13: 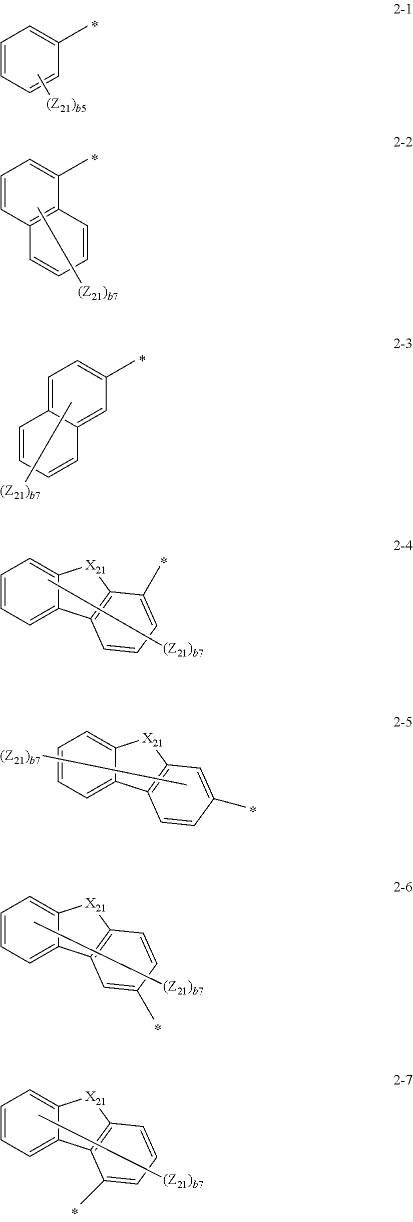 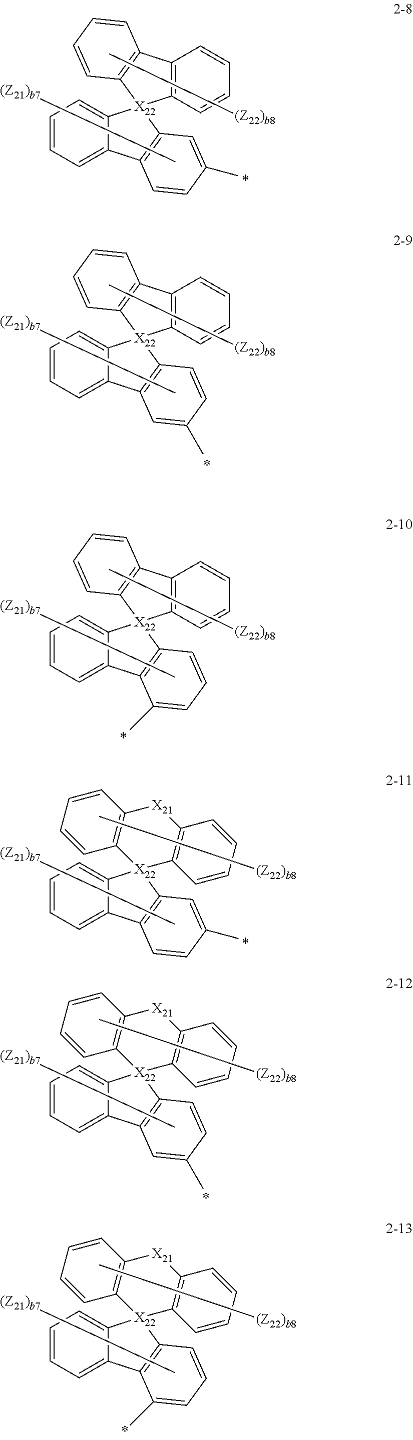
wherein, in Formulae 2-1 to 2-13,
X₂₁ is C(Z₂₃)(Z₂₄), Si(Z₂₃)(Z₂₄), N(Z₂₃), P(Z₂₃), O, or S,
X₂₂ is C or Si,
Z₂₁, Z₂₂, Z₂₃, and Z₂₄ are each as described in connection with R₁₀ₐ in claim 12,
b5 is 0, 1, 2, 3, 4, or 5,
b7 is 0, 1, 2, 3, 4, 5, 6, or 7,
b8 is 0, 1, 2, 3, 4, 5, 6, 7, or 8, and
* indicates a binding site to a neighboring atom.

18. The amine-containing compound of claim 12, wherein L₂ to L₅ are each independently a group represented by one of Formulae 3-1 to 3-3: 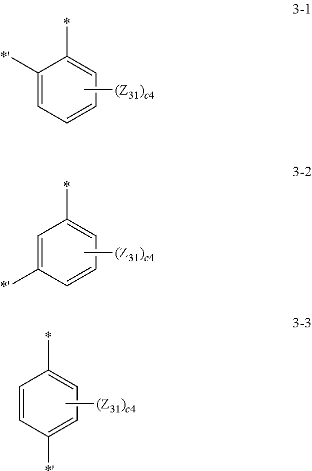
wherein, in Formulae 3-1 to 3-3,
Z₃₁ is as described in connection with R₁₀ₐ in claim 12,
c4 is 0, 1, 2, 3, or 4, and
* and *′ each indicate a binding site to a neighboring atom.

19. The amine-containing compound of claim 12, wherein the amine-containing compound is represented by Formula 1-1: 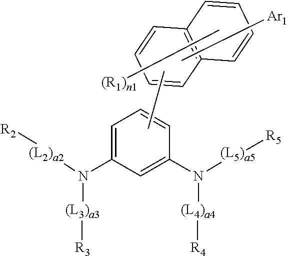
wherein Ar₁, L₂ to L₅, a2 to a5, n1, and R₁ to R₅ are each as described in claim 12.

20. The amine-containing compound of claim 12, wherein the amine-containing compound is selected from Compounds 1 to 120: 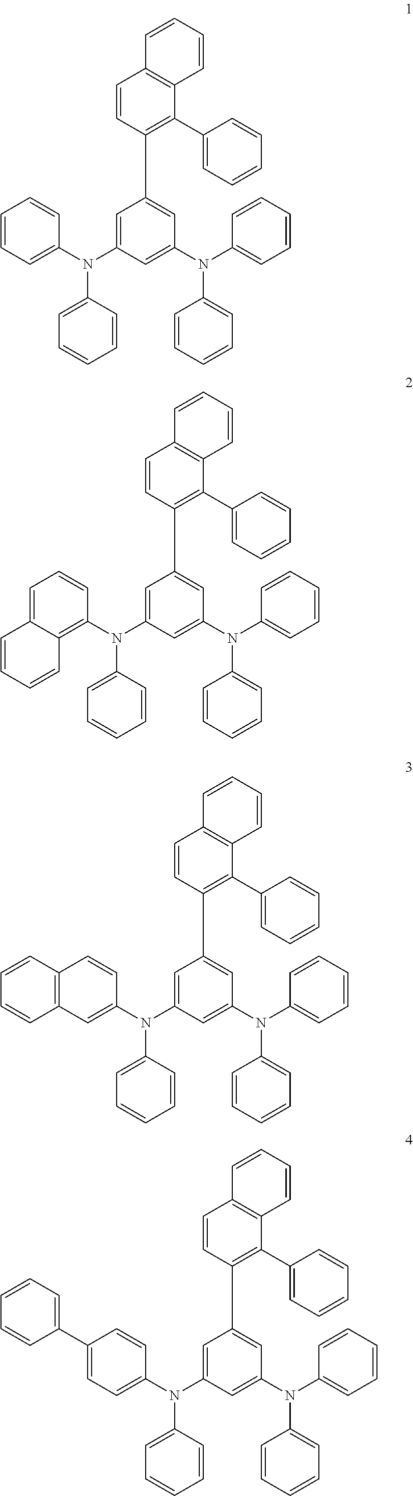 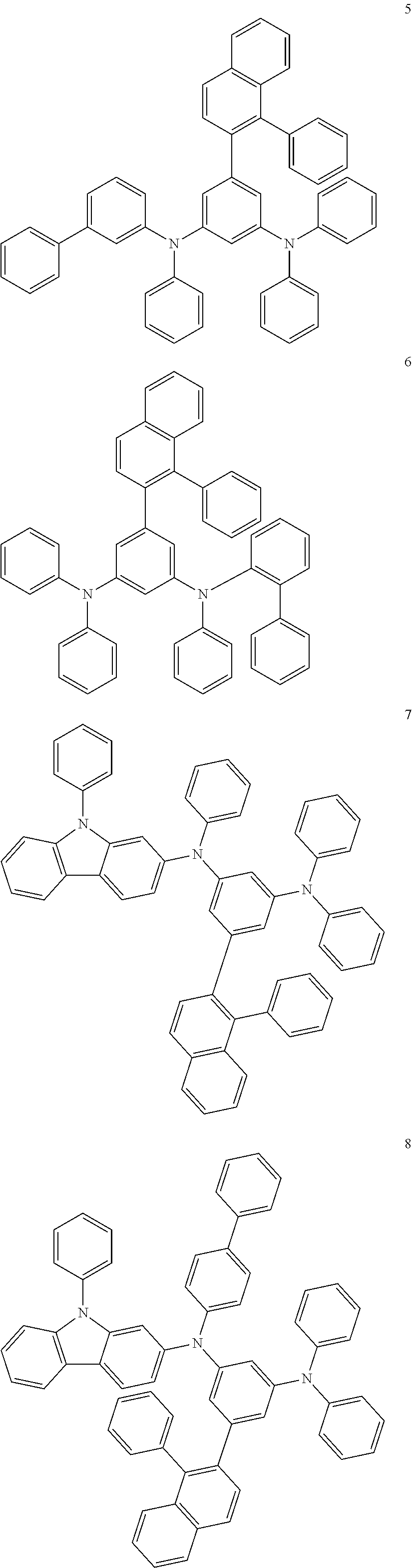 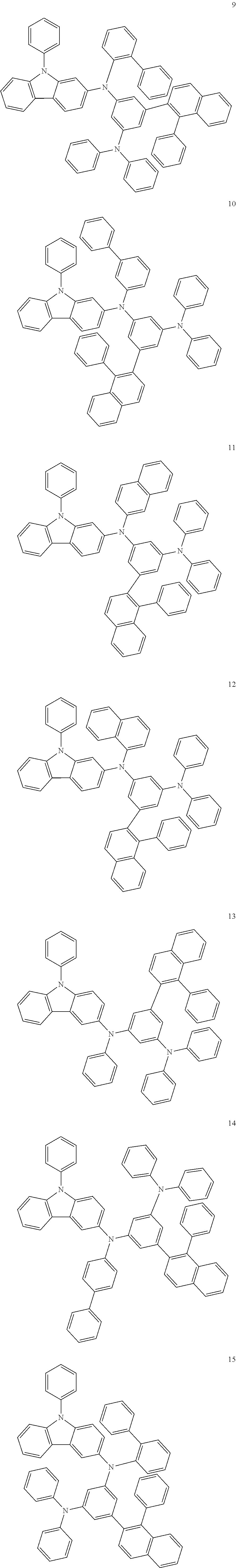 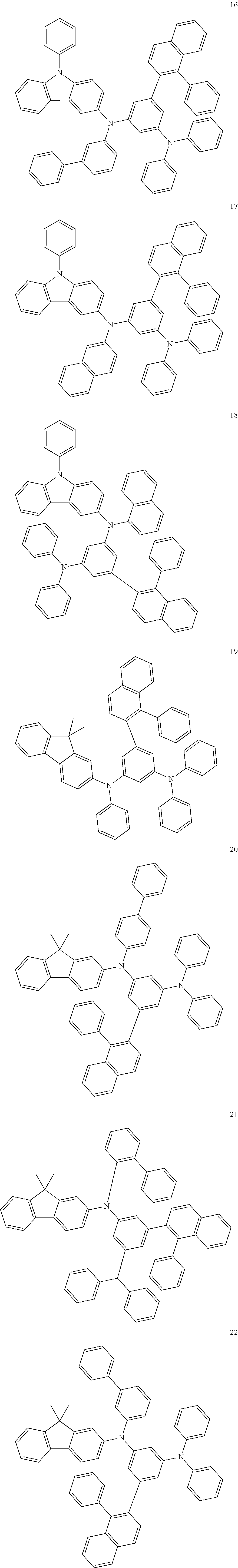 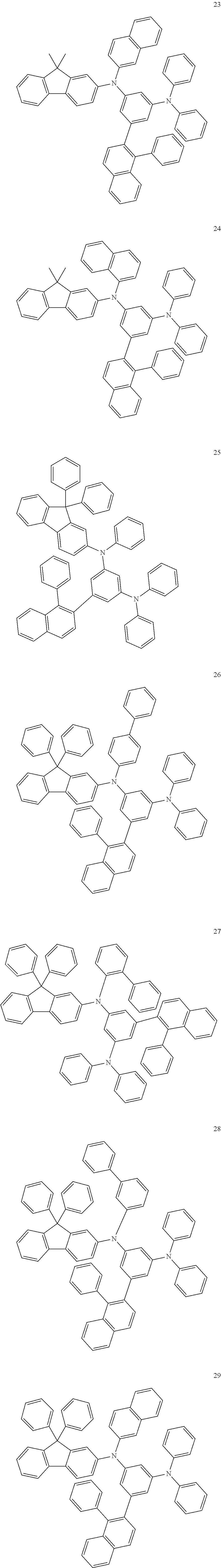 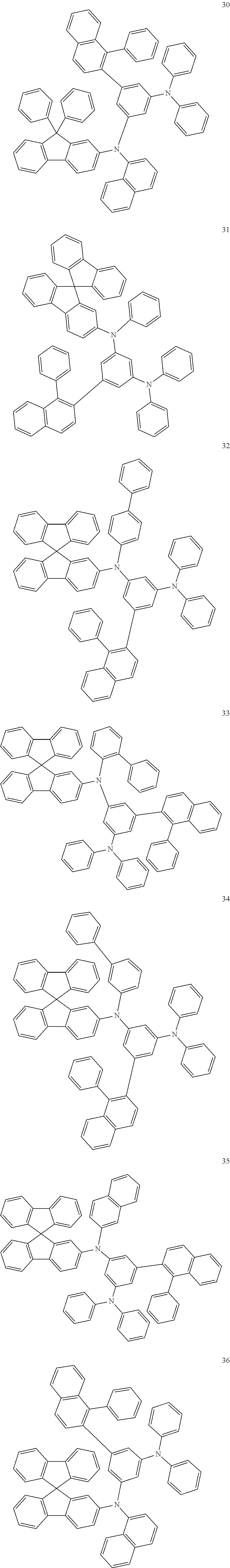 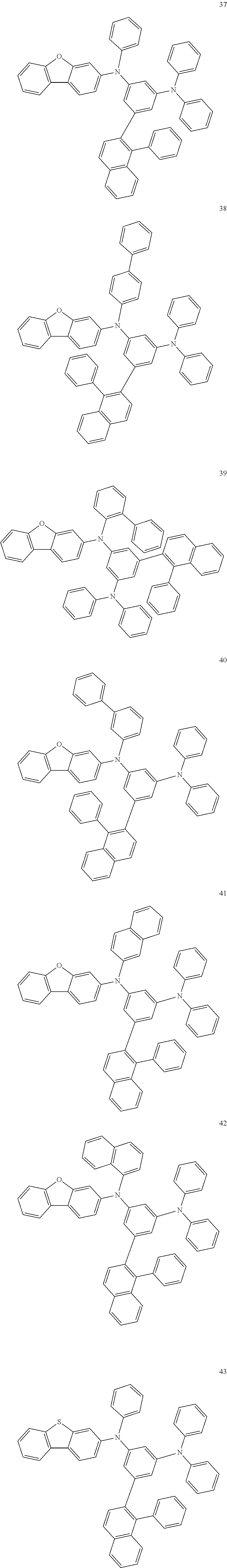 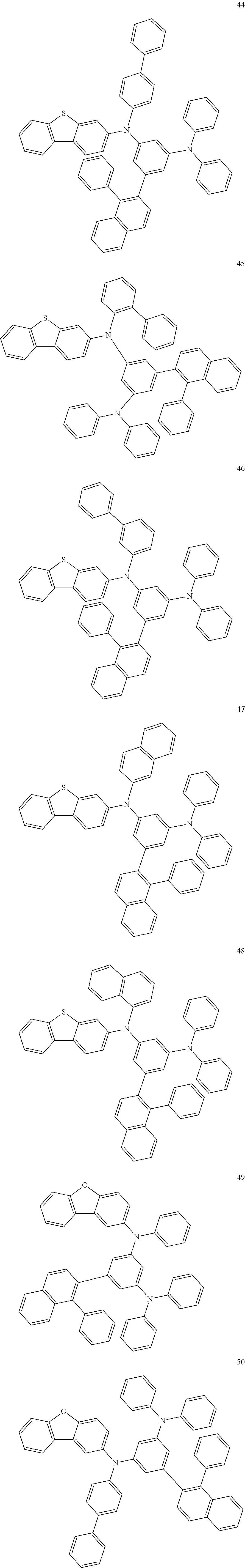 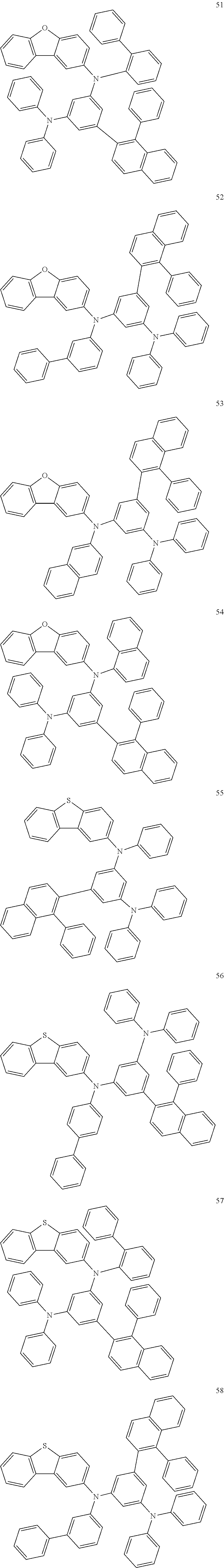 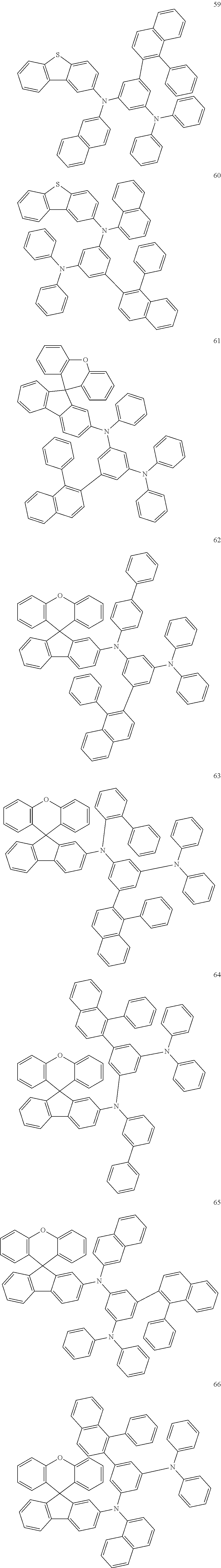 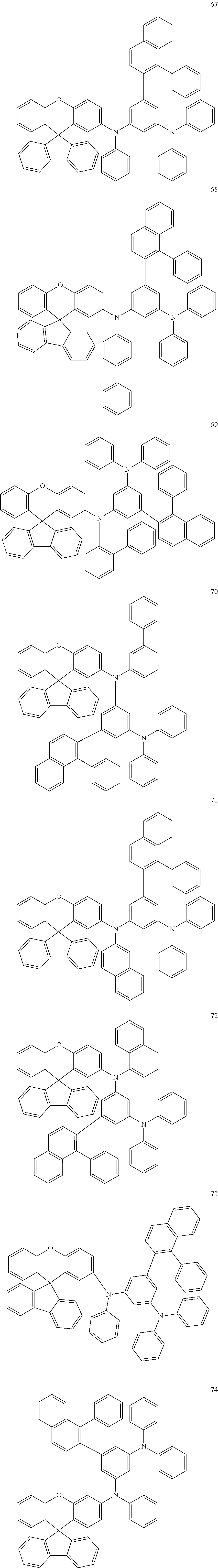 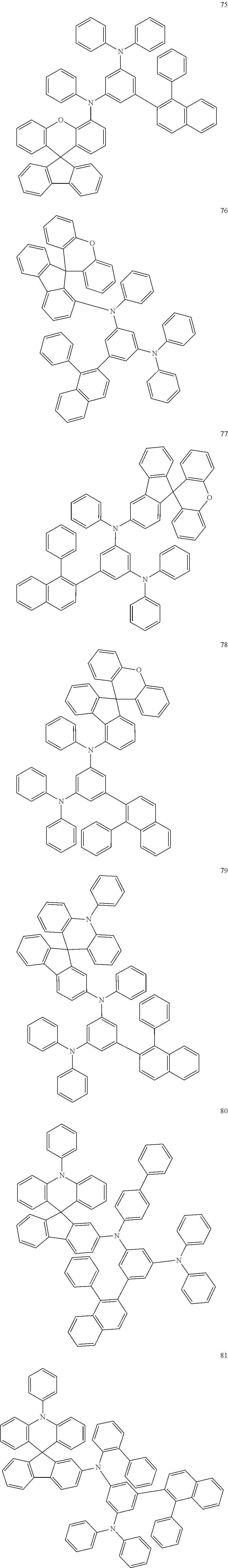 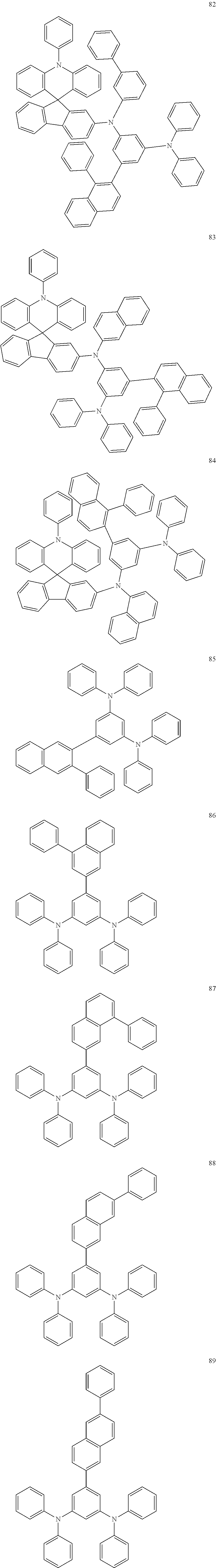 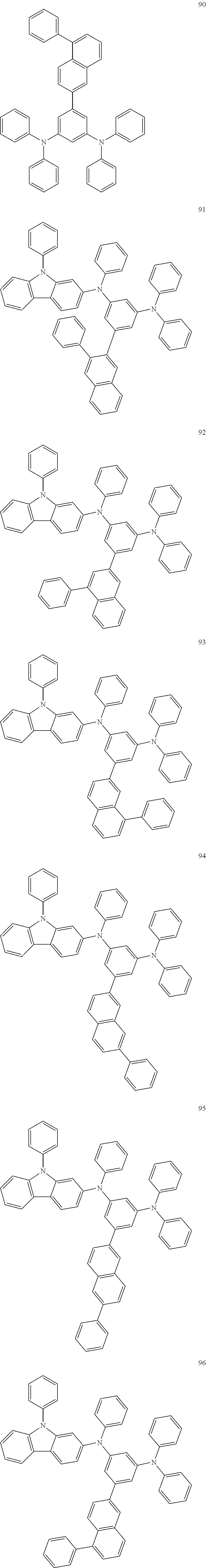 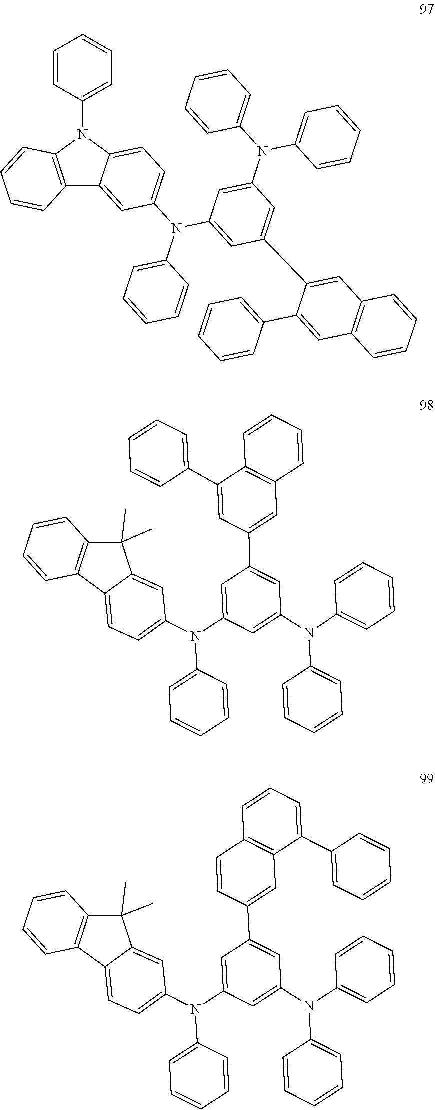 [SMILES: c1ccc(-c2cc3ccccc3cc2-c2cc(N(c3ccccc3)c3ccccc3)cc(N(c3ccccc3)c3ccc4c(c3)c3ccccc3n4-c3ccccc3)c2)cc1 ; CC1(C)c2ccccc2-c2ccc(N(c3ccccc3)c3cc(-c4cc(-c5ccccc5)c5ccccc5c4)cc(N(c4ccccc4)c4ccccc4)c3)cc21 ; CC1(C)c2ccccc2-c2ccc(N(c3ccccc3)c3cc(-c4ccc5cccc(-c6ccccc6)c5c4)cc(N(c4ccccc4)c4ccccc4)c3)cc21] 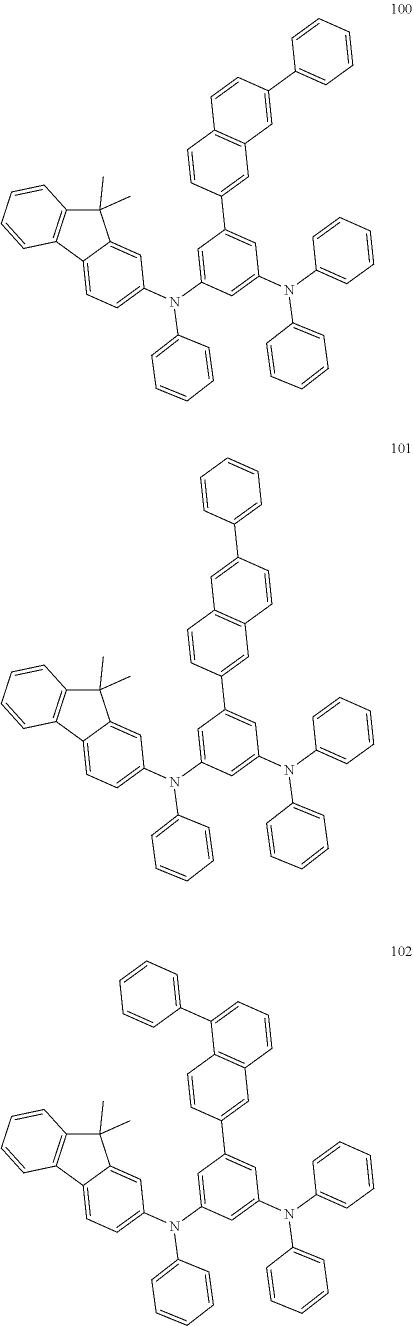 [SMILES: CC1(C)c2ccccc2-c2ccc(N(c3ccccc3)c3cc(-c4ccc5ccc(-c6ccccc6)cc5c4)cc(N(c4ccccc4)c4ccccc4)c3)cc21 ; CC1(C)c2ccccc2-c2ccc(N(c3ccccc3)c3cc(-c4ccc5cc(-c6ccccc6)ccc5c4)cc(N(c4ccccc4)c4ccccc4)c3)cc21 ; CC1(C)c2ccccc2-c2ccc(N(c3ccccc3)c3cc(-c4ccc5c(-c6ccccc6)cccc5c4)cc(N(c4ccccc4)c4ccccc4)c3)cc21] 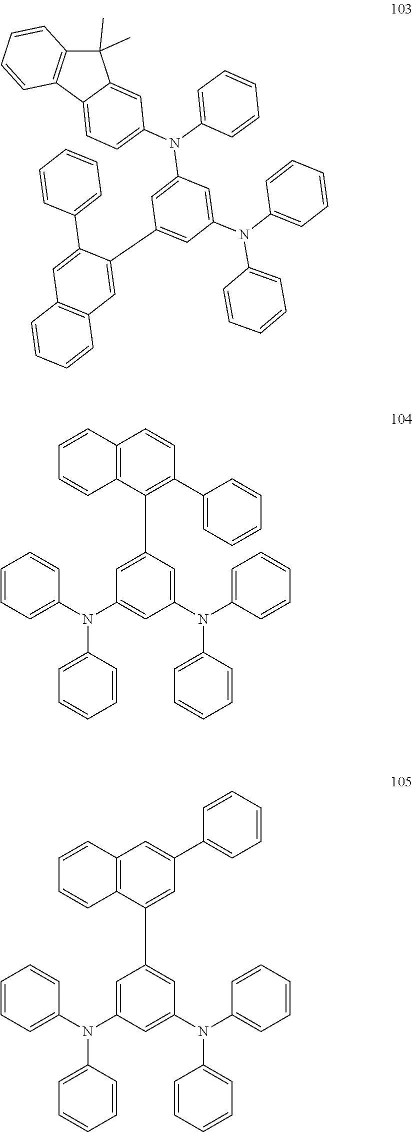 [SMILES: CC1(C)c2ccccc2-c2ccc(N(c3ccccc3)c3cc(-c4cc5ccccc5cc4-c4ccccc4)cc(N(c4ccccc4)c4ccccc4)c3)cc21 ; c1ccc(-c2ccc3ccccc3c2-c2cc(N(c3ccccc3)c3ccccc3)cc(N(c3ccccc3)c3ccccc3)c2)cc1 ; c1ccc(-c2cc(-c3cc(N(c4ccccc4)c4ccccc4)cc(N(c4ccccc4)c4ccccc4)c3)c3ccccc3c2)cc1] 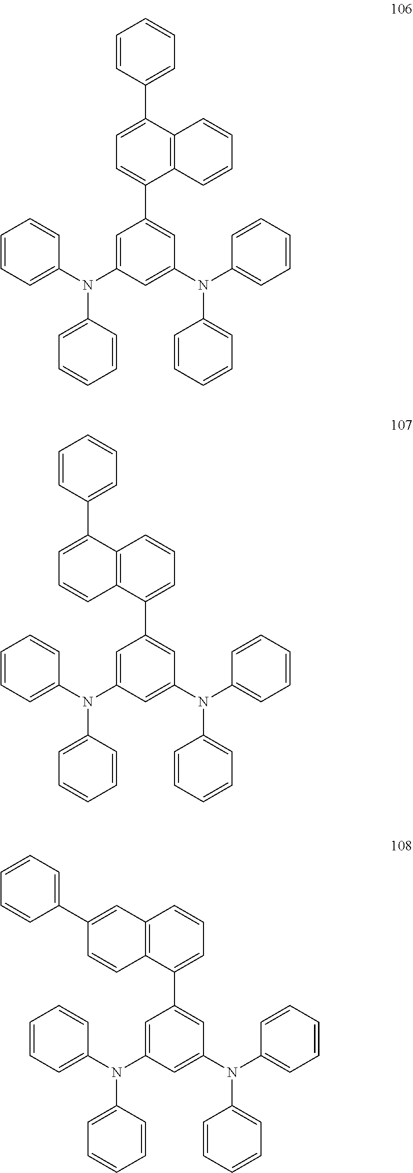 [SMILES: c1ccc(-c2ccc(-c3cc(N(c4ccccc4)c4ccccc4)cc(N(c4ccccc4)c4ccccc4)c3)c3ccccc23)cc1 ; c1ccc(-c2cccc3c(-c4cc(N(c5ccccc5)c5ccccc5)cc(N(c5ccccc5)c5ccccc5)c4)cccc23)cc1 ; c1ccc(-c2ccc3c(-c4cc(N(c5ccccc5)c5ccccc5)cc(N(c5ccccc5)c5ccccc5)c4)cccc3c2)cc1] 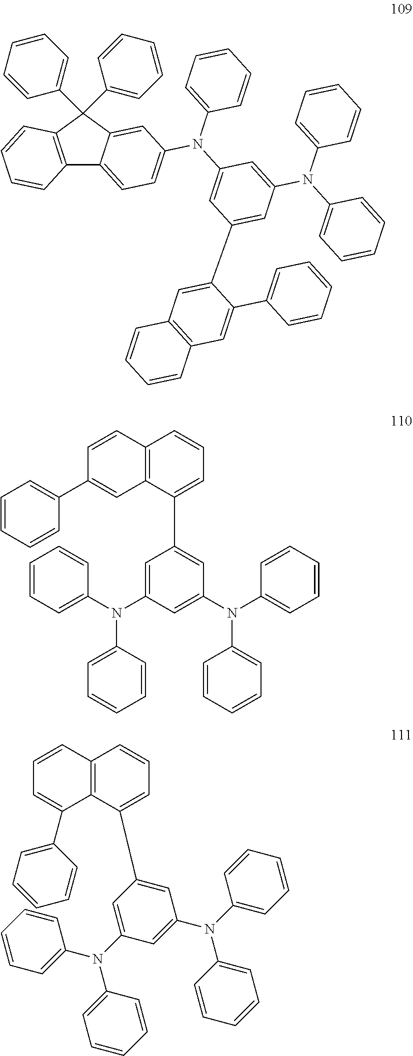 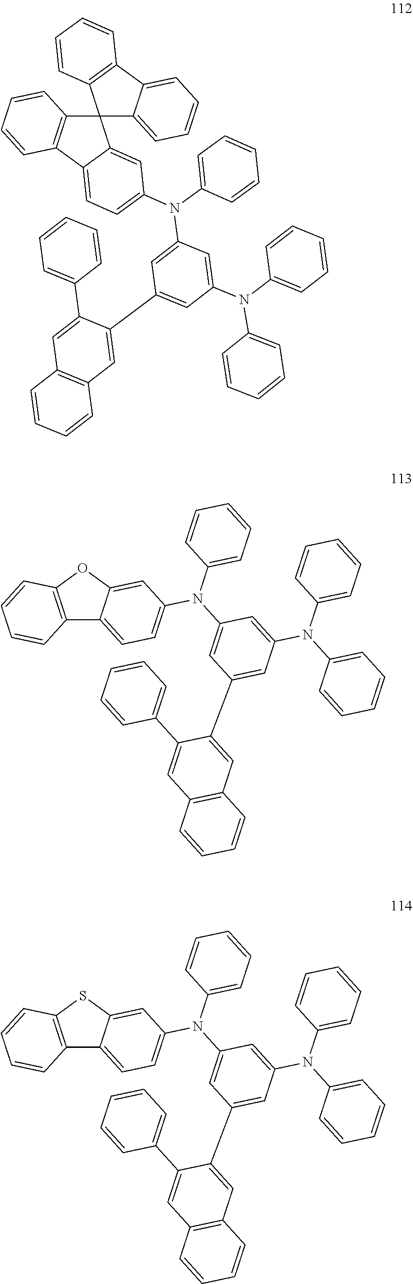 [SMILES: c1ccc(-c2cc3ccccc3cc2-c2cc(N(c3ccccc3)c3ccccc3)cc(N(c3ccccc3)c3ccc4c(c3)C3(c5ccccc5-c5ccccc53)c3ccccc3-4)c2)cc1 ; c1ccc(-c2cc3ccccc3cc2-c2cc(N(c3ccccc3)c3ccccc3)cc(N(c3ccccc3)c3ccc4c(c3)oc3ccccc34)c2)cc1 ; c1ccc(-c2cc3ccccc3cc2-c2cc(N(c3ccccc3)c3ccccc3)cc(N(c3ccccc3)c3ccc4c(c3)sc3ccccc34)c2)cc1] 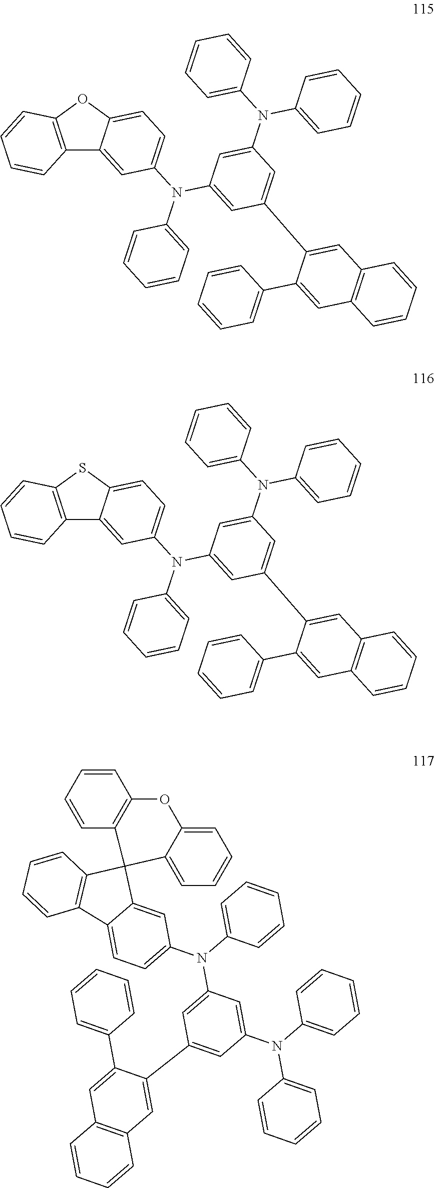 [SMILES: c1ccc(-c2cc3ccccc3cc2-c2cc(N(c3ccccc3)c3ccccc3)cc(N(c3ccccc3)c3ccc4oc5ccccc5c4c3)c2)cc1 ; c1ccc(-c2cc3ccccc3cc2-c2cc(N(c3ccccc3)c3ccccc3)cc(N(c3ccccc3)c3ccc4sc5ccccc5c4c3)c2)cc1 ; c1ccc(-c2cc3ccccc3cc2-c2cc(N(c3ccccc3)c3ccccc3)cc(N(c3ccccc3)c3ccc4c(c3)C3(c5ccccc5Oc5ccccc53)c3ccccc3-4)c2)cc1] 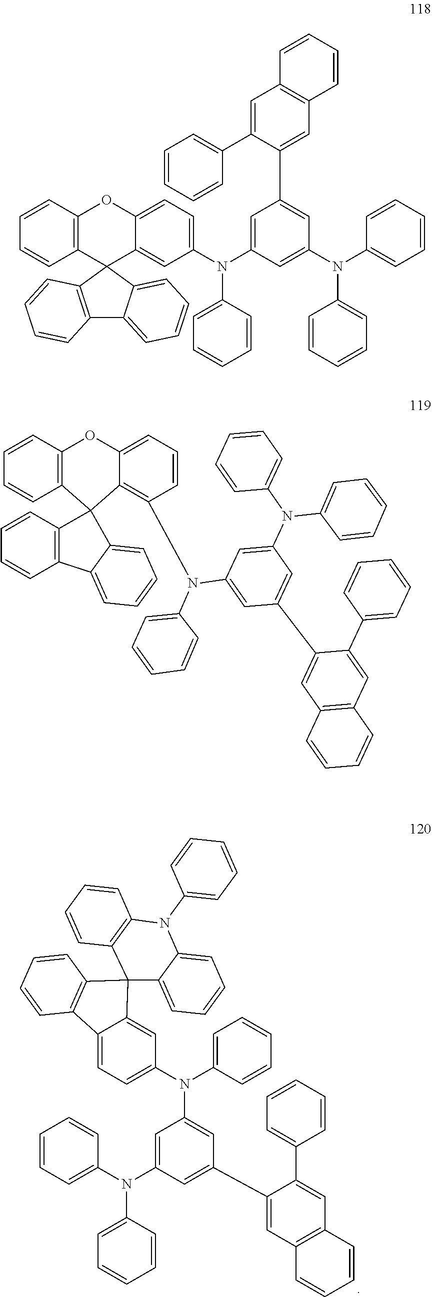 [SMILES: c1ccc(-c2cc3ccccc3cc2-c2cc(N(c3ccccc3)c3ccccc3)cc(N(c3ccccc3)c3ccc4c(c3)C3(c5ccccc5O4)c4ccccc4-c4ccccc43)c2)cc1 ; c1ccc(-c2cc3ccccc3cc2-c2cc(N(c3ccccc3)c3ccccc3)cc(N(c3ccccc3)c3cccc4c3C3(c5ccccc5O4)c4ccccc4-c4ccccc43)c2)cc1 ; c1ccc(-c2cc3ccccc3cc2-c2cc(N(c3ccccc3)c3ccccc3)cc(N(c3ccccc3)c3ccc4c(c3)C3(c5ccccc5-4)c4ccccc4N(c4ccccc4)c4ccccc43)c2)cc1]
